(19) United States
(12) Patent Application Publication
(10) Pub. No.: US 2015/0284452 A1
(43) Pub. Date: Oct. 8, 2015

(54) ANTIMICROBIAL COMPOSITIONS

(71) Applicant: IOGENETICS, LLC, Madison, WI (US)

(72) Inventors: Robert Duane Bremel, Hillpoint, WI (US); Jane Homan, Hillpoint, WI (US); Michael Imboden, Madison, WI (US); Brendan Keough, Madison, WI (US)

(73) Assignee: IOGENETICS, LLC, Madison, WI (US)

(21) Appl. No.: 14/442,035

(22) PCT Filed: Nov. 13, 2013
(86) PCT No.: PCT/US2013/069828

§ 371 Date: May 11, 2015

Related U.S. Application Data
(60) Provisional application No. 61/725,838, filed on Nov. 13, 2012.
(60) Provisional application No. 61/869,209, filed on Aug. 23, 2013.

(51) Int. Cl. C07K 16/12; C12N 9/52

(52) U.S. Cl. CPC C07K 16/1271; C12N 9/52; C07K 2319/33; C12Y 304/24075

(57) ABSTRACT

The present invention relates antimicrobial compositions, and in particular to antigen binding proteins comprising one or more domains that provide antimicrobial activity.

### FIELD OF THE INVENTION

The present invention relates antimicrobial compositions, and in particular to antigen binding proteins comprising one or more domains that provide antimicrobial activity.

### BACKGROUND OF THE INVENTION

Importance of Staphylococcal and Streptococcal Infection

Staphylococcus aureus is an important pathogen for both human and animal health. Staphylococcus species are ubiquitous in the flora of skin and human contact surfaces and are frequent opportunist pathogens of wounds, secondary complications of viral pneumonias, and a cause of food poisoning. Antibiotic resistant strains of Staphylococcus aureus are widespread and rapidly spreading worldwide (1) both as a community associate infection and as a hospital associated infection. Staphylococci have become the leading cause of nosocomial infections (Kuehnert et al. 2005. Emerg. Infect. Dis. 11:868-872).

S. aureus is the most common infection of surgical wounds, responsible for increased inpatient time, with increased costs mortality rates. Outcome is particularly severe with methicillin resistant S. aureus (MRSA) (Anderson and Kaye. 2009. Infect. Dis. Clin. North Am. 23:53-72) (2). In 2005 MRSA caused almost 100,000 reported cases and 18,650 deaths in the United States, exceeding the number of deaths directly attributed to AIDs (Klevens et al. 2006. Emerg. Infect. Dis. 12:1991-1993; Klevens et al. 2007. JAMA 298:1763-1771). MRSA infections are also commonly associated with catheters, ulcers, ventilators, and prostheses (3, 4). Staphylococcal infection is a common complication of implant associated infections, for instance around bone plates, screws and nails used in fracture repair. S. aureus and S. epidermidis are the most common species here (Harris and Richards, Injury, 37 S3-S14, 2006). Patients affected by cancer and subject to long term hospital stays are particularly at risk (5, 6) as are neonates (7). MRSA infections are increasingly prevalent in HIV patients (Thompson and Torriani. 2006. Curr. HIV./AIDS Rep. 3:107-112). MRSA infections are now disseminated in the community with infections arising as a result of surface contact in schools, gyms and childcare facilities (Kellner et al. 2009. 2007. Morbidity and Mortality Weekly Reports 58:52-55; Klevans, 2006; Miller and Kaplan. 2009. Infect. Dis. Clin. North Am. 23:35-52). One emerging result of this is that MRSA is also associated with severe pneumonia (8). Staphylococcus is recognized as a serious complication of influenza viral pneumonia contributing to increased mortality (Kallen et al. 2009. Ann. Emerg. Med. 53:358-365). The impact of MRSA in tropical and developing countries is under-documented but clearly widespread (Nickerson et al. 2009 Lancet Infect. Dis. 9:130-135). Staphylococcal infections are also frequently found in the eye and are a complication of ocular surgery (9, 10).

In animal health Staphylococcus aureus is also an important pathogen. Mastitis arising from S. aureus infection of the bovine mammary gland is a major source of economic loss to the dairy industry. Antibiotic resistance is increasing among strains of S. aureus isolated from mastitis cases. MRSA strains have been demonstrated, but so far only limited spread to humans has been confirmed from mastitis cases, with wider spread to the consuming public a potential threat. As in human health S. aureus is a frequent secondary opportunist invader in wounds and following a variety of primary infections in cattle. The need for alternative antimicrobial therapies is thus as acute in the livestock industry as it is in the human population.

The Streptococcus spp encompass another important group of gram positive pathogens. The Streptococcus spp are broadly divided into two groups: the alpha hemolytic Streptococcus spp comprising Strep. pneumoniae a major respiratory pathogen as well as causing otitis andmeningitis, and the beta hemolytic Streptococcus spp. which comprise several groups including Group A Strep. pyogenes, frequently associated with pharyngitis “Strep Throat” and upper respiratory and ear infections. The betahemolytic Streptococcus spp comprise a number of other groups including many important pathogens of both humans and animals. Strep. pneumoniae also known as pneumococcus, is the leading cause of childhood deaths due to pneumonia, and is also a common cause of bacterial meningitis. It has been calculated that in 2000, when there was very little vaccine prevention of Strep. pneumoniae infections in children, about 11.5 million episodes of serious pneumococcal disease occurred worldwide in children under five years old and led to over 800,000 deaths (Esposito et al, Expert Opin. Pharmacother. (2013) 14(1):65-77). Strep. pyogenes are responsible for a minimally estimated 616 million cases of throat infection (pharyngitis, tonsillitis) worldwide per year, and 111 million cases of skin infection in children of less developed countries (Bessen, DE, Infect Genet Evol. 2009 July; 9(4): 581-593). Streptococci are also important animal pathogens causing mastitis (Strep agalactiae and Strep uberis) and equine strangles (Strep equi).

### Increasing Antimicrobial Resistance

Antimicrobial resistance is a growing global problem and an emerging public health emergency. Certain species of antibiotic resistant bacteria are contributing disproportionately to increased morbidity, mortality and costs of treatment and surveillance (11-13). Methicillin resistant Staphylococcus aureus (MRSA) is a leading cause of nosocomial infections. Factors contributing to the emergence of antimicrobial resistance include broad spectrum antibiotics which place commensal flora, as well as pathogens, under selective pressure. Current broad spectrum antibiotics target a relatively small number of bacterial metabolic pathways. Most of the few recently approved new antimicrobials depend on these same pathways, exacerbating the rapid development of resistance, and vulnerability to bioterrorist microbial engineering (Spellberg et al., Jr. 2004. Clin. Infect. Dis. 38:1279-1286). New strategies for antimicrobial development are urgently needed which move beyond dependence on the same pathways and which enable elimination of specific pathogens without placing selective pressure on the antimicrobial flora more broadly.

Antibiotic resistance is a growing problem in management of Streptococcal infections. The extent of this varies from country to country. Penicillin resistant strains account for more than 50% of isolates in Asia (Esposito et al, Expert Opin. Pharmacother. (2013) 14(1):65-77). As application of the multi serotype polysaccharide vaccine has lead to emergence a different balance of pneumococcal strains the prevalence of penicillin resistant strains has increased. Resistance is also increasing to the macrolide antibiotics in some cases well over 30% of isolates are resistant (Farrell et al Pediatr Infect Dis J2007; 26:123-8). In order to overcome the problem of multidrug resistant Strep. pneumoniae, new antibiotics have been developed. Most of them are not yet licensed for pediatric use. Vancomycin tolerant strains of Strep pneumoniae have been isolated from meningitis cases (Rodriguez et al, J Infect Dis 2004; 190:1481-7).

Antibiotics resistance is a concern for many other bacteria including, among the gram positives Enterococcus (vancomycin resistant enterococci or VRE) and Bacillus anthracis, As well as Mycobacterium and many gram negative bacteria such as Neisseria gonorheae and N. meningitidis, Klebsiella spp, Acinitobacter spp, Pseudomonas spp. The examples cited herein should thus not be considered limiting.

### SUMMARY OF THE INVENTION

The present invention relates antimicrobial compositions, and in particular to antigen binding proteins comprising one or more domains that provide antimicrobial activity.

In some embodiments, the present invention provides an antigen binding protein comprising a pair of polypeptides corresponding to antibody heavy and light chain variable regions, wherein the heavy and light chain variable regions of the pair of polypeptides have amino acid sequences at least 90%, 95%, 97%, 99% or 100% identical to light and heavy chain variable region pairs selected from the group consisting of: amino acids 21-130 of SEQ ID NO:254 and amino acids 21-146 of SEQ ID NO:256, amino acids 21-136 of SEQ ID NO: 2 and amino acids 21-139 of SEQ ID NO:4, amino acids 21-136 of SEQ ID NO:6 and amino acids 21-138 SEQ ID NO: 8, amino acids 21-131 of SEQ ID NO:10 and amino acids 21-140 of SEQ ID NO:12, amino acids 21-137 of SEQ ID NO:14 and amino acids 21-140 of SEQ ID NO:16, amino acids 21-127 of SEQ ID NO:18 and amino acids 21-144 of SEQ ID NO:20, amino acids 21-131 of SEQ ID NO:22 and amino acids 21-136 of SEQ ID NO:24, amino acids 21-131 of SEQ ID NO:26 and amino acids 21-144 of SEQ ID NO:28, amino acids 21-131 of SEQ ID NO:30 and amino acids 21-144 of SEQ ID NO:32, amino acids 21-131 of SEQ ID NO:238 and amino acids 21-144 of SEQ ID NO:240, amino acids 21-130 of SEQ ID NO:242 and amino acids 21-145 of SEQ ID NO:244, amino acids 21-130 of SEQ ID NO:246 and amino acids 21-137 of SEQ ID NO:248, amino acids 21-137 of SEQ ID NO:250 and amino acids 21-140 of SEQ ID NO:252, amino acids 21-130 of SEQ ID NO:258 and amino acids 21-142 of SEQ ID NO:260, amino acids 21-132 of SEQ ID NO:262 and amino acids 21-142 of SEQ ID NO:264, amino acids 21-136 of SEQ ID NO:266 and amino acids 21-140 of SEQ ID NO:268, amino acids 21-136 of SEQ ID NO:270 and amino acids 21-146 of SEQ ID NO:272, and amino acids 21-131 of SEQ ID NO:274 and amino acids 21-139 of SEQ ID NO:276. In some embodiments, the antigen binding protein is selected from the group consisting of an immunoglobulin, an scFV, a Fab fragment, a diabody, and a triabody. In some embodiments, the antigen binding protein is a fusion with a heterologous polypeptide. In some embodiments, the antigen binding protein binds to a Staphylococcus aureus peptide. In some embodiments, the antigen binding protein binds to a peptide conserved in more than five strains of Staphylococcus aureus and presented on the surface of Staphylococcus aureus. In some embodiments, the antigen binding protein binds to a peptide of Staphylococcus aureus selected from the group consisting of penicillin binding protein peptides, iron sensitive determinant peptides, and peptides from proteins involved in septum formation. In some embodiments, the antigen binding protein binds to a peptide of Staphylococcus aureus selected from the group consisting of SEQ ID NOs:93-123 and 224. In some embodiments, the present invention provides a vector encoding the antigen binding protein described above. In some embodiments, the present invention provides a host cell expressing an antigen binding protein as described above. In yet other embodiments the invention provides a host cell expressing an antigen binding protein targeting another bacterial pathogen of interest.

In some embodiments, the present invention provides an antigen binding protein comprising a pair of polypeptides corresponding to antibody heavy and light chain variable regions, wherein the antigen binding protein binds to an epitope of a protein of Staphylococcus aureus selected from the group consisting of penicillin binding protein, iron sensitive determinants and proteins involved in septum formation. In some embodiments, the epitope is conserved in more than 10 strains of S. aureus. In some embodiments, the heavy and light chain variable regions of the pair of polypeptides have amino acid sequences selected from the group consisting of amino acid sequences at least 90%, 95%, 97%, 98% or 100% identical to light and heavy chain variable region pairs selected from the group consisting of: amino acids 21-130 of SEQ ID NO:254 and amino acids 21-146 of SEQ ID NO:256, amino acids 21-136 of SEQ ID NO: 2 and amino acids 21-139 of SEQ ID NO:4, amino acids 21-136 of SEQ ID NO:6 and amino acids 21-138 SEQ ID NO: 8, amino acids 21-131 of SEQ ID NO:10 and amino acids 21-140 of SEQ ID NO:12, amino acids 21-137 of SEQ ID NO:14 and amino acids 21-140 of SEQ ID NO:16, amino acids 21-127 of SEQ ID NO:18 and amino acids 21-144 of SEQ ID NO:20, amino acids 21-131 of SEQ ID NO:22 and amino acids 21-136 of SEQ ID NO:24, amino acids 21-131 of SEQ ID NO:26 and amino acids 21-144 of SEQ ID NO:28, amino acids 21-131 of SEQ ID NO:30 and amino acids 21-144 of SEQ ID NO:32, amino acids 21-131 of SEQ ID NO:238 and amino acids 21-144 of SEQ ID NO:240, amino acids 21-130 of SEQ ID NO:242 and amino acids 21-145 of SEQ ID NO:244, amino acids 21-130 of SEQ ID NO:246 and amino acids 21-137 of SEQ ID NO:248, amino acids 21-137 of SEQ ID NO:250 and amino acids 21-140 of SEQ ID NO:252, amino acids 21-130 of SEQ ID NO:258 and amino acids 21-142 of SEQ ID NO:260, amino acids 21-132 of SEQ ID NO:262 and amino acids 21-142 of SEQ ID NO:264, amino acids 21-136 of SEQ ID NO:266 and amino acids 21-140 of SEQ ID NO:268, amino acids 21-136 of SEQ ID NO:270 and amino acids 21-146 of SEQ ID NO:272, and amino acids 21-131 of SEQ ID NO:274 and amino acids 21-139 of SEQ ID NO:276. In some embodiments, the antigen binding protein binds to an epitope encoded in a peptide selected from the group consisting of SEQ ID NOs: 93-123 and 224. In some embodiments, the immunoglobulin is a fusion with a heterologous polypeptide. In some embodiments, the immunoglobulin is a fusion with a heterologous polypeptide. In some embodiments, the present invention provides a vector encoding the antigen binding protein described above. In some embodiments, the present invention provides a host cell expressing an antigen binding protein as described above. In yet other embodiments the invention provides a vector encoding or a host cell expressing an antigen binding protein targeting another bacterial pathogen of interest.

In some embodiments, the present invention provides an antigen binding protein fusion protein comprising at least a first microbiocide operably linked to a pair of polypeptides corresponding to antibody heavy and light chain variable regions, wherein the variable regions of the pair of polypeptides have amino acid sequences at least 90%, 95%, 97%, 98% or 100% identical to light and heavy chain variable region pairs selected from the group consisting of: amino acids 21-130 of SEQ ID NO:254 and amino acids 21-146 of SEQ ID NO:256, amino acids 21-136 of SEQ ID NO: 2 and amino acids 21-139 of SEQ ID NO:4, amino acids 21-136 of SEQ ID NO:6 and amino acids 21-138 SEQ ID NO: 8, amino acids 21-131 of SEQ ID NO:10 and amino acids 21-140 of SEQ ID NO:12, amino acids 21-137 of SEQ ID NO:14 and amino acids 21-140 of SEQ ID NO:16, amino acids 21-127 of SEQ ID NO:18 and amino acids 21-144 of SEQ ID NO:20, amino acids 21-131 of SEQ ID NO:22 and amino acids 21-136 of SEQ ID NO:24, amino acids 21-131 of SEQ ID NO:26 and amino acids 21-144 of SEQ ID NO:28, amino acids 21-131 of SEQ ID NO:30 and amino acids 21-144 of SEQ ID NO:32, amino acids 21-131 of SEQ ID NO:238 and amino acids 21-144 of SEQ ID NO:240, amino acids 21-130 of SEQ ID NO:242 and amino acids 21-145 of SEQ ID NO:244, amino acids 21-130 of SEQ ID NO:246 and amino acids 21-137 of SEQ ID NO:248, amino acids 21-137 of SEQ ID NO:250 and amino acids 21-140 of SEQ ID NO:252, amino acids 21-130 of SEQ ID NO:258 and amino acids 21-142 of SEQ ID NO:260, amino acids 21-132 of SEQ ID NO:262 and amino acids 21-142 of SEQ ID NO:264, amino acids 21-136 of SEQ ID NO:266 and amino acids 21-140 of SEQ ID NO:268, amino acids 21-136 of SEQ ID NO:270 and amino acids 21-146 of SEQ ID NO:272, and amino acids 21-131 of SEQ ID NO:274 and amino acids 21-139 of SEQ ID NO:276. In some embodiments, the antigen binding protein fusion protein further comprises a second microbiocide. In some embodiments, the microbiocide is selected from the group consisting of a peptidoglycan hydrolase, human beta-defensin 2, human beta-defensin 3, cathelicidin, magainin, and phospholipase. In some embodiments, the peptidoglycan hydrolase is a lysostaphin. In some embodiments, the first microbiocide is lysostaphin and the fusion protein further comprises a second microbiocide selected from the group comprising a peptidoglycan hydrolase, human beta-defensin 2 human beta defensin 3, cathelicidin, phospholipase and magainin. In some embodiments, the peptidoglycan hydrolase is a lysostaphin. In some embodiments, the microbiocide is fused to the light chain. In some embodiments, the microbiocide is fused to the heavy chain. In some embodiments, the first microbiocide is fused to the heavy chain and the second microbiocide is fused to the light chain. In some embodiments, the first microbiocide is fused to the C terminus of the heavy chain and the second microbiocide is fused to the N terminus of the light chain. In some embodiments, the lysostaphin is fused to the N terminal of the immunoglobulin heavy or light chain. In some embodiments, the lysostaphin is at least 90%, 95%, 97%, 98% or 100% identical to amino acids 21-266 of SEQ ID NO:34. In some embodiments, the fusion protein is antistaphylococcal. In some embodiments, the fusion protein is bactericidal in vitro at a concentration 1 nanomolar to about 100 nanomolar. In some embodiments, the fusion protein is antistaphylococcal against MRSA strains of S. aureus. In some embodiments, the fusion protein comprises lysostaphin and the fusion protein is bactericidal to methicillin resistant S. aureus at a MIC of 1 to 100 nanomolar. In some embodiments, the present invention provides a vector encoding the antigen binding protein fusion protein described above. In some embodiments, the present invention provides a host cell expressing an antigen binding protein fusion protein as described above.

In some embodiments, the present invention provides a recombinant fusion polypeptide selected from the group consisting of: a recombinant fusion polypeptide comprising a first polypeptide having N and C terminals and second and third polypeptides operably linked to the first polypeptide at the N and C terminals, wherein the second and third polypeptides are microbiocides and the recombinant fusion polypeptide has bacteriocidal activity; a recombinant fusion polypeptide composition comprising an immunoglobulin light chain operably linked to a microbiocide at its N or C terminal and an immunoglobulin heavy chain operably linked to a microbiocide at its N or C terminal, wherein the immunoglobulin heavy chain and immunoglobulin light chain are covalently bound to each other and the composition has bacteriocidal activity; and a recombinant fusion polypeptide composition comprising two immunoglobulin light chains operably linked to a microbiocide at its N or C terminal and two immunoglobulin heavy chains operably linked to a microbiocide at its N or C terminal, wherein each the immunoglobulin heavy chain is covalently bound to an immunoglobulin light chain, and the two immunoglobulin heavy chains are covalently bound to each other, and the composition has bacteriocidal activity. In some embodiments, the first polypeptide is from about 100 amino acids to 700 amino acids in length. In some embodiments, the first polypeptide is selected from the group consisting of an immunoglobulin polypeptide or an albumin polypeptide. In some embodiments, the immunoglobulin polypeptide is an immunoglobulin heavy chain or portion thereof. In some embodiments, the immunoglobulin polypeptide is an immunoglobulin light chain or portion thereof. In some embodiments, microbiocide is selected from the group consisting of a peptidoglycan hydrolase, human beta defensin 2, human beta defensin 3, cathelicidin, magainin, and phospholipase. In some embodiments, the peptidoglycan hydrolase is lysostaphin. In some embodiments, the second polypeptide is a peptidoglycan hydrolase. In some embodiments, the peptidoglycan hydrolase is lysostaphin. In some embodiments, the peptidoglycan hydrolase is fused at the N terminus of the first polypeptide, the light chain or the heavy chain. In some embodiments, the recombinant fusion polypeptide has antimicrobial activity. In some embodiments, the recombinant fusion polypeptide is bacteriocidal and bacteriostatic. In some embodiments, the recombinant fusion polypeptide is bacteriocidal and or bacteriastatic to Staphlyococcus spp. or Streptococcus spp. In some embodiments, the recombinant fusion polypeptide is bacteriocidal and or bacteriastatic to MRSA strains of S. aureus. In some embodiments, the recombinant fusion polypeptide is bacteriocidal in vitro at a concentration of 1 nanomolar to 100 nanomolar. In some embodiments, the present invention provides a vector encoding the fusion polypeptide described above. In some embodiments, the present invention provides a host cell expressing a fusion polypeptide as described above.

In some embodiments, the present invention provides a method of treating a subject comprising contacting the subject suspected of being infected with, infected with, or at risk of being infected with S. aureus with a pharmaceutical composition comprising a recombinant fusion protein, antigen binding protein, or antigen binding protein fusion protein as described above. In some embodiments, the pharmaceutical composition is administered by a route selected from the group consisting of oral administration, parenteral administration and topical administration. In some embodiments, the pharmaceutical composition is applied intraocularly. In some embodiments, the pharmaceutical composition is applied prophylactically or therapeutically. In some embodiments, the pharmaceutical composition is applied to a surgical site. In some embodiments, the subject is human. In some embodiments, the subject is a livestock species or a companion animal. In some embodiments, the methods further comprise coadministering an antibiotic. In some embodiments, the antibiotic is selected from the group consisting of beta lactams, cephalosporins, daptomycin, vancomycin, linezolid, tigecycline. In some embodiments, the pharmaceutical composition comprises a pharmaceutically accepted carrier. In some embodiments, the pharmaceutical composition is applied to a biofilm.

In some embodiments, the present invention provides a method of treating a subject comprising contacting the subject suspected of being infected with, infected with, or at risk of being infected with Streptococcus spp with a pharmaceutical composition comprising a recombinant fusion polypeptide as described above. In some embodiments, the pharmaceutical composition is administered by a route selected from the group consisting of oral administration, parenteral administration and topical administration. In some embodiments, the subject is human. In some embodiments, the subject is a livestock species or a companion animal. In some embodiments, the pharmaceutical composition comprises a pharmaceutically accepted carrier.

In some embodiments, the present invention provides a method of treating an object, comprising: contacting an object suspected of being contaminated with, contaminated with, or at risk of being contaminated with S. aureus with protein composition comprising a recombinant fusion protein, antigen binding protein, or antigen binding protein fusion protein as described above. In some embodiments, the object is an object introduced into a subject by a medical or surgical procedure. In some embodiments, the object is selected from the group consisting of a prosthesis, a suture, a wound filler, a catheter, or a medical device. In some embodiments, the object is an object applied to the skin or mucosa of a living subject. In some embodiments, the object is selected from the group consisting of a bandage, a suture, wound closure, a catheter, or a medical device. In some embodiments, the protein composition is incorporated into a coating. In some embodiments, the coating is a polymer. In some embodiments, the coating is hydroxyapatite or calcium phosphate. In some embodiments, the coating further comprises an antibiotic. In some embodiments, the object has a biofilm thereon or is at risk of developing a biofilm.

In some embodiments, the present invention provides a recombinant fusion protein comprising an active recombinant lysostaphin protein fused to a fusion partner protein. In some embodiments, the recombinant fusion polypeptide is secreted by a mammalian cell. In some embodiments, the fusion protein partner is an immunoglobulin molecule or fragment thereof. In some embodiments, the lysostaphin protein is fused to the N terminal of the immunoglobulin molecule or fragment thereof. In some embodiments, the lysostaphin protein is fused to the C terminal of the immunoglobulin molecule or fragment thereof. In some embodiments, the lysostaphin protein is connected to the immunoglobulin molecule or fragment thereof via a peptide linker. In some embodiments, the active recombinant lysostaphin is encoded by an amino acid sequence found in hosts which naturally express lysostaphin. In some embodiments, the active recombinant lysostaphin is encoded by an amino acid sequence selected from the group consisting of amino acids 21-266 of SEQ ID NO:278, amino acids 21-266 of SEQ ID NO:284, and amino acids 21-266 of SEQ ID NO: 290. In some embodiments, the lysostaphin sequences are at least 80%, 90%, 95%, 97% or 98% identical to the wild-type lysostaphin sequence (e.g., amino acids 21-266 of SEQ ID NO: 278, amino acids 21-266 of SEQ ID NO:284, and amino acids 21-266 of SEQ ID NO:290). In some embodiments, the active recombinant lysostaphin is encoded by an amino acid sequence altered from that found in a host naturally secreting lysostaphin. In some embodiments, the amino acid sequence of the active recombinant lysostaphin has been altered to eliminate one or more glycosylation sites. In some embodiments, the active recombinant lysostaphin is encoded by an amino acid sequence selected from the group consisting of amino acids 21-266 of Seq 280, amino acids 21-266 of Seq 282, amino acids 21-266 of Seq 286, amino acids 21-266 of Seq 288, and amino acids 21-266 of Seq 292. In some embodiments, the lysostaphin sequences are at least 80%, 90%, 95%, 97% or 98% identical to the lysostaphin sequences mutated at one or both of positions 125 and 232 (e.g., amino acids 21-266 of SEQ ID NO: 280, amino acids 21-266 of SEQ ID NO: 282, amino acids 21-266 of SEQ ID NO: 286, amino acids 21-266 of SEQ ID NO:288, amino acids 21-266 of SEQ ID NO:292). In some embodiments, the recombinant fusion protein is expressed by mammalian cells and harvested from supernatant at more than about 1 ug/ml. In some embodiments, the lysostaphin protein comprises a preprolysostaphin. In some embodiments, the lysostaphin protein comprises a mature lysostaphin. In some embodiments, the active lysostaphin is bactericidal. In some embodiments, the active lysostaphin is bactericidal at a MIC of 1-100 nanomolar. In some embodiments, the active lysostaphin is bactericidal to methicillin resistant S. aureus at a MIC of 1-100 nanomolar. In some embodiments, the enzymatically active half-life of the recombinant active lysostaphin in vivo is greater than 1 hour. In some embodiments, the recombinant active lysostaphin is stable at 2-8 C for over 3 months. In some embodiments, the active lysostaphin is bactericidal to methicillin resistant S. aureus at a MIC of 1-100 nanomolar.

In some embodiments, the present invention provides a pharmaceutical preparation comprising the fusion protein comprising an active recombinant lysostaphin as described above. In some embodiments, the present invention provides a mammalian host cell comprising a vector encoding a recombinant fusion polypeptide comprising an active recombinant lysostaphin protein, wherein the active recombinant lysostaphin protein is stably secreted. In some embodiments, the mammalian host cell is a stable cell line in which expression of the recombinant fusion polypeptide comprising an active recombinant lysostaphin protein is maintained through at least 20 passages.

### DESCRIPTION OF THE FIGURES

FIG. 1: Assembly of mouse-human chimeric DB coding sequence. A, Amplification of variable region using degenerate 5′ primer and constant region 3′ primer, resulting product is cloned and sequenced. B, Amplification of mature murine variable region with addition of restriction sites; C, Amplification of human constant region from human blood cDNA (Invitrogen, Carlsbad, Calif.) and addition of restriction sites; D, restriction site mediated ligation of hC_{H} into retroviral backbone containing 3 different linker-biocide portions (We have constructed 3 different retroviral backbones for each biocide, LL37, PLA2 and HBD2); E, ligation of mV_{H} into retrovector backbone containing human constant heavy chain linked to various biocides. mV_{H}=murine variable heavy chain, hC_{H1-3}=human constant heavy chain region 1-3, Koz=Kozak element, SP=signal peptide.

FIG. 2: Example of a genetic construct for making mouse-human chimeric DB using the MLV-based retroviral vector. LTR=long terminal repeat, EPR=extended packaging region, sCMV=simian cytomegalo virus promoter, SP=signal peptide, mV_{H}=murine heavy chain variable region, mV_{L}=murine light chain variable region, hC_{H}=human heavy chain constant region, hC_{L}=human light chain constant region, EX=RNA export signal, (G₄S)₃=glycine-serine linker, Bioc=biocide.

FIG. 3: Results of efficacy testing for anti-staphylococcal antigen binding fusion proteins. Efficacy is expressed as positive log reduction in Staphylococcal growth; a negative value indicates continued growth. Lysostaphin was included at various concentrations as a control. Also shown is a cartoon of the configuration of each construct

FIG. 4: Results of efficacy testing for anti-staphylococcal antigen binding fusion proteins.

FIG. 5: Results of efficacy testing for anti-staphylococcal antigen binding fusion proteins.

FIG. 6: Structures for tethered microbiocides

FIG. 7: Tethered microbiocides comprising immunoglobulins

FIG. 8: Results of efficacy testing for anti-staphylococcal antigen binding fusion proteins.

FIG. 9: Results of Minimum Inhibitory Concentration (MIC) testing according to CSLI guidelines. Concentrations are shown in nanomolar units and compared to a lysostaphin control.

FIG. 10: Time kill curve under MIC conditions, i.e. each product is at its MIC concentration (as listed) and with 5E5 cfu/ml USA300 target cells.

FIG. 11: Table presenting MIC (minimal inhibitory concentration) for selected recombinant fusion proteins.

FIG. 12: Kaplan-Meier survival plot of mice treated with test substance and challenged with S. aureus.

### DEFINITIONS

To facilitate an understanding of the present invention, a number of terms and phrases are defined below:

“A recombinant antibody that binds to a surface epitope of Staphylococcus sp.” refers to a recombinantly expressed monoclonal antibody that binds to a specific epitope on the surface of Staphylococcus sp. Exemplary Staphylococcus spp. epitopes include, but are not limited to, epitopes encoded by SEQ ID NOs:93-123 and 224.

“Antigen binding protein” refers to proteins that bind to a specific antigen. “Antigen binding proteins” include, but are not limited to, immunoglobulins, including polyclonal, monoclonal, chimeric, single chain, and humanized antibodies, Fab fragments, F(ab′)2 fragments, and Fab expression libraries.

Various procedures known in the art are used for the production of polyclonal antibodies. For the production of antibody, various host animals can be immunized by injection with the peptide corresponding to the desired epitope including but not limited to rabbits, mice, rats, sheep, goats, etc. In a preferred embodiment, the peptide is conjugated to an immunogenic carrier (e.g., diphtheria toxoid, bovine serum albumin (BSA), or keyhole limpet hemocyanin (KLH)). Various adjuvants are used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum. For preparation of monoclonal antibodies, any technique that provides for the production of antibody molecules by continuous cell lines in culture may be used (See e.g., Harlow and Lane, Antibodies: A Laboratory Manual, Cold Spring Harbor Laboratory Press, Cold Spring Harbor, N.Y.). These include, but are not limited to, the hybridoma technique originally developed by Köhler and Milstein (Köhler and Milstein, Nature, 256:495-497 [1975]), as well as the trioma technique, the human B-cell hybridoma technique (See e.g., Kozbor et al., Immunol. Today, 4:72 [1983]), and the EBV-hybridoma technique to produce human monoclonal antibodies (Cole et al., in Monoclonal Antibodies and Cancer Therapy, Alan R. Liss, Inc., pp. 77-96 [1985]).

In other embodiments, suitable monoclonal antibodies, including recombinant chimeric monoclonal antibodies and chimeric monoclonal antibody fusion proteins are prepared as described herein. According to the invention, techniques described for the production of single chain antibodies (U.S. Pat. No. 4,946,778; herein incorporated by reference) can be adapted to produce specific single chain antibodies as desired. An additional embodiment of the invention utilizes the techniques known in the art for the construction of Fab expression libraries (Huse et al., Science, 246:1275-1281 [1989]) to allow rapid and easy identification of monoclonal Fab fragments with the desired specificity. In some embodiments, monoclonal antibodies are generated using the ABL-MYC method (See e.g., U.S. Pat. Nos. 5,705,150 and 5,244,656, each of which is herein incorporated by reference) (Neoclone, Madison, Wis.). ABL-MYC is a recombinant retrovirus that constitutively expresses v-abl and c-myc oncogenes. When used to infect antigen-activated splenocytes, this retroviral system rapidly induces antigen-specific plasmacytomas. ABL-MYC targets antigen-stimulated (Ag-stimulated) B-cells for transformation. Antibody fragments that contain the idiotype (antigen binding region) of the antibody molecule can be generated by known techniques. For example, such fragments include but are not limited to: the F(ab′)2 fragment that can be produced by pepsin digestion of an antibody molecule; the Fab′ fragments that can be generated by reducing the disulfide bridges of an F(ab′)2 fragment, and the Fab fragments that can be generated by treating an antibody molecule with papain and a reducing agent. Genes encoding antigen-binding proteins can be isolated by methods known in the art. In the production of antibodies, screening for the desired antibody can be accomplished by techniques known in the art (e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), Western Blots, precipitation reactions, agglutination assays (e.g., gel agglutination assays, hemagglutination assays, etc.), complement fixation assays, immunofluorescence assays, protein A assays, and immunoelectrophoresis assays, etc.) etc.

“Biocide” or “biocides,” or “microbiocides” as used herein, refer to at least a portion of a naturally occurring or synthetic molecule (e.g., peptides) that directly kills or promotes the death and/or attenuation of, or otherwise neutralizes infectivity without killing (e.g., prevents growth and/or replication) of biological targets (e.g., bacteria, parasites, yeast, viruses, fungi, protozoans and the like). Examples of biocides include, but are not limited to, bactericides, viricides, fungicides, parasiticides, and the like.

“Cell type specific” as applied to a regulatory element refers to a regulatory element which is capable of directing selective expression of a nucleotide sequence of interest in a specific type of cell in the relative absence of expression of the same nucleotide sequence of interest in a different type of cell within the same tissue (e.g., cells infected with retrovirus, and more particularly, cells infected with BLV or HTLV). The term “cell type specific” when applied to a regulatory element also means a regulatory element capable of promoting selective expression of a nucleotide sequence of interest in a region within a single tissue. The cell type specificity of a regulatory element may be assessed using methods well known in the art (e.g., immunohistochemical staining and/or Northern blot analysis). Briefly, for immunohistochemical staining, tissue sections are embedded in paraffin, and paraffin sections are reacted with a primary antibody specific for the polypeptide product encoded by the nucleotide sequence of interest whose expression is regulated by the regulatory element. A labeled (e.g., peroxidase conjugated) secondary antibody specific for the primary antibody is allowed to bind to the sectioned tissue and specific binding detected (e.g., with avidin/biotin) by microscopy. Briefly, for Northern blot analysis, RNA is isolated from cells and electrophoresed on agarose gels to fractionate the RNA according to size followed by transfer of the RNA from the gel to a solid support (e.g., nitrocellulose or a nylon membrane). The immobilized RNA is then probed with a labeled oligo-deoxyribonucleotide probe or DNA probe to detect RNA species complementary to the probe used. Northern blots are a standard tool of molecular biologists.

“Co-administration” refers to administration of more than one agent or therapy to a subject. Co-administration may be concurrent or, alternatively, the chemical compounds described herein may be administered in advance of or following the administration of the other agent(s). One skilled in the art can readily determine the appropriate dosage for co-administration. When co-administered with another therapeutic agent, both the agents may be used at lower dosages. Thus, co-administration is especially desirable where the claimed compounds are used to lower the requisite dosage of known toxic agents.

“Staphylococcus sp.” refers to any species of Staphylococcus, including multidrug resistant species.

“Streptococcus spp” refers to any species of Streptococcus, including multidrug resistant species.

“Fusion protein”, as used herein, refers to a single polypeptide that comprises one or more distinct functional units (e.g., polypeptides, linkers, etc.) joined in the same polypeptide chain. In some embodiments, fusion proteins comprise an immunoglobulin and a biocide. In some embodiments, fusion proteins comprise additional components such as, for example, linkers, signal sequences, etc. Fusion protein polypeptides may be assembled with other polypeptides to provide a functional protein (e.g., a fusion protein immunoglobulin heavy chain with an immunoglobulin light chain).

In some embodiments a fusion protein is expressed as a single polypeptide from a single polynucleotide in a cell; in yet other embodiments a fusion protein is assembled by chemical synthesis from multiple polypeptides.

“Genome,” as used herein, refers to the genetic material (e.g., chromosomes) of an organism or a host cell.

“Halfmer” or “halfmer immunoglobulin,” as used herein refers to an immunoglobin comprising one light chain and one heavy chain. Halfmer immunoglobulins may be derived from an IgM or IgG or any other immunoglobulin (e.g., an immunoglobulin that normally assembles as units of two or more light chains and two or more heavy chains). To achieve the assembly as a halfmer three substitutions are made in each of the heavy and light chains from Cysteine to serine to remove the disulphide bonds.

“Host cell,” as used herein, refers to any eukaryotic cell (e.g., mammalian cells, avian cells, amphibian cells, plant cells, fish cells, insect cells, yeast cells, and bacteria cells, and the like), whether located in vitro or in vivo (e.g., in a transgenic organism).

“Intrabuccal” as used herein means delivery into the mouth for uptake through the buccal mucosa or dissolution in the mouth. This may be by means of liquid drops or inclusion in a carrier such as, but not limited to, a gelatin or starch based substrate lozenge or strip.

“In operable combination,” “in operable order,” and “operably linked,” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

“Lysostaphin” as used herein refers to glycylglycine endopeptidases which are capable of cleaving the crosslinking pentaglycin bridges in the cell wall of Staphylococci. As used herein, an “active lysostaphin” is an enzyme or fusion thereof which lyses the cell wall of Staphylococci. “Preprolysostaphin” as used herein means the entire transcription product of the lysostaphin gene comprising typically around 480 amino acids. The lysostaphin gene consists of a N terminal signal peptide, a series of tandem repeats and a mature active peptidoglycan hydrolase enzyme, typically of 246 amino acids. The preprolysostaphin secreted by S. simulans and converted extracellularly to the active mature enzyme. “Mature lysostaphin” as used herein means the active enzyme comprising approximately 246 amino acids released from the preprolysostaphin

MIC when used herein is the minimum inhibitory concentration determined according to the guidelines of the Clinical Laboratory Standards Institute.

“Wildtype” when used herein in reference to lysostaphin means lysostaphin with an amino acid sequence the same as is secreted from S. simulans.

“Methicillin resistant S. aureus” or “MRSA” as used herein refers to a strain of Staphylococcus aureus which is not neutralized by methicillin, Examples of such strains include but are not limited to BAA-44, NRS282(USA 100), NRS383(USA 200), NRS384(USA 300), NRS123(USA 400), NRS 22(USA 600) obtained from the Network on Antimicrobial Resistance in Staphylococcus aureus (NARSA)

“Methicillin sensitive S. aureus” or “MSSA” as used herein refers to a strain of Staphylococcus aureus which is neutralized by methicillin. Examples of such strains include but are not limited to Newman 25904, FDA 25923, Sanger 476 obtained from the Network on Antimicrobial Resistance in Staphylococcus aureus (NARSA)

“Vancomycin intermediate resistant S. aureus” or “VISA” as used herein refers to a strain of Staphylococcus aureus which is only partially neutralized by vancomycin. Examples of such strains include but are not limited to NRS385(USA500), NRS79 (IL) and NRS1 (Mu50) obtained from the Network on Antimicrobial Resistance in Staphylococcus aureus (NARSA)

“Metaphylactic,” as used herein, is used to describe the administration of a therapy or treatment (e.g., drug product) both before and during the active course of a disease. For example, metaphylactic it is used to describe a course of treatment which encompasses the period of potential exposure to the organism and the period of active parasite infection.

“Microorganism targeting molecule,” as used herein, refers to any molecule (e.g., protein) that interacts with a microorganism (e.g., parasite). In preferred embodiments, the microorganism targeting molecule specifically interacts with microorganisms at the exclusion of non-microorganism host cells. Preferred microorganism targeting molecules interact with broad classes of microorganism (e.g., all bacteria or all gram positive or negative bacteria). However, the present invention also contemplates microorganism targeting molecules that interact with a specific species or sub-species of microorganism. In some embodiments, microorganism targeting molecules are antibodies (e.g., monoclonal antibodies directed towards PAMPS or monoclonal antibodies directed to specific organisms or serotype specific epitopes).

“Monomer IgM,” as used herein, is used to describe the immunoglobulin structure which comprises two light chains and two heavy chains of immunoglobulin M in which two substitutions of cysteine for serine results in abrogation of the disulphide bond, and prevents the normal assembly into a hexamer (in absence of a J chain) or pentamer (if a J chain is present).

“Neutralization” and “pathogen neutralization,” as used herein refer to destruction or inactivation (e.g., loss of virulence or infectivity) of a “pathogen” (e.g., Cryptosporidium spp.) thus preventing the pathogen's ability to initiate a disease state in a subject or cause degradation of a food product.

“Non-specific binding” and “background binding” when used in reference to the interaction of an antibody and an antigen refer to an interaction that is not dependent on the presence of a particular structure (i.e., the antibody is binding to antigens in general rather that a particular structure such as an epitope).

“Peptidoglycan hydrolase” as used herein means an enzyme capable of cleaving amide or peptide bonds in polymeric peptidoglycan and/or its soluble fragments. Peptidoglycan hydrolases are also known as murein hydrolases. Peptidoglycan hydrolases are expressed by a wide variety of organisms and may be bacterial or phage in origin. Peptidoglycan hydrolases include but are not limited to N-Acetylmuramyl-L-alanine amidases (including for example but not limited to E. coli AmiA, AmiB, and AmiC enzymes), endopeptidases and carboxypeptidases (including for example but not limited to lysostaphin and zoocin), N-Acetyl-b-D-muramidases, lysozymes, lytic transglycosylases, N-Acetyl-b-D-glucosaminidases.

“Pharmaceutical composition” is intended to include the combination of an active agent with a carrier, inert or active, making the composition suitable for diagnostic or therapeutic use in vivo, in vivo or ex vivo.

“Pharmaceutically acceptable carrier” encompasses any of the standard pharmaceutical carriers, such as a phosphate buffered saline solution, water, and an emulsion, such as an oil/water or water/oil emulsion, and various types of wetting agents. The compositions also can include stabilizers and preservatives. For examples of carriers, stabilizers and adjuvants see Martin, Remington's Pharmaceutical Sciences, 15th Ed., Mack Publ. Co., Easton, Pa. (1975).

“Pharmaceutically acceptable salt” as used herein, relates to any pharmaceutically acceptable salt (acid or base) of a compound of the present invention, which, upon administration to a recipient, is capable of providing a compound of this invention or an active metabolite or residue thereof. As is known to those of skill in the art, “salts” of the compounds of the present invention may be derived from inorganic or organic acids and bases. Examples of acids include hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic and benzenesulfonic acid. Other acids, such as oxalic, while not in themselves pharmaceutically acceptable, may be employed in the preparation of salts useful as intermediates in obtaining the compounds of the invention and their pharmaceutically acceptable acid.

“Protein biocide” and “protein biocides,” and “protein microbiocides” as used herein, refer to at least a portion of a naturally occurring or synthetic peptide or protein molecule that directly kills or promotes the death and/or attenuation of, or otherwise neutralizes infectivity without killing (e.g., prevents growth and/or replication) of biological targets (e.g., bacteria, parasites, yeast, viruses, fungi, protozoans and the like). Examples of biocides include, but are not limited to, bactericides, viricides, fungicides, parasiticides, and the like.

“Protein of interest,” as used herein, refers to a protein encoded by a nucleic acid of interest.

“Purified” or “to purify,” as used herein, refers to the removal of undesired components from a sample. As used herein, the term “substantially purified” refers to molecules, either nucleic or amino acid sequences, that are removed from their natural environment, isolated or separated, and are at least 60% free, preferably 75% free, and most preferably 90% free from other components with which they are naturally associated. An “isolated polynucleotide” is therefore a substantially purified polynucleotide.

“Specific binding” or “specifically binding” when used in reference to the interaction of an antibody and an antigen means that the interaction is dependent upon the presence of a particular structure (i.e., the antigenic determinant or epitope) on the antigen; in other words the antibody is recognizing and binding to a specific structure rather than to antigens in general. For example, if an antibody is specific for epitope “A,” the presence of a protein containing epitope A (or free, unlabelled A) in a reaction containing labeled “A” and the antibody will reduce the amount of labeled A bound to the antibody.

“Subject” is an animal such as vertebrate, including a mammal, a bird (e.g., a chicken) or a fish. In some embodiments, the vertebrate is a mammal (e.g., a human or a bovine). Mammals, however, are understood to include, but are not limited to, murines, simians, humans, bovines, cervids, equines, porcines, canines, felines etc.).

“Tether” and “tethering” as used herein refers to the operable linkage of two molecular components either by expression as a single genetic fusion or as two genetic fusions the products of which are bound to each other. Hence two microbiocides may be tethered by expression at the N terminus and C terminus of a single immunoglobulin, or by expression as a fusion to an immunoglobulin light chain and a second fusion to an immunoglobulin heavy chain such that in the assembled immunoglobulin the microbiocides are tethered by the binding of the light chain and heavy chain to each other.

“Vector,” as used herein, refers to any genetic element, such as a plasmid, phage, transposon, cosmid, chromosome, retrovirus, virion, etc., which is capable of replication when associated with the proper control elements and which can transfer gene sequences between cells. Thus, the term includes cloning and expression vehicles, as well as viral vectors.

### DETAILED DESCRIPTION OF THE INVENTION

In the present invention we describe the identification of conserved epitopes on the surfome of S. aureus and the production of recombinant antibodies to these. Such antibodies may have direct antimicrobial efficacy when acting alone, providing one antimicrobial embodiment of the present invention. In another preferred embodiment the antibodies are further expressed as genetic fusions biocides comprising antimicrobial peptides and/or enzymes such that the antibody serves to deliver said biocides to the surface of the S. aureus bacterium. In some preferred embodiments the specific binding is a contributing feature of the fusion microbiocides; however in yet other embodiments an immunoglobulin may be used as a linker between two microbiocides. In yet other preferred embodiments both the specificity of targeting and the tethering of two microbiocides to coordinate their action are factors contributing to antimicrobial efficacy.

Fundamental to the successful design of specifically targeted antibody fusion proteins which are active against S. aureus is the ability to identify and target epitopes on the surface of Staphylococcus which (a) lie within a short distance of the site of action of the selected biocide, such that binding of the antibody can bring the biocide into proximity with the surface, and (b) so that the immunoglobulin and linker do not create steric interference preventing the biocide from reaching the surface. Suitable epitopes are those which are conserved between strains of S. aureus (both MRSA and MSSA) and which are expressed consistently and in vivo and in both planktonic and stationary phase cells. Examples include, but not limited to, adhesins, Iron sensitive determinants, the penicillin binding proteins, and proteins involved in septum formation. Other surface proteins also provide good targets so these should not be considered limiting.

In addition to the growing antimicrobial resistance of S. aureus strains, a further challenge to their control with antibiotics is the desirability of not harming and disrupting the commensal microflora of the body. Hence an approach which specifically eliminates S. aureus while leaving other commensal flora unharmed, is a desirable goal.

The present invention comprises the design and expression of novel antibodies to conserved peptide epitopes of S. aureus. It further comprises the production of antibody fusion microbiocides. In some embodiment this product the antibody targets a selected conserved surface epitope on S. aureus and is genetically fused via linker to a microbiocide capable of killing said S. aureus bacterium. In some preferred embodiments the linker is an elastin linker in others it is a serine glycine chain linker. In some embodiments the microbiocide is a lysostaphin. In others it is a cathelicidin, a defensin, a magainin or a phopholipase. None of these are considered limiting and a broad array or antibody targets on S. aureus as well as a variety of linkers and microbiocides may be employed.

The epitope selected in Staphylococcus for targeting the antibody may be selected by immunoinformatic analysis to identify exposed surface epitopes and hence to identify peptides which comprise epitope dense regions. In some preferred embodiments the selected proteins may be from the group comprising, but not limited to those proteins shown in Table 1.

TABLE 1
Penicillin-binding protein 2
LPXTG cell wall surface anchor protein
Penicillin-binding protein 3
sdrC protein, C-terminus of bacterial fibrinogen-binding adhesin
Fibronectin-binding protein A
Trans-sulfuration enzyme family protein
Cell wall associated fibronectin-binding protein
capsular polysaccharide biosynthesis galactosyltransferase Cap5M
Multimodular transpeptidase-transglycosylase/Penicillin-binding protein
1A/1B (PBP1)
D-alanyl-D-alanine carboxypeptidase/Penicillin binding protein PBP4
Penicillin-binding protein PBP2a, methicillin resistance determinant
MecA, transpeptidase
Cell surface receptor IsdH for hemoglobin-haptoglobin complexes
Cell division protein FtsI [Peptidoglycan synthetase]
FtsZ-interacting protein related to cell division
Multimodular transpeptidase-transglycosylase/Penicillin-binding protein
1A/1B (PBP1) [Staphylococcus aureus 04-02981]
Multimodular transpeptidase-transglycosylase/Penicillin-binding protein
1A/1B (PBP1) [Staphylococcus aureus 04-02981]
Multimodular transpeptidase-transglycosylase/Penicillin-binding protein
1A/1B (PBP1) [Staphylococcus aureus 04-02981]
Iron compound ABC transporter iron compound-binding protein
[Staphylococcus aureus subsp. aureus COL]

In yet other embodiments epitopes may be identified for targeting on the surface of other bacteria of interest including, but not limited to, Streptococcus spp, Enterococcus spp, and other pathogenic species.

In another embodiment, the invention encompasses antimicrobial fusion proteins (and the production thereof) in which two microbiocides are tethered together by a polypeptide to facilitate their joint action and to extend their half-life. In some cases, the tether may be an immunoglobulin molecule but other polypeptides of an appropriate length amino acids also function as tethers.

Accordingly, in some embodiments, the present invention provides a recombinant fusion polypeptide comprising a first polypeptide having N and C terminals and second and third polypeptides operably linked to said first polypeptide at said N and C terminals, wherein said second and third polypeptides are microbiocides and said recombinant fusion polypeptide has bacteriocidal activity. In some embodiments, the first polypeptide is from about 100 amino acids to 700 amino acids in length. In some embodiments, the first polypeptide is selected from the group consisting of an immunoglobulin polypeptide or an albumin polypeptide. In some embodiments, the immunoglobulin polypeptide is an immunoglobulin heavy chain or portion thereof. In some embodiments, the immunoglobulin polypeptide is an immunoglobulin light chain or portion thereof. In some embodiments, the microbiocides are selected from the group consisting of a peptidoglycan hydrolase, including but not limited to lysostaphin, a human beta defensin 2, human beta defensin 3, cathelicidin, magainin, and phospholipase. In some embodiments, the fusion proteins comprise two of more biocides. In some preferred embodiments, the first biocide is a peptidoglycan hydrolase, wherein said peptidoglycan hydrolase may be lysostaphin, and the second microbiocide drawn from the group comprising lysostaphin, human beta defensin 2 human beta defensin 3, cathelicidin, phospholipase. In some embodiments, the first microbiocide is fused to the N terminus of the first polypeptide and the second microbiocide is fused to the C terminus of the first polypeptide. In some preferred embodiments, the peptidoglycan hydrolase is fused to the N terminal of the first polypeptide; in some embodiments this peptidoglycan hydrolase may be lysostaphin, or it may be another peptidoglycan hydrolase. In some particularly preferred embodiments, lysostaphin comprises amino acids 21-266 of SEQ ID NO:34. In some embodiments, the fusion protein has antimicrobial activity. In some embodiments, the fusion is bacteriocidal and bacteriostatic. In some embodiments, the fusion protein is bacteriocidal and/or bacteriastatic to S. aureus. In some embodiments, the fusion protein is bacteriocidal and/or bacteriastatic to MRSA strains of S. aureus.

In some embodiments, the present invention provides fusion proteins comprising an antigen binding protein domain and one or more biocidal domains. In some embodiments, the antigen binding domain comprises at least light chain and heavy chain variable regions from an immunoglobulin that binds to an epitope of interest, and in particularly preferred embodiments to epitopes from S. aureus as described above. In some particularly preferred embodiments, the epitopes of interest are selected from SEQ ID NOs: 93-123 and 224.

In yet other embodiments the immunoglobulin binds to epitopes in other bacterial pathogens of interest, including but not limited to, Streptococcus spp, Enterococcus spp, and other pathogenic species.

In some embodiments, the antigen binding domain comprises the entire heavy and light chain, including constant domains, from a recombinant antibody, and in particularly preferred embodiments from an antibody that binds to a surface epitope of Staphylococcus sp. The present invention is not limited to the antigen binding fusion proteins that bind to Staphylococcus sp. The present invention encompasses antigen binding fusion proteins that bind to unrelated epitopes including those on a variety of other organisms including bacteria, viruses (e.g., influenza viruses), and protozoans (e.g., Cryptosporidium parvum). The antigen binding domain may be an immunoglobulin, polyclonal antibody, monoclonal antibody, chimeric antibody, single chain antibody, humanized antibody, Fab fragment, F(ab′)2 fragment, or scFV or indeed any antigen binding protein comprising heavy and light chain variable regions arranged so that the protein binds to an epitope of interest. In some embodiments, the antigen binding domain is a single chain antibody, scFv or halfmer immunoglobulin, while in other embodiments, the antigen binding domain comprises two immunoglobulin heavy chains and two immunoglobulin light chains covalently linked through disulfide bonds. In some embodiments, the antigen binding domain is monovalent, while in other embodiments, the antigen binding domain is divalent or polyvalent. The present invention is not limited to the use of any particular heavy and light chain variable regions. In some embodiments, suitable heavy and light chain variable region pairs correspond to those represented by SEQ ID NOs. 1-32 and 238-276. It will be understood that the variable region pair sequences corresponding to SEQ ID NOs. 1-32 comprise signal sequences in addition to the variable region sequences. The signal sequences may be varied. In some embodiments, preferred variable regions are at least 80%, 90%, 95% 97% or 98% identical to light and heavy chain variable region pairs selected from the group consisting of: amino acids 21-136 of SEQ ID NO: 2 and amino acids 21-139 of SEQ ID NO:4, amino acids 21-136 of SEQ ID NO:6 and amino acids 21-138 SEQ ID NO: 8, amino acids 21-131 of SEQ ID NO:10 and amino acids 21-140 of SEQ ID NO:12, amino acids 21-137 of SEQ ID NO:14 and amino acids 21-140 of SEQ ID NO:16, amino acids 21-127 of SEQ ID NO:18 and amino acids 21-144 of SEQ ID NO:20, amino acids 21-131 of SEQ ID NO:22 and amino acids 21-136 of SEQ ID NO:24, amino acids 21-131 of SEQ ID NO:26 and amino acids 21-144 of SEQ ID NO:28, amino acids 21-131 of SEQ ID NO:30 and amino acids 21-144 of SEQ ID NO:32, amino acids 21-130 of SEQ ID NO:254 and amino acids 21-146 of SEQ ID NO:256, amino acids 21-131 of SEQ ID NO:238 and amino acids 21-144 of SEQ ID NO:240, amino acids 21-130 of SEQ ID NO:242 and amino acids 21-145 of SEQ ID NO:244, amino acids 21-130 of SEQ ID NO:246 and amino acids 21-137 of SEQ ID NO:248, amino acids 21-137 of SEQ ID NO:250 and amino acids 21-140 of SEQ ID NO:252, amino acids 21-130 of SEQ ID NO:258 and amino acids 21-142 of SEQ ID NO:260, amino acids 21-132 of SEQ ID NO:262 and amino acids 21-142 of SEQ ID NO:264, amino acids 21-136 of SEQ ID NO:266 and amino acids 21-140 of SEQ ID NO:268, amino acids 21-136 of SEQ ID NO:270 and amino acids 21-146 of SEQ ID NO:272, and amino acids 21-131 of SEQ ID NO:274 and amino acids 21-139 of SEQ ID NO:276.

In some embodiments, the antigen binding protein fusion proteins comprise one or more biocide domains operably linked to the antigen binding protein domain. The present invention is not limited to any particular biocidal domain. In some embodiments, the biocidal domain is a protein biocide. Suitable protein biocides are listed in Table 2 below. In some preferred embodiments, biocides are selected from a peptidoglycan hydrolase, including but not limited to lysostaphin, human beta defensin 2, human beta defensin 3, cathelicidin, magainin, and phospholipase. In some embodiments, the protein biocides comprise a linker segment. Particularly preferred protein biocides and linker segments correspond to SEQ ID NOs: 33, 34, 39, 40, 41, 42, 43, 44, 45 and 46. It will be understood that the linker segments may be varied. It will be further understood that the protein biocide segments may be at least 80%, 90%, 95%, 87% or 98% identical to the protein biocide segments specified in SEQ ID NOs: 33, 34, 39, 40, 41, 42, 43, 44, 45 and 46. In some embodiments, the fusion proteins comprise two of more biocides. In some preferred embodiments, the first biocide is a peptidoglycan hydrolase such as lysostaphin and the second microbiocide drawn from the group comprising a peptidoglycan hydrolase, including but not limited to lysostaphin, human beta defensin 2 human beta defensin 3, cathelicidin, phospholipase or magainin. In some embodiments, the microbiocide is fused to the light chain. In some embodiments, the microbiocide is fused to the heavy chain. In some embodiments, the first microbiocide is fused to the heavy chain and the second microbiocide is fused to the light chain. In some embodiments, the first microbiocide is fused to the N terminus of the heavy chain and the second microbiocide is fused to the C terminus of the heavy chain. In some preferred embodiments, a peptidoglycan hydrolase such as lysostaphin is fused to the N terminal of a polypeptide comprising the heavy or light chain. In some particularly preferred embodiments, lysostaphin comprises amino acids 21-266 of SEQ ID NO:34. In some embodiments, the lysostaphin is at least 80%, 90%, 95%, 97% or 98% identical to amino acids 21-266 of SEQ ID NO:34. In some embodiments, the lysostaphin sequence is altered to eliminate one or more glycosylation sites.

In some embodiments, preferred heavy and or light chain fusions are encoded by SEQ ID NOs.:51-88 (full chains) and 89-92 (Fab's).

In some embodiments, the antigen binding protein fusion protein has antimicrobial activity. In some embodiments, the fusion is bacteriocidal and bacteriostatic. In some embodiments, the fusion protein is bacteriocidal and/or bacteriastatic to S. aureus. In some embodiments, the fusion protein is bacteriocidal and/or bacteriastatic to MRSA strains of S. aureus. In yet other embodiments the fusion protein is bacteriocidal and/or bacteriastatic to other bacterial pathogens of interest, including but not limited to o, Streptococcus spp, Enterococcus spp, and other pathogenic species.

Microbiocides

The role of the innate defenses in combating bacterial infection, including S. aureus, is well documented (14, 15). Cationic antimicrobial peptides such as defensins and cathelicidins have multiple modes of action, including causing direct structural damage to bacterial surface membranes (16) but also immunomodulation (15, 17, 18).

The examples which follow describe some examples from among many antimicrobial peptides and enzymes which can be employed in the construction of anti staphylococcal fusion proteins; these examples should not be considered limiting.

Cathelicidin derived peptide LL37 is an alphahelical peptide derived from human cathelicidin; LL37 is capable of puncturing bacterial membranes resulting in osmotic disruption (16, 19, 20). Recombinant forms of the LL37 peptide have shown activity against a broad range of bacteria including Listeria monocytogenes, S. aureus, E coli, (21, 22),(24), and trypanosomes (25). Overexpression in transgenic mice offered enhanced protection against bacterial disease (26). We have engineered and expressed fusion proteins containing LL37 linked to monoclonal antibodies and shown efficacy against Cryptosporidium parvum (23); see also U.S. application Ser. Nos. 12/686,879, 12/536,291, 11/545,601, and 11/254,500, each of which are incorporated herein by reference in their entirety).

Defensins are small cationic peptides characterized by three disulfide bonds. Several types of defensins are recognized (alpha, beta and theta) and all have microbicidal activity against bacteria, and some also have activity against fungi, viruses and protozoa (24). The primary mode of action is thought to be membrane disruption. Alpha and beta defensins are active against a variety of bacteria (25, 26). Both HBD2 and HBD3 are active against S. aureus (27, 28). Both are found at epithelial surfaces, including gastrointestinal mucosa (24).

Secretory Phospholipase A2 (sPLA2) is a 14 kD enzyme which hydrolyzes membrane phospholipids of microorganisms and is synthesized in a number of gland cells (29, 30). Human tears under non-inflammatory conditions contain 54 μg/ml of sPLA2 (31). sPLA2 is a very effective antimicrobial (32), and mammalian cells are generally highly resistant to sPLA2, as composition of the surface phospholipids on the organism determines susceptibility to sPLA2 (32),(33). sPLA2 shows activity at nanomolar concentrations against Listeria monocytogenes (32), and at micromolar concentrations against other gram positive bacteria (34). sPLA2 has been shown highly bactericidal against Bacillus anthracis (35) at micromolar levels. It has a lesser, but clearly destructive, effect on Gram negative bacteria (32, 34, 36-38). We have engineered and expressed fusion proteins containing sPLA2 linked to monoclonal antibodies and shown efficacy against Cryptosporidium parvum (See, e.g., U.S. application Ser. Nos. 12/686,879, 12/536,291, 11/545,601, and 11/254,500, each of which are incorporated herein by reference in their entirety). sPLA2 molecules are small, compact and are very stable to extremes of temperature and pH which may be an advantage for applications ex vivo.

Many other animal species harbor innate antimicrobials on their skin and mucosae which may be of utility on constructing recombinant antimicrobial proteins. Examples include those found in amphibians such as magainin and ranalexin (39, 40). Many other antimicrobial peptides have been discovered and may be useful in antimicrobial formulations or as we describe herein in fusion protein antimicrobials.

Microbiocides of Bacterial Origin

In addition to the microbiocides outlined above many bacteria produce antimicrobial products either as a function of constitutive expression or through expression by bacteriophages.

Among these are the peptidoglycan hydrolases, which are enzymes capable of cleaving amide or peptide bonds in polymeric peptidoglycan which makes up bacterial cell walls (Vollmer et a,l FEMS Microbiol Rev 32 (2008) 259-286). The peptidoglycan hydrolases encompasses a diverse group which include N-acetylmuramyl-L-alanine amidases (including for example but not limited to E. coli AmiA AmiB and AmiC enzymes), endopeptidases and carboxypeptidases (including for example but not limited to lysostaphin and zoocin), N-Acetyl-b-D-muramidases, lysozymes, Lytic transglycosylases, N-Acetyl-b-D-glucosaminidases. Suitable peptidoglycan hydrolases are described in Gilmer et al., Antimicrob. Agents and Chemo., 57(6):2743-64 (2013); Simmonds et al., App. Enviro. Microbiol., 62(12):4536-41 (1996), WO9926969A1; WO2C145630A2; WO2C145573A2, and U.S. Pat. No. 7,982,003: all of which are incorporated herein by reference in their entirety. One particular example of a peptidoglycan hydrolase, which should not be considered limiting, is lysostaphin, a 27 KD glycylglycine endopeptidase, which is an antibacterial enzyme capable of cleaving the pentaglycine bridges in peptidoglycan comprising the cell wall of Staphylococci. S. aureus cell walls contain high proportions of pentaglycine, making lysostaphin a highly effective agent against both actively growing and quiescent bacteria. Lysostaphin has shown to be effective against methicillin resistant Staphylococcus. (Dajcs, Joseph J.; Emma B. H. Hume, Judy M. Moreau, Armando R. Caballero, Bennetta M. Cannon, Richard J. O'Callaghan (May 2000). “Lysostaphin Treatment of Methicillin-Resistant Staphylococcus aureus Keratitis in the Rabbit”. Investigative Ophthalmology and Visual Science 41 (6): 1432-1437) (41). Other peptidoglycan hydrolases include AmiA, AmiB, AmiC, AmiD, PLY endolysin, penicillin binding proteins, MepA, ALE-1, d-ALA-d-ALA carboxypeptidase VanX, CWlK, LytH, CWlO, CWlS, MpaA, lysozymes, lytic transglycolases, zoocin, and N Acetyl beta d glucosaminidases. Many peptidoglycan hydrolases are reviewed by Vollmer et al (Vollmer et a, 1 FEMS Microbiol Rev 32 (2008) 259-286) and can be considered exemplary but not limiting.

Lysostaphin is a natural product of Staphylococcus simulans in which it is encoded by a plasmid, naturally secreted as a preproprotein of approximately 480 amino acids and subject to postranslational processing to yield a mature form of 246 amino acid (48). The molecular organization of the lysostaphin gene and its sequences repeated in tandem. Several forms of lysostaphin are known (48-50) which differ by one or more amino acids. Mature lysostaphin is recognized as more enxymatically active in its peptidoglycan hydrolysis than the preprolysostaphin (50).

Lysostaphin has long been of interest as a potential antibacterial agent (51-54). It has been successfully expressed in bacterial expression systems (55-56). However, it has proven very difficult to express in eukaryotic bioreactor systems in its natural form as secreted by S. simulans (57) (see also U.S. Pat. No. 7,091,332 incorporated herein by reference); in the rare cases where expression in mammalian cell culture is reported it has been transient and at a low level.

In order to achieve secretion in mammalian cells of active mature lysostaphin it has been found necessary to modify the lysostaphin sequence to remove glycosylation sites. (See e.g., Huang et al, 2013 Animal Biotechnology 24:129-147 and U.S. Pat. No. 7,091,332) In particular mutations of N125Q (numbering from start of lysostaphin component) have been employed. Only with these modifications has it been possible to express active recombinant lysostaphin from mammalian cells.

In some embodiments, the present invention provides a composition and a method for the production of recombinant mature lysostaphin secreted by mammalian cells as a fusion, preferably with an immunoglobulin, which is stable and which retains the enzymatic peptidoglycan hydrolase function of wildtype lysostaphin active, and which is produced at commercially useful levels. The immunoglobulin fusion polypeptide comprising a recombinant, stable, active, mature lysostaphin is secreted either comprising either lysostaphin encoded by a natural amino acid sequence or encoded by a mutated amino acid sequence in which glycosylation sites have been mutated. Preferred embodiments of fusions of an immunoglobulin and a wild-type lysostaphin are provided by SEQ ID NOs: 278, 284 and 290, with the wild-type lysostaphin corresponding to amino acids 21-266 of SEQ ID NO: 278, amino acids 21-266 of SEQ ID NO:284, and amino acids 21-266 of SEQ ID NO:290. Examples of preferred embodiments of fusions of an immunoglobulin with a mutated lysostaphin protein in which glycosylation sites have been removed are provided by SEQ ID NOs:280, 282, 286, 288 and 292, with the mutated lysostaphin corresponding to amino acids 21-266 of SEQ ID NO: 280, amino acids 21-266 of SEQ ID NO: 282, amino acids 21-266 of SEQ ID NO: 286, amino acids 21-266 of SEQ ID NO:288, amino acids 21-266 of SEQ ID NO:292. In some embodiments, the lysostaphin sequences are at least 80%, 90%, 95%, 97% or 98% identical to the wild-type lysostaphin sequence (e.g., amino acids 21-266 of SEQ ID NO: 278, amino acids 21-266 of SEQ ID NO:284, and amino acids 21-266 of SEQ ID NO:290). In some preferred embodiments, the lysostaphin component comprises mutations as follows that eliminate a glycosylation site. In some embodiments, the lysostaphin is mutated to substitute the asparagine residues found at one or both of the 125 and 232 positions of the wild-type lysostaphin with an amino acid that is not glycosylated, for example glycine. In some embodiments, the lysostaphin is mutated to substitute the asparagine residues found at position 125 in wild-type lysostaphin with an amino acid that is not glycosylated, for example glycine. In some embodiments, the lysostaphin sequences are at least 80%, 90%, 95%, 97% or 98% identical to the lysostaphin sequences mutated at one or both of positions 125 and 232 (e.g., amino acids 21-266 of SEQ ID NO: 280, amino acids 21-266 of SEQ ID NO: 282, amino acids 21-266 of SEQ ID NO: 286, amino acids 21-266 of SEQ ID NO:288, amino acids 21-266 of SEQ ID NO:292).

In some embodiments the present invention provides a recombinant polypeptide comprising an active peptidoglycan hydrolase sequence secreted from a mammalian cell. In preferred embodiments said peptidoglycan hydrolase molecule is expressed as a fusion to an immunoglobulin molecule or a component of an immunoglobulin molecule. In some such embodiments the immunoglobulin molecule is a heavy chain, in others it is a light chain and in yet others it is a FAb or other immunoglobulin fragment. In further preferred embodiments the immunoglobulin molecule may be coexpressed with a second immunoglobulin molecule. The immunoglobulin molecules may be of any isotype or species. Preferred peptidoglycan hydrolase molecules include but are not limited to lysostaphin and other endopeptidases, carboxypeptidases, N-acetylmuramyl-L-alanine amidases, N-Acetyl-b-D-muramidases, lysozymes, Lytic transglycosylases, and N-Acetyl-b-D-glucosaminidases. When lysostaphin is the peptidoglycan hydrolase it may be present as a precursor form or as a mature form. In preferred embodiments, expression of the recombinant polypeptides comprises a lysostpahin protein in a mature active enzyme form. In some embodiments the peptidoglycan hydrolase is present in its native sequence. In yet other embodiments the peptidoglycan hydrolase is modified to change its glycosylation pattern; in some cases said modifications are substitutions of amino acids to remove potential glycosylation sites. The fusion polypeptides are secreted from stably expressing mammalian cell cultures in an active and stable form, enabling their formulation for administration to a subject as a biotherapeutic drug.

In some embodiments, the antimicrobial peptide or pore forming agent is a compound or peptide selected from the following: magainin (e.g., magainin I, magainin II, xenopsin, xenopsin precursor fragment, caerulein precursor fragment), magainin I and II analogs (PGLa, magainin A, magainin G, pexiganin, Z-12, pexigainin acetate, D35, MSI-78A, MG0 [K10E, K11E, F12W-magainin 2], MG2+ [K10E, F12W-magainin-2], MG4+[F12W-magainin 2], MG6+[f12W, E19Q-magainin 2 amide], MSI-238, reversed magainin II analogs [e.g., 53D, 87-ISM, and A87-ISM], Ala-magainin II amide, magainin II amide), cecropin P1, cecropin A, cecropin B, indolicidin, nisin, ranalexin, lactoferricin B, poly-L-lysine, cecropin A (1-8)-magainin II (1-12), cecropin A (1-8)-melittin (1-12), CA(1-13)-MA(1-13), CA(1-13)-ME(1-13), gramicidin, gramicidin A, gramicidin D, gramicidin S, alamethicin, protegrin, histatin, dermaseptin, lentivirus amphipathic peptide or analog, parasin I, lycotoxin I or II, globomycin, gramicidin S, surfactin, ralinomycin, valinomycin, polymyxin B, PM2 [(+/−) 1-(4-aminobutyl)-6-benzylindane], PM2c [(+/−)-6-benzyl-1-(3-carboxypropyl)indane], PM3 [(+/−)1-benzyl-6-(4-aminobutyl)indane], tachyplesin, buforin I or II, misgurin, melittin, PR-39, PR-26, 9-phenylnonylamine, (KLAKKLA)n (SEQ ID NO:225), (KLAKLAK)n (SEQ ID NO:226), where n=1, 2, or 3, (KALKALK)3 (SEQ ID NO:227), KLGKKLG)n (SEQ ID NO:228), and KAAKKAA)n (SEQ ID NO:229), wherein N=1, 2, or 3, paradaxin, Bac 5, Bac 7, ceratoxin, mdelin 1 and 5, bombin-like peptides, PGQ, cathelicidin, HD-5, Oabac5alpha, ChBac5, SMAP-29, Bac7.5, lactoferrin, granulysin, thionin, hevein and knottin-like peptides, MPG1, 1bAMP, snakin, lipid transfer proteins, and plant defensins. Exemplary sequences for the above compounds are provided in Table 2. In some embodiments, the antimicrobial peptides are synthesized from L-amino acids, while in other embodiments, the peptides are synthesized from or comprise D-amino acids.

TABLE 2
Antimicrobial Peptides

| SEQ ID | | | |
|---|---|---|---|
| NO: | Name | Organism | Sequence |
| 124 | lingual antimicrobial | Bos taurus | MRLHHLLLALLFLVLSAGSGFTQGV |
| | peptide precursor | | RNSQSCRRNKGICVP |
| | | | IRCPGSMRQIGTCLGAQVKCCRRK |
| 125 | antimicrobial peptide | Xenopus | GVLSNVIGYLKKLGTGALNAVLKQ |
| | PGQ | laevis | |
| 126 | Xenopsin | Xenopus | MYKGIFLCVLLAVICANSLATPSSDA |
| | | laevis | DEDNDEVERYVRGW |
| | | | ASKIGQTLGKIAKVGLKELIQPKREA |
| | | | MLRSAEAQGKRPWIL |
| 127 | Magainin 2 | Xenopus | GIGKFLHSAKKFGKAFVGEIMNS |
| | | laevis | |
| 212 | Magainin 1 | Xenopus | GIGKFLHSAGKFGKAFVGEIMKS |
| | | laevis | |
| 128 | tachyplesin I | Tachypleus | KWCFRVCYRGICYRRCR |
| | | gigas | |
| 129 | tachyplesin II | Tachypleus | RWCFRVCYRGICYRKCR |
| | | gigas | |
| 130 | buforin I | Bufo bufo | MSGRGKQGGKVRAKAKTRSSRAGL |
| | | gagarizans | QFPVGRVHRLLRKGNYAQRVGAGA |
| | | | PVYLAAVLEYLTAEILELAGNAARD |
| | | | NKKTRIIPRHLQLAVRNDEELNKLLG |
| | | | GVTIAQGGVLPNIQAVLLPKT |
| | | | ESSKPAKSK |
| 131 | buforin II | Bufo bufo | TRSSRAGLQFPVGRVHRLLRK |
| | | gagarizans | |
| 132 | cecropin A | Bombyx | MNFVRILSFVFALVLALGAVSAAPEP |
| | | mori | RWKLFKKIEKVGRNVRDGLIKAGPAI |
| | | | AVIGQAKSLGK |
| 133 | cecropin B | Bombyx | MNFAKILSFVFALVLALSMTSAAPEP |
| | | mori | RWKIFKKIEKMGRNIRDGIVKAGPAI |
| | | | EVLGSAKAIGK |
| 134 | cecropin C | Drosophila | MNFYKIFVFVALILAISIGQSEAGWLK |
| | | melanogaster | KLGKRIERIGQHTRDATIQGLGIAQQ |
| | | | AANVAATARG |
| 135 | cecropin P1 | Sus scrofa | SWLSKTAKKLENSAKKRISEGIAIAIQ |
| | | | GGPR |
| 136 | indolicidin | Bos taurus | ILPWKWPWWPWRR |
| 137 | nisin | Lactococcus | ITSISLCTPGCKTGALMGCNMKTATC |
| | | lactis | HCSIHVSK |
| 138 | ranalexin | Rana | FLGGLIKIVPAMICAVTKKC |
| | | catesbeiana | |
| 139 | lactoferricin B | Bos taurus | FKCRRWQWRMKKLGAPSITCVRRAF |
| 140 | protegrin-1 | Sus scrofa | RGGRLCYCRRRFCVCVGRX |
| 141 | protegrin-2 | Sus scrofa | GGRLCYCRRRFCICVG |
| 142 | histatin precursor | Homo | MKFFVFALILALMLSMTGADSHAKR |
| | | sapiens | HHGYKRKFHEKHHSHRGYRSNYLY |
| | | | DN |
| 143 | histatin 1 | Macaca | DSHEERHHGRHGHHKYGRKFHEKH |
| | | fascicularis | HSHRGYRSNYLYDN |
| 144 | dermaseptin | Phyllomedusa | ALWKTMLKKLGTMALHAGKAALG |
| | | sauvagei | AAADTISQTQ |
| 145 | dermaseptin 2 | Phyllomedusa | ALWFTMLKKLGTMALHAGKAALGA |
| | | sauvagei | AANTISQGTQ |
| 146 | dermaseptin 3 | Phyllomedusa | ALWKNMLKGIGKLAGKAALGAVKK |
| | | sauvagei | LVGAES |
| 147 | misgurin | Misgurnus | RQRVEELSKFSKKGAAARRRK |
| | | anguillicau- | |
| | | datus | |
| 148 | melittin | Apis | GIGAVLKVLTTGLPALISWISRKKRQ |
| | | mellifera | Q |
| 149 | pardaxin-1 | Pardachirus | GFFALIPKIISSPLFKTLLSAVGSALSS |
| | | pavoninus | SGEQE |
| 150 | pardaxin-2 | Pardachirus | GFFALIPKIISSPIFKTLLSAVGSALSSS |
| | | pavoninus | GGQE |
| 151 | bactenecin 5 precursor | Bos taurus | METQRASLSLGRCSLWLLLLGLVLPS |
| | | | ASAQALSYREAVLRAVDQFNERSSE |
| | | | ANLYRLLELDPTPNDDLDPGTRKPVS |
| | | | FRVKETDCPRTSQQPLEQCDFKENGL |
| | | | VKQCVGTVTLDPSNDQFDINCNELQS |
| | | | VRFRPPIRRPPIRPPFYPPFRPPIRPPIFP |
| | | | PIRPPFRPPLGPFPGRR |
| 152 | bactenecin precursor | Bos taurus | METPRASLSLGRWSLWLLLLGLALPS |
| | | | ASAQALSYREAVLRAVDQLNEQSSE |
| | | | PNIYRLLELDQPPQDDEDPDSPKRVS |
| | | | FRVKETVCSRTTQQPPEQCDFKENGL |
| | | | LKRCEGTVTLDQVRGNFDITCNNHQ |
| | | | SIRITKQPWAPPQAARLCRIVVIRVCR |
| 153 | ceratotoxin A | Ceratitis | SIGSALKKALPVAKKIGKIALPIAKAA |
| | | capitata | LP |
| 154 | ceratotoxin B | Ceratitis | SIGSAFKKALPVAKKIGKAALPIAKA |
| | | capitata | ALP |
| 155 | cathelicidin | Homo | MKTQRNGHSLGRWSLVLLLLGLVM |
| | antimicrobial peptide | sapiens | PLAIIAQVLSYKEAVLRAIDGINQRSS |
| | | | DANLYRLLDLDPRPTMDGDPDTPKP |
| | | | VSFTVKETVCPRTTQQSPEDCDFKKD |
| | | | GLVKRCMGTVTLNQARGSFDISCDK |
| | | | DNKRFALLGDFFRKSKEKIGKEFKRI |
| | | | VQRIKDFLRNLVPRTES |
| 156 | myeloid cathelicidin 3 | Equus | METQRNTRCLGRWSPLLLLLGLVIPP |
| | | caballus | ATTQALSYKEAVLRAVDGLNQRSSD |
| | | | ENLYRLLELDPLPKGDKDSDTPKPVS |
| | | | FMVKETVCPRIMKQTPEQCDFKENG |
| | | | LVKQCVGTVILDPVKDYFDASCDEP |
| | | | QRVKRFHSVGSLIQRHQQMIRDKSEA |
| | | | TRHGIRIITRPKLLLAS |
| 157 | myeloid antimicrobial | Bos taurus | METQRASLSLGRWSLWLLLLGLALP |
| | peptide BMAP-28 | | SASAQALSYREAVLRAVDQLNEKSS |
| | | | EANLYRLLELDPPPKEDDENPNIPKP |
| | | | VSFRVKETVCPRTSQQSPEQCDFKEN |
| | | | GLLKECVGTVTLDQVGSNFDITCAVP |
| | | | QSVGGLRSLGRKILRAWKKYGPIIVPI |
| | | | IRIG |
| 158 | myeloid cathelicidin 1 | Equus | METQRNTRCLGRWSPLLLLLGLVIPP |
| | | caballus | ATTQALSYKEAVLRAVDGLNQRSSD |
| | | | ENLYRLLELDPLPKGDKDSDTPKPVS |
| | | | FMVKETVCPRIMKQTPEQCDFKENG |
| | | | LVKQCVGTVILGPVKDHFDVSCGEP |
| | | | QRVKRFGRLAKSFLRMRILLPRRKIL |
| | | | LAS |
| 159 | SMAP 29 | Ovis aries | METQRASLSLGRCSLWLLLLGLALPS |
| | | | ASAQVLSYREAVLRAADQLNEKSSE |
| | | | ANLYRLLELDPPPKQDDENSNIPKPV |
| | | | SFRVKETVCPRTSQQPAEQCDFKENG |
| | | | LLKECVGTVTLDQVRNNFDITCAEPQ |
| | | | SVRGLRRLGRKIAHGVKKYGPTVLRI |
| | | | IRIAG |
| 160 | BNP-1 | Bos taurus | RLCRIVVIRVCR |
| 161 | HNP-1 | Homo | ACYCRIPACIAGERRYGTCIYQGRLW |
| | | sapiens | AFCC |
| 162 | HNP-2 | Homo | CYCRIPACIAGERRYGTCIYQGRLWA |
| | | sapiens | FCC |
| 163 | HNP-3 | Homo | DCYCRIPACIAGERRYGTCIYQGRLW |
| | | sapiens | AFCC |
| 164 | HNP-4 | Homo | VCSCRLVFCRRTELRVGNCLIGGVSF |
| | | sapiens | TYCCTRV |
| 165 | NP-1 | Oryctolagus | VVCACRRALCLPRERRAGFCRIRGRI |
| | | cuniculus | HPLCCRR |
| 166 | NP-2 | Oryctolagus | VVCACRRALCLPLERRAGFCRIRGRI |
| | | cuniculus | HPLCCRR |
| 167 | NP-3A | Oryctolagus | GICACRRRFCPNSERFSGYCRVNGAR |
| | | cuniculus | YVRCCSRR |
| 168 | NP-3B | Oryctolagus | GRCVCRKQLLCSYRERRIGDCKIRGV |
| | | cuniculus | RFPFCCPR |
| 169 | NP-4 | Oryctolagus | VSCTCRRFSCGFGERASGSCTVNGGV |
| | | cuniculus | RHTLCCRR |
| 170 | NP-5 | Oryctolagus | VFCTCRGFLCGSGERASGSCTINGVR |
| | | cuniculus | HTLCCRR |
| 171 | RatNP-1 | Rattus | VTCYCRRTRCGFRERLSGACGYRGRI |
| | | norvegicus | YRLCCR |
| 172 | Rat-NP-3 | Rattus | CSCRYSSCRFGERLLSGACRLNGRIY |
| | | norvegicus | RLCC |
| 173 | Rat-NP-4 | Rattus | ACTCRIGACVSGERLTGACGLNGRIY |
| | | norvegicus | RLCCR |
| 174 | GPNP | Guinea pig | RRCICTTRTCRFPYRRLGTCIFQNRVY |
| | | | TFCC |
| 175 | beta defensin-3 | Homo | GIINTLQKYYCRVRGGRCAVLSCLPK |
| | | sapiens | EEQIGKCSTRGRKCCRRKK |
| 176 | theta defensin-1 | Macaca | RCICTRGFCRCLCRRGVC |
| | | mulatta | |
| 177 | defensin CUA1 | Helianthus | MKSSMKMFAALLLVVMCLLANEMG |
| | | annuus | GPLVVEARTCESQSHKFKGTCLSDTN |
| | | | CANVCHSERFSGGKCRGFRRRCFCTT |
| | | | HC |
| 178 | defensin SD2 | Helianthus | MKSSMKMFAALLLVVMCLLANEMG |
| | | annuus | GPLVVEARTCESQSHKFKGTCLSDTN |
| | | | CANVCHSERFSGGKCRGFRRRCFCTT |
| | | | HC |
| 179 | neutrophil defensin 2 | Macaca | ACYCRIPACLAGERRYGTCFYMGRV |
| | | mulatta | WAFCC |
| 180 | 4 KDA defensin | Androctonus | GFGCPFNQGACHRHCRSIRRRGGYC |
| | | australis | AGLFKQTCTCYR |
| | | hector | |
| 181 | defensin | Mytilus | GFGCPNNYQCHRHCKSIPGRCGGYC |
| | | galloprovin | GGXHRLRCTCYRC |
| | | cialis | |
| 182 | defensin AMP1 | Heuchera | DGVKLCDVPSGTWSGHCGSSSKCSQ |
| | | sanguinea | QCKDREHFAYGGACH |
| | | | YQFPSVKCFCKRQC |
| 183 | defensin AMP1 | Clitoria | NLCERASLTWTGNCGNTGHCDTQCR |
| | | ternatea | NWESAKHGACHKRGN |
| | | | WKCFCYFNC |
| 184 | cysteine-rich | Mus | MKKLVLLFALVLLAFQVQADSIQNT |
| | cryptdin-1 homolog | musculus | DEETKTEEQPGEKDQAVSVSFGDPQ |
| | | | GSALQDAALGWGRRCPQCPRCPSCP |
| | | | SCPRC PRCPRCKCNPK |
| 185 | beta-defensin-9 | Bos taurus | QGVRNFVTCRINRGFCVPIRCPGHRR |
| | | | QIGTCLGPQIKCCR |
| 186 | beta-defensin-7 | Bos taurus | QGVRNFVTCRINRGFCVPIRCPGHRR |
| | | | QIGTCLGPRIKCCR |
| 187 | beta-defensin-6 | Bos taurus | QGVRNHVTCRIYGGFCVPIRCPGRTR |
| | | | QIGTCFGRPVKCCRRW |
| 188 | beta-defensin-5 | Bos taurus | QVVRNPQSCRWNMGVCIPISCPGNM |
| | | | RQIGTCFGPRVPCCR |
| 189 | beta-defensin-4 | Bos taurus | QRVRNPQSCRWNMGVCIPFLCRVGM |
| | | | RQIGTCFGPRVPCCRR |
| 190 | beta-defensin-3 | Bos taurus | QGVRNHVTCRINRGFCVPIRCPGRTR |
| | | | QIGTCFGPRIKCCRSW |
| 191 | beta-defensin-10 | Bos taurus | QGVRSYLSCWGNRGICLLNRCPGRM |
| | | | RQIGTCLAPRVKCCR |
| 192 | beta-defensin-13 | Bos taurus | SGISGPLSCGRNGGVCIPIRCPVPMRQ |
| | | | IGTCFGRPVKCCRSW |
| 193 | beta-defensin-1 | Bos taurus | DFASCHTNGGICLPNRCPGHMIQIGIC |
| | | | FRPRVKCCRSW |
| 194 | coleoptericin | Zophobas | SLQGGAPNFPQPSQQNGGWQVSPDL |
| | | atratus | GRDDKGNTRGQIEIQNKGKDHDFNA |
| | | | GWGKVIRGPNKAKPTWHVGGTYRR |
| 195 | beta defensin-3 | Homo | GIINTLQKYYCRVRGGRCAVLSCLPK |
| | | sapiens | EEQIGKCSTRGRKCCRRKK |
| 196 | defensin C | Aedes | ATCDLLSGFGVGDSACAAHCIARGN |
| | | aegypti | RGGYCNSKKVCVCRN |
| 197 | defensin B | Mytilus | GFGCPNDYPCHRHCKSIPGRYGGYC |
| | | edulis | GGXHRLRCTC |
| 198 | sapecin C | Sarcophaga | ATCDLLSGIGVQHSACALHCVFRGN |
| | | peregrina | RGGYCTGKGICVCRN |
| 199 | macrophage antibiotic | Oryctolagus | MRTLALLAAILLVALQAQAEHVSVSI |
| | peptide MCP-1 | cuniculus | DEVVDQQPPQAEDQDVAIYVKEHES |
| | | | SALEALGVKAGVVCACRRALCLPRE |
| | | | RRAG FCRIRGRIHPLCCRR |
| 200 | cryptdin-2 | Mus | MKPLVLLSALVLLSFQVQADPIQNTD |
| | | musculus | EETKTEEQSGEEDQAVSVSFGDREGA |
| | | | SLQEESLRDLVCYCRTRGCKRRERM |
| | | | NGT CRKGHLMYTLCC |
| 201 | cryptdin-5 | Mus | MKTFVLLSALVLLAFQVQADPIHKT |
| | | musculus | DEETNTEEQPGEEDQ |
| | | | AVSISFGGQEGSALHEELSKKLICYCR |
| | | | IRGCKRRERVFGT CRNLFLTFVFCCS |
| 202 | cryptdin 12 | Mus | LRDLVCYCRARGCKGRERMNGTCR |
| | | musculus | KGHLLYMLCCR |
| 203 | defensin | Pyrrhocoris | ATCDILSFQSQWVTPNHAGCALHCVI |
| | | apterus | KGYKGGQCKITVCHCRR |
| 204 | defensin R-5 | Rattus | VTCYCRSTRCGFRERLSGACGYRGRI |
| | | norvegicus | YRLCCR |
| 205 | defensin R-2 | Rattus | VTCSCRTSSCRFGERLSGACRLNGRI |
| | | norvegicus | YRLCC |
| 206 | defensin NP-6 | Oryctolagus | GICACRRRFCLNFEQFSGYCRVNGAR |
| | | cuniculus | YVRCCSRR |
| 207 | beta-defensin-2 | Pan | MRVLYLLFSFLFIFLMPLPGVFGGISD |
| | | troglodytes | PVTCLKSGAICHP |
| | | | VFCPRRYKQIGTCGLPGTKCCKKP |
| 208 | beta-defensin-2 | Homo | GIGDPVTCLKSGAICHPVF CPRRYKQI |
| | | sapiens | GTCGLPGTKCCKKP |
| 209 | beta-defensin-1 | Homo | MRTSYLLLFTLCLLLSEMASGGNFLT |
| | | sapiens | GLGHRSDHYNCVSS |
| | | | GGQCLYSACPIFTKIQGTCYRGKAKC |
| | | | CK |
| 210 | beta-defensin-1 | Capra | MRLHHLLLVLFFLVLSAGSGFTQGIR |
| | | hircus | SRRSCHRNKGVCAL |
| | | | TRCPRNMRQIGTCFGPPVKCCRKK |
| 211 | beta defensin-2 | Capra | MRLHHLLLALFFLVLSAGSGFTQGII |
| | | hircus | NHRSCYRNKGVCAP |
| | | | ARCPRNMRQIGTCHGPPVKCCRKK |
| 213 | defensin-3 | Macaca | MRTLVILAAILLVALQAQAEPLQART |
| | | mulatta | DEATAAQEQIPTDNPEVVVSLAWDE |
| | | | SLAPKDSVPGLRKNMACYCRIPACL |
| | | | AGER RYGTCFYRRRVWAFCC |
| 214 | defensin-1 | Macaca | MRTLVILAAILLVALQAQAEPLQART |
| | | mulatta | DEATAAQEQIPTDNPEVVVSLAWDE |
| | | | SLAPKDSVPGLRKNMACYCRIPACL |
| | | | AGER RYGTCFYLGRVWAFCC |
| 215 | neutrophil defensin 1 | Mesocricetus | VTCFCRRRGCASRERHIGYCRFGNTI |
| | | auratus | YRLCCRR |
| 216 | neutrophil defensin 1 | Mesocricetus | CFCKRPVCDSGETQIGYCRLGNTFYR |
| | | auratus | LCCRQ |
| 217 | Gallinacin 1-alpha | Gallus | GRKSDCFRKNGFCAFLKCPYLTLISG |
| | | gallus | KCSRFHLCCKRIW |
| 218 | defensin | Allomyrina | VTCDLLSFEAKGFAANHSLCAAHCL |
| | | dichotoma | AIGRRGGSCERGVCICRR |
| 219 | neutrophil cationic | Cavia | RRCICTTRTCRFPYRRLGTCIFQNRVY |
| | peptide 1 | porcellus | TFCC |

The peptidoglycan hydrolases are enzyme typically with sequences of 100-550 aa, thus the following table provides Genbank indices for a selection of peptidoglycan hydrolases but not their full sequences. Several thousand bacterial phage peptidoglycan hydrolases have been described (Vollmer et a,l FEMS Microbiol Rev 32 (2008) 259-286). The listing in the table should be considered exemplary but not limiting. Similarly the sequences listed may comprise the precursor or the active domains and may be used in whole or in part.

TABLE 3

| | | |
|---|---|---|
| Lysostaphin (Glycyl- | Staphylococcus simulans | GI: 3287967 |
| glycine endopeptidase) | biovar staphylolyticus | |
| B30 endolysin | Streptococcus phage B30 | GI: 31407686 |
| Zoocin A | Streptococcus equi subsp. | GI: 194371883 |
| | zooepidemicus | |
| Zoocin A | Streptococcus agalactiae | GI: 529474642 |
| | MRI Z1-038 | |
| lysozyme | Escherichia coli ‘BL21- | GI: 253774252 |
| | Gold(DE3)pLysS AG’ | |
| Phage lysin, glycosyl | Streptococcus suis phage | GI: 505459466 |
| hydrolase, family 25 | | |
| Streptococcus phage | Streptococcus phage | GI: 451937399 |
| phiS10 | | |
| lysin PlyGBS | Streptococcus phage | GI: 41078771 |
| phage lysin N- | Bacillus phage WBeta | GI: 85701396 |
| acetylmuramoyl-L-alanine | | |
| amidase | | |
| N-acetylmuramoyl-L- | E. coli phage | GI: 190906800 |
| alanine amidase AmiA | | |
| N-acetylmuramoyl-L- | E coli phage | GI: 194421544 |
| alanine amidase AmiB | | |
| Membrane-bound lytic | Escherichia coli YfhD | GI: 189047094 |
| murein transglycosylase F | | |

Antimicrobial peptides are therefore a promising option in the development of novel anti-infective strategies. Many attempts have been made, with mixed results, to use antimicrobial peptides as systemic antimicrobial agents, both in natural form and as modifications. Several such peptides have entered Phase 3 clinical trials and a number of related peptide products are in development (18). Generally, doses needed are high and toxicity is a problem. Cost of manufacture has also been a significant challenge. In some cases the short in vivo half-life is insufficient (42). Nevertheless, given their broad spectrum and ubiquity, there has been an on-going interest in potential applications of antimicrobial peptides as antibiotics (18) if these challenges can be overcome.

Antibodies to Staphylococcus

Antibodies to Staphylococcus have demonstrated efficacy in vivo (43) but are difficult to prepare given the vary variable genome of S. aureus and the prevalence of epitopes which are non neutralizing.

Staphylococcus aureus Strains

S. aureus comprise a large and diverse group of strains, including both antibiotic resistant and antibiotic susceptible strains. This is evidenced by the cataloguing of genomes by Patric (http://patricbrc.org/) which currently documents 260 genomes of which 121 are complete, and the cataloguing by NARSA (Network on Antimicrobial Resistance in Staphylococcus aureus www.narsa.net) of over 200 antibiotic resistant strains.

S. aureus is prone to form biofilms bringing multiple strains of S. aureus into close proximity with each other and other bacteria. While recombination between Staphylococci is not as prolific as with other bacteria there is a high degree of diversity (44, 45).

A consideration in selecting an antibody for targeting S. aureus is therefore to select an antigen that is derived from a gene that is conserved across most or all Staphylococcus spp. A further consideration is to select epitopes that are not only conserved but which are exposed on the outer surface of the bacterium at some point in its life cycle. Two criteria can be used as indicative of surface exposed proteins. Bacterial proteins with single transmembrane helices necessarily have a part of the protein exposed. Secondly proteins with LPxTG motifs were identified. This indicates a sortase cleavage site which leads to binding of the protein to the surface peptidoglycan (46).

Applications

The inventions comprised herein include novel antibodies directed to bacterial epitopes, novel antibodies fused with microbiocides and dual microbiocides linked by a polypeptide, including but not limited to immunoglobulin molecule linkers.

The immunoglobulins in each of the above groups of inventions may be from the group comprising, but not limited to, a murine immunoglobulin, a human immunoglobulin or a human-murine chimeric immunoglobulin. In some embodiments said antibody is a complete immunoglobulin G comprising two heavy chains and two light chains; in other embodiments other isotypes of immunoglobulin are employed, or fragments or multiples of immunoglobulin molecules. In yet other embodiments other configurations of synthetic constructs which comprise the antibody variable region are employed.

In some instances the epitopes targeted and microbiocides delivered are selected such that the microbiocidal effect is synergistic with that of antibiotics. For instance in one embodiment, as a non-limiting example, methicillin is supplemented by the antibody fusion microbiocide effective against methicillin resistant S. aureus, and the resultant control of microbial growth is enhanced. Other antibiotics may be employed contemporaneously with the antibody fusion microbiocide including for instance, gentamycin, ciprofloxacin, and vancomycin as examples.

Proteins were selected based on their conservation across multiple strains of Staphylococcus based on FigFam analysis. Each individual FIGfam is a set of protein sequences, along with a decision procedure. All of the protein sequences that make up a single FIGfam are believed to implement the same functional role, and all of the sequences are easily recognizably similar over at least 70% of the length of the protein sequences (http://www.nmpdr.org/FIG/wiki/view.cgi/FIG/FIGfamDescription). Epitopes located in surface proteins were characterized and selected to be used to immunize mice to create the antibodies of the present invention. Epitope characterization was performed using a principal component based in silico prediction system described in U.S. patent application Ser. No. 13/052,733 and PCT/US2012/055038, each of which is incorporated herein by reference in its entirety.

In some embodiments the epitope targeted is conserved not only in S. aureus but also on other pathogenic Staphylococci such as S. epidermidis, S. pseudintermedius, S. intermedius, S. hycius, S. lugdunensis, and S. saprophyticus. In some instances the peptide epitopes of interest are conserved beyond Staphylococcus in other bacterial genera such as Streptococcus and Clostridium spp. In yet other embodiments the epitope targeted is from another bacterial genera such as Streptococcus, Enterococcus, Clostridium or another bacterial genus of interest. Hence antimicrobial compounds may provide for efficacy against a broader range of bacteria.

In some instances immunization for production of hybridomas was achieved by administering the peptide epitope of interest as a fusion with a longer poly peptide or polypeptides. In some instances said polypeptide fusion is an immunoglobulin. Other embodiments are possible. In other instances the whole protein or a combination of proteins are used in the immunization and a specific peptide is used to detect and select antibodies to the peptide epitope of interest. In some cases, an adjuvant may be used in mimmunization and such adjuvant may be drawn, for example, form the group comprising Sigma Adjuvant System (S6322), unmethylated murine stimulatory CpG motif (Invivogen, San Diego), Titermax (TiterMax USA, Inc, Norcross, Ga.), Freund's Complete Adjuvants.

In preparation of dual microbiocides tethered together, the length of the linking molecule and its ease of expression are useful attributes. In some preferred embodiments a polypeptide of 100 to 500 amino acids is used as the linker. In some preferred embodiments the linking polypeptide is an immunoglobulin without any particular binding specificity for Staphylococcus, although specificity is an additive benefit. In yet other embodiments other polypeptides may be used as a linker including, but not limited to, albumin.

The antimicrobial compounds described herein share many potential applications, whether they be antibodies, antibody fusions, or tethered dual microbiocides. These applications are described below, grouping all of the above and referring to them as “the antimicrobial compounds”.

In one embodiment the antimicrobial compounds may be applied parenterally to treat or prevent infection by a bacterial (e.g., Staphylococcus spp., especially S. aureus), viral (e.g., influenza virus), or protozoan pathogens (e.g., Cryptosporidium). Such application may be intravenous, intramuscular, subcutaneous or intraperitoneally or by any other parenteral route. In a preferred embodiment the antimicrobial compound is applied topically either to skin, to a mucosal surface or to the surface of an incision or wound. Staphylococcal infections may affect the eye and in another preferred embodiment the antimicrobial compound is applied to the eye or to the surface of objects contacting the eye including but not limited to contact lenses.

For topical and mucosal application the antibody fusion microbiocide may be incorporated into a spray-on solution, cream, gel or aqueous solution, or contained in a suppository, tampon, or pessary. The antimicrobial compound may be applied per os to treat or prevent infection with Staphylococcus aureus or another bacterial species. Such delivery may be by means of a solution or a powdered preparation encased in an enteric capsule to be swallowed and deliver the antimicrobial compound to the intestinal mucosa. It may alternatively be by delivery of liquid drops to the buccal mucosa or by incorporation in a gel or starch substrate chewable or suckable lozenge or strip intended to be retained in the mouth. The antimicrobial compound may also be delivered by means of ear drops to treat otitis infections.

The antimicrobial compound may be applied prophylactically. For instance in anticipation of surgery, a topical cream or spray containing said product may be applied around the intended incision site, or a dose may be administered parenterally on admission to hospital. The antimicrobial compound may be administered during surgery by incorporation into a coating on a prosthesis or by inclusion in a cavity filler (e.g., a gel wound filler). The antimicrobial compound may be used to treat infections with Staphylococcus, including MRSA, which arise naturally or nosocomially. This application should not however be considered limiting as in some embodiments the composition may be used to treat other bacterial infections.

While the above examples address applications in human medicine this should not be considered limiting. Indeed, there are a wide array of veterinary applications which mirror the applications in humans. Among the examples of specific veterinary applications are in the prophylaxis and treatment of skin infections with Staph pseudintermedius, an organism which shares conserved proteins with S. aureus and may be antibiotic resistant. A further example is in the treatment of mastitis caused by S. aureus or Streptococcus spp. In this instance the antimicrobial compound may be administered parenterally, by intramammary infusion, topically at another mucosal site or by application to the exterior surface of the teat as a teat dip. In each of these non-limiting examples the application may be either prophylactic or therapeutic.

The aforementioned examples of routes of administration to a subject are illustrative examples and should not be considered limiting. While these examples apply to use in a living subjects, the antimicrobial compound may also be applied to inanimate objects, wherein said objects may be, but are not limited to, biomedical devices, prostheses, dressings, surgical wraps or work surfaces or any other object in a hospital environment. Further embodiments are in the control of community associated infection where surfaces in gymnasiums, locker rooms, residential facilities or in any other highly trafficked areas may be treated.

In one particular desired embodiment the antimicrobial compound is applied as a coating. Said coating may be layered on the exterior or other surface of a prosthesis such as a hip replacement or a breast implant, to a cardiovascular device such as a stent or pacemaker or to another biomedical device such as an endoscope, bone plate or screw. It may be applied directly as a component of a coating applied to the surface of fractured or reconstructed bone. A coating containing the antimicrobial compound may be applied to a bandage, wound dressing or suture material.

The coating may be comprised of any number of materials compatible with their use in a living organism, including but not limited to a calcium based material such as calcium phosphate or hydroxylapatite, ceramic, silica, a polyvinyl alcohol, polyvinyl chloride, polyacrilamide or other polymeric coating material. In yet other embodiments a protein coating is used for example albumin, heparin, fibronectin, or collagen. Another coating which may be used is hyaluronic acid.

Under some circumstances an extended release of the antimicrobial compound may be desired to provide protection over time. This may be achieved by application of the fusion protein as a coating of nanoparticles, fibers, or by inclusion in a porous material such as silica or hydroxylapatite.

In another preferred embodiment the antibody fusion microbiocide is incorporated in a filler. Said filler may be a gel or jelly or paste used to fill a void in a wound or surgical field or prosthesis. Some of the materials which are used as fillers and into which the antibody fusion microbiocide can be introduced include, but are not limited to various polymeric materials and gums.

As is the case for treatment of a subject, the use of the antimicrobial compound as a treatment or coating for an object may be combined with antibiotic treatment. Hence coatings of devices may comprise both the antimicrobial compound and one or more antibiotics.

### EXAMPLES

### Example 1

### Selection of Conserved Surface Exposed Proteins from Staphylococcus Spp. and Epitopes Therein

Eighty four genomes of Staphylococcus aureus were assembled from the Patric database (http://patricbrc.org/). Genomes for all proteins were assembled and FigFams for each analyzed. FigFams describe functional features of each protein. Unique FigFams were identified and a master list for all proteins assembled. Using a missing data pattern, the master list was reviewed to determine that no superfluous FigFams were present. Cross referencing of FigFams from each Staphylococcus isolate identified FigFams present or absent. Using this process FigFams conserved in all isolates were identified.

In order to identify proteins which are surface exposed, two groups were then assembled. The first had a single transmembrane helix (TMH) which was determined by searching consensus sequences using topological program (examples of programs which are applicable include but are not limited to Phobius® and Memsat®). Proteins with a single TMH were considered to have an extracellular component. From 4588 unique FigFams in the 84 Staphylococcus strains, 1779 FigFams are conserved in all 84 strains; of those conserved, 81 FigFams were determined to have a single TMH. Secondly, to identify proteins which are anchored to the cell wall envelope by a transpeptidation mechanism and which are characterized by a sortase cleavage site, we searched conserved FigFams for a LPxTG motif Of the 1779 conserved FigFams, 10 bore a LPxTG motif.

We have previously described a bioinformatics approach to identification of conserved epitopes (uTOPE filing incorporated by reference). The bioinformatics analysis was applied to the 91 proteins identified by FigFam analysis. Regions of these proteins which were predicted to be epitope dense, comprising B cell epitopes, close to or overlapping with MHC binding sites were identified. These are known as coincident epitope groups or CEGs. Peptides were identified to include and span CEGS and adjacent flanking regions. These were selected for preparation of hybridomas and are shown in Table 4 below.

Proteins included in those from which CEGs were selected included penicillin binding proteins, iron sensitive determinants (Isd) and adhesins. This list is included to provide examples of classes of proteins and should not be considered limiting or restrictive.

TABLE 4

| Protein source of | | | |
|---|---|---|---|
| peptides | Peptide SEQ | Link | GI |
| penicillin-binding | KDVVNRNQATDPHPTG | http://www.ncbi.nhn.nih.gov/ | 57650405 |
| protein 2 | SSLKPFLAYGPAIENMK | protein/57650405 | |
| | WATNHAIQDESSYQVD | | |
| | GSTFRNYDTKSHGTV | | |
| | (SEQ ID NO: 93) | | |
| LPXTG cell wall | VKMTNAGQSVTYYFTD | http://www.ncbi.nhn.nih.gov/ | 57652394 |
| surface anchor | VKAPTVTVGNQTIEVG | protein/57652394 | |
| protein | KTMNPIVLTTTDNGTG | | |
| | TVTNTVTGLPSGLSYDS | | |
| | ATNSIIGTPTKIGQSTVT | | |
| | (SEQ ID NO: 94) | | |
| Penicillin-binding | KMDTKKITERDKKDFW | http://www.ncbi.nlm.nih.gov/ | 57651945 |
| protein 3 | IQLHPKKAKAMMTKEQ | protein/57651945 | |
| | (SEQ ID NO: 95) | | |
| Penicillin-binding | MVDEPLHFQGGLTKRS | http://www.ncbi.nlm.nih.gov/ | 57651945 |
| protein 3 | YFNKNGHVTINDKQAL | protein/57651945 | |
| | MHSSNVYMFKTALKLA | | |
| | GDPYYSGMALPSDISSP | | |
| | AQK (SEQ ID NO: 224) | | |
| sdrC protein, C- | TTETDENGKYRFDNLD | http://www.ncbi,nlm.nih.gov/ | 57651437 |
| terminus of | SGKYKVIFEKPAGLTQT | protein/57651437 | |
| bacterial | GTNTTEDDKDADGGE | | |
| fibrinogen-binding | (SEQ ID NO: 96) | | |
| adhesin | | | |
| Fibronectin-binding | GLGTENGHGNYDVIEEI | http://www.ncbi,nlm.nih.gov/ | 57651010 |
| protein A | EENSHVDIKSELGYEGG | protein/57651010 | |
| | QNSGNQSFEEDTEEDKP | | |
| | KYEQGGNIVDIDFDSVP | | |
| | (SEQ ID NO: 97) | | |
| Trans-sulfuration | AALPEEVRQERGITFGL | http://www.ncbi.nlm.nih.gov/ | 57652618 |
| enzyme family | FRLSVGLEDPDELIADI | protein/57652618 | |
| protein | (SEQ ID NO: 98) | | |
| Cell wall associated | DGETTPITKTATYKVVR | http://www.ncbi.nlm.nih.gov/ | 57651379 |
| fibronectin-binding | TVPKHVFETARGVLYP | protein/57651379 | |
| protein | GVSDMYDAKQYVKPV | | |
| | NNSWSTN (SEQ ID | | |
| | NO: 99) | | |
| capsular | VVLSPILLITALLIKMES | http://www.ncbi.nlm.nih.gov/ | 57651165 |
| polysaccharide | PGPAIFKQKRPTINNELF | protein/57651165 | |
| biosynthesis | NIYKFRSMKIDTPNV | | |
| galactosyltrans- | (SEQ ID NO: 100) | | |
| ferase Cap5M | | | |
| Multimodular | KNGNNGGKSNSKKNR | ZP_06335586.1 | 282927977 |
| transpeptidase- | NVKRTIIKIIGFMIIAFFV | | |
| transglycosylase / | VLLLGILLFAYYAWKA | | |
| Penicillin-binding | PAFTEAKLQDPIPAK | | |
| protein 1A/1B | (SEQ ID NO: 101) | | |
| (PBP1) | | | |
| Multimodular | LATEDNRFYEHGALDY | ZP_06335586.1 | 282927977 |
| transpeptidase- | KRLFGAIGKNLTGGFGS | | |
| transglycosylase / | EGASTLTQ (SEQ ID | | |
| Penicillin-binding | NO: 102) | | |
| protein 1A/1B | | | |
| (PBP1) | | | |
| Multimodular | QEYSKDDIFQVYLNKIY | ZP_06335586.1 | 282927977 |
| transpeptidase- | YSDGVTGIKA (SEQ ID | | |
| transglycosylase / | NO: 103) | | |
| Penicillin-binding | | | |
| protein 1A/1B | | | |
| (PBP1) | | | |
| Multimodular | NRNQATDPHPTGSSLKP | ZP_06335586.1 | 282927977 |
| transpeptidase- | FLAYGPAIENMKWATN | | |
| transglycosylase / | HAIQDESSYQVDGSTFR | | |
| Penicillin-binding | NYDTKSHGTV (SEQ ID | | |
| protein 1A/1B | NO: 104) | | |
| (PBP1) | | | |
| D-alanyl-D-alanine | ELSNTKLYPGQVWTIA | http://www.ncbi.nlm.nih.gov/ | 57651483 |
| carboxypeptidase / | DLLQITVSNSSNAA | protein/57651483 | |
| Penicillin binding | (SEQ ID NO: 105) | | |
| protein PBP4 | | | |
| Cell surface | VIELGLKTASTWKKFE | EJU82039.1 | 402346969 |
| receptor IsdH for | VYE (SEQ ID NO: 106) | | |
| hemoglobin- | | | |
| haptoglobin | | | |
| complexes | | | |
| Cell surface | SEENSESVMDGFVEHPF | EJU82039.1 | 402346969 |
| receptor IsdH for | YTATLNGQKYVVMKT | | |
| hemoglobin- | KDDSYWKDLIVEGKRV | | |
| haptoglobin | TTVSKDPKNNSRTLIFP | | |
| complexes | YIPDKAVYNAIVKVVV | | |
| | A (SEQ ID NO: 107) | | |
| Cell division | EIVQNTINKRINFIFGVI | YP_005297996.1 | 379021334 |
| protein FtsI | VFIFAVLVLRLGYLQIA | | |
| [Peptidoglycan | QGSHYKQI (SEQ ID | | |
| synthetase] | NO: 108) | | |
| Cell division | QNGEPRVNSTYIGYAPI | YP_005297996.1 | 379021334 |
| protein FtsI | DDPKLAFSIVYTNQPVP | | |
| [Peptidoglycan | PPWLTGGDLG (SEQ ID | | |
| synthetase] | NO: 109) | | |
| FtsZ-interacting | DTQDIADELKNRRATL | YP_004149052.1 | 319892177 |
| protein related to | VNLQRIDKVSAKRIIDF | | |
| cell division | LSGTVYAIGGDIQRV | | |
| | (SEQ ID NO: 110) | | |
| Penicillin-binding | IQDRKIKKVSKNKKRV | AEQ76893.1 | 353260578 |
| protein PBP2a | DAQYKIKTNYGNIDRN | | |
| | VQFNFVKEDGMWKLD | | |
| | WDHSVIIPGMQKDQSIH | | |
| | IENLKSERGKIL (SEQ ID | | |
| | NO: 111) | | |
| Penicillin-binding | AHTLIEKKKKDGKDIQL | AEQ76893.1 | 353260578 |
| protein PBP2a | TIDAKVQKSIYNNMKN | | |
| | DYGSGTAIHPQTGELLA | | |
| | LVSTPSYDVYPFMYGM | | |
| | SNEEYNKLTEDKKEPLL | | |
| | NKFQITTSPGSTQKILTA | | |
| | (SEQ ID NO: 112) | | |
| Penicillin-binding | IHPQTGELLALVSTPSY | ACO24829.1 | 225729844 |
| protein PBP2a | DVYPFMYGMSNEEYN | | |
| | KLTEDKKEPLLNKFQIT | | |
| | TSPGSTQKILTAMIGLN | | |
| | NKTLDDKTSYKIDGKG | | |
| | WQKDKSWGGYNVTRY | | |
| | EVVNGNIDLKQAIESSD | | |
| | NIFFARVALELGSKKFE | | |
| | KGMKKLGVGEDIPSDY | | |
| | PFYNAQISNKNLDNEIL | | |
| | LADSGYGQGEILINPVQ | | |
| | ILSIYSALENNGNINAPH | | |
| | LLKDTKNKVWKKNIIS | | |
| | KENINLLTDGMQQVVN | | |
| | KTHKEDIYRSYANLIGK | | |
| | SGTAELK (SEQ ID | | |
| | NO: 113) | | |
| Penicillin-binding | NNKTLDDKTSYKIDGK | ACO24829.1 | 225729844 |
| protein PBP2a | GWQKDKSWGGYNVTR | | |
| | YEVVNGNIDLKQAIESS | | |
| | DNIFFARVALELGSKKF | | |
| | EKGMKK (SEQ ID | | |
| | NO: 114) | | |
| Penicillin-binding | KLGVGEDIPSDYPFYNA | AEQ76893.1 | 353260578 |
| protein PBP2a | QISNKNLDNEILLADSG | | |
| | YGQGEILINPVQILSIYS | | |
| | ALENNGNIN (SEQ ID | | |
| | NO: 115) | | |
| Penicillin-binding | IGKSGTAELKMKQGET | AEQ76893.1 | 353260578 |
| protein PBP2a | GRQIGWFISYDKDNPN | | |
| | MMMAINVKDVQDKG | | |
| | MASYNAKISG (SEQ ID | | |
| | NO: 116) | | |
| Penicillin-binding | LILIVVVVGFGIYFYAS | AEQ76893.1 | 353260578 |
| protein PBP2a | KDKEINNT (SEQ ID | | |
| | NO: 117) | | |
| Penicillin-binding | VSTPSYDVYPFMYGMS | ACO24829.1 | 225729844 |
| protein PBP2a | NEEYNKLTEDKKEPL | | |
| | (SEQ ID NO: 118) | | |
| SA00645-IsdB - | ETTNDDYWKDFMVEG | http://www.ncbi.nlm.nih.gov/ | 57651738 |
| COL-ctrl | QRVRTISKDAKNNTRTI | protein/57651738 | |
| | IFPYVEGKTLYDAIVKV | | |
| | HVKTIDYDGQYH (SEQ | | |
| | ID NO: 119) | | |
| Heme transporter | KQKIENKAAKQKKHPK | http://www.ncbi.nlm.nih.gov/ | 365167023 |
| IsdDEF, lipoprotein | VLILMGVPGSYLVATD | protein/365167023?report= | |
| IsdE | KSYI (SEQ ID NO: 120) | genbank&log5=prottop&blast_rank | |
| | | =1&RID=7GNCY53W01N | |
| Cell division | EIVQNTINKRINFIFGVI | http:/www.ncbi.nlm.nih.gov/ | 379021334 |
| protein FtsI | VFIFAVLVLRLGYLQIA | protein/379021334?report= | |
| [Peptidoglycan | QGSHYKQI (SEQ ID | genbank&log$=prottop&blast_rank | |
| synthetase] | NO: 121) | =8&RID=7GHWV4Y601N | |
| Cell division | QNGEPRVNSTYIGYAPI | http:/www.ncbi.nlm.nih.gov/ | |
| protein FtsI | DDPKLAFSIVYTNQPVP | protein/379021334?report= | 379021334 |
| [Peptidoglycan | PPWLTGGDLG (SEQ ID | genbank&log$=prottop&blast_rank | |
| synthetase] | NO: 122) | =8&RID=7GHWV4Y601N | |
| Cell division | FFNDGNMLKPWFVNSV | http://wvww.ncbi.nlm.nih.gov/ | |
| protein FtsI | ENPVSK (SEQ ID | protein/386728860?report= | |
| [Peptidoglycan | NO: 123) | genbank&log$=prottop&blast_rank | |
| synthetase] | | =26&RID=7S9EHHXS01N | |

### Preparation of Immunogens

Two approaches can be taken to developing recombinant antibodies to the specific peptides of interest. In one approach the specific peptide, alone or mounted in an appropriate carrier or fusions, for instance incorporating an Fc fusion, is used as the immunogen. In a second approach a whole bacterial lysate suspension or a bacterial surface protein preparation using partial Trypsin digestion is used as the immunogen and the resultant antibodies selected by using the specific peptide of interest. Either of these approaches can lead to the desired end result of a hybridoma secreting antibodies targeting the peptide of interest, and said antibody can then be engineered into a recombinant form.

### Production of Hybridomas

Mice are inoculated subcutaneously in one or both of their hocks as described by Kamala (Kamala T. Hock immunization: a humane alternative to mouse footpad injections. J Immunol Methods 2007; 328(1-2): 204-14). Alternatively, a number of other commonly used injection sites such as base of tail, neck, foot pad, intraperitoneal, intravenous etc can be used for the immunization. A volume consistant with the maximum injectable volume for the route chosen is injected using a 27 g needle. For hock injections up to 50 microliters can be injected. An initial inoculation on Day 0 is followed by 3-4 boost injections in 2-3 week intervals, depending on seroconversion of the animals. Blood samples are collected via maxilliary vein puncture 7 days after each booster to monitor antigen-specific antibody titer. Antibody titers are determined via whole cell ELISA using fixed S. aureus cells. Acceptable antibody titers are at least 25,000-fold above pre-immunization levels, preferably greater than 200,000. Four days after the last booster, mice are sacrificed by CO2 asphyxiation.

Following euthanasia harvesting of iliac and inguinal lymph nodes as well as the spleen is performed as described by Van den Broeck et al (47).

Production of hybridoma lines is done following the methods initially described by Kohler and Milstein Nature 1975 Aug. 7; 256(5517):495-7

Harvested tissue is homogenized manually using frosted microscope slides, the resulting suspension is filtered, if needed red blood cells are removed via hypotonic shock and glass wool adsorption. Resulting leukocytes are counted and subjected to cell fusion with SP2/0 cells (ATCC CRL-1581) using a polyethylene (PEG) based fusion procedure. We are using components and procedure from the Clonacell Hybridoma Cloning kit (Stemcell Technologies, Vancouver, Canada). Following the fusion, cells are plated into semi-solid methylcellulose medium HT that selects for fusion events. After an incubation of 10-14 days fusion colonies are visible and are picked using a pipette tip. Each clone is placed into a 96-well and grown for 3-7 days. At that point cell supernatant is removed for ELISA analysis. At this point the screening procedure is started including parameters for antibody expression, binding to whole fixed staphylococcus cells, binding to live staphylococcus cells as well as binding to peptide formulations. Typically only the top 10% of all clones obtained are further analyzed for specificity. Isotyping is done via a commercial isotyping kit (Isoquick, Sigma, ISOQ5).

### Construction of Recombinant Antibody Fusion Proteins

The process of producing recombinant antibodies from hybridomas has been described in U.S. application Ser. Nos. 12/686,879, 12/536,291, 11/545,601, and 11/254,500, each of which are incorporated herein by reference in their entirety.

The following description provides one example of the construction process. Variants of this approach are used and other preferred embodiments provide for alternative constructs which include microbiocides in different positions (N vs C terminal, or attached to light chain vs heavy chain) or include more than one biocide molecule, (which may be the same or different); such alternatives are therefore also included although their constriction is not described in detail.

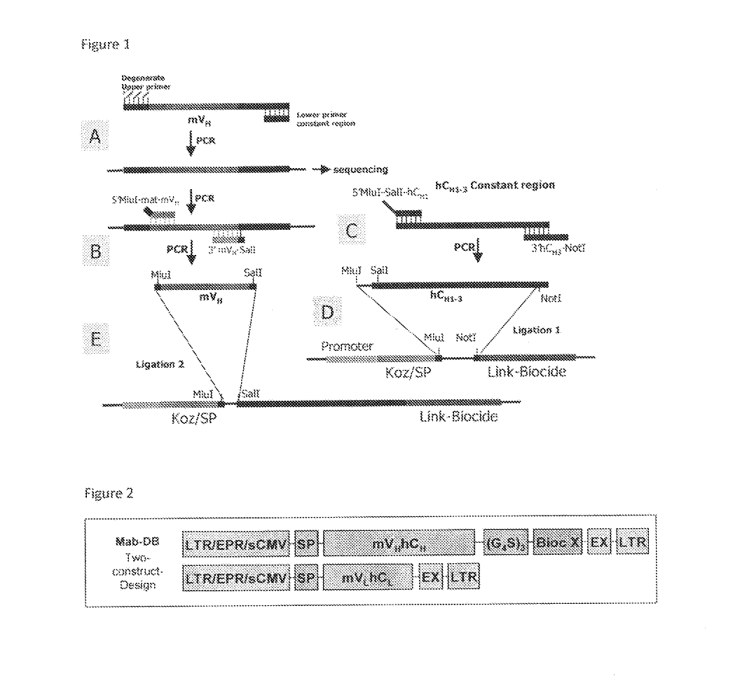

Total RNA is extracted from freshly grown hybridoma cells. RNA is reverse transcribed using oligo dT primer to generate cDNA from mRNA transcripts. This cDNA is used for extraction of the immunoglobulin variable coding region of the heavy and light chains. The use of degenerate PCR primers (FIG. 1A) allows the extraction of variable region DNA for both heavy and light chain from reverse transcribed RNA (cDNA). The PCR products obtained are cloned and sequences are verified.

SEQs of the variable regions we identified by this process are provided as SEQ ID NOs:1-32.

In the next step (FIG. 1B) the mature variable region coding sequence is defined and restriction sites are added to both ends for cloning using mutagenesis PCR. The human constant region is PCR-amplified out of human blood cDNA and restriction digested (FIG. 1C). The constant region is restriction enzyme digested and ligated in-frame into a set of existing retrovector constructs (FIG. 1D) that already contain the linker-biocide portion (LL37, LYS, PLA2, HBD2 or HBD3). The constant region is restriction enzyme digested and ligated in-frame into a set of existing retrovector constructs (FIG. 2D) that already contain the linker-biocide portion. The biocide portions from these vectors where obtained either by DNA synthesis (Blue Heron Biotechnology, Bothell, Wash.) based on Genbank information (accession numbers: LL37=NM 004345; HBD2=AF071216, HBD3=NM 018661 LYST=lysostaphin=GI:291246386) or obtained from the ATCC mammalian gene collection (hPLA2 group IIA=MGC-14516). These constructs become the destination plasmids for the variable regions. FIG. 1E shows the final cloning step of adding the variable region to the human heavy chain-biocide destination construct. The light chain is isolated from hybridoma cDNA in a similar fashion. Given the shortness of the light chain sequence, the murine variable region ise fused to the human constant light chain region by overlap extension PCR and the chimeric light chain cloned into the retrovector backbone. The basic elements of the retroviral vector are shown in FIG. 2. The light chain and heavy chains are cloned into separate vectors. Every construct is sequenced, analyzed and compared to the theoretical maps.

Retrovector from both HC and LC constructs is produced to do separate transductions of host cells as desired. Briefly, retrovector particles are made using a packaging cell line that produces the capsid, and reverse transcriptase and integrase enzymes. Retrovector constructs for the transgene and VSVg construct for the pseudotype are co-transfected into the packaging cell line which produces pseudotyped retrovector particles. These are harvested using supra-speed centrifugation and concentrated vector is used to transduce Chinese hamster ovary (CHO) cells. The transduced cell pools are subjected to limiting dilution cloning to locate a single cell into each well of a microtiter plate. Following two weeks of incubation the resulting clones are analyzed by product quantification in their supernatant. Typically about 200 clones are analyzed and the top-producing clones are selected and expanded. A clonal cell line usually contains multiple copies of the transgene and is stable over at least 60 passages. As soon as a clone is identified as a “top clone” it is immediately cryopreserved and backed up at two locations. Established clonal cell lines are then grown at volumes that meet the demands of the downstream tests.

### Efficacy Testing In Vitro

### Demonstration of Staphylococcal Binding

Binding of the recombinant antibody-microbiocide fusions is done using two different assays: ELISA using fixed whole Staphylococcus aureus cells as capture or Western blot assay using bacterial lysates that were obtained by a combination of sonication and lysostaphin digestion. These two assays detect binding under either native (ELISA) or denaturing (Western blot) conditions giving us further information whether the epitope is conformational or linear.

### Demonstration of Efficacy

The procedure for testing in vitro efficacy is based on the standard MIC (minimal inhibitory concentration) assay as described in detail in the CSLI (Clinical Laboratory Standards Institute) protocols, and by Steinberg and Lehrer (Steinberg, D., and R. I. Lehrer. 1997 Designer assays for antimicrobial peptides. Methods Mol. Biol. 78:169-186) and by Turner (Turner, J., Y. Cho, N-N. Dinh, A. J. Waring, and R. I. Lehrer. 1998. Activities of LL-37, a cathelicidin antimicrobial peptide of human neutrophils. Antimicrob. Agents Chemother. 42:2206-2214). Briefly, log phase S. aureus cells (ATCC: MRSA BA-44, MSSA 25923) are exposed to different concentrations of affinity purified antibody-microbiocide fusions, standalone antibody or various positive and negative controls including recombinant standalone antimicrobial peptide in low-salt killing assay buffer at different temperatures and incubation times. After these incubations, the bacteria are plated out using different dilution schemes and incubated overnight at 37 C. The following day, the cfu (colony forming units) count is determined and efficacy calculated based on concentration of test protein and titer present in the killing well. Further iterations of this assay include determining the concentration of test protein that is needed to inhibit over night bacterial growth in a well containing a certain titer of log phase S. aureus cells.

TABLE 5
Examples of constructs and their in vitro efficacy in reducing the growth of
S. aureus strain BA-44

| | Tested | |
|---|---|---|
| | Conc | Log |
| Construct | (nM) | reduction |
| LYST(1-246)-I7-3019-mVhC-LC-I7-3019-G1-LYST(1-246)-HC-mVhC | 43.3 | 5.3 |
| LYST(1-246)-I7-3019-mVhC-LC-I7-3019-G1-PLA2-mVhC-HC | 87.8 | 3.9 |
| LYST(1-246)-I7-3019-mVhC-LC-I7-3019-G1-LL37-mVhC-HC | 33.0 | 1.5 |
| LYST(1-246)-I8-1024-mVhC-LC-I8-1024-G1-HBD2-mVhC-HC | 9.9 | 3.5 |
| LYST(1-246)-I8-1024-mVhC-LC-I8-1024-G1-HBD2-mVhC-HC | 86.4 | 1.9 |
| LYST(1-246)-I8-1024-mVhC-LC-I8-1024-G1-HBD3-mVhC-HC | 0.9 | 1.4 |
| LYST(1-246)-I8-1024-mVhC-LC-I8-1024-G1-LL37-mVhC-HC | 0.1 | below titer |
| LYST(1-246)-I8-1024-mVhC-LC-I8-1024-G1-PLA2-mVhC-HC | 1.1 | below titer |
| LYST(1-246)-I8-1029-mVhC-LC-I8-1029-G1-LL37-mVhC-HC | 27 | 4.1 |
| LYST(1-246)-I8-1029-mVhC-LC-I8-1029-G1-HBD3-mVhC-HC | 6.4 | 2.3 |
| LYST(1-246)-I8-1029-mVhC-LC-I8-1029-G1-HBD2-mVhC-HC | 21.4 | 3.5 |
| LYST(1-246)-I8-1029-mVhC-LC-I8-1029-G1-PLA2-mVhC-HC | 1.1 | 1.2 |
| LYST(1-246)-I8-1017-mVhC-LC-I8-1017-G1-mVhC-HC | 118.3 | 2.6 |
| LYST(1-246)-I8-1031-mVhC-LC-I8-1031-G1-HBD3-mVhC | 3.6 | 2.8 |
| LYST(1-246)-I8-1031-mVhC-LC-I8-1031-G1-PLA2-mVhC | 1.2 | below titer |
| LYST(1-246)-I9-6001-mVhC-LC-I9-6001-G1-LL37-mVhC-HC | 55.4 | 4.2 |
| LYST(1-246)-I9-6001-mVhC-LC-I9-6001-G1-LL37-mVhC-HC | 1.6 | 3.8 |
| LYST(1-246)-I9-6001-mVhC-LC-I9-6001-G1-HBD2-mVhC-HC | 20.1 | 3.8 |
| LYST(1-246)-I9-6001-mVhC-LC-I9-6001-G1-HBD3-mVhC-HC | 2.6 | 3.2 |
| LYST(1-246)-I9-6001-mVhC-LC-I9-6001-G1-PLA2-mVhC-HC | 1.8 | 2.8 |
| LYST(1-246)-I9-6014-mVhC-LC-I9-6014-G1-PLA2-mVhC-HC | <1 nM | 1.3 |
| LYST(1-246)-I9-7002-mVhC-LC-I9-7002-G1-HBD2-mVhC-HC | 1.2 | 1.9 |
| LYST(1-246)-I9-7002-mVhC-LC-I9-7002-G1-HBD3-mVhC-HC | 1.9 | 1.3 |
| LYST(1-246)-1A9-mVhC-LC-1A9-G1-HBD2-mVhC-HC | 18.5 | 0.6 |
| LYST(1-246)-1A9-mVhC-LC-1A9-G1-HBD2-mVhC-HC | 3 | 1 |
| LYST(1-246)-1A9-mVhC-LC-1A9-G1-HBD2-mVhC-HC | 13.5 | below titer |
| LYST(1-246)-G2a(CH2-3)-LL37 | 43.7 | 0.7 |
| LYST(1-246)-G2a(CH2-3)-HBD3 | 61.6 | 0.9 |
| LYST(1-246)-I8-1024-mVhC-LC-his-I8-1024-G1-HBD3-mVhC-Fab-HC | 475.0 | below titer |
| LYST(1-246)-I8-1024-mVhC-LC-his-I8-1024-G1-HBD3-his-mVhC-Fab2-HC | 479.0 | 1 |
| LYST(1-246)-I8-1024-mVhC-LC-his-I8-1024-G1-HBD3-his-mVhC-Fab-HC | 832.0 | 0.9 |
| LYST(1-246)-I8-1024-mVhC-LC-his-I8-1024-G1-HBD3-mVhC-Fab2-HC | 630.5 | 1.6 |
| LYST(1-246)-I9-6001-mVhC-LC-his-I9-6001-G1-HBD3-mVhC-Fab2-HC | 258.1 | 3.6 |
| LYST(1-246)-I9-6001-mVhC-LC-his-I9-60001-G1-HBD3-mVhC-Fab-HC | 97.2 | 0.1 |
| LYST(1-246)-I9-6001-mVhC-LC-his-I9-6001-G1-HBD3-his-mVhC-Fab2-HC | 128.6 | 3 |
| LYST(1-246)-I9-6001-mVhC-LC-his-I9-6001-G1-HBD3-his-mVhC-Fab-HC | 185.3 | 3 |
| his-hG1-Fc-PLA2 | 346.3 | below titer |

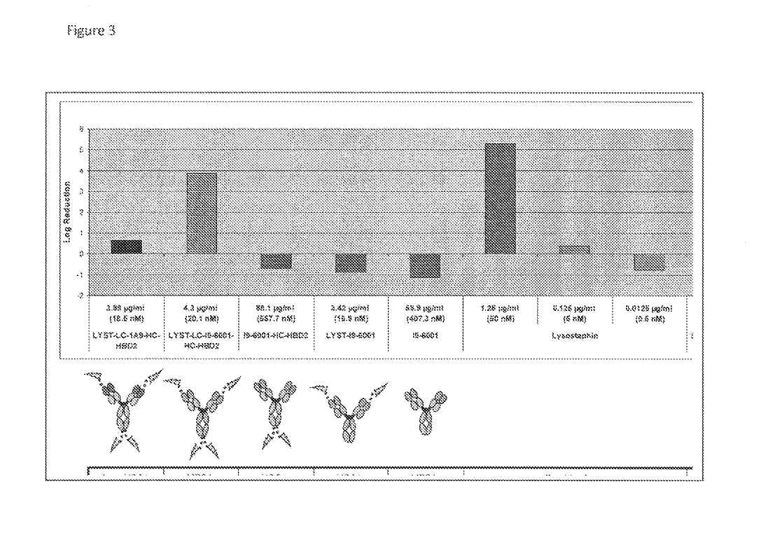

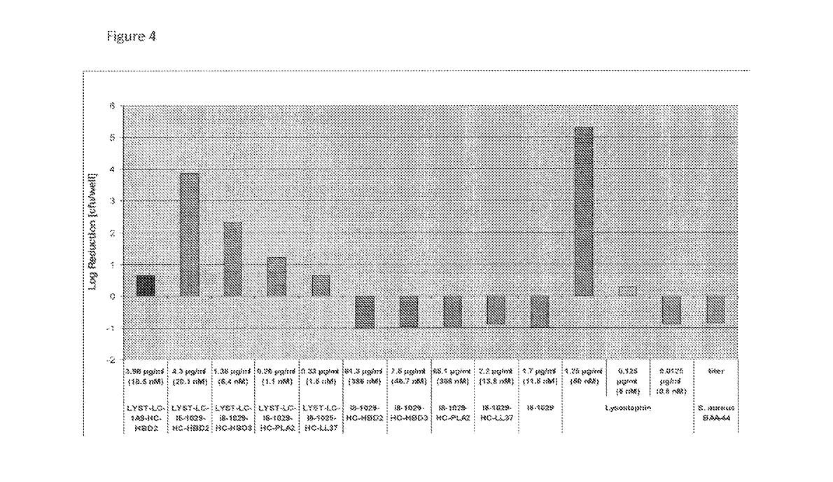

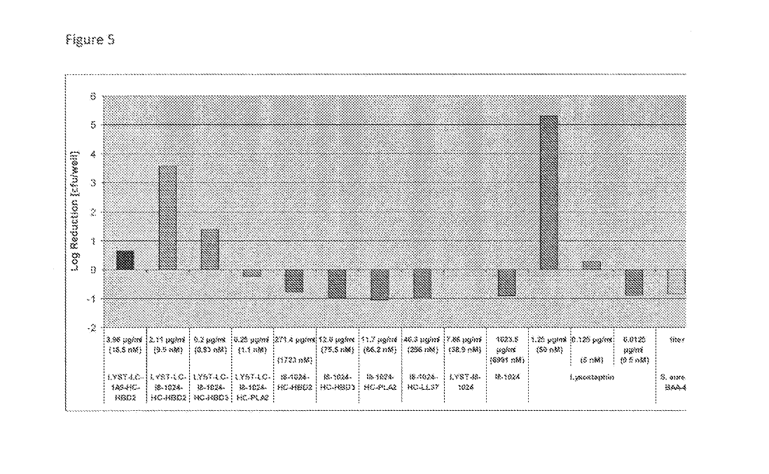

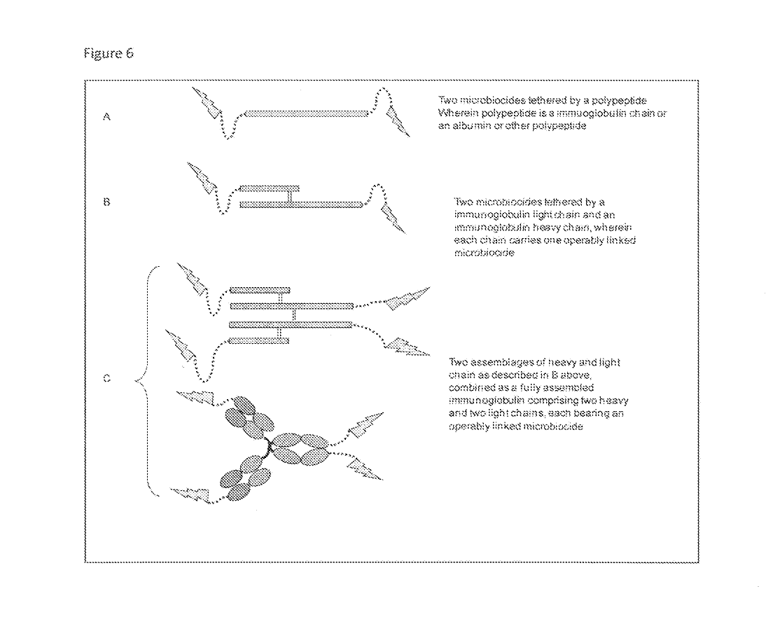

FIGS. 3-5 provide examples of the results of efficacy testing. Also evident from these figures is the differential activity between multiple constructs based on the same antibody; not every antibody—microbiocide construct was effective in killing S. aureus and the conformations which were shown to be active were not the same for each antibody.

### Example 2

### Antimicrobial Efficacy In Vitro

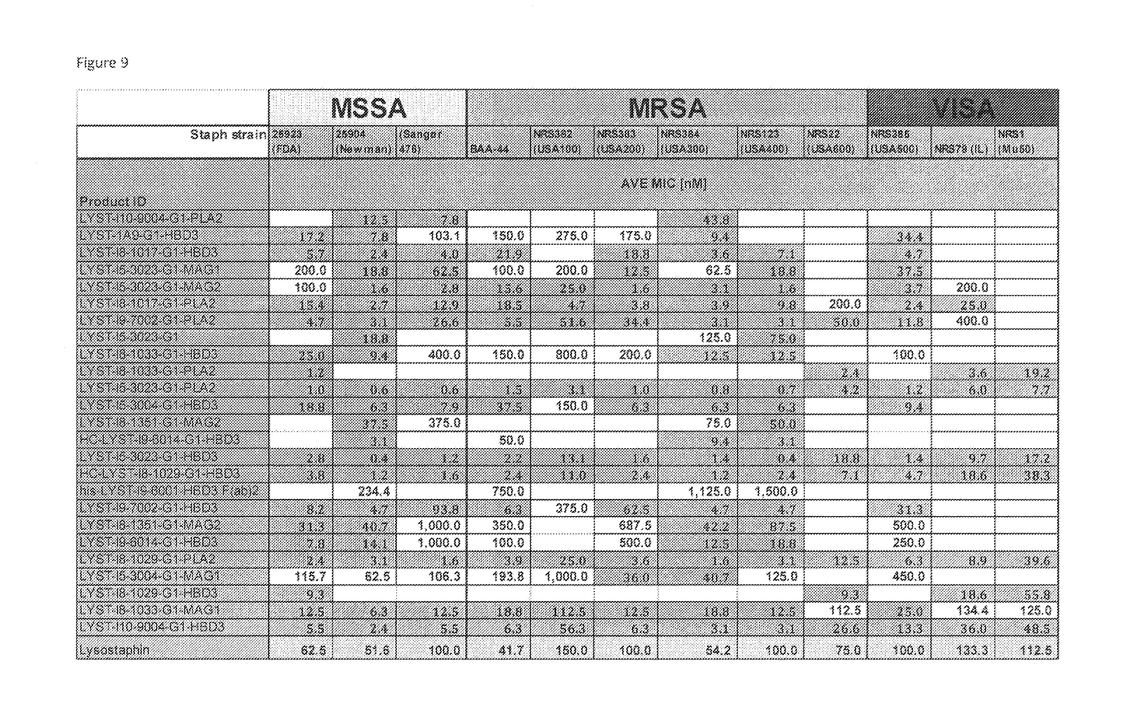

DBs were tested for efficacy against 12 strains of S. aureus using a standard Clinical and Laboratory Standards Institute (CLSI) MIC/MBC tests (2012. Antimicrobial Susceptibility Tests for Bacteria That Grow Aerobically: Approved Standard M07-A9, Ninth ed. Clinical and Laboratory Standards Institute, Wayne, Pa.; Steinberg et al. 1997. Methods Mol. Biol. 78:169-186). An overnight culture was diluted and grown in TSB to log phase. Cells were harvested, and titered to 2.5×10⁷ CFU/ml. Serial dilutions of DB in PBS were mixed with huFc pre-blocked Staph cells, incubated for 45 min at 37° C. & 250 RPM shaking before 100 μl of 2×TSB was added followed by incubation overnight. Plates were visually scored for turbidity after overnight growth and MIC determined. At least 3 independent MIC assays were performed for each product tested with the 12 different staph strains. FIG. 9 summarizes MIC results for the products tested, showing the S. aureus strains tested which include methicillin sensitive (MSSA), methicillin resistant (MRSA) and vancomycin intermediate resistant (VISA) strains compared to a lysostaphin control (Sigma L9043).

### Example 3

### Oral and Parenteral Uptake

Test product is administered by oral gavage to groups of 6 week old mice, which have previously been administered 100 microliters (10 mg/kg) of cimetidine orally, an approved antacid to reduce the stomach pH. Up to 100 microliters of product will be administered at concentrations of up to 30 mg/kg as a single dose. Products for intraperitoneal injection are either sterile affinity purified or PEG-precipitated product derived from CHO cell supernatant. For the injection procedure the mouse is restrained and a 25 g needle is inserted into the lower left quadrant of the abdominal area. The mouse is held at a downward angle and the syringe is injected at a 45° angle relative to the peritoneum. Up to 500 microliters (based on a 25 g mouse) of product are administered.

At different time points past dosing, individual mice are removed from the group and terminally bled to obtain the maximum amount of serum for subsequent testing. Serum will be will be tested for the presence of product using a standard sandwich ELISA using anti-human Fc antibody (Bethyl Laboratories, Montgomery, Tex.) as capture and anti-human kappa light chain HRP as secondary reagent (Bethyl Laboratories, Montgomery, Tex.). Serum containing product harvested from animals will also be tested in a MIC test as above to determine if an antimicrobial effective concentration is present.

Pharmacodynamics will be determined by comparing concentrations of detected product in circulation. Depending on serum presence of the product, a dosing regimen will be determined to maintain an effective concentration of product in circulation for the duration of several days.

In a preliminary experiment, intraperitoneal injections of 200 ug per mouse (10 mg/kg) and mice showed no toxicity over 24 hours. Following terminal bleed out of mice and were able to detect 10 ug/ml in serum. However the resulting concentration in serum harvested from the mice was below the expected MIC for this product.

Product exposed to whole mouse serum in vitro for 24 hours and then tested under standard MIC conditions did not show reduced activity. Hence serum components do not inactivate the product.

### Example 4

### Time-Kill Curve In Vitro

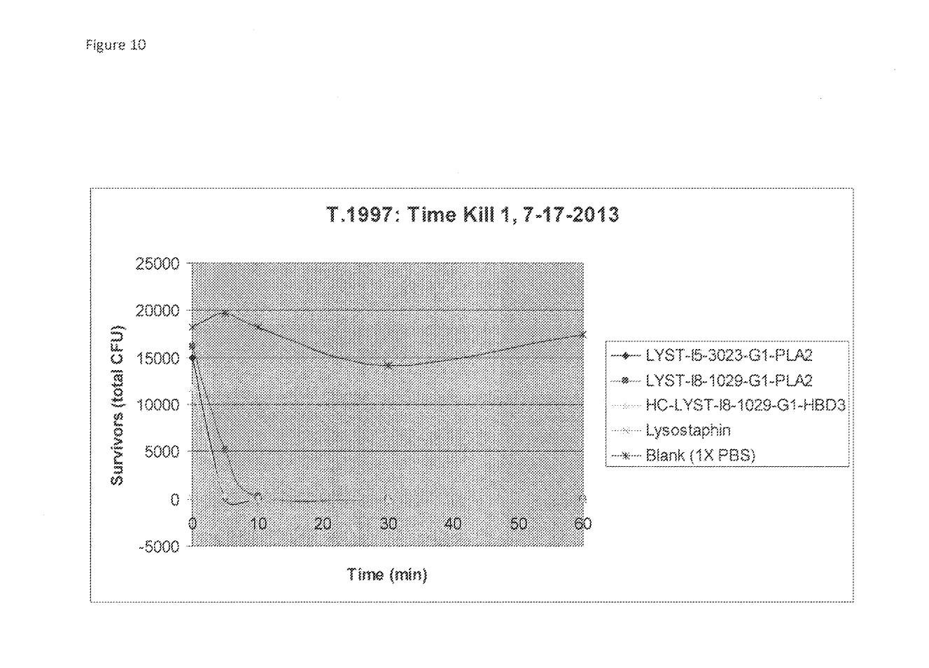

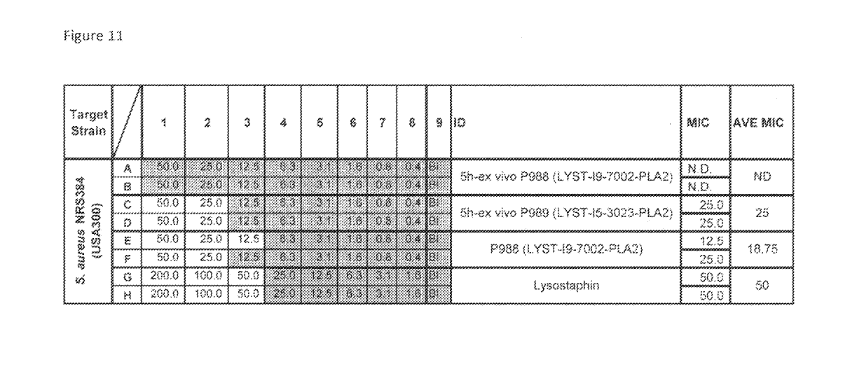

For S. aureus strain NRS384 (USA300) we evaluated exposure times from T₀ to T₃₀. Briefly, log-phase bacteria at 2.5×10⁶ cfu/ml were exposed to concentrations of product previously determined to kill 50,000 cfu/ml in a MIC test and incubated at 37° C. in PBS. At different time points, a small amount of the suspension was removed, and growth arrested by immediate dilution in chilled PBS. Aliquots were then plated on TSA. After plating all time points, plates are incubated overnight and cfu counts used to calculate kill curves. Results are shown in FIG. 10, indicating that at a standard MIC concentration and inoculum a complete bacterial kill is achieved in less than 10 minutes.

### Example 5

### Evaluate the Impact of Inoculum Size on Bactericidal Efficacy

Inoculum size has been shown to have a significant impact on antimicrobial efficacy (Lee et al. Antimicrobial agents and chemotherapy 57:1434-1441. 2013). To evaluate if larger innocula offer a survival advantage to S. aureus in the presence of DB, we used our MIC assay format to conduct killing tests with the test antistaphylococcal products and controls including standalone lysostaphin and vancomycin at multiple innoculum concentrations starting at 2.5×10⁴ cfu/ml with several 10-fold increases. Tables 6 and 7 shows results indicating that some antimicrobial products still achieve a MIC of low nanomolarity. For example product 701223 has an MIC of 4.8 nanomolar against an inoculum of. 5×10⁶ cfu/ml and 75 nanomolar against an inoculum of 5×10⁸ cfu/ml.

TABLE 6

| Target in all cases is S. aureus | | | | High Titer | | | Low Titer | | |
|---|---|---|---|---|---|---|---|---|---|
| NRS384 (USA300) Data points are | | | | 5,000,000 | | | 50,000 | | |
| MIC scores for individual wells | | | | CFU/well | | | CFU/well | | |
| P# | M# | Event #+ | Name | Rep 1 | Rep 2 | Ave | Rep 1 | Rep 2 | Ave |
| P970 | M1785 | 701223c147 | LYST-I5- | 6.4 | 3.2 | 4.8 | 1.6 | 0.8 | 1.2 |
| | | | 3023-G1-PLA2 | | | | | | |
| P972 | M1776 | 701313c6 | LYST-I8- | ND | 12.8 | 12.8 | 1.6 | 1.6 | 1.6 |
| | | | 1029-G1-PLA2 | | | | | | |
| P959 | M1762 | 701316c126 | HC-LYST-I8- | 4.8 | 4.8 | 4.8 | 1.2 | 1.2 | 1.2 |
| | | | 1029-G1-HBD3 | | | | | | |
| | | | Lysostaphin | 27.1 | 27.1 | 27.1 | 54 | 27.1 | 40.7 |
| | | | (Sigma L9043) | | | | | | |

ND = not determined, i.e., higher than highest tested concentration.
Concentrations shown are nanomolar

TABLE 7

| Target in all cases is S. aureus | | | | High Titer | | | Low Titer | | |
|---|---|---|---|---|---|---|---|---|---|
| NRS384 (USA300) Data points are | | | | 500,000,000 | | | 50,000,000 | | |
| MIC scores for individual wells | | | | CFU/well | | | CFU/well | | |
| P# | M# | Event #+ | Name | Rep 1 | Rep 2 | Ave | Rep 1 | Rep 2 | Ave |
| P970 | M1785 | 701223c147 | LYST-I5- | 100 | 50 | 75 | 6.3 | 12.5 | 9.4 |
| | | | 3023-G1-PLA2 | | | | | | |
| P972 | M1776 | 701313c6 | LYST-I8- | ND | ND | ND | ND | ND | ND |
| | | | 1029-G1-PLA2 | | | | | | |
| P959 | M1762 | 701316c126 | HC-LYST-I8- | ND | ND | ND | 25 | 25 | 25 |
| | | | 1029-G1-HBD3 | | | | | | |
| | | | Lysostaphin | ND | 200 | 200 | 200 | 200 | 200 |
| | | | (Sigma L9043) | | | | | | |

ND = not determined, i.e., higher than highest tested concentration.
Concentrations shown are nanomolar

### Example 6

### In Vitro Determination of Synergy with Antibiotics

A synergistic effect of DB with standard antibiotic therapies may allow lower doses of both antibiotics and the antibody fusion product thus lowering cost, and achieving more rapid clearance. We will start with sublethal (below MIC) concentrations of both the antibody fusion product and the antibiotic. A range of concentrations and combination ratios will be tested to evaluate the best synergistic effect. These tests will also include the combination of different antistaphylococcal products, as it is possible that having different C-terminal biocides in combination enhances the overall pressure on the bacterial target. We will perform these tests on all 3 S. aureus groups including MSSA, MRSA and VISA. The antibiotics will be chosen according the resistance pattern, to include methicillin, vancomycin, cephalosporins and other antibiotics. We will include combinations with antibiotics to which the target bacteria are resistant to detect the potential of our product to reverse antibiotic resistance.

### Example 7

### Monitor for Emergence of Resistant Organisms In Vitro

Lysostaphin hydrolyses the pentaglycine bridge of PGN, it has been reported that generation of lysostaphin resistant S. aureus result in mutants that feature monoglycine bridges which lysostaphin is unable to cleave, however, these originally multiresistant mutants relapse to beta-lactam susceptibility (Climo et al Antimicrob. Agents Chemother. 45:1431-1437. 2001). We will conduct repeated passage of S. aureus (4 selected strains with varied MIC) for each test antibody fusion product at sub MIC concentrations.

### Example 8

### Evaluate Efficacy in Eliminating Established Biofilms In Vitro

Efficacy of antibody fusion products against biofilms of S. aureus will be established for 6, 12 and 24 hours cultures using the MBEC Physiology & Genetics (P&G) Assay (Innovotech), a commercial example of the Calgary Biofilm Device (Ceri et al 1999 J Clin. Microbiol 37:1771-1776). This assay uses 96-well plates designed with specialized lids that have pegs that protrude into the medium in the wells and allow colonization of biofilms. The device can be used in an assay very similar to CLSI MIC assays with only slight modification and successful antimicrobials generate a value of MBEC (minimum biofilm eradicating concentration). Target cultures of S. aureus are grown to 1.0 MacFarland standard (˜3×10⁸ CFU/ml), which is diluted 1:30 (1×10⁷ CFU/ml) and used to inoculate wells of the specialized microtiter plate. After incubation at 37° C. and 100 RPM for 6, 12, and 24 h growth times, the pegged lid is removed from the media and the pegs are rinsed in sterile saline. Initial inoculum density, and final well and peg density are determined by serial dilution in saline and provide a measure or the relative proportion of bacterial target that is planktonic or in the biofilm. Rinsed pegs are then placed into a test plate containing antibody fusion products or controls diluted in a twofold series. Biofilms are exposed for 1 h, pegs are rinsed twice in sterile saline, and then transferred to a recovery plate containing a suitable medium and a biocide neutralization solution. Biofilms are sonicated into this plate using a sonicating water bath and then incubated for 24-72 h, whereupon MBEC is scored in the same manner as CLSI MIC assays.

| SEQ ID NO: 1. I7-3019 light chain variable region, | |
|---|---|
| nucleotide sequence, ID: 500685n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACacgcgtGATGTTTTGATGACCCAAACTCCACTCTCCC |
| 101 | TGCCTGTCAGTCTTGGAGATCAAGCCTCCATCTCTTGCAGATCTAGTCAG |
| 151 | AGCATTGTACATACTAATGGAAACACCTATTTAGAATGGTACCTGCAGAA |
| 201 | ACCGGGCCAGTCTCCAAAGCTCCTGATCTACAAAGTTTCCAACCGATTTT |
| 251 | CTGGGGTCCCAGACAGGTTCAGTGGCAGTGGATCAGGGACAGATTTCACA |
| 301 | CTCAAGATCAGCAGAGTGGAGGCTGAGGATCTGGGAGTTTATTACTGCTT |
| 351 | TCAAGGTTCACATATTCCGTGGACGTTCGGTGGAGGCACCAAGCTGGAAA |
| 401 | TCAAACGG |
| 1-60 | Signal peptide |
| 61-408 | Light chain variable region |
| SEQ ID NO: 2. I7-3019 light chain variable region, | |
| amino acid sequence, ID: 500685p | |
| | .........o.........o.........o.........o.........o |
| 1 | METDTLLLWVLLLWVPGSTGDTRDVLMTQTPLSLPVSLGDQASISCRSSQ |
| 51 | SIVHTNGNTYLEWYLQKPGQSPKLLIYKVSNRFSGVPDRFSGSGSGTDFT |
| 101 | LKISRVEAEDLGVYYCFQGSHIPWTFGGGTKLEIKR |
| 1-20 | Signal peptide |
| 21-136 | Light chain variable region |
| SEQ ID NO: 3. I7-3019 heavy chain variable region, | |
| nucleotide sequence, ID: 500686n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACacgcgtCAGGTCCAACTGCAGCAGCCTGGGGCTGAGC |
| 101 | TGGTGAGGCCTGGGGCTTCAGTGAACCTGTCCTGCAGGGCTTCTGGCTAC |
| 151 | ACCTTCACCACCTACTGGATGATCTGGGTGAAGCAGAGGCCTGGACAAGG |
| 201 | CCTTGAATGGATTGGTATGATTGATCCTTCAGACAGTGAGACTCACTACA |
| 251 | ATCAAATGTTCAAGGACAAGGCCACATTGACTGTAGACAAATCCTCCACC |
| 301 | ACAGCCTACATGCAGTTCAGCAGCCTAACATCTGAGGACTCTGCGGTCTA |
| 351 | TTACTGTGCAAGATGGAACTTCGGTAAGGGCTACTGGGGCCAAGGCACCA |
| 401 | CTCTCACGGTCTCCTCA |
| 1-60 | Signal peptide |
| 61-417 | Heavy chain variable region |
| SEQ ID NO: 4. I7-3019 heavy chain variable region, | |
| amino acid sequence, ID: 500686p | |
| | .........o.........o.........o.........o.........o |
| 1 | METDTLLLWVLLLWVPGSTGDTRQVQLQQPGAELVRPGASVNLSCRASGY |
| 51 | TFTTYWMIWVKQRPGQGLEWIGMIDPSDSETHYNQMFKDKATLTVDKSST |
| 101 | TAYMQFSSLTSEDSAVYYCARWNFGKGYWGQGTTLTVSS |
| 1-20 | Signal peptide |
| 21-139 | Heavy chain variable region |
| SEQ ID NO: 5. I8-1017 light chain variable region, | |
| nucleotide sequence, ID: 500661n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACacgcgtGACATTGTGATGTCACAGTCTCCATCCTCCC |
| 101 | TGGCTGTGTCAGCAGGAGAGAAGGTCACTATGAGCTGCAAATCCAGTCAG |
| 151 | AGTCTGCTCAACAGTAGAACCCGAAAGAACTACTTGGCTTGGTACCAGCA |
| 201 | GAAACCAGGGCAGTCTCCTAAACTGCTGATCTACTGGGCATCCACTAGGG |
| 251 | AATCTGGGGTCCCTGATCGCTTCACAGGCAGTGGATCTGGGACAGATTTC |
| 301 | ACTCTCACCATCAGCAGTGTGCAGGCTGAAGACCTGGCAGTTTATTACTG |
| 351 | CAAGCAATCTTATAATCTGTGGACGTTCGGTGGAGGCACCAAGCTGGAAA |
| 401 | TCAAACGG |
| 1-60 | Signal peptide |
| 61-408 | Light chain variable region |
| SEQ ID NO: 6. I8-1017 light chain variable region, | |
| amino acid sequence, ID: 500661p | |
| | .........o.........o.........o.........o.........o |
| 1 | METDTLLLWVLLLWVPGSTGDTRDIVMSQSPSSLAVSAGEKVTMSCKSSQ |
| 51 | SLLNSRTRKNYLAWYQQKPGQSPKLLIYWASTRESGVPDRFTGSGSGTDF |
| 101 | TLTISSVQAEDLAVYYCKQSYNLWTFGGGTKLEIKR |
| 1-20 | Signal peptide |
| 21-136 | Light chain variable region |
| SEQ ID NO: 7. I8-1017 heavy chain variable region, | |
| nucleotide sequence, ID: 500662n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACacgcgtGAGGTTCAGCTGCAGCAGTCTGGGGCAGAGC |
| 101 | TTGTGAAGCCAGGGGCCTCAGTCAAGTTGTCCTGCACAGCTTCTGGCTTC |
| 151 | AACATTAAAGACACCTATATGCACTGGGTGAAGCAGAGGCCTGAACAGGG |
| 201 | CCTGGAGTGGATTGGAAGGATTGATCCTGCGAATGGTAATACTAAATATG |
| 251 | ACCCGAAGTTCCAGGGCAAGGCCACTATAACAGCAGACACATCCTCCAAC |
| 301 | ACAGCCTACCTGCAGCTCAGCAGCCTGACATCTGAGGACACTGCCGTCTA |
| 351 | TTACTGTGCTGGTAACCACTACTTTGACTACTGGGGCCAAGGCACCACTC |
| 401 | TCACAGTCTCCTCA |
| 1-60 | Signal peptide |
| 61-414 | Heavy chain variable region |
| SEQ ID NO: 8. I8-1017 heavy chain variable region, | |
| amino acid sequence, ID: 500662p | |
| | .........o.........o.........o.........o.........o |
| 1 | METDTLLLWVLLLWVPGSTGDTREVQLQQSGAELVKPGASVKLSCTASGF |
| 51 | NIKDTYMHWVKQRPEQGLEWIGRIDPANGNTKYDPKFQGKATITADTSSN |
| 101 | TAYLQLSSLTSEDTAVYYCAGNHYFDYWGQGTTLTVSS |
| 1-20 | Signal peptide |
| 21-138 | Heavy chain variable region |
| SEQ ID NO: 9. I8-1024 light chain variable region, | |
| nucleotide sequence, ID: 500667n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACacgcgtGACATTGTGATGACCCAGTCTCAAAAATTCA |
| 101 | TGTCCACATCAGTAGGAGACAGGGTCAGCGTCACCTGCAAGGCCAGTCAG |
| 151 | AATGTGGGTACTAATGTAGCCTGGTATCAACAGAAACCAGGGCAATCTCC |
| 201 | TAAAGCACTGATTTACTCGGCATCCTACCGGTACAGTGGAGTCCCTGATC |
| 251 | GCTTCACAGGCAGTGGATCTGGGACAGATTTCACTCTCACCATCAGCAAT |
| 301 | GTGCAGTCTGAAGACTTGGCAGAGTATTTCTGTCAGCAATATAACAGCTA |
| 351 | TCCTCTCACGTTCGGTGCTGGGACCAAGCTGGAGCTGAAACGG |
| 1-60 | Signal peptide |
| 61-393 | Light chain variable region |
| SEQ ID NO: 10. I8-1024 light chain variable | |
| region, amino acid sequence, ID: 500667p | |
| | .........o.........o.........o.........o.........o |
| 1 | METDTLLLWVLLLWVPGSTGDTRDIVMTQSQKFMSTSVGDRVSVTCKASQ |
| 51 | NVGTNVAWYQQKPGQSPKALIYSASYRYSGVPDRFTGSGSGTDFTLTISN |
| 101 | VQSEDLAEYFCQQYNSYPLTFGAGTKLELKR |
| 1-20 | Signal peptide |
| 21-131 | Light chain variable region |
| SEQ ID NO: 11. I8-1024 heavy chain variable | |
| region, nucleotide sequence, ID: 500668n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACacgcgtGAAGTGAAGCTGGTGGAGTCTGGGGGAGGTT |
| 101 | TAGTGCAGCCTGGAGGGTCCCTGAAACTCTCCTGTGCAGCCTCTGGATTC |
| 151 | ACTTTCAGTAGCTATACCATGTCTTGGGTTCGCCAGACTCCAGAGAAGAG |
| 201 | GCTGGAGTGGGTCGCATACATTAGTAATGGTGGTGGTAGCACCTACTATC |
| 251 | CAGACACTGTAAAGGGCCGATTCACCATCTCCAGAGACAATGCCAAGAAC |
| 301 | ACCCTGTACCTGCAAATGAGCAGTCTGAAGTCTGAGGACACGGCCATGTA |
| 351 | TTACTGTGCAAGACAGGTACGACGGGGGATGGACTACTGGGGTCAAGGAA |
| 401 | CCTCAGTCACCGTCTCCTCA |
| 1-60 | Signal peptide |
| 61-420 | Heavy chain variable region |
| SEQ ID NO: 12. I8-1024 heavy chain variable | |
| region, amino acid sequence, ID: 500668p | |
| | .........o.........o.........o.........o.........o |
| 1 | METDTLLLWVLLLWVPGSTGDTREVKLVESGGGLVQPGGSLKLSCAASGF |
| 51 | TFSSYTMSWVRQTPEKRLEWVAYISNGGGSTYYPDTVKGRFTISRDNAKN |
| 101 | TLYLQMSSLKSEDTAMYYCARQVRRGMDYWGQGTSVTVSS |
| 1-20 | Signal peptide |
| 21-140 | Heavy chain variable region |
| SEQ ID NO: 13. I8-1029 light chain variable | |
| region, nucleotide sequence, ID: 500710n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACacgcgtGACATTGTGATGACACAGTCTCCATCCTCCC |
| 101 | TGACTGTGACAGCAGGAGAGAAGGTCACTATGAGCTGCAAGTCCAGTCAG |
| 151 | AGTCTGTTAAACAGTGGAAATCAAAAGAaCTACTTGACCTGGTACCAGCA |
| 201 | GAAACCAGGGCAGCCTCCTAAACTGTTGATCTACTGGGCATCCACTAGGG |
| 251 | AATCTGGGGTCCCTGATCGCTTCACAGGCAGTGGATCTGGAACAGATTTC |
| 301 | ACTCTCACCATCAGCAGTGTGCAGGCTGAAGACCTGGCAGTTTATTACTG |
| 351 | TCAGAATGATTATAGTTATCCTTTCACGTTCGGCTCGGGGACAAAGTTGG |
| 401 | AAATAAAACGG |
| 1-60 | Signal peptide |
| 61-411 | Light chain variable region |
| SEQ ID NO: 14. I8-1029 light chain variable | |
| region, amino acid sequence, ID: 500710p | |
| | .........o.........o.........o.........o.........o |
| 1 | METDTLLLWVLLLWVPGSTGDTRDIVMTQSPSSLTVTAGEKVTMSCKSSQ |
| 51 | SLLNSGNQKNYLTWYQQKPGQPPKLLIYWASTRESGVPDRFTGSGSGTDF |
| 101 | TLTISSVQAEDLAVYYCQNDYSYPFTFGSGTKLEIKR |
| 1-20 | Signal peptide |
| 21-137 | Light chain variable region |
| SEQ ID NO: 15. I8-1029 heavy chain variable | |
| region, nucleotide sequence, ID: 500711n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACacgcgtGAGGTCCAGCTGCAGCAGTCTGGACCTGAGC |
| 101 | TAGTGAAGACTGGGGCTTCAGTGAAGATATCCTGCAAGGCTTCTGGTTAC |
| 151 | TCATTCACTGGTTACTACATGCACTGGGTCAAGCAGAGCCATGGAAAGAG |
| 201 | CCTTGAGTGGATTGGATATATTAGTTGTTACAATGGTGCTACTAGCTACA |
| 251 | ACCAGAAGTTCAAGGGCAAGGCCACATTTACTGTAGACACATCCTCCAGC |
| 301 | ACAGCCTACATGCAGTTCAACAGCCTGACATCTGAAGACTCTGCGGTCTA |
| 351 | TTACTGTGCAAGATCGAGGACTGGAGCCTGGTTTGCTTACTGGGGCCAAG |
| 401 | GGACTCTGGTCACTGTCTCT |
| 1-60 | Signal peptide |
| 61-420 | Heavy chain variable region |
| SEQ ID NO: 16. I8-1029 heavy chain variable | |
| region, amino acid sequence, ID: 500711p | |
| | .........o.........o.........o.........o.........o |
| 1 | METDTLLLWVLLLWVPGSTGDTREVQLQQSGPELVKTGASVKISCKASGY |
| 51 | SFTGYYMHWVKQSHGKSLEWIGYISCYNGATSYNQKFKGKATFTVDTSSS |
| 101 | TAYMQFNSLTSEDSAVYYCARSRTGAWFAYWGQGTLVTVS |
| 1-20 | Signal peptide |
| 21-140 | Heavy chain variable region |
| SEQ ID NO: 17. I8-1031 light chain variable | |
| region, nucleotide sequence, ID: 500724n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACacgcgtGATATCCAGATGACACAGAcTACATCCTCCC |
| 101 | TGTCTGCCTCTCTGGGAGACAGAGTCACCATCAGTTGCAGGGCAAGTCAG |
| 151 | GACATTAGCAATTATTTAAACTGGTATCAGCAGAAACCAGATGGAACTGT |
| 201 | TAAACTCCTGATCTACTACACATCAAGATTACACTCAGGAGTCCCATCAA |
| 251 | GGTTCAGTGGCAGTGGGTCTGGAACAGATTATTCTCTCACCATTAGCAAC |
| 301 | CTGGAGCAAGAAGATATTGCCACTTACTTTTGCCAACAGGGTAATACGCT |
| 351 | TCCGTGGACGTTCGGTGGAGGtACCAAGCTGGAAATcAAACGG |
| 1-60 | Signal peptide |
| 61-393 | Light chain variable region |
| SEQ ID NO: 18. I8-1031 light chain variable | |
| region, amino acid sequence, ID: 500724p | |
| | .........o.........o.........o.........o.........o |
| 1 | METDTLLLWVLLLWVPGSTGDTRDIQMTQTTSSLSASLGDRVTISCRASQ |
| 51 | DISNYLNWYQQKPDGTVKLLIYYTSRLHSGVPSRFSGSGSGTDYSLTISN |
| 101 | LEQEDIATYFCQQGNTLPWTFGGGTKLEIKR |
| 1-20 | Signal peptide |
| 21-137 | Light chain variable region |
| SEQ ID NO: 19. I8-1031 heavy chain variable | |
| region, nucleotide sequence, ID: 500725n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACacgcgtGAAGTGAAGCTGGTGGAGTCTGGGGGAGGCT |
| 101 | TAGTGAAGCCTGGAGGGTCCCTGAAACTCTCCTGTGCAGCCTCTGGATTC |
| 151 | GCTTTCAGTAGCTATGACATGTCTTGGGTTCGCCAGACTCCGGAGAAGAG |
| 201 | GCTGGAGTGGGTCGCAACCATTAGTAGTGGTGGTAGTTACACCTACTATC |
| 251 | CAGACAGTGTGAAGGGCCGATTCACCATCTCCAGAGACAATGCCAGGAAC |
| 301 | ACCCTGTACCTGCAAATGAGCAGTCTGAGGTCTGAGGACACGGCCTTGTA |
| 351 | TTACTGTGCAAGACCACGGTTACAGCTCGGGTCGCCTGCCTGGTTTGCTT |
| 401 | ACTGGGGCCAAGGGACTCTGGTCACTGTCTCT |
| 1-60 | Signal peptide |
| 61-432 | Heavy chain variable region |
| SEQ ID NO: 20. I8-1031 heavy chain variable | |
| region, amino acid sequence, ID: 500725p | |
| | .........o.........o.........o.........o.........o |
| 1 | METDTLLLWVLLLWVPGSTGDTREVKLVESGGGLVKPGGSLKLSCAASGF |
| 51 | AFSSYDMSWVRQTPEKRLEWVATISSGGSYTYYPDSVKGRFTISRDNARN |
| 101 | TLYLQMSSLRSEDTALYYCARPRLQLGSPAWFAYWGQGTLVTVS |
| 1-20 | Signal peptide |
| 21-144 | Heavy chain variable region |
| SEQ ID NO: 21. I8-1239 light chain variable | |
| region, nucleotide sequence, ID: 500635n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACacgcgtGACATCCTGATGACCCAATCTCCATCCTCCA |
| 101 | TGTCTGTATCTCTGGGAGACACAGTCAGCATCACTTGCCATGCAAGTCAG |
| 151 | GGCATTAGCAGTAATATAGGGTGGTTGCAGCAGAAACCAGGGAAATCATT |
| 201 | TAAGGGCCTGATCTATCATGGAACCAACTTGGAAGATGGAGTTCCATCAA |
| 251 | GGTTCAGTGGCAGTGGATCTGGAGCAGATTATTCTCTCACCATCAGCAGC |
| 301 | CTGGAATCTGAAGATTTTGCAGACTATTACTGTGTACAGTATGCTCAGTT |
| 351 | TCCTCGGACGTTCGGTGGAGGCACCAAGCTGGAAATCAAACGG |
| 1-60 | Signal peptide |
| 61-393 | Light chain variable region |
| SEQ ID NO: 22. I8-1239 light chain variable region | |
| amino acid sequence, ID: 500635p | |
| | .........o.........o.........o.........o.........o |
| 1 | METDTLLLWVLLLWVPGSTGDTRDILMTQSPSSMSVSLGDTVSITCHASQ |
| 51 | GISSNIGWLQQKPGKSFKGLIYHGTNLEDGVPSRFSGSGSGADYSLTISS |
| 101 | LESEDFADYYCVQYAQFPRTFGGGTKLEIKR |
| 1-20 | Signal peptide |
| 21-131 | Light chain variable region |
| SEQ ID NO: 23. I8-1239 heavy chain variable | |
| region, nucleotide sequence, ID: 500636n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACacgcgtCAGGTGCAGCTGAAGCAGTCAGGACCTGGCC |
| 101 | TAGTGCAGCCCTCACAGAGCCTGTCCATAACCTGCACAGTCTCTGGTTTC |
| 151 | TCATTAACTAGCTATGGTGTACACTGGGTTCGCCAGTCTCCAGGAAAGGG |
| 201 | TCTGGAGTGGCTGGGAGTGATATGGAGAGGTGGAAGCACAGACTACAATG |
| 251 | CAGCTTTCATGTCCAGACTGAGCATCACCAAGGACAACTCCAAGAGCCAA |
| 301 | GTTTTCTTTAAAATGAACAGTCTGCAAGCTGATGACACTGCCATATACTA |
| 351 | CTGTGCCAAAGAGGACTTGCTTGCTTACTGGGGCCAAGGGACTCTGGTCA |
| 401 | CTGTCTCT |
| 1-60 | Signal peptide |
| 61-408 | Heavy chain variable region |
| SEQ ID NO: 24. I8-1239 heavy chain variable | |
| region, amino acid sequence, ID: 500636p | |
| | .........o.........o.........o.........o.........o |
| 1 | METDTLLLWVLLLWVPGSTGDTRQVQLKQSGPGLVQPSQSLSITCTVSGF |
| 51 | SLTSYGVHWVRQSPGKGLEWLGVIWRGGSTDYNAAFMSRLSITKDNSKSQ |
| 101 | VFFKMNSLQADDTAIYYCAKEDLLAYWGQGTLVTVS |
| 1-20 | Signal peptide |
| 21-136 | Heavy chain variable region |
| SEQ ID NO: 25. I9-6001 light chain variable | |
| region, nucleotide sequence, ID: 500734n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACacgcgtGACATCCAGATGACTCAGTCTCCAGCCTCCC |
| 101 | TATCTGCATCTGTGGGAGAAACTGTCACCATCACATGTCGAGCAAGTGGG |
| 151 | AATATTCACAATTATTTAGCATGGTATCAGCAGAAACAGGGAAAATCTCC |
| 201 | TCAGCTCCTGGTCTATAATGCAAAAACCTTAGCAGATGGTGTGCCATCAA |
| 251 | GGTTCAGTGGCAGTGGATCAGGAACACAATATTCTCTCAAGATCAACAGC |
| 301 | CTGCAGCCTGAAGATTTTGGGAGTTATTACTGTCAACATTTTTGGAGTAC |
| 351 | TCCGTGGACGTTCGGTGGAGGCACCAAGCTGGAAATCAAACGG |
| 1-60 | Signal peptide |
| 61-393 | Light chain variable region |
| SEQ ID NO: 26. I9-6001 light chain variable | |
| region, amino acid sequence, ID: 500734p | |
| | .........o.........o.........o.........o.........o |
| 1 | METDTLLLWVLLLWVPGSTGDTRDIQMTQSPASLSASVGETVTITCRASG |
| 51 | NIHNYLAWYQQKQGKSPQLLVYNAKTLADGVPSRFSGSGSGTQYSLKINS |
| 101 | LQPEDFGSYYCQHFWSTPWTFGGGTKLEIKR |
| 1-20 | Signal peptide |
| 21-131 | Light chain variable region |
| SEQ ID NO: 27. I9-6001 heavy chain variable | |
| region, nucleotide sequence, ID: 500735n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACacgcgtCAGGTCCAGCTGCAGCAGTCTGGAGCTGAGC |
| 101 | TGGTAAGGCCTGGGACTTCAGTGAAGGTGTCCTGCAAGGCTTCTGGATAC |
| 151 | GCCTTCACTAATTACTTGATAGAGTGGGTAAAGCAGAGGCCTGGACAGGG |
| 201 | CCTTGAGTGGATTGGAGTGATTAATCCTGGAAGTGGTGGTACTAACTACA |
| 251 | ATGAGAAGTTCAAGGGCAAGGCAACACTGACTGCAGACAAATCCTCCAGC |
| 301 | ACTGCCTACATGCAGCTCAGCAGCCTGACATCTGATGACTCTGCGGTCTA |
| 351 | TTTCTGTGCAAGATGGGACTACGGTAGTAGCTACGAACGTGCTATGGACT |
| 401 | ACTGGGGTCAAGGAACCTCAGTCACCGTCTCC |
| 1-60 | Signal peptide |
| 61-432 | Heavy chain variable region |
| SEQ ID NO: 28. I9-6001 heavy chain variable | |
| region, amino acid sequence, ID: 500735p | |
| | .........o.........o.........o.........o.........o |
| 1 | METDTLLLWVLLLWVPGSTGDTRQVQLQQSGAELVRPGTSVKVSCKASGY |
| 51 | AFTNYLIEWVKQRPGQGLEWIGVINPGSGGTNYNEKFKGKATLTADKSSS |
| 101 | TAYMQLSSLTSDDSAVYFCARWDYGSSYERAMDYWGQGTSVTVS |
| 1-20 | Signal peptide |
| 21-144 | Heavy chain variable region |
| SEQ ID NO: 29. I9-7002 light chain variable | |
| region, nucleotide sequence, ID: 500744n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACacgcgtAGTATTGTGATGACCCAGACTCCCAAATTCC |
| 101 | TGCTTGTATCAGCAGGAGACAGGGTTACCATAACCTGCAAGGCCAGTCAG |
| 151 | AGTGTGAGTAATGATGTAGCTTGGTACCAACAGAAGCCAGGGCAGTCTCC |
| 201 | TAAACTGCTGATATACTATGCATCCAATCGCTACACTGGAGTCCCTGATC |
| 251 | GCTTCACTGGCAGTGGATATGGGACGGATTTCACTTTCACCATCAGCACT |
| 301 | GTGCAGGCTGAAGACCTGGCAGTTTATTTCTGTCAGCAGGATTATAGCTC |
| 351 | TCCTCTCACGTTCGGCTCGGGGACAAAGTTGGAAATAAAACGG |
| 1-60 | Signal peptide |
| 61-393 | Light chain variable region |
| SEQ ID NO: 30. I9-7002 light chain variable | |
| region, amino acid sequence, ID: 500744p | |
| | .........o.........o.........o.........o.........o |
| 1 | METDTLLLWVLLLWVPGSTGDTRSIVMTQTPKFLLVSAGDRVTITCKASQ |
| 51 | SVSNDVAWYQQKPGQSPKLLIYYASNRYTGVPDRFTGSGYGTDFTFTIST |
| 101 | VQAEDLAVYFCQQDYSSPLTFGSGTKLEIKR |
| 1-20 | Signal peptide |
| 21-131 | Light chain variable region |
| SEQ ID NO: 31. I9-7002 heavy chain variable | |
| region, nucleotide sequence, ID: 500745n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACacgcgtCAGATCCAGTTGGTGCAGTCTGGACCTGAGC |
| 101 | TGAAGAAGCCTGGAGAGACAGTCAAGATCTCCTGCAAGGCTTCTGGGTAT |
| 151 | ACCTTCACAAACTATGGAATGAACTGGGTGAAGCAGGCTCCAGGAAAGGG |
| 201 | TTTAAAGTGGATGGGCTGGATAAACACCTACACTGGAGAGCCAACATATG |
| 251 | CTGATGACTTCAAGGGACGGTTTGCCTTCTCTTTGGAAACCTCTGCCAGC |
| 301 | ACTGCCTATTTGCAGATCAACAACCTCAAAAATGAGGACATGGCTACATA |
| 351 | TTTCTGTGCAAGAACGGCGGATCTACTATGGTTACGACGTCGGTTTGCTT |
| 401 | ACTGGGGCCAAGGGACTCTGGTCACTGTCTCT |
| 1-60 | Signal peptide |
| 61-432 | Heavy chain variable region |
| SEQ ID NO: 32. I9-7002 heavy chain variable | |
| region, amino acid sequence, ID: 500745p | |
| | .........o.........o.........o.........o.........o |
| 1 | METDTLLLWVLLLWVPGSTGDTRQIQLVQSGPELKKPGETVKISCKASGY |
| 51 | TFTNYGMNWVKQAPGKGLKWMGWINTYTGEPTYADDFKGRFAFSLETSAS |
| 101 | TAYLQINNLKNEDMATYFCARTADLLWLRRRFAYWGQGTLVTVS |
| 1-20 | Signal peptide |
| 21-144 | Heavy chain variable region |
| SEQ ID NO: 33. Lysostaphin-linker, nucleotide, | |
| ID: 500693n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACGCCACCCACGAGCACTCCGCCCAGTGGCTGAACAACT |
| 101 | ACAAGAAGGGCTACGGCTACGGCCCCTACCCCCTGGGCATCAACGGCGGC |
| 151 | ATGCACTACGGCGTGGACTTCTTCATGAACATCGGCACCCCCGTGAAGGC |
| 201 | CATCTCCTCCGGCAAGATCGTGGAGGCCGGCTGGTCCAACTACGGCGGCG |
| 251 | GCAACCAGATCGGCCTGATCGAGAACGACGGCGTGCACCGCCAGTGGTAC |
| 301 | ATGCACCTGTCCAAGTACAACGTGAAGGTGGGCGACTACGTGAAGGCCGG |
| 351 | CCAGATCATCGGCTGGTCCGGCTCCACCGGCTACTCCACCGCCCCCCACC |
| 401 | TGCACTTCCAGCGCATGGTGAACTCCTTCTCCAACTCCACCGCCCAGGAC |
| 451 | CCCATGCCCTTCCTGAAGTCCGCCGGCTACGGCAAGGCCGGCGGCACCGT |
| 501 | GACCCCCACCCCCAACACCGGCTGGAAGACCAACAAGTACGGCACCCTGT |
| 551 | ACAAGTCCGAGTCCGCCTCCTTCACCCCCAACACCGACATCATCACCCGC |
| 601 | ACCACCGGCCCCTTCCGCTCCATGCCCCAGTCCGGCGTGCTGAAGGCCGG |
| 651 | CCAGACCATCCACTACGACGAGGTGATGAAGCAGGACGGCCACGTGTGGG |
| 701 | TGGGCTACACCGGCAACTCCGGCCAGCGCATCTACCTGCCCGTGCGCACC |
| 751 | TGGAACAAGTCCACCAACACCCTGGGCGTGCTGTGGGGCACCATCAAGGG |
| 801 | TGGTGGCGGTTCAGGCGGAGGTGGCTCTGGCGGTGGCGGATC |
| 1-60 | Signal peptide |
| 64-798 | Lysostaphin GI: 291246386 |
| 799-843 | Linker |
| SEQ ID NO: 34. Lysostaphin-linker, amino acid, | |
| ID: 500693n | |
| | .........o.........o.........o.........o.........o |
| 1 | METDTLLLWVLLLWVPGSTGDATHEHSAQWLNNYKKGYGYGPYPLGINGG |
| 51 | MHYGVDFFMNIGTPVKAISSGKIVEAGWSNYGGGNQIGLIENDGVHRQWY |
| 101 | MHLSKYNVKVGDYVKAGQIIGWSGSTGYSTAPHLHFQRMVNSFSNSTAQD |
| 151 | PMPFLKSAGYGKAGGTVTPTPNTGWKTNKYGTLYKSESASFTPNTDIITR |
| 201 | TTGPFRSMPQSGVLKAGQTIHYDEVMKQDGHVWVGYTGNSGQRIYLPVRT |
| 251 | WNKSTNTLGVLWGTIKGGGGSGGGGSGGGGS |
| 1-20 | Signal peptide |
| 21-266 | Lysostaphin GI: 291246386 |
| 267-281 | Linker |
| SEQ ID NO: 35. Human kappa light chain constant | |
| region, nucleotide, ID: 500693n | |
| | .........o.........o.........o.........o.........o |
| 1 | ACTGTGGCTGCACCATCTGTCTTCATCTTCCCGCCATCTGATGAGCAGTT |
| 51 | GAAATCTGGAACTGCCTCTGTTGTGTGCCTGCTGAATAACTTCTATCCCA |
| 101 | GAGAGGCCAAAGTACAGTGGAAGGTGGATAACGCCCTCCAATCGGGTAAC |
| 151 | TCCCAGGAGAGTGTCACAGAGCAGGACAGCAAGGACAGCACCTACAGCCT |
| 201 | CAGCAGCACCCTGACGCTGAGCAAAGCAGACTACGAGAAACACAAAGTCT |
| 251 | ACGCCTGCGAAGTCACCCATCAGGGCCTGAGCTCGCCCGTCACAAAGAGC |
| 301 | TTCAACAGGGGAGAGTGTTAG |
| 1-321 | Human kappa light chain constant region |
| SEQ ID NO: 36. Human kappa light chain constant | |
| region, amino acid, ID: 500693n | |
| | .........o.........o.........o.........o.........o |
| 1 | TVAAPSVFIFPPSDEQLKSGTASVVCLLNNFYPREAKVQWKVDNALQSGN |
| 51 | SQESVTEQDSKDSTYSLSSTLTLSKADYEKHKVYACEVTHQGLSSPVTKS |
| 101 | FNRGEC |
| 1-106 | Human kappa light chain constant region |
| SEQ ID NO: 37. Human gamma 1 heavy chain constant | |
| region, nucleotide, ID: 500686n | |
| | .........o.........o.........o.........o.........o |
| 1 | GCGTCGACCAAGGGCCCATCGGTCTTCCCCCTGGCACCCTCTAGCAAGAG |
| 51 | CACCTCTGGGGGCACAGCGGCCCTGGGCTGCCTGGTCAAGGACTACTTCC |
| 101 | CCGAACCGGTGACGGTGTCGTGGAACTCAGGCGCCCTGACCAGCGGCGTG |
| 151 | CACACCTTCCCGGCTGTCCTACAGTCCTCAGGACTCTACTCCCTCAGCAG |
| 201 | CGTGGTGACCGTGCCCTCCAGCAGCTTGGGCACCCAGACCTACATCTGCA |
| 251 | ACGTGAATCACAAGCCCAGCAACACCAAGGTGGACAAGAGAGTTGAGCCC |
| 301 | AAATCTTGTGACAAAACTCACACATGCCCACCGTGCCCAGCACCTGAACT |
| 351 | CCTGGGGGGACCGTCAGTCTTCCTCTTCCCCCCAAAACCCAAGGACACCC |
| 401 | TCATGATCTCCCGGACCCCTGAGGTCACATGCGTGGTGGTGGACGTGAGC |
| 451 | CACGAAGACCCTGAGGTCAAGTTCAACTGGTACGTGGACGGCGTGGAGGT |
| 501 | GCATAATGCCAAGACAAAGCCGCGGGAGGAGCAGTACAACAGCACGTACC |
| 551 | GTGTGGTCAGCGTCCTCACCGTCCTGCACCAGGACTGGCTGAATGGCAAG |
| 601 | GAGTACAAGTGCAAGGTCTCCAACAAAGCCCTCCCAGCCCCCATCGAGAA |
| 651 | AACCATCTCCAAAGCCAAAGGGCAGCCCCGAGAACCACAGGTGTACACCC |
| 701 | TGCCCCCATCCCGGGAGGAGATGACCAAGAACCAGGTCAGCCTGACCTGC |
| 751 | CTGGTCAAAGGCTTCTATCCCAGCGACATCGCCGTGGAGTGGGAGAGCAA |
| 801 | TGGGCAGCCGGAGAACAACTACAAGACCACGCCTCCCGTGCTGGACTCCG |
| 851 | ACGGCTCCTTCTTCCTCTATAGCAAGCTCACCGTGGACAAGAGCAGGTGG |
| 901 | CAGCAGGGGAACGTCTTCTCATGCTCCGTGATGCATGAGGCTCTGCACAA |
| 951 | CCACTACACGCAGAAGAGCCTCTCCCTGTCTCCGGGTAAATGA |
| 1-993 | Human gamma 1 heavy chain constant region |
| SEQ ID NO: 38. Human gamma 1 heavy chain constant | |
| region, amino acid, ID: 500686p | |
| | .........o.........o.........o.........o.........o |
| 1 | ASTKGPSVFPLAPSSKSTSGGTAALGCLVKDYFPEPVTVSWNSGALTSGV |
| 51 | HTFPAVLQSSGLYSLSSVVTVPSSSLGTQTYICNVNHKPSNTKVDKRVEP |
| 101 | KSCDKTHTCPPCPAPELLGGPSVFLFPPKPKDTLMISRTPEVTCVVVDVS |
| 151 | HEDPEVKFNWYVDGVEVHNAKTKPREEQYNSTYRVVSVLTVLHQDWLNGK |
| 201 | EYKCKVSNKALPAPIEKTISKAKGQPREPQVYTLPPSREEMTKNQVSLTC |
| 251 | LVKGFYPSDIAVEWESNGQPENNYKTTPPVLDSDGSFFLYSKLTVDKSRW |
| 301 | QQGNVFSCSVMHEALHNHYTQKSLSLSPGK |
| 1-330 | Human gamma 1 heavy chain constant region |
| SEQ ID NO: 39. Linker-human cathelicidin (LL-37), | |
| nucleotide ID: 500669n | |
| | .........o.........o.........o.........o.........o |
| 1 | GCGGCCGCAGGTGGTGGCGGTTCAGGCGGAGGTGGCTCTGGCGGTGGCGG |
| 51 | ATCCCTGCTGGGGGATTTCTTCCGGAAGTCTAAAGAGAAGATTGGGAAAG |
| 101 | AGTTTAAAAGAATTGTCCAGAGAATCAAGGATTTTTTGCGGAATCTTGTG |
| 151 | CCCAGGACAGAATCCTAG |
| 1-54 | Linker |
| 55-168 | Human cathelicidin (LL-37) |
| SEQ ID NO: 40. Linker-human cathelicidin (LL-37), | |
| amino acid, ID: 500669p | |
| | .........o.........o.........o.........o.........o |
| 1 | AAAGGGGSGGGGSGGGGSLLGDFFRKSKEKIGKEFKRIVQRIKDFLRNLV |
| 51 | PRTES |
| 1-18 | Linker |
| 19-55 | Human cathelicidin (LL-37) |
| SEQ ID NO: 41. Linker-human beta defensin 2 | |
| (HBD2), nucleotide, ID: 500670n | |
| | .........o.........o.........o.........o.........o |
| 1 | GCGGCCGCAGGTGGTGGCGGTTCAGGCGGAGGTGGCTCTGGCGGTGGCGG |
| 51 | ATCCGGTATAGGCGATCCTGTTACCTGCCTTAAGAGTGGAGCCATATGTC |
| 101 | ATCCAGTCTTTTGCCCTAGAAGGTATAAACAAATTGGCACCTGTGGTCTC |
| 151 | CCTGGAACAAAATGCTGCAAAAAGCCATGA |
| 1-54 | Linker |
| 55-180 | Human beta defensin 2 |
| SEQ ID NO: 42. Linker-human beta defensin 2 | |
| (HBD2), amino acid, ID: 500670p | |
| | .........o.........o.........o.........o.........o |
| 1 | AAAGGGGSGGGGSGGGGSLLGDFFRKSKEKIGKEFKRIVQRIKDFLRNLV |
| 51 | PRTES |
| 1-18 | Linker |
| 19-55 | Human beta defensin 2 |
| SEQ ID NO: 43. Linker-human beta defensin 3 | |
| (HBD3), nucleotide, ID: 500671n | |
| | .........o.........o.........o.........o.........o |
| 1 | GCGGCCGCAGGTGGTGGCGGTTCAGGCGGAGGTGGCTCTGGCGGTGGCGG |
| 51 | ATCCGGAATCATAAACACATTACAGAAATATTATTGCAGAGTCAGAGGCG |
| 101 | GCCGGTGTGCTGTGCTCAGCTGCCTTCCAAAGGAGGAACAGATCGGCAAG |
| 151 | TGCTCGACGCGTGGCCGAAAATGCTGCCGAAGAAAGAAATAA |
| 1-54 | Linker |
| 55-192 | Human beta defensin 3 |
| SEQ ID NO: 44. Linker-human beta defensin 3 | |
| (HBD3), amino acid, ID: 500671p | |
| | .........o.........o.........o.........o.........o |
| 1 | AAAGGGGSGGGGSGGGGSGIINTLQKYYCRVRGGRCAVLSCLPKEEQIGK |
| 51 | CSTRGRKCCRRKK |
| 1-18 | Linker |
| 19-63 | Human beta defensin 3 |
| SEQ ID NO: 45. Linker-human phospholipase A2, | |
| group IIa, nucleotide, ID: 500729n | |
| | .........o.........o.........o.........o.........o |
| 1 | gcggccgCAGGTGGTGGCGGTTCAGGCGGAGGTGGCTCTGGCGGTGGCGG |
| 51 | ATCGAATTTGGTGAATTTCCACAGAATGATCAAGTTGACGACAGGAAAGG |
| 101 | AAGCCGCACTCAGTTATGGCTTCTACGGCTGCCACTGTGGCGTGGGTGGC |
| 151 | AGAGGATCCCCCAAGGATGCAACGGATCGCTGCTGTGTCACTCATGACTG |
| 201 | TTGCTACAAACGTCTGGAGAAACGTGGATGTGGCACCAAATTTCTGAGCT |
| 251 | ACAAGTTTAGCAACTCGGGGAGCAGAATCACCTGTGCAAAACAGGACTCC |
| 301 | TGCAGAAGTCAACTGTGTGAGTGTGATAAGGCTGCTGCCACCTGTTTTGC |
| 351 | TAGAAACAAGACGACCTACAATAAAAAGTACCAGTACTATTCCAATAAAC |
| 401 | ACTGCAGAGGGAGCACCCCTCGTTGCTGA |
| 1-54 | Linker |
| 55-429 | Human PLA2 IIa |
| SEQ ID NO: 46. Linker-human phospholipase A2, | |
| group IIa, amino acid, ID: 500729p | |
| | .........o.........o.........o.........o.........o |
| 1 | AAAGGGGSGGGGSGGGGSNLVNFHRMIKLTTGKEAALSYGFYGCHCGVGG |
| 51 | RGSPKDATDRCCVTHDCCYKRLEKRGCGTKFLSYKFSNSGSRITCAKQDS |
| 101 | CRSQLCECDKAAATCFARNKTTYNKKYQYYSNKHCRGSTPRC |
| 1-18 | Linker |
| 19-142 | Human PLA2 IIa |
| SEQ ID NO: 47. Antibody IA9 light chain variable | |
| region, nucleotide sequence, ID: 500266n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACaCGCGTGATGTTGTGATGACCCAAATTCCACTCTCCC |
| 101 | TGCCTGTCAGTCTTGGAGATCAAGCCTCCATCTCTTGCAGATCTAGTCAG |
| 151 | AGCCTTGTACACAGTAATGGAAACACCTATTTACATTGGTACCTGCAGAA |
| 201 | GCCAGGCCAGTCTCCAAAGCTCCTGATCTACAAAGTTTCCAACCGATTTT |
| 251 | CTGGGGTCCCAGACAGGTTCAGTGGCAGTGGATCAGGGACAGATTTCACA |
| 301 | CTCAAGATCAGCAGAGTGGAGGCTGAGGATCTGGGAGTTTATTTCTGCTC |
| 351 | TCAAAGTACACATGTTCCTCCGTGGACGTTTGGTGGAGGCACCAAGCTGG |
| 401 | AAATCAAACGG |
| 1-60 | Signal peptide |
| 61-411 | Light chain variable region |
| SEQ ID NO: 48. Antibody IA9 light chain variable | |
| region, amino acid sequence, ID: 500266p | |
| | .........o.........o.........o.........o.........o |
| 1 | METDTLLLWVLLLWVPGSTGDTRDVVMTQIPLSLPVSLGDQASISCRSSQ |
| 51 | SLVHSNGNTYLHWYLQKPGQSPKLLIYKVSNRFSGVPDRFSGSGSGTDFT |
| 101 | LKISRVEAEDLGVYFCSQSTHVPPWTFGGGTKLEIKR |
| 1-20 | Signal peptide |
| 21-137 | Light chain variable region |
| SEQ ID NO: 49. IA9 heavy chain variable region, | |
| nucleotide sequence, ID: 500302n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACaCGCGTCAGATCCAGTTGGTGCAGTCTGGACCTGAGC |
| 101 | TGAAGAAGCCTGGAGAGACAGTCAAGATCTCCTGCAAGGCTTCTGGGTAT |
| 151 | ACCTTCACAAACTATGGAATGAACTGGGTGAAGCAGGCTCCAGGAAAGGG |
| 201 | TTTAAAGTGGATGGGCTGGATAAACACCAACACTGGAGAGCCAACATATG |
| 251 | CTGAAGAGTTCAAGGGGCGGTTTGCCTTCTCTTTGGAAACCTCTGCCAGC |
| 301 | ACTGCCTATTTGCAGATCAACAACCTCAAAAATGAGGACACGGCTACATA |
| 351 | TTTCTGTGCAAGACACGGTGGTAGGAGCTGGTACTTCGATGTCTGGGGCG |
| 401 | CAGGGACCACGGTCACCGTCTCCTCAGCG |
| 1-60 | Signal peptide |
| 61-429 | Heavy chain variable region |
| SEQ ID NO: 50. Antibody IA9 heavy chain variable | |
| region, amino acid sequence, ID: 500302p | |
| | .........o.........o.........o.........o.........o |
| 1 | METDTLLLWVLLLWVPGSTGDTRQIQLVQSGPELKKPGETVKISCKASGY |
| 51 | TFTNYGMNWVKQAPGKGLKWMGWINTNTGEPTYAEEFKGRFAFSLETSAS |
| 101 | TAYLQINNLKNEDTATYFCARHGGRSWYFDVWGAGTTVTVSSA |
| 1-20 | Signal peptide |
| 21-143 | Heavy chain variable region |

### Examples for Complete Constructs, Biocide-LC and HC-Biocide

| SEQ ID NO: 51. LYST-LC-I7-3019, Lysostaphin-light | |
|---|---|
| chain chimeric murine-human fusion, nucleotide | |
| sequence, ID: 500693n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACGCCACCCACGAGCACTCCGCCCAGTGGCTGAACAACT |
| 101 | ACAAGAAGGGCTACGGCTACGGCCCCTACCCCCTGGGCATCAACGGCGGC |
| 151 | ATGCACTACGGCGTGGACTTCTTCATGAACATCGGCACCCCCGTGAAGGC |
| 201 | CATCTCCTCCGGCAAGATCGTGGAGGCCGGCTGGTCCAACTACGGCGGCG |
| 251 | GCAACCAGATCGGCCTGATCGAGAACGACGGCGTGCACCGCCAGTGGTAC |
| 301 | ATGCACCTGTCCAAGTACAACGTGAAGGTGGGCGACTACGTGAAGGCCGG |
| 351 | CCAGATCATCGGCTGGTCCGGCTCCACCGGCTACTCCACCGCCCCCCACC |
| 401 | TGCACTTCCAGCGCATGGTGAACTCCTTCTCCAACTCCACCGCCCAGGAC |
| 451 | CCCATGCCCTTCCTGAAGTCCGCCGGCTACGGCAAGGCCGGCGGCACCGT |
| 501 | GACCCCCACCCCCAACACCGGCTGGAAGACCAACAAGTACGGCACCCTGT |
| 551 | ACAAGTCCGAGTCCGCCTCCTTCACCCCCAACACCGACATCATCACCCGC |
| 601 | ACCACCGGCCCCTTCCGCTCCATGCCCCAGTCCGGCGTGCTGAAGGCCGG |
| 651 | CCAGACCATCCACTACGACGAGGTGATGAAGCAGGACGGCCACGTGTGGG |
| 701 | TGGGCTACACCGGCAACTCCGGCCAGCGCATCTACCTGCCCGTGCGCACC |
| 751 | TGGAACAAGTCCACCAACACCCTGGGCGTGCTGTGGGGCACCATCAAGGG |
| 801 | TGGTGGCGGTTCAGGCGGAGGTGGCTCTGGCGGTGGCGGATCcacgcgtG |
| 851 | ATGTTTTGATGACCCAAACTCCACTCTCCCTGCCTGTCAGTCTTGGAGAT |
| 901 | CAAGCCTCCATCTCTTGCAGATCTAGTCAGAGCATTGTACATACTAATGG |
| 951 | AAACACCTATTTAGAATGGTACCTGCAGAAACCGGGCCAGTCTCCAAAGC |
| 1001 | TCCTGATCTACAAAGTTTCCAACCGATTTTCTGGGGTCCCAGACAGGTTC |
| 1051 | AGTGGCAGTGGATCAGGGACAGATTTCACACTCAAGATCAGCAGAGTGGA |
| 1101 | GGCTGAGGATCTGGGAGTTTATTACTGCTTTCAAGGTTCACATATTCCGT |
| 1151 | GGACGTTCGGTGGAGGCACCAAGCTGGAAATCAAACGGACTGTGGCTGCA |
| 1201 | CCATCTGTCTTCATCTTCCCGCCATCTGATGAGCAGTTGAAATCTGGAAC |
| 1251 | TGCCTCTGTTGTGTGCCTGCTGAATAACTTCTATCCCAGAGAGGCCAAAG |
| 1301 | TACAGTGGAAGGTGGATAACGCCCTCCAATCGGGTAACTCCCAGGAGAGT |
| 1351 | GTCACAGAGCAGGACAGCAAGGACAGCACCTACAGCCTCAGCAGCACCCT |
| 1401 | GACGCTGAGCAAAGCAGACTACGAGAAACACAAAGTCTACGCCTGCGAAG |
| 1451 | TCACCCATCAGGGCCTGAGCTCGCCCGTCACAAAGAGCTTCAACAGGGGA |
| 1501 | GAGTGTTAG |
| 1-60 | Signal peptide |
| 61-1509 | Lysostaphin-Light chain fusion |
| SEQ ID NO: 52. LYST-LC-I7-3019, Lysostaphin-light | |
| chain fusion, murine-human chimeric, amino acid | |
| sequence, ID: 500693p | |
| | .........o.........o.........o.........o.........o |
| 1 | METDTLLLWVLLLWVPGSTGDATHEHSAQWLNNYKKGYGYGPYPLGINGG |
| 51 | MHYGVDFFMNIGTPVKAISSGKIVEAGWSNYGGGNQIGLIENDGVHRQWY |
| 101 | MHLSKYNVKVGDYVKAGQIIGWSGSTGYSTAPHLHFQRMVNSFSNSTAQD |
| 151 | PMPFLKSAGYGKAGGTVTPTPNTGWKTNKYGTLYKSESASFTPNTDIITR |
| 201 | TTGPFRSMPQSGVLKAGQTIHYDEVMKQDGHVWVGYTGNSGQRIYLPVRT |
| 251 | WNKSTNTLGVLWGTIKGGGGSGGGGSGGGGSTRDVLMTQTPLSLPVSLGD |
| 301 | QASISCRSSQSIVHTNGNTYLEWYLQKPGQSPKLLIYKVSNRFSGVPDRF |
| 351 | SGSGSGTDFTLKISRVEAEDLGVYYCFQGSHIPWTFGGGTKLEIKRTVAA |
| 401 | PSVFIFPPSDEQLKSGTASVVCLLNNFYPREAKVQWKVDNALQSGNSQES |
| 451 | VTEQDSKDSTYSLSSTLTLSKADYEKHKVYACEVTHQGLSSPVTKSFNRG |
| 501 | EC |
| 1-20 | Signal peptide |
| 21-502 | Lysostaphin-Light chain fusion |
| SEQ ID NO: 53. I7-3019-HC-Lyst, heavy chain- | |
| lysostaphin chimeric murine-human fusion, | |
| nucleotide sequence, ID: 500691n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACACGCGTCAGGTCCAACTGCAGCAGCCTGGGGCTGAGC |
| 101 | TGGTGAGGCCTGGGGCTTCAGTGAACCTGTCCTGCAGGGCTTCTGGCTAC |
| 151 | ACCTTCACCACCTACTGGATGATCTGGGTGAAGCAGAGGCCTGGACAAGG |
| 201 | CCTTGAATGGATTGGTATGATTGATCCTTCAGACAGTGAGACTCACTACA |
| 251 | ATCAAATGTTCAAGGACAAGGCCACATTGACTGTAGACAAATCCTCCACC |
| 301 | ACAGCCTACATGCAGTTCAGCAGCCTAACATCTGAGGACTCTGCGGTCTA |
| 351 | TTACTGTGCAAGATGGAACTTCGGTAAGGGCTACTGGGGCCAAGGCACCA |
| 401 | CTCTCACGGTCTCCTCAGCGTCGACCAAGGGCCCATCGGTCTTCCCCCTG |
| 451 | GCACCCTCTAGCAAGAGCACCTCTGGGGGCACAGCGGCCCTGGGCTGCCT |
| 501 | GGTCAAGGACTACTTCCCCGAACCGGTGACGGTGTCGTGGAACTCAGGCG |
| 551 | CCCTGACCAGCGGCGTGCACACCTTCCCGGCTGTCCTACAGTCCTCAGGA |
| 601 | CTCTACTCCCTCAGCAGCGTGGTGACCGTGCCCTCCAGCAGCTTGGGCAC |
| 651 | CCAGACCTACATCTGCAACGTGAATCACAAGCCCAGCAACACCAAGGTGG |
| 701 | ACAAGAGAGTTGAGCCCAAATCTTGTGACAAAACTCACACATGCCCACCG |
| 751 | TGCCCAGCACCTGAACTCCTGGGGGGACCGTCAGTCTTCCTCTTCCCCCC |
| 801 | AAAACCCAAGGACACCCTCATGATCTCCCGGACCCCTGAGGTCACATGCG |
| 851 | TGGTGGTGGACGTGAGCCACGAAGACCCTGAGGTCAAGTTCAACTGGTAC |
| 901 | GTGGACGGCGTGGAGGTGCATAATGCCAAGACAAAGCCGCGGGAGGAGCA |
| 951 | GTACAACAGCACGTACCGTGTGGTCAGCGTCCTCACCGTCCTGCACCAGG |
| 1001 | ACTGGCTGAATGGCAAGGAGTACAAGTGCAAGGTCTCCAACAAAGCCCTC |
| 1051 | CCAGCCCCCATCGAGAAAACCATCTCCAAAGCCAAAGGGCAGCCCCGAGA |
| 1101 | ACCACAGGTGTACACCCTGCCCCCATCCCGGGAGGAGATGACCAAGAACC |
| 1151 | AGGTCAGCCTGACCTGCCTGGTCAAAGGCTTCTATCCCAGCGACATCGCC |
| 1201 | GTGGAGTGGGAGAGCAATGGGCAGCCGGAGAACAACTACAAGACCACGCC |
| 1251 | TCCCGTGCTGGACTCCGACGGCTCCTTCTTCCTCTATAGCAAGCTCACCG |
| 1301 | TGGACAAGAGCAGGTGGCAGCAGGGGAACGTCTTCTCATGCTCCGTGATG |
| 1351 | CATGAGGCTCTGCACAACCACTACACGCAGAAGAGCCTCTCCCTGTCTCC |
| 1401 | GGGTAAAGCGGCCGCAGGTGGTGGCGGTTCAGGCGGAGGTGGCTCTGGCG |
| 1451 | GTGGCGGATCCGCCACCCACGAGCACTCCGCCCAGTGGCTGAACAACTAC |
| 1501 | AAGAAGGGCTACGGCTACGGCCCCTACCCCCTGGGCATCAACGGCGGCAT |
| 1551 | GCACTACGGCGTGGACTTCTTCATGAACATCGGCACCCCCGTGAAGGCCA |
| 1601 | TCTCCTCCGGCAAGATCGTGGAGGCCGGCTGGTCCAACTACGGCGGCGGC |
| 1651 | AACCAGATCGGCCTGATCGAGAACGACGGCGTGCACCGCCAGTGGTACAT |
| 1701 | GCACCTGTCCAAGTACAACGTGAAGGTGGGCGACTACGTGAAGGCCGGCC |
| 1751 | AGATCATCGGCTGGTCCGGCTCCACCGGCTACTCCACCGCCCCCCACCTG |
| 1801 | CACTTCCAGCGCATGGTGAACTCCTTCTCCAACTCCACCGCCCAGGACCC |
| 1851 | CATGCCCTTCCTGAAGTCCGCCGGCTACGGCAAGGCCGGCGGCACCGTGA |
| 1901 | CCCCCACCCCCAACACCGGCTGGAAGACCAACAAGTACGGCACCCTGTAC |
| 1951 | AAGTCCGAGTCCGCCTCCTTCACCCCCAACACCGACATCATCACCCGCAC |
| 2001 | CACCGGCCCCTTCCGCTCCATGCCCCAGTCCGGCGTGCTGAAGGCCGGCC |
| 2051 | AGACCATCCACTACGACGAGGTGATGAAGCAGGACGGCCACGTGTGGGTG |
| 2101 | GGCTACACCGGCAACTCCGGCCAGCGCATCTACCTGCCCGTGCGCACCTG |
| 2151 | GAACAAGTCCACCAACACCCTGGGCGTGCTGTGGGGCACCATCAAGTGA |
| 1-60 | Signal peptide |
| 61-2151 | Heavy chain-lysostaphin fusion |
| SEQ ID NO: 54. I7-3019-HC-Lyst, heavy chain- | |
| lysostaphin chimeric murine-human fusion, amino | |
| acid sequence, ID: 500691p | |
| | .........o.........o.........o.........o.........o |
| 1 | METDTLLLWVLLLWVPGSTGDTRQVQLQQPGAELVRPGASVNLSCRASGY |
| 51 | TFTTYWMIWVKQRPGQGLEWIGMIDPSDSETHYNQMFKDKATLTVDKSST |
| 101 | TAYMQFSSLTSEDSAVYYCARWNFGKGYWGQGTTLTVSSASTKGPSVFPL |
| 151 | APSSKSTSGGTAALGCLVKDYFPEPVTVSWNSGALTSGVHTFPAVLQSSG |
| 201 | LYSLSSVVTVPSSSLGTQTYICNVNHKPSNTKVDKRVEPKSCDKTHTCPP |
| 251 | CPAPELLGGPSVFLFPPKPKDTLMISRTPEVTCVVVDVSHEDPEVKFNWY |
| 301 | VDGVEVHNAKTKPREEQYNSTYRVVSVLTVLHQDWLNGKEYKCKVSNKAL |
| 351 | PAPIEKTISKAKGQPREPQVYTLPPSREEMTKNQVSLTCLVKGFYPSDIA |
| 401 | VEWESNGQPENNYKTTPPVLDSDGSFFLYSKLTVDKSRWQQGNVFSCSVM |
| 451 | HEALHNHYTQKSLSLSPGKAAAGGGGSGGGGSGGGGSATHEHSAQWLNNY |
| 501 | KKGYGYGPYPLGINGGMHYGVDFFMNIGTPVKAISSGKIVEAGWSNYGGG |
| 551 | NQIGLIENDGVHRQWYMHLSKYNVKVGDYVKAGQIIGWSGSTGYSTAPHL |
| 601 | HFQRMVNSFSNSTAQDPMPFLKSAGYGKAGGTVTPTPNTGWKTNKYGTLY |
| 651 | KSESASFTPNTDIITRTTGPFRSMPQSGVLKAGQTIHYDEVMKQDGHVWV |
| 701 | GYTGNSGQRIYLPVRTWNKSTNTLGVLWGTIK |
| 1-20 | Signal peptide |
| 21-732 | chimeric heavy chain lysostaphin fusion |
| SEQ ID NO: 55. I7-3019-HC-PLA2, heavy chain-PLA2 | |
| chimeric murine-human fusion, nucleotide sequence, | |
| ID: 500690n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACACGCGTCAGGTCCAACTGCAGCAGCCTGGGGCTGAGC |
| 101 | TGGTGAGGCCTGGGGCTTCAGTGAACCTGTCCTGCAGGGCTTCTGGCTAC |
| 151 | ACCTTCACCACCTACTGGATGATCTGGGTGAAGCAGAGGCCTGGACAAGG |
| 201 | CCTTGAATGGATTGGTATGATTGATCCTTCAGACAGTGAGACTCACTACA |
| 251 | ATCAAATGTTCAAGGACAAGGCCACATTGACTGTAGACAAATCCTCCACC |
| 301 | ACAGCCTACATGCAGTTCAGCAGCCTAACATCTGAGGACTCTGCGGTCTA |
| 351 | TTACTGTGCAAGATGGAACTTCGGTAAGGGCTACTGGGGCCAAGGCACCA |
| 401 | CTCTCACGGTCTCCTCAGCGTCGACCAAGGGCCCATCGGTCTTCCCCCTG |
| 451 | GCACCCTCTAGCAAGAGCACCTCTGGGGGCACAGCGGCCCTGGGCTGCCT |
| 501 | GGTCAAGGACTACTTCCCCGAACCGGTGACGGTGTCGTGGAACTCAGGCG |
| 551 | CCCTGACCAGCGGCGTGCACACCTTCCCGGCTGTCCTACAGTCCTCAGGA |
| 601 | CTCTACTCCCTCAGCAGCGTGGTGACCGTGCCCTCCAGCAGCTTGGGCAC |
| 651 | CCAGACCTACATCTGCAACGTGAATCACAAGCCCAGCAACACCAAGGTGG |
| 701 | ACAAGAGAGTTGAGCCCAAATCTTGTGACAAAACTCACACATGCCCACCG |
| 751 | TGCCCAGCACCTGAACTCCTGGGGGGACCGTCAGTCTTCCTCTTCCCCCC |
| 801 | AAAACCCAAGGACACCCTCATGATCTCCCGGACCCCTGAGGTCACATGCG |
| 851 | TGGTGGTGGACGTGAGCCACGAAGACCCTGAGGTCAAGTTCAACTGGTAC |
| 901 | GTGGACGGCGTGGAGGTGCATAATGCCAAGACAAAGCCGCGGGAGGAGCA |
| 951 | GTACAACAGCACGTACCGTGTGGTCAGCGTCCTCACCGTCCTGCACCAGG |
| 1001 | ACTGGCTGAATGGCAAGGAGTACAAGTGCAAGGTCTCCAACAAAGCCCTC |
| 1051 | CCAGCCCCCATCGAGAAAACCATCTCCAAAGCCAAAGGGCAGCCCCGAGA |
| 1101 | ACCACAGGTGTACACCCTGCCCCCATCCCGGGAGGAGATGACCAAGAACC |
| 1151 | AGGTCAGCCTGACCTGCCTGGTCAAAGGCTTCTATCCCAGCGACATCGCC |
| 1201 | GTGGAGTGGGAGAGCAATGGGCAGCCGGAGAACAACTACAAGACCACGCC |
| 1251 | TCCCGTGCTGGACTCCGACGGCTCCTTCTTCCTCTATAGCAAGCTCACCG |
| 1301 | TGGACAAGAGCAGGTGGCAGCAGGGGAACGTCTTCTCATGCTCCGTGATG |
| 1351 | CATGAGGCTCTGCACAACCACTACACGCAGAAGAGCCTCTCCCTGTCTCC |
| 1401 | GGGTAAAGCGGCCGCAGGTGGTGGCGGTTCAGGCGGAGGTGGCTCTGGCG |
| 1451 | GTGGCGGATCGAATTTGGTGAATTTCCACAGAATGATCAAGTTGACGACA |
| 1501 | GGAAAGGAAGCCGCACTCAGTTATGGCTTCTACGGCTGCCACTGTGGCGT |
| 1551 | GGGTGGCAGAGGATCCCCCAAGGATGCAACGGATCGCTGCTGTGTCACTC |
| 1601 | ATGACTGTTGCTACAAACGTCTGGAGAAACGTGGATGTGGCACCAAATTT |
| 1651 | CTGAGCTACAAGTTTAGCAACTCGGGGAGCAGAATCACCTGTGCAAAACA |
| 1701 | GGACTCCTGCAGAAGTCAACTGTGTGAGTGTGATAAGGCTGCTGCCACCT |
| 1751 | GTTTTGCTAGAAACAAGACGACCTACAATAAAAAGTACCAGTACTATTCC |
| 1801 | AATAAACACTGCAGAGGGAGCACCCCTCGTTGCTGA |
| 1-60 | Signal peptide |
| 61-1836 | Chimeric murine-human Heavy chain-PLA2 fusion |
| SEQ ID NO: 56. I7-3019-HC-PLA2, heavy chain-PLA2 | |
| chimeric murine-human fusion, amino acid sequence, | |
| ID: 500690 | |
| | .........o.........o.........o.........o.........o |
| 1 | METDTLLLWVLLLWVPGSTGDTRQVQLQQPGAELVRPGASVNLSCRASGY |
| 51 | TFTTYWMIWVKQRPGQGLEWIGMIDPSDSETHYNQMFKDKATLTVDKSST |
| 101 | TAYMQFSSLTSEDSAVYYCARWNFGKGYWGQGTTLTVSSASTKGPSVFPL |
| 151 | APSSKSTSGGTAALGCLVKDYFPEPVTVSWNSGALTSGVHTFPAVLQSSG |
| 201 | LYSLSSVVTVPSSSLGTQTYICNVNHKPSNTKVDKRVEPKSCDKTHTCPP |
| 251 | CPAPELLGGPSVFLFPPKPKDTLMISRTPEVTCVVVDVSHEDPEVKFNWY |
| 301 | VDGVEVHNAKTKPREEQYNSTYRVVSVLTVLHQDWLNGKEYKCKVSNKAL |
| 351 | PAPIEKTISKAKGQPREPQVYTLPPSREEMTKNQVSLTCLVKGFYPSDIA |
| 401 | VEWESNGQPENNYKTTPPVLDSDGSFFLYSKLTVDKSRWQQGNVFSCSVM |
| 451 | HEALHNHYTQKSLSLSPGKAAAGGGGSGGGGSGGGGSNLVNFHRMIKLTT |
| 501 | GKEAALSYGFYGCHCGVGGRGSPKDATDRCCVTHDCCYKRLEKRGCGTKF |
| 551 | LSYKFSNSGSRITCAKQDSCRSQLCECDKAAATCFARNKTTYNKKYQYYS |
| 601 | NKHCRGSTPRC |
| 1-20 | Signal peptide |
| 21-611 | chimeric murine-human heavy chain PLA2 fusion |
| SEQ ID NO: 57. I7-3019-HC-LL37, heavy chain-PLA2 | |
| chimeric murine-human fusion, nucleotide sequence, | |
| ID: 500690n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACACGCGTCAGGTCCAACTGCAGCAGCCTGGGGCTGAGC |
| 101 | TGGTGAGGCCTGGGGCTTCAGTGAACCTGTCCTGCAGGGCTTCTGGCTAC |
| 151 | ACCTTCACCACCTACTGGATGATCTGGGTGAAGCAGAGGCCTGGACAAGG |
| 201 | CCTTGAATGGATTGGTATGATTGATCCTTCAGACAGTGAGACTCACTACA |
| 251 | ATCAAATGTTCAAGGACAAGGCCACATTGACTGTAGACAAATCCTCCACC |
| 301 | ACAGCCTACATGCAGTTCAGCAGCCTAACATCTGAGGACTCTGCGGTCTA |
| 351 | TTACTGTGCAAGATGGAACTTCGGTAAGGGCTACTGGGGCCAAGGCACCA |
| 401 | CTCTCACGGTCTCCTCAGCGTCGACCAAGGGCCCATCGGTCTTCCCCCTG |
| 451 | GCACCCTCTAGCAAGAGCACCTCTGGGGGCACAGCGGCCCTGGGCTGCCT |
| 501 | GGTCAAGGACTACTTCCCCGAACCGGTGACGGTGTCGTGGAACTCAGGCG |
| 551 | CCCTGACCAGCGGCGTGCACACCTTCCCGGCTGTCCTACAGTCCTCAGGA |
| 601 | CTCTACTCCCTCAGCAGCGTGGTGACCGTGCCCTCCAGCAGCTTGGGCAC |
| 651 | CCAGACCTACATCTGCAACGTGAATCACAAGCCCAGCAACACCAAGGTGG |
| 701 | ACAAGAGAGTTGAGCCCAAATCTTGTGACAAAACTCACACATGCCCACCG |
| 751 | TGCCCAGCACCTGAACTCCTGGGGGGACCGTCAGTCTTCCTCTTCCCCCC |
| 801 | AAAACCCAAGGACACCCTCATGATCTCCCGGACCCCTGAGGTCACATGCG |
| 851 | TGGTGGTGGACGTGAGCCACGAAGACCCTGAGGTCAAGTTCAACTGGTAC |
| 901 | GTGGACGGCGTGGAGGTGCATAATGCCAAGACAAAGCCGCGGGAGGAGCA |
| 951 | GTACAACAGCACGTACCGTGTGGTCAGCGTCCTCACCGTCCTGCACCAGG |
| 1001 | ACTGGCTGAATGGCAAGGAGTACAAGTGCAAGGTCTCCAACAAAGCCCTC |
| 1051 | CCAGCCCCCATCGAGAAAACCATCTCCAAAGCCAAAGGGCAGCCCCGAGA |
| 1101 | ACCACAGGTGTACACCCTGCCCCCATCCCGGGAGGAGATGACCAAGAACC |
| 1151 | AGGTCAGCCTGACCTGCCTGGTCAAAGGCTTCTATCCCAGCGACATCGCC |
| 1201 | GTGGAGTGGGAGAGCAATGGGCAGCCGGAGAACAACTACAAGACCACGCC |
| 1251 | TCCCGTGCTGGACTCCGACGGCTCCTTCTTCCTCTATAGCAAGCTCACCG |
| 1301 | TGGACAAGAGCAGGTGGCAGCAGGGGAACGTCTTCTCATGCTCCGTGATG |
| 1351 | CATGAGGCTCTGCACAACCACTACACGCAGAAGAGCCTCTCCCTGTCTCC |
| 1401 | GGGTAAAGCGGCCGCAGGTGGTGGCGGTTCAGGCGGAGGTGGCTCTGGCG |
| 1451 | GTGGCGGATCGAATTTGGTGAATTTCCACAGAATGATCAAGTTGACGACA |
| 1501 | GGAAAGGAAGCCGCACTCAGTTATGGCTTCTACGGCTGCCACTGTGGCGT |
| 1551 | GGGTGGCAGAGGATCCCCCAAGGATGCAACGGATCGCTGCTGTGTCACTC |
| 1601 | ATGACTGTTGCTACAAACGTCTGGAGAAACGTGGATGTGGCACCAAATTT |
| 1651 | CTGAGCTACAAGTTTAGCAACTCGGGGAGCAGAATCACCTGTGCAAAACA |
| 1701 | GGACTCCTGCAGAAGTCAACTGTGTGAGTGTGATAAGGCTGCTGCCACCT |
| 1751 | GTTTTGCTAGAAACAAGACGACCTACAATAAAAAGTACCAGTACTATTCC |
| 1801 | AATAAACACTGCAGAGGGAGCACCCCTCGTTGCTGA |
| 1-60 | Signal peptide |
| 61-1836 | Chimeric murine-human Heavy chain-PLA2 fusion |
| SEQ ID NO: 58. I7-3019-HC-LL37, heavy chain-PLA2 | |
| chimeric murine-human fusion, amino acid sequence, | |
| ID: 500690 | |
| p | |
| | .........o.........o.........o.........o.........o |
| 1 | METDTLLLWVLLLWVPGSTGDTRQVQLQQPGAELVRPGASVNLSCRASGY |
| 51 | TFTTYWMIWVKQRPGQGLEWIGMIDPSDSETHYNQMFKDKATLTVDKSST |
| 101 | TAYMQFSSLTSEDSAVYYCARWNFGKGYWGQGTTLTVSSASTKGPSVFPL |
| 151 | APSSKSTSGGTAALGCLVKDYFPEPVTVSWNSGALTSGVHTFPAVLQSSG |
| 201 | LYSLSSVVTVPSSSLGTQTYICNVNHKPSNTKVDKRVEPKSCDKTHTCPP |
| 251 | CPAPELLGGPSVFLFPPKPKDTLMISRTPEVTCVVVDVSHEDPEVKFNWY |
| 301 | VDGVEVHNAKTKPREEQYNSTYRVVSVLTVLHQDWLNGKEYKCKVSNKAL |
| 351 | PAPIEKTISKAKGQPREPQVYTLPPSREEMTKNQVSLTCLVKGFYPSDIA |
| 401 | VEWESNGQPENNYKTTPPVLDSDGSFFLYSKLTVDKSRWQQGNVFSCSVM |
| 451 | HEALHNHYTQKSLSLSPGKAAAGGGGSGGGGSGGGGSNLVNFHRMIKLTT |
| 501 | GKEAALSYGFYGCHCGVGGRGSPKDATDRCCVTHDCCYKRLEKRGCGTKF |
| 551 | LSYKFSNSGSRITCAKQDSCRSQLCECDKAAATCFARNKTTYNKKYQYYS |
| 601 | NKHCRGSTPRC |
| 1-20 | Signal peptide |
| 21-611 | chimeric murine-human heavy chain LL37 fusion |
| SEQ ID NO: 59. LYST-LC-I8-1024, Lysostaphin-light | |
| chain chimeric murine-human fusion, nucleotide | |
| sequence, ID: 500675n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACGCCACCCACGAGCACTCCGCCCAGTGGCTGAACAACT |
| 101 | ACAAGAAGGGCTACGGCTACGGCCCCTACCCCCTGGGCATCAACGGCGGC |
| 151 | ATGCACTACGGCGTGGACTTCTTCATGAACATCGGCACCCCCGTGAAGGC |
| 201 | CATCTCCTCCGGCAAGATCGTGGAGGCCGGCTGGTCCAACTACGGCGGCG |
| 251 | GCAACCAGATCGGCCTGATCGAGAACGACGGCGTGCACCGCCAGTGGTAC |
| 301 | ATGCACCTGTCCAAGTACAACGTGAAGGTGGGCGACTACGTGAAGGCCGG |
| 351 | CCAGATCATCGGCTGGTCCGGCTCCACCGGCTACTCCACCGCCCCCCACC |
| 401 | TGCACTTCCAGCGCATGGTGAACTCCTTCTCCAACTCCACCGCCCAGGAC |
| 451 | CCCATGCCCTTCCTGAAGTCCGCCGGCTACGGCAAGGCCGGCGGCACCGT |
| 501 | GACCCCCACCCCCAACACCGGCTGGAAGACCAACAAGTACGGCACCCTGT |
| 551 | ACAAGTCCGAGTCCGCCTCCTTCACCCCCAACACCGACATCATCACCCGC |
| 601 | ACCACCGGCCCCTTCCGCTCCATGCCCCAGTCCGGCGTGCTGAAGGCCGG |
| 651 | CCAGACCATCCACTACGACGAGGTGATGAAGCAGGACGGCCACGTGTGGG |
| 701 | TGGGCTACACCGGCAACTCCGGCCAGCGCATCTACCTGCCCGTGCGCACC |
| 751 | TGGAACAAGTCCACCAACACCCTGGGCGTGCTGTGGGGCACCATCAAGGG |
| 801 | TGGTGGCGGTTCAGGCGGAGGTGGCTCTGGCGGTGGCGGATCcacgcgtG |
| 851 | ACATTGTGATGACCCAGTCTCAAAAATTCATGTCCACATCAGTAGGAGAC |
| 901 | AGGGTCAGCGTCACCTGCAAGGCCAGTCAGAATGTGGGTACTAATGTAGC |
| 951 | CTGGTATCAACAGAAACCAGGGCAATCTCCTAAAGCACTGATTTACTCGG |
| 1001 | CATCCTACCGGTACAGTGGAGTCCCTGATCGCTTCACAGGCAGTGGATCT |
| 1051 | GGGACAGATTTCACTCTCACCATCAGCAATGTGCAGTCTGAAGACTTGGC |
| 1101 | AGAGTATTTCTGTCAGCAATATAACAGCTATCCTCTCACGTTCGGTGCTG |
| 1151 | GGACCAAGCTGGAGCTGAAACGGACTGTGGCTGCACCATCTGTCTTCATC |
| 1201 | TTCCCGCCATCTGATGAGCAGTTGAAATCTGGAACTGCCTCTGTTGTGTG |
| 1251 | CCTGCTGAATAACTTCTATCCCAGAGAGGCCAAAGTACAGTGGAAGGTGG |
| 1301 | ATAACGCCCTCCAATCGGGTAACTCCCAGGAGAGTGTCACAGAGCAGGAC |
| 1351 | AGCAAGGACAGCACCTACAGCCTCAGCAGCACCCTGACGCTGAGCAAAGC |
| 1401 | AGACTACGAGAAACACAAAGTCTACGCCTGCGAAGTCACCCATCAGGGCC |
| 1451 | TGAGCTCGCCCGTCACAAAGAGCTTCAACAGGGGAGAGTGTTAG |
| 1-60 | Signal peptide |
| 61-1494 | Lysostaphin-Light chain fusion |
| SEQ ID NO: 60. LYST-LC-I8-1024, Lysostaphin-light | |
| chain fusion, murine-human chimeric, amino acid | |
| sequence, ID: 500675p | |
| | .........o.........o.........o.........o.........o |
| 1 | METDTLLLWVLLLWVPGSTGDATHEHSAQWLNNYKKGYGYGPYPLGINGG |
| 51 | MHYGVDFFMNIGTPVKAISSGKIVEAGWSNYGGGNQIGLIENDGVHRQWY |
| 101 | MHLSKYNVKVGDYVKAGQIIGWSGSTGYSTAPHLHFQRMVNSFSNSTAQD |
| 151 | PMPFLKSAGYGKAGGTVTPTPNTGWKTNKYGTLYKSESASFTPNTDIITR |
| 201 | TTGPFRSMPQSGVLKAGQTIHYDEVMKQDGHVWVGYTGNSGQRIYLPVRT |
| 251 | WNKSTNTLGVLWGTIKGGGGSGGGGSGGGGSTRDIVMTQSQKFMSTSVGD |
| 301 | RVSVTCKASQNVGTNVAWYQQKPGQSPKALIYSASYRYSGVPDRFTGSGS |
| 351 | GTDFTLTISNVQSEDLAEYFCQQYNSYPLTFGAGTKLELKRTVAAPSVFI |
| 401 | FPPSDEQLKSGTASVVCLLNNFYPREAKVQWKVDNALQSGNSQESVTEQD |
| 451 | SKDSTYSLSSTLTLSKADYEKHKVYACEVTHQGLSSPVTKSFNRGEC |
| 1-20 | Signal peptide |
| 21-497 | Lysostaphin-Light chain fusion |
| SEQ ID NO: 61. I8-1024-HC-HBD2, heavy chain-HBD2 | |
| chimeric murine-human fusion, nucleotide sequence, | |
| ID: 500670n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACACGCGTGAAGTGAAGCTGGTGGAGTCTGGGGGAGGTT |
| 101 | TAGTGCAGCCTGGAGGGTCCCTGAAACTCTCCTGTGCAGCCTCTGGATTC |
| 151 | ACTTTCAGTAGCTATACCATGTCTTGGGTTCGCCAGACTCCAGAGAAGAG |
| 201 | GCTGGAGTGGGTCGCATACATTAGTAATGGTGGTGGTAGCACCTACTATC |
| 251 | CAGACACTGTAAAGGGCCGATTCACCATCTCCAGAGACAATGCCAAGAAC |
| 301 | ACCCTGTACCTGCAAATGAGCAGTCTGAAGTCTGAGGACACGGCCATGTA |
| 351 | TTACTGTGCAAGACAGGTACGACGGGGGATGGACTACTGGGGTCAAGGAA |
| 401 | CCTCAGTCACCGTCTCCTCAGCGTCGACCAAGGGCCCATCGGTCTTCCCC |
| 451 | CTGGCACCCTCTAGCAAGAGCACCTCTGGGGGCACAGCGGCCCTGGGCTG |
| 501 | CCTGGTCAAGGACTACTTCCCCGAACCGGTGACGGTGTCGTGGAACTCAG |
| 551 | GCGCCCTGACCAGCGGCGTGCACACCTTCCCGGCTGTCCTACAGTCCTCA |
| 601 | GGACTCTACTCCCTCAGCAGCGTGGTGACCGTGCCCTCCAGCAGCTTGGG |
| 651 | CACCCAGACCTACATCTGCAACGTGAATCACAAGCCCAGCAACACCAAGG |
| 701 | TGGACAAGAGAGTTGAGCCCAAATCTTGTGACAAAACTCACACATGCCCA |
| 751 | CCGTGCCCAGCACCTGAACTCCTGGGGGGACCGTCAGTCTTCCTCTTCCC |
| 801 | CCCAAAACCCAAGGACACCCTCATGATCTCCCGGACCCCTGAGGTCACAT |
| 851 | GCGTGGTGGTGGACGTGAGCCACGAAGACCCTGAGGTCAAGTTCAACTGG |
| 901 | TACGTGGACGGCGTGGAGGTGCATAATGCCAAGACAAAGCCGCGGGAGGA |
| 951 | GCAGTACAACAGCACGTACCGTGTGGTCAGCGTCCTCACCGTCCTGCACC |
| 1001 | AGGACTGGCTGAATGGCAAGGAGTACAAGTGCAAGGTCTCCAACAAAGCC |
| 1051 | CTCCCAGCCCCCATCGAGAAAACCATCTCCAAAGCCAAAGGGCAGCCCCG |
| 1101 | AGAACCACAGGTGTACACCCTGCCCCCATCCCGGGAGGAGATGACCAAGA |
| 1151 | ACCAGGTCAGCCTGACCTGCCTGGTCAAAGGCTTCTATCCCAGCGACATC |
| 1201 | GCCGTGGAGTGGGAGAGCAATGGGCAGCCGGAGAACAACTACAAGACCAC |
| 1251 | GCCTCCCGTGCTGGACTCCGACGGCTCCTTCTTCCTCTATAGCAAGCTCA |
| 1301 | CCGTGGACAAGAGCAGGTGGCAGCAGGGGAACGTCTTCTCATGCTCCGTG |
| 1351 | ATGCATGAGGCTCTGCACAACCACTACACGCAGAAGAGCCTCTCCCTGTC |
| 1401 | TCCGGGTAAAGCGGCCGCAGGTGGTGGCGGTTCAGGCGGAGGTGGCTCTG |
| 1451 | GCGGTGGCGGATCCGGTATAGGCGATCCTGTTACCTGCCTTAAGAGTGGA |
| 1501 | GCCATATGTCATCCAGTCTTTTGCCCTAGAAGGTATAAACAAATTGGCAC |
| 1551 | CTGTGGTCTCCCTGGAACAAAATGCTGCAAAAAGCCATGA |
| 1-60 | Signal peptide |
| 61-1590 | Chimeric murine-human Heavy chain-HBD2 fusion |
| SEQ ID NO: 62. I8-1024-HC-HBD2, heavy chain-HBD2 | |
| chimeric murine-human fusion, amino acid sequence, | |
| ID: 500670p | |
| | .........o.........o.........o.........o.........o |
| 1 | METDTLLLWVLLLWVPGSTGDTREVKLVESGGGLVQPGGSLKLSCAASGF |
| 51 | TFSSYTMSWVRQTPEKRLEWVAYISNGGGSTYYPDTVKGRFTISRDNAKN |
| 101 | TLYLQMSSLKSEDTAMYYCARQVRRGMDYWGQGTSVTVSSASTKGPSVFP |
| 151 | LAPSSKSTSGGTAALGCLVKDYFPEPVTVSWNSGALTSGVHTFPAVLQSS |
| 201 | GLYSLSSVVTVPSSSLGTQTYICNVNHKPSNTKVDKRVEPKSCDKTHTCP |
| 251 | PCPAPELLGGPSVFLFPPKPKDTLMISRTPEVTCVVVDVSHEDPEVKFNW |
| 301 | YVDGVEVHNAKTKPREEQYNSTYRVVSVLTVLHQDWLNGKEYKCKVSNKA |
| 351 | LPAPIEKTISKAKGQPREPQVYTLPPSREEMTKNQVSLTCLVKGFYPSDI |
| 401 | AVEWESNGQPENNYKTTPPVLDSDGSFFLYSKLTVDKSRWQQGNVFSCSV |
| 451 | MHEALHNHYTQKSLSLSPGKAAAGGGGSGGGGSGGGGSGIGDPVTCLKSG |
| 501 | AICHPVFCPRRYKQIGTCGLPGTKCCKKP |
| 1-20 | Signal peptide |
| 21-529 | chimeric murine-human heavy chain HBD2 fusion |
| SEQ ID NO: 63. I8-1024-HC-HBD3, heavy chain-HBD3 | |
| chimeric murine-human fusion, nucleotide sequence, | |
| ID: 500671n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACACGCGTGAAGTGAAGCTGGTGGAGTCTGGGGGAGGTT |
| 101 | TAGTGCAGCCTGGAGGGTCCCTGAAACTCTCCTGTGCAGCCTCTGGATTC |
| 151 | ACTTTCAGTAGCTATACCATGTCTTGGGTTCGCCAGACTCCAGAGAAGAG |
| 201 | GCTGGAGTGGGTCGCATACATTAGTAATGGTGGTGGTAGCACCTACTATC |
| 251 | CAGACACTGTAAAGGGCCGATTCACCATCTCCAGAGACAATGCCAAGAAC |
| 301 | ACCCTGTACCTGCAAATGAGCAGTCTGAAGTCTGAGGACACGGCCATGTA |
| 351 | TTACTGTGCAAGACAGGTACGACGGGGGATGGACTACTGGGGTCAAGGAA |
| 401 | CCTCAGTCACCGTCTCCTCAGCGTCGACCAAGGGCCCATCGGTCTTCCCC |
| 451 | CTGGCACCCTCTAGCAAGAGCACCTCTGGGGGCACAGCGGCCCTGGGCTG |
| 501 | CCTGGTCAAGGACTACTTCCCCGAACCGGTGACGGTGTCGTGGAACTCAG |
| 551 | GCGCCCTGACCAGCGGCGTGCACACCTTCCCGGCTGTCCTACAGTCCTCA |
| 601 | GGACTCTACTCCCTCAGCAGCGTGGTGACCGTGCCCTCCAGCAGCTTGGG |
| 651 | CACCCAGACCTACATCTGCAACGTGAATCACAAGCCCAGCAACACCAAGG |
| 701 | TGGACAAGAGAGTTGAGCCCAAATCTTGTGACAAAACTCACACATGCCCA |
| 751 | CCGTGCCCAGCACCTGAACTCCTGGGGGGACCGTCAGTCTTCCTCTTCCC |
| 801 | CCCAAAACCCAAGGACACCCTCATGATCTCCCGGACCCCTGAGGTCACAT |
| 851 | GCGTGGTGGTGGACGTGAGCCACGAAGACCCTGAGGTCAAGTTCAACTGG |
| 901 | TACGTGGACGGCGTGGAGGTGCATAATGCCAAGACAAAGCCGCGGGAGGA |
| 951 | GCAGTACAACAGCACGTACCGTGTGGTCAGCGTCCTCACCGTCCTGCACC |
| 1001 | AGGACTGGCTGAATGGCAAGGAGTACAAGTGCAAGGTCTCCAACAAAGCC |
| 1051 | CTCCCAGCCCCCATCGAGAAAACCATCTCCAAAGCCAAAGGGCAGCCCCG |
| 1101 | AGAACCACAGGTGTACACCCTGCCCCCATCCCGGGAGGAGATGACCAAGA |
| 1151 | ACCAGGTCAGCCTGACCTGCCTGGTCAAAGGCTTCTATCCCAGCGACATC |
| 1201 | GCCGTGGAGTGGGAGAGCAATGGGCAGCCGGAGAACAACTACAAGACCAC |
| 1251 | GCCTCCCGTGCTGGACTCCGACGGCTCCTTCTTCCTCTATAGCAAGCTCA |
| 1301 | CCGTGGACAAGAGCAGGTGGCAGCAGGGGAACGTCTTCTCATGCTCCGTG |
| 1351 | ATGCATGAGGCTCTGCACAACCACTACACGCAGAAGAGCCTCTCCCTGTC |
| 1401 | TCCGGGTAAAGCGGCCGCAGGTGGTGGCGGTTCAGGCGGAGGTGGCTCTG |
| 1451 | GCGGTGGCGGATCCGGAATCATAAACACATTACAGAAATATTATTGCAGA |
| 1501 | GTCAGAGGCGGCCGGTGTGCTGTGCTCAGCTGCCTTCCAAAGGAGGAACA |
| 1551 | GATCGGCAAGTGCTCGACGCGTGGCCGAAAATGCTGCCGAAGAAAGAAAT |
| 1601 | AA |
| 1-60 | Signal peptide |
| 61-1602 | Chimeric murine-human Heavy chain-HBD3 fusion |
| SEQ ID NO: 64. I8-1024-HC-HBD3, heavy chain-HBD3 | |
| chimeric murine-human fusion, amino acid sequence, | |
| ID: 500671p | |
| | .........o.........o.........o.........o.........o |
| 1 | METDTLLLWVLLLWVPGSTGDTREVKLVESGGGLVQPGGSLKLSCAASGF |
| 51 | TFSSYTMSWVRQTPEKRLEWVAYISNGGGSTYYPDTVKGRFTISRDNAKN |
| 101 | TLYLQMSSLKSEDTAMYYCARQVRRGMDYWGQGTSVTVSSASTKGPSVFP |
| 151 | LAPSSKSTSGGTAALGCLVKDYFPEPVTVSWNSGALTSGVHTFPAVLQSS |
| 201 | GLYSLSSVVTVPSSSLGTQTYICNVNHKPSNTKVDKRVEPKSCDKTHTCP |
| 251 | PCPAPELLGGPSVFLFPPKPKDTLMISRTPEVTCVVVDVSHEDPEVKFNW |
| 301 | YVDGVEVHNAKTKPREEQYNSTYRVVSVLTVLHQDWLNGKEYKCKVSNKA |
| 351 | LPAPIEKTISKAKGQPREPQVYTLPPSREEMTKNQVSLTCLVKGFYPSDI |
| 401 | AVEWESNGQPENNYKTTPPVLDSDGSFFLYSKLTVDKSRWQQGNVFSCSV |
| 451 | MHEALHNHYTQKSLSLSPGKAAAGGGGSGGGGSGGGGSGIINTLQKYYCR |
| 501 | VRGGRCAVLSCLPKEEQIGKCSTRGRKCCRRKK |
| 1-20 | Signal peptide |
| 21-533 | chimeric murine-human heavy chain HBD3 fusion |
| SEQ ID NO: 65. I8-1024-HC-LL37, heavy chain-LL37 | |
| chimeric murine-human fusion, nucleotide sequence, | |
| ID: 500669n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACACGCGTGAAGTGAAGCTGGTGGAGTCTGGGGGAGGTT |
| 101 | TAGTGCAGCCTGGAGGGTCCCTGAAACTCTCCTGTGCAGCCTCTGGATTC |
| 151 | ACTTTCAGTAGCTATACCATGTCTTGGGTTCGCCAGACTCCAGAGAAGAG |
| 201 | GCTGGAGTGGGTCGCATACATTAGTAATGGTGGTGGTAGCACCTACTATC |
| 251 | CAGACACTGTAAAGGGCCGATTCACCATCTCCAGAGACAATGCCAAGAAC |
| 301 | ACCCTGTACCTGCAAATGAGCAGTCTGAAGTCTGAGGACACGGCCATGTA |
| 351 | TTACTGTGCAAGACAGGTACGACGGGGGATGGACTACTGGGGTCAAGGAA |
| 401 | CCTCAGTCACCGTCTCCTCAGCGTCGACCAAGGGCCCATCGGTCTTCCCC |
| 451 | CTGGCACCCTCTAGCAAGAGCACCTCTGGGGGCACAGCGGCCCTGGGCTG |
| 501 | CCTGGTCAAGGACTACTTCCCCGAACCGGTGACGGTGTCGTGGAACTCAG |
| 551 | GCGCCCTGACCAGCGGCGTGCACACCTTCCCGGCTGTCCTACAGTCCTCA |
| 601 | GGACTCTACTCCCTCAGCAGCGTGGTGACCGTGCCCTCCAGCAGCTTGGG |
| 651 | CACCCAGACCTACATCTGCAACGTGAATCACAAGCCCAGCAACACCAAGG |
| 701 | TGGACAAGAGAGTTGAGCCCAAATCTTGTGACAAAACTCACACATGCCCA |
| 751 | CCGTGCCCAGCACCTGAACTCCTGGGGGGACCGTCAGTCTTCCTCTTCCC |
| 801 | CCCAAAACCCAAGGACACCCTCATGATCTCCCGGACCCCTGAGGTCACAT |
| 851 | GCGTGGTGGTGGACGTGAGCCACGAAGACCCTGAGGTCAAGTTCAACTGG |
| 901 | TACGTGGACGGCGTGGAGGTGCATAATGCCAAGACAAAGCCGCGGGAGGA |
| 951 | GCAGTACAACAGCACGTACCGTGTGGTCAGCGTCCTCACCGTCCTGCACC |
| 1001 | AGGACTGGCTGAATGGCAAGGAGTACAAGTGCAAGGTCTCCAACAAAGCC |
| 1051 | CTCCCAGCCCCCATCGAGAAAACCATCTCCAAAGCCAAAGGGCAGCCCCG |
| 1101 | AGAACCACAGGTGTACACCCTGCCCCCATCCCGGGAGGAGATGACCAAGA |
| 1151 | ACCAGGTCAGCCTGACCTGCCTGGTCAAAGGCTTCTATCCCAGCGACATC |
| 1201 | GCCGTGGAGTGGGAGAGCAATGGGCAGCCGGAGAACAACTACAAGACCAC |
| 1251 | GCCTCCCGTGCTGGACTCCGACGGCTCCTTCTTCCTCTATAGCAAGCTCA |
| 1301 | CCGTGGACAAGAGCAGGTGGCAGCAGGGGAACGTCTTCTCATGCTCCGTG |
| 1351 | ATGCATGAGGCTCTGCACAACCACTACACGCAGAAGAGCCTCTCCCTGTC |
| 1401 | TCCGGGTAAAGCGGCCGCAGGTGGTGGCGGTTCAGGCGGAGGTGGCTCTG |
| 1451 | GCGGTGGCGGATCCCTGCTGGGGGATTTCTTCCGGAAGTCTAAAGAGAAG |
| 1501 | ATTGGGAAAGAGTTTAAAAGAATTGTCCAGAGAATCAAGGATTTTTTGCG |
| 1551 | GAATCTTGTGCCCAGGACAGAATCCTAG |
| 1-60 | Signal peptide |
| 61-1578 | Chimeric murine-human Heavy chain-LL37 fusion |
| SEQ ID NO: 66. I8-1024-HC-LL37, heavy chain-LL37 | |
| chimeric murine-human fusion, amino acid sequence, | |
| ID: 500669p | |
| | .........o.........o.........o.........o.........o |
| 1 | METDTLLLWVLLLWVPGSTGDTREVKLVESGGGLVQPGGSLKLSCAASGF |
| 51 | TFSSYTMSWVRQTPEKRLEWVAYISNGGGSTYYPDTVKGRFTISRDNAKN |
| 101 | TLYLQMSSLKSEDTAMYYCARQVRRGMDYWGQGTSVTVSSASTKGPSVFP |
| 151 | LAPSSKSTSGGTAALGCLVKDYFPEPVTVSWNSGALTSGVHTFPAVLQSS |
| 201 | GLYSLSSVVTVPSSSLGTQTYICNVNHKPSNTKVDKRVEPKSCDKTHTCP |
| 251 | PCPAPELLGGPSVFLFPPKPKDTLMISRTPEVTCVVVDVSHEDPEVKFNW |
| 301 | YVDGVEVHNAKTKPREEQYNSTYRVVSVLTVLHQDWLNGKEYKCKVSNKA |
| 351 | LPAPIEKTISKAKGQPREPQVYTLPPSREEMTKNQVSLTCLVKGFYPSDI |
| 401 | AVEWESNGQPENNYKTTPPVLDSDGSFFLYSKLTVDKSRWQQGNVFSCSV |
| 451 | MHEALHNHYTQKSLSLSPGKAAAGGGGSGGGGSGGGGSLLGDFFRKSKEK |
| 501 | IGKEFKRIVQRIKDFLRNLVPRTES |
| 1-20 | Signal peptide |
| 21-525 | chimeric murine-human heavy chain LL37 fusion |
| SEQ ID NO: 67. I8-1024-HC-PLA2, heavy chain-PLA2 | |
| chimeric murine-human fusion, nucleotide sequence, | |
| ID: 500672n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACACGCGTGAAGTGAAGCTGGTGGAGTCTGGGGGAGGTT |
| 101 | TAGTGCAGCCTGGAGGGTCCCTGAAACTCTCCTGTGCAGCCTCTGGATTC |
| 151 | ACTTTCAGTAGCTATACCATGTCTTGGGTTCGCCAGACTCCAGAGAAGAG |
| 201 | GCTGGAGTGGGTCGCATACATTAGTAATGGTGGTGGTAGCACCTACTATC |
| 251 | CAGACACTGTAAAGGGCCGATTCACCATCTCCAGAGACAATGCCAAGAAC |
| 301 | ACCCTGTACCTGCAAATGAGCAGTCTGAAGTCTGAGGACACGGCCATGTA |
| 351 | TTACTGTGCAAGACAGGTACGACGGGGGATGGACTACTGGGGTCAAGGAA |
| 401 | CCTCAGTCACCGTCTCCTCAGCGTCGACCAAGGGCCCATCGGTCTTCCCC |
| 451 | CTGGCACCCTCTAGCAAGAGCACCTCTGGGGGCACAGCGGCCCTGGGCTG |
| 501 | CCTGGTCAAGGACTACTTCCCCGAACCGGTGACGGTGTCGTGGAACTCAG |
| 551 | GCGCCCTGACCAGCGGCGTGCACACCTTCCCGGCTGTCCTACAGTCCTCA |
| 601 | GGACTCTACTCCCTCAGCAGCGTGGTGACCGTGCCCTCCAGCAGCTTGGG |
| 651 | CACCCAGACCTACATCTGCAACGTGAATCACAAGCCCAGCAACACCAAGG |
| 701 | TGGACAAGAGAGTTGAGCCCAAATCTTGTGACAAAACTCACACATGCCCA |
| 751 | CCGTGCCCAGCACCTGAACTCCTGGGGGGACCGTCAGTCTTCCTCTTCCC |
| 801 | CCCAAAACCCAAGGACACCCTCATGATCTCCCGGACCCCTGAGGTCACAT |
| 851 | GCGTGGTGGTGGACGTGAGCCACGAAGACCCTGAGGTCAAGTTCAACTGG |
| 901 | TACGTGGACGGCGTGGAGGTGCATAATGCCAAGACAAAGCCGCGGGAGGA |
| 951 | GCAGTACAACAGCACGTACCGTGTGGTCAGCGTCCTCACCGTCCTGCACC |
| 1001 | AGGACTGGCTGAATGGCAAGGAGTACAAGTGCAAGGTCTCCAACAAAGCC |
| 1051 | CTCCCAGCCCCCATCGAGAAAACCATCTCCAAAGCCAAAGGGCAGCCCCG |
| 1101 | AGAACCACAGGTGTACACCCTGCCCCCATCCCGGGAGGAGATGACCAAGA |
| 1151 | ACCAGGTCAGCCTGACCTGCCTGGTCAAAGGCTTCTATCCCAGCGACATC |
| 1201 | GCCGTGGAGTGGGAGAGCAATGGGCAGCCGGAGAACAACTACAAGACCAC |
| 1251 | GCCTCCCGTGCTGGACTCCGACGGCTCCTTCTTCCTCTATAGCAAGCTCA |
| 1301 | CCGTGGACAAGAGCAGGTGGCAGCAGGGGAACGTCTTCTCATGCTCCGTG |
| 1351 | ATGCATGAGGCTCTGCACAACCACTACACGCAGAAGAGCCTCTCCCTGTC |
| 1401 | TCCGGGTAAAGCGGCCGCAGGTGGTGGCGGTTCAGGCGGAGGTGGCTCTG |
| 1451 | GCGGTGGCGGATCGAATTTGGTGAATTTCCACAGAATGATCAAGTTGACG |
| 1501 | ACAGGAAAGGAAGCCGCACTCAGTTATGGCTTCTACGGCTGCCACTGTGG |
| 1551 | CGTGGGTGGCAGAGGATCCCCCAAGGATGCAACGGATCGCTGCTGTGTCA |
| 1601 | CTCATGACTGTTGCTACAAACGTCTGGAGAAACGTGGATGTGGCACCAAA |
| 1651 | TTTCTGAGCTACAAGTTTAGCAACTCGGGGAGCAGAATCACCTGTGCAAA |
| 1701 | ACAGGACTCCTGCAGAAGTCAACTGTGTGAGTGTGATAAGGCTGCTGCCA |
| 1751 | CCTGTTTTGCTAGAAACAAGACGACCTACAATAAAAAGTACCAGTACTAT |
| 1801 | TCCAATAAACACTGCAGAGGGAGCACCCCTCGTTGCTGA |
| 1-60 | Signal peptide |
| 61-1839 | Chimeric murine-human Heavy chain-PLA2 fusion |
| SEQ ID NO: 68. I8-1024-HC-PLA2, heavy chain-PLA2 | |
| chimeric murine-human fusion, amino acid sequence, | |
| ID: 500672p | |
| | .........o.........o.........o.........o.........o |
| 1 | METDTLLLWVLLLWVPGSTGDTREVKLVESGGGLVQPGGSLKLSCAASGF |
| 51 | TFSSYTMSWVRQTPEKRLEWVAYISNGGGSTYYPDTVKGRFTISRDNAKN |
| 101 | TLYLQMSSLKSEDTAMYYCARQVRRGMDYWGQGTSVTVSSASTKGPSVFP |
| 151 | LAPSSKSTSGGTAALGCLVKDYFPEPVTVSWNSGALTSGVHTFPAVLQSS |
| 201 | GLYSLSSVVTVPSSSLGTQTYICNVNHKPSNTKVDKRVEPKSCDKTHTCP |
| 251 | PCPAPELLGGPSVFLFPPKPKDTLMISRTPEVTCVVVDVSHEDPEVKFNW |
| 301 | YVDGVEVHNAKTKPREEQYNSTYRVVSVLTVLHQDWLNGKEYKCKVSNKA |
| 351 | LPAPIEKTISKAKGQPREPQVYTLPPSREEMTKNQVSLTCLVKGFYPSDI |
| 401 | AVEWESNGQPENNYKTTPPVLDSDGSFFLYSKLTVDKSRWQQGNVFSCSV |
| 451 | MHEALHNHYTQKSLSLSPGKAAAGGGGSGGGGSGGGGSNLVNFHRMIKLT |
| 501 | TGKEAALSYGFYGCHCGVGGRGSPKDATDRCCVTHDCCYKRLEKRGCGTK |
| 551 | FLSYKFSNSGSRITCAKQDSCRSQLCECDKAAATCFARNKTTYNKKYQYY |
| 601 | SNKHCRGSTPRC |
| 1-20 | Signal peptide |
| 21-612 | chimeric murine-human heavy chain-PLA2 fusion |
| SEQ ID NO: 69. LYST-LC-I8-1029, Lysostaphin-light | |
| chain chimeric murine-human fusion, nucleotide | |
| sequence, ID: 500718n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACGCCACCCACGAGCACTCCGCCCAGTGGCTGAACAACT |
| 101 | ACAAGAAGGGCTACGGCTACGGCCCCTACCCCCTGGGCATCAACGGCGGC |
| 151 | ATGCACTACGGCGTGGACTTCTTCATGAACATCGGCACCCCCGTGAAGGC |
| 201 | CATCTCCTCCGGCAAGATCGTGGAGGCCGGCTGGTCCAACTACGGCGGCG |
| 251 | GCAACCAGATCGGCCTGATCGAGAACGACGGCGTGCACCGCCAGTGGTAC |
| 301 | ATGCACCTGTCCAAGTACAACGTGAAGGTGGGCGACTACGTGAAGGCCGG |
| 351 | CCAGATCATCGGCTGGTCCGGCTCCACCGGCTACTCCACCGCCCCCCACC |
| 401 | TGCACTTCCAGCGCATGGTGAACTCCTTCTCCAACTCCACCGCCCAGGAC |
| 451 | CCCATGCCCTTCCTGAAGTCCGCCGGCTACGGCAAGGCCGGCGGCACCGT |
| 501 | GACCCCCACCCCCAACACCGGCTGGAAGACCAACAAGTACGGCACCCTGT |
| 551 | ACAAGTCCGAGTCCGCCTCCTTCACCCCCAACACCGACATCATCACCCGC |
| 601 | ACCACCGGCCCCTTCCGCTCCATGCCCCAGTCCGGCGTGCTGAAGGCCGG |
| 651 | CCAGACCATCCACTACGACGAGGTGATGAAGCAGGACGGCCACGTGTGGG |
| 701 | TGGGCTACACCGGCAACTCCGGCCAGCGCATCTACCTGCCCGTGCGCACC |
| 751 | TGGAACAAGTCCACCAACACCCTGGGCGTGCTGTGGGGCACCATCAAGGG |
| 801 | TGGTGGCGGTTCAGGCGGAGGTGGCTCTGGCGGTGGCGGATCcacgcgtG |
| 851 | ACATTGTGATGACACAGTCTCCATCCTCCCTGACTGTGACAGCAGGAGAG |
| 901 | AAGGTCACTATGAGCTGCAAGTCCAGTCAGAGTCTGTTAAACAGTGGAAA |
| 951 | TCAAAAGAaCTACTTGACCTGGTACCAGCAGAAACCAGGGCAGCCTCCTA |
| 1001 | AACTGTTGATCTACTGGGCATCCACTAGGGAATCTGGGGTCCCTGATCGC |
| 1051 | TTCACAGGCAGTGGATCTGGAACAGATTTCACTCTCACCATCAGCAGTGT |
| 1101 | GCAGGCTGAAGACCTGGCAGTTTATTACTGTCAGAATGATTATAGTTATC |
| 1151 | CTTTCACGTTCGGCTCGGGGACAAAGTTGGAAATAAAACGGACTGTGGCT |
| 1201 | GCACCATCTGTCTTCATCTTCCCGCCATCTGATGAGCAGTTGAAATCTGG |
| 1251 | AACTGCCTCTGTTGTGTGCCTGCTGAATAACTTCTATCCCAGAGAGGCCA |
| 1301 | AAGTACAGTGGAAGGTGGATAACGCCCTCCAATCGGGTAACTCCCAGGAG |
| 1351 | AGTGTCACAGAGCAGGACAGCAAGGACAGCACCTACAGCCTCAGCAGCAC |
| 1401 | CCTGACGCTGAGCAAAGCAGACTACGAGAAACACAAAGTCTACGCCTGCG |
| 1451 | AAGTCACCCATCAGGGCCTGAGCTCGCCCGTCACAAAGAGCTTCAACAGG |
| 1501 | GGAGAGTGTTAG |
| 1-60 | Signal peptide |
| 61-1512 | Lysostaphin-Light chain fusion |
| SEQ ID NO: 70. LYST-LC-I8-1029, Lysostaphin-light | |
| chain fusion, murine-human chimeric, amino acid | |
| sequence, ID: 500718p | |
| | .........o.........o.........o.........o.........o |
| 1 | METDTLLLWVLLLWVPGSTGDATHEHSAQWLNNYKKGYGYGPYPLGINGG |
| 51 | MHYGVDFFMNIGTPVKAISSGKIVEAGWSNYGGGNQIGLIENDGVHRQWY |
| 101 | MHLSKYNVKVGDYVKAGQIIGWSGSTGYSTAPHLHFQRMVNSFSNSTAQD |
| 151 | PMPFLKSAGYGKAGGTVTPTPNTGWKTNKYGTLYKSESASFTPNTDIITR |
| 201 | TTGPFRSMPQSGVLKAGQTIHYDEVMKQDGHVWVGYTGNSGQRIYLPVRT |
| 251 | WNKSTNTLGVLWGTIKGGGGSGGGGSGGGGSTRDIVMTQSPSSLTVTAGE |
| 301 | KVTMSCKSSQSLLNSGNQKNYLTWYQQKPGQPPKLLIYWASTRESGVPDR |
| 351 | FTGSGSGTDFTLTISSVQAEDLAVYYCQNDYSYPFTFGSGTKLEIKRTVA |
| 401 | APSVFIFPPSDEQLKSGTASVVCLLNNFYPREAKVQWKVDNALQSGNSQE |
| 451 | SVTEQDSKDSTYSLSSTLTLSKADYEKHKVYACEVTHQGLSSPVTKSFNR |
| 501 | GEC |
| 1-20 | Signal peptide |
| 21-503 | Lysostaphin-Light chain fusion |
| SEQ ID NO: 71. I8-1029-HC-HBD2, heavy chain-HBD2 | |
| chimeric murine-human fusion, nucleotide sequence, | |
| ID: 500713n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACACGCGTGAGGTCCAGCTGCAGCAGTCTGGACCTGAGC |
| 101 | TAGTGAAGACTGGGGCTTCAGTGAAGATATCCTGCAAGGCTTCTGGTTAC |
| 151 | TCATTCACTGGTTACTACATGCACTGGGTCAAGCAGAGCCATGGAAAGAG |
| 201 | CCTTGAGTGGATTGGATATATTAGTTGTTACAATGGTGCTACTAGCTACA |
| 251 | ACCAGAAGTTCAAGGGCAAGGCCACATTTACTGTAGACACATCCTCCAGC |
| 301 | ACAGCCTACATGCAGTTCAACAGCCTGACATCTGAAGACTCTGCGGTCTA |
| 351 | TTACTGTGCAAGATCGAGGACTGGAGCCTGGTTTGCTTACTGGGGCCAAG |
| 401 | GGACTCTGGTCACTGTCTCTGCGTCGACCAAGGGCCCATCGGTCTTCCCC |
| 451 | CTGGCACCCTCTAGCAAGAGCACCTCTGGGGGCACAGCGGCCCTGGGCTG |
| 501 | CCTGGTCAAGGACTACTTCCCCGAACCGGTGACGGTGTCGTGGAACTCAG |
| 551 | GCGCCCTGACCAGCGGCGTGCACACCTTCCCGGCTGTCCTACAGTCCTCA |
| 601 | GGACTCTACTCCCTCAGCAGCGTGGTGACCGTGCCCTCCAGCAGCTTGGG |
| 651 | CACCCAGACCTACATCTGCAACGTGAATCACAAGCCCAGCAACACCAAGG |
| 701 | TGGACAAGAGAGTTGAGCCCAAATCTTGTGACAAAACTCACACATGCCCA |
| 751 | CCGTGCCCAGCACCTGAACTCCTGGGGGGACCGTCAGTCTTCCTCTTCCC |
| 801 | CCCAAAACCCAAGGACACCCTCATGATCTCCCGGACCCCTGAGGTCACAT |
| 851 | GCGTGGTGGTGGACGTGAGCCACGAAGACCCTGAGGTCAAGTTCAACTGG |
| 901 | TACGTGGACGGCGTGGAGGTGCATAATGCCAAGACAAAGCCGCGGGAGGA |
| 951 | GCAGTACAACAGCACGTACCGTGTGGTCAGCGTCCTCACCGTCCTGCACC |
| 1001 | AGGACTGGCTGAATGGCAAGGAGTACAAGTGCAAGGTCTCCAACAAAGCC |
| 1051 | CTCCCAGCCCCCATCGAGAAAACCATCTCCAAAGCCAAAGGGCAGCCCCG |
| 1101 | AGAACCACAGGTGTACACCCTGCCCCCATCCCGGGAGGAGATGACCAAGA |
| 1151 | ACCAGGTCAGCCTGACCTGCCTGGTCAAAGGCTTCTATCCCAGCGACATC |
| 1201 | GCCGTGGAGTGGGAGAGCAATGGGCAGCCGGAGAACAACTACAAGACCAC |
| 1251 | GCCTCCCGTGCTGGACTCCGACGGCTCCTTCTTCCTCTATAGCAAGCTCA |
| 1301 | CCGTGGACAAGAGCAGGTGGCAGCAGGGGAACGTCTTCTCATGCTCCGTG |
| 1351 | ATGCATGAGGCTCTGCACAACCACTACACGCAGAAGAGCCTCTCCCTGTC |
| 1401 | TCCGGGTAAAGCGGCCGCAGGTGGTGGCGGTTCAGGCGGAGGTGGCTCTG |
| 1451 | GCGGTGGCGGATCCGGTATAGGCGATCCTGTTACCTGCCTTAAGAGTGGA |
| 1501 | GCCATATGTCATCCAGTCTTTTGCCCTAGAAGGTATAAACAAATTGGCAC |
| 1551 | CTGTGGTCTCCCTGGAACAAAATGCTGCAAAAAGCCATGA |
| 1-60 | Signal peptide |
| 61-1590 | Chimeric murine-human Heavy chain-HBD2 fusion |
| SEQ ID NO: 72. I8-1029-HC-HBD2, heavy chain-HBD2 | |
| chimeric murine-human fusion, amino acid sequence, | |
| ID: 500713p | |
| | .........o.........o.........o.........o.........o |
| 1 | METDTLLLWVLLLWVPGSTGDTREVQLQQSGPELVKTGASVKISCKASGY |
| 51 | SFTGYYMHWVKQSHGKSLEWIGYISCYNGATSYNQKFKGKATFTVDTSSS |
| 101 | TAYMQFNSLTSEDSAVYYCARSRTGAWFAYWGQGTLVTVSASTKGPSVFP |
| 151 | LAPSSKSTSGGTAALGCLVKDYFPEPVTVSWNSGALTSGVHTFPAVLQSS |
| 201 | GLYSLSSVVTVPSSSLGTQTYICNVNHKPSNTKVDKRVEPKSCDKTHTCP |
| 251 | PCPAPELLGGPSVFLFPPKPKDTLMISRTPEVTCVVVDVSHEDPEVKFNW |
| 301 | YVDGVEVHNAKTKPREEQYNSTYRVVSVLTVLHQDWLNGKEYKCKVSNKA |
| 351 | LPAPIEKTISKAKGQPREPQVYTLPPSREEMTKNQVSLTCLVKGFYPSDI |
| 401 | AVEWESNGQPENNYKTTPPVLDSDGSFFLYSKLTVDKSRWQQGNVFSCSV |
| 451 | MHEALHNHYTQKSLSLSPGKAAAGGGGSGGGGSGGGGSGIGDPVTCLKSG |
| 501 | AICHPVFCPRRYKQIGTCGLPGTKCCKKP |
| 1-20 | Signal peptide |
| 21-529 | chimeric murine-human heavy chain HBD2 fusion |
| SEQ ID NO: 73. I8-1029-HC-HBD3, heavy chain-HBD3 | |
| chimeric murine-human fusion, nucleotide sequence, | |
| ID: 500714n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACACGCGTGAGGTCCAGCTGCAGCAGTCTGGACCTGAGC |
| 101 | TAGTGAAGACTGGGGCTTCAGTGAAGATATCCTGCAAGGCTTCTGGTTAC |
| 151 | TCATTCACTGGTTACTACATGCACTGGGTCAAGCAGAGCCATGGAAAGAG |
| 201 | CCTTGAGTGGATTGGATATATTAGTTGTTACAATGGTGCTACTAGCTACA |
| 251 | ACCAGAAGTTCAAGGGCAAGGCCACATTTACTGTAGACACATCCTCCAGC |
| 301 | ACAGCCTACATGCAGTTCAACAGCCTGACATCTGAAGACTCTGCGGTCTA |
| 351 | TTACTGTGCAAGATCGAGGACTGGAGCCTGGTTTGCTTACTGGGGCCAAG |
| 401 | GGACTCTGGTCACTGTCTCTGCGTCGACCAAGGGCCCATCGGTCTTCCCC |
| 451 | CTGGCACCCTCTAGCAAGAGCACCTCTGGGGGCACAGCGGCCCTGGGCTG |
| 501 | CCTGGTCAAGGACTACTTCCCCGAACCGGTGACGGTGTCGTGGAACTCAG |
| 551 | GCGCCCTGACCAGCGGCGTGCACACCTTCCCGGCTGTCCTACAGTCCTCA |
| 601 | GGACTCTACTCCCTCAGCAGCGTGGTGACCGTGCCCTCCAGCAGCTTGGG |
| 651 | CACCCAGACCTACATCTGCAACGTGAATCACAAGCCCAGCAACACCAAGG |
| 701 | TGGACAAGAGAGTTGAGCCCAAATCTTGTGACAAAACTCACACATGCCCA |
| 751 | CCGTGCCCAGCACCTGAACTCCTGGGGGGACCGTCAGTCTTCCTCTTCCC |
| 801 | CCCAAAACCCAAGGACACCCTCATGATCTCCCGGACCCCTGAGGTCACAT |
| 851 | GCGTGGTGGTGGACGTGAGCCACGAAGACCCTGAGGTCAAGTTCAACTGG |
| 901 | TACGTGGACGGCGTGGAGGTGCATAATGCCAAGACAAAGCCGCGGGAGGA |
| 951 | GCAGTACAACAGCACGTACCGTGTGGTCAGCGTCCTCACCGTCCTGCACC |
| 1001 | AGGACTGGCTGAATGGCAAGGAGTACAAGTGCAAGGTCTCCAACAAAGCC |
| 1051 | CTCCCAGCCCCCATCGAGAAAACCATCTCCAAAGCCAAAGGGCAGCCCCG |
| 1101 | AGAACCACAGGTGTACACCCTGCCCCCATCCCGGGAGGAGATGACCAAGA |
| 1151 | ACCAGGTCAGCCTGACCTGCCTGGTCAAAGGCTTCTATCCCAGCGACATC |
| 1201 | GCCGTGGAGTGGGAGAGCAATGGGCAGCCGGAGAACAACTACAAGACCAC |
| 1251 | GCCTCCCGTGCTGGACTCCGACGGCTCCTTCTTCCTCTATAGCAAGCTCA |
| 1301 | CCGTGGACAAGAGCAGGTGGCAGCAGGGGAACGTCTTCTCATGCTCCGTG |
| 1351 | ATGCATGAGGCTCTGCACAACCACTACACGCAGAAGAGCCTCTCCCTGTC |
| 1401 | TCCGGGTAAAGCGGCCGCAGGTGGTGGCGGTTCAGGCGGAGGTGGCTCTG |
| 1451 | GCGGTGGCGGATCCGGAATCATAAACACATTACAGAAATATTATTGCAGA |
| 1501 | GTCAGAGGCGGCCGGTGTGCTGTGCTCAGCTGCCTTCCAAAGGAGGAACA |
| 1551 | GATCGGCAAGTGCTCGACGCGTGGCCGAAAATGCTGCCGAAGAAAGAAAT |
| 1601 | AA |
| 1-60 | Signal peptide |
| 61-1602 | Chimeric murine-human Heavy chain-HBD3 fusion |
| SEQ ID NO: 74. I8-1029-HC-HBD3, heavy chain-HBD3 | |
| chimeric murine-human fusion, amino acid sequence, | |
| ID: 500714p | |
| | .........o.........o.........o.........o.........o |
| 1 | METDTLLLWVLLLWVPGSTGDTREVQLQQSGPELVKTGASVKISCKASGY |
| 51 | SFTGYYMHWVKQSHGKSLEWIGYISCYNGATSYNQKFKGKATFTVDTSSS |
| 101 | TAYMQFNSLTSEDSAVYYCARSRTGAWFAYWGQGTLVTVSASTKGPSVFP |
| 151 | LAPSSKSTSGGTAALGCLVKDYFPEPVTVSWNSGALTSGVHTFPAVLQSS |
| 201 | GLYSLSSVVTVPSSSLGTQTYICNVNHKPSNTKVDKRVEPKSCDKTHTCP |
| 251 | PCPAPELLGGPSVFLFPPKPKDTLMISRTPEVTCVVVDVSHEDPEVKFNW |
| 301 | YVDGVEVHNAKTKPREEQYNSTYRVVSVLTVLHQDWLNGKEYKCKVSNKA |
| 351 | LPAPIEKTISKAKGQPREPQVYTLPPSREEMTKNQVSLTCLVKGFYPSDI |
| 401 | AVEWESNGQPENNYKTTPPVLDSDGSFFLYSKLTVDKSRWQQGNVFSCSV |
| 451 | MHEALHNHYTQKSLSLSPGKAAAGGGGSGGGGSGGGGSGIINTLQKYYCR |
| 501 | VRGGRCAVLSCLPKEEQIGKCSTRGRKCCRRKK |
| 1-20 | Signal peptide |
| 21-533 | chimeric murine-human heavy chain HBD3 |
| | fusion |
| SEQ ID NO: 75. I8-1029-HC-LL37, heavy chain-LL37 | |
| chimeric murine-human fusion, nucleotide sequence, | |
| ID: 500712n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACACGCGTGAGGTCCAGCTGCAGCAGTCTGGACCTGAGC |
| 101 | TAGTGAAGACTGGGGCTTCAGTGAAGATATCCTGCAAGGCTTCTGGTTAC |
| 151 | TCATTCACTGGTTACTACATGCACTGGGTCAAGCAGAGCCATGGAAAGAG |
| 201 | CCTTGAGTGGATTGGATATATTAGTTGTTACAATGGTGCTACTAGCTACA |
| 251 | ACCAGAAGTTCAAGGGCAAGGCCACATTTACTGTAGACACATCCTCCAGC |
| 301 | ACAGCCTACATGCAGTTCAACAGCCTGACATCTGAAGACTCTGCGGTCTA |
| 351 | TTACTGTGCAAGATCGAGGACTGGAGCCTGGTTTGCTTACTGGGGCCAAG |
| 401 | GGACTCTGGTCACTGTCTCTGCGTCGACCAAGGGCCCATCGGTCTTCCCC |
| 451 | CTGGCACCCTCTAGCAAGAGCACCTCTGGGGGCACAGCGGCCCTGGGCTG |
| 501 | CCTGGTCAAGGACTACTTCCCCGAACCGGTGACGGTGTCGTGGAACTCAG |
| 551 | GCGCCCTGACCAGCGGCGTGCACACCTTCCCGGCTGTCCTACAGTCCTCA |
| 601 | GGACTCTACTCCCTCAGCAGCGTGGTGACCGTGCCCTCCAGCAGCTTGGG |
| 651 | CACCCAGACCTACATCTGCAACGTGAATCACAAGCCCAGCAACACCAAGG |
| 701 | TGGACAAGAGAGTTGAGCCCAAATCTTGTGACAAAACTCACACATGCCCA |
| 751 | CCGTGCCCAGCACCTGAACTCCTGGGGGGACCGTCAGTCTTCCTCTTCCC |
| 801 | CCCAAAACCCAAGGACACCCTCATGATCTCCCGGACCCCTGAGGTCACAT |
| 851 | GCGTGGTGGTGGACGTGAGCCACGAAGACCCTGAGGTCAAGTTCAACTGG |
| 901 | TACGTGGACGGCGTGGAGGTGCATAATGCCAAGACAAAGCCGCGGGAGGA |
| 951 | GCAGTACAACAGCACGTACCGTGTGGTCAGCGTCCTCACCGTCCTGCACC |
| 1001 | AGGACTGGCTGAATGGCAAGGAGTACAAGTGCAAGGTCTCCAACAAAGCC |
| 1051 | CTCCCAGCCCCCATCGAGAAAACCATCTCCAAAGCCAAAGGGCAGCCCCG |
| 1101 | AGAACCACAGGTGTACACCCTGCCCCCATCCCGGGAGGAGATGACCAAGA |
| 1151 | ACCAGGTCAGCCTGACCTGCCTGGTCAAAGGCTTCTATCCCAGCGACATC |
| 1201 | GCCGTGGAGTGGGAGAGCAATGGGCAGCCGGAGAACAACTACAAGACCAC |
| 1251 | GCCTCCCGTGCTGGACTCCGACGGCTCCTTCTTCCTCTATAGCAAGCTCA |
| 1301 | CCGTGGACAAGAGCAGGTGGCAGCAGGGGAACGTCTTCTCATGCTCCGTG |
| 1351 | ATGCATGAGGCTCTGCACAACCACTACACGCAGAAGAGCCTCTCCCTGTC |
| 1401 | TCCGGGTAAAGCGGCCGCAGGTGGTGGCGGTTCAGGCGGAGGTGGCTCTG |
| 1451 | GCGGTGGCGGATCCCTGCTGGGGGATTTCTTCCGGAAGTCTAAAGAGAAG |
| 1501 | ATTGGGAAAGAGTTTAAAAGAATTGTCCAGAGAATCAAGGATTTTTTGCG |
| 1551 | GAATCTTGTGCCCAGGACAGAATCCTAG |
| 1-60 | Signal peptide |
| 61-1578 | Chimeric murine-human Heavy chain-LL37 fusion |
| SEQ ID NO: 76. I8-1029-HC-LL37, heavy chain-LL37 | |
| chimeric murine-human fusion, amino acid sequence, | |
| ID: 500712p | |
| | .........o.........o.........o.........o.........o |
| 1 | METDTLLLWVLLLWVPGSTGDTREVQLQQSGPELVKTGASVKISCKASGY |
| 51 | SFTGYYMHWVKQSHGKSLEWIGYISCYNGATSYNQKFKGKATFTVDTSSS |
| 101 | TAYMQFNSLTSEDSAVYYCARSRTGAWFAYWGQGTLVTVSASTKGPSVFP |
| 151 | LAPSSKSTSGGTAALGCLVKDYFPEPVTVSWNSGALTSGVHTFPAVLQSS |
| 201 | GLYSLSSVVTVPSSSLGTQTYICNVNHKPSNTKVDKRVEPKSCDKTHTCP |
| 251 | PCPAPELLGGPSVFLFPPKPKDTLMISRTPEVTCVVVDVSHEDPEVKFNW |
| 301 | YVDGVEVHNAKTKPREEQYNSTYRVVSVLTVLHQDWLNGKEYKCKVSNKA |
| 351 | LPAPIEKTISKAKGQPREPQVYTLPPSREEMTKNQVSLTCLVKGFYPSDI |
| 401 | AVEWESNGQPENNYKTTPPVLDSDGSFFLYSKLTVDKSRWQQGNVFSCSV |
| 451 | MHEALHNHYTQKSLSLSPGKAAAGGGGSGGGGSGGGGSLLGDFFRKSKEK |
| 501 | IGKEFKRIVQRIKDFLRNLVPRTES |
| 1-20 | Signal peptide |
| 21-525 | chimeric murine-human heavy chain LL37 fusion |
| SEQ ID NO: 77. I8-1029-HC-PLA2, heavy chain-PLA2 | |
| chimeric murine-human fusion, nucleotide sequence, | |
| ID: 500715n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACACGCGTGAGGTCCAGCTGCAGCAGTCTGGACCTGAGC |
| 101 | TAGTGAAGACTGGGGCTTCAGTGAAGATATCCTGCAAGGCTTCTGGTTAC |
| 151 | TCATTCACTGGTTACTACATGCACTGGGTCAAGCAGAGCCATGGAAAGAG |
| 201 | CCTTGAGTGGATTGGATATATTAGTTGTTACAATGGTGCTACTAGCTACA |
| 251 | ACCAGAAGTTCAAGGGCAAGGCCACATTTACTGTAGACACATCCTCCAGC |
| 301 | ACAGCCTACATGCAGTTCAACAGCCTGACATCTGAAGACTCTGCGGTCTA |
| 351 | TTACTGTGCAAGATCGAGGACTGGAGCCTGGTTTGCTTACTGGGGCCAAG |
| 401 | GGACTCTGGTCACTGTCTCTGCGTCGACCAAGGGCCCATCGGTCTTCCCC |
| 451 | CTGGCACCCTCTAGCAAGAGCACCTCTGGGGGCACAGCGGCCCTGGGCTG |
| 501 | CCTGGTCAAGGACTACTTCCCCGAACCGGTGACGGTGTCGTGGAACTCAG |
| 551 | GCGCCCTGACCAGCGGCGTGCACACCTTCCCGGCTGTCCTACAGTCCTCA |
| 601 | GGACTCTACTCCCTCAGCAGCGTGGTGACCGTGCCCTCCAGCAGCTTGGG |
| 651 | CACCCAGACCTACATCTGCAACGTGAATCACAAGCCCAGCAACACCAAGG |
| 701 | TGGACAAGAGAGTTGAGCCCAAATCTTGTGACAAAACTCACACATGCCCA |
| 751 | CCGTGCCCAGCACCTGAACTCCTGGGGGGACCGTCAGTCTTCCTCTTCCC |
| 801 | CCCAAAACCCAAGGACACCCTCATGATCTCCCGGACCCCTGAGGTCACAT |
| 851 | GCGTGGTGGTGGACGTGAGCCACGAAGACCCTGAGGTCAAGTTCAACTGG |
| 901 | TACGTGGACGGCGTGGAGGTGCATAATGCCAAGACAAAGCCGCGGGAGGA |
| 951 | GCAGTACAACAGCACGTACCGTGTGGTCAGCGTCCTCACCGTCCTGCACC |
| 1001 | AGGACTGGCTGAATGGCAAGGAGTACAAGTGCAAGGTCTCCAACAAAGCC |
| 1051 | CTCCCAGCCCCCATCGAGAAAACCATCTCCAAAGCCAAAGGGCAGCCCCG |
| 1101 | AGAACCACAGGTGTACACCCTGCCCCCATCCCGGGAGGAGATGACCAAGA |
| 1151 | ACCAGGTCAGCCTGACCTGCCTGGTCAAAGGCTTCTATCCCAGCGACATC |
| 1201 | GCCGTGGAGTGGGAGAGCAATGGGCAGCCGGAGAACAACTACAAGACCAC |
| 1251 | GCCTCCCGTGCTGGACTCCGACGGCTCCTTCTTCCTCTATAGCAAGCTCA |
| 1301 | CCGTGGACAAGAGCAGGTGGCAGCAGGGGAACGTCTTCTCATGCTCCGTG |
| 1351 | ATGCATGAGGCTCTGCACAACCACTACACGCAGAAGAGCCTCTCCCTGTC |
| 1401 | TCCGGGTAAAGCGGCCGCAGGTGGTGGCGGTTCAGGCGGAGGTGGCTCTG |
| 1451 | GCGGTGGCGGATCGAATTTGGTGAATTTCCACAGAATGATCAAGTTGACG |
| 1501 | ACAGGAAAGGAAGCCGCACTCAGTTATGGCTTCTACGGCTGCCACTGTGG |
| 1551 | CGTGGGTGGCAGAGGATCCCCCAAGGATGCAACGGATCGCTGCTGTGTCA |
| 1601 | CTCATGACTGTTGCTACAAACGTCTGGAGAAACGTGGATGTGGCACCAAA |
| 1651 | TTTCTGAGCTACAAGTTTAGCAACTCGGGGAGCAGAATCACCTGTGCAAA |
| 1701 | ACAGGACTCCTGCAGAAGTCAACTGTGTGAGTGTGATAAGGCTGCTGCCA |
| 1751 | CCTGTTTTGCTAGAAACAAGACGACCTACAATAAAAAGTACCAGTACTAT |
| 1801 | TCCAATAAACACTGCAGAGGGAGCACCCCTCGTTGCTGA |
| 1-60 | Signal peptide |
| 61-1839 | Chimeric murine-human Heavy chain-PLA2 fusion |
| SEQ ID NO: 78. I8-1029-HC-PLA2, heavy chain-PLA2 | |
| chimeric murine-human fusion, amino acid sequence, | |
| ID: 500715p | |
| | .........o.........o.........o.........o.........o |
| 1 | METDTLLLWVLLLWVPGSTGDTREVQLQQSGPELVKTGASVKISCKASGY |
| 51 | SFTGYYMHWVKQSHGKSLEWIGYISCYNGATSYNQKFKGKATFTVDTSSS |
| 101 | TAYMQFNSLTSEDSAVYYCARSRTGAWFAYWGQGTLVTVSASTKGPSVFP |
| 151 | LAPSSKSTSGGTAALGCLVKDYFPEPVTVSWNSGALTSGVHTFPAVLQSS |
| 201 | GLYSLSSVVTVPSSSLGTQTYICNVNHKPSNTKVDKRVEPKSCDKTHTCP |
| 251 | PCPAPELLGGPSVFLFPPKPKDTLMISRTPEVTCVVVDVSHEDPEVKFNW |
| 301 | YVDGVEVHNAKTKPREEQYNSTYRVVSVLTVLHQDWLNGKEYKCKVSNKA |
| 351 | LPAPIEKTISKAKGQPREPQVYTLPPSREEMTKNQVSLTCLVKGFYPSDI |
| 401 | AVEWESNGQPENNYKTTPPVLDSDGSFFLYSKLTVDKSRWQQGNVFSCSV |
| 451 | MHEALHNHYTQKSLSLSPGKAAAGGGGSGGGGSGGGGSNLVNFHRMIKLT |
| 501 | TGKEAALSYGFYGCHCGVGGRGSPKDATDRCCVTHDCCYKRLEKRGCGTK |
| 551 | FLSYKFSNSGSRITCAKQDSCRSQLCECDKAAATCFARNKTTYNKKYQYY |
| 601 | SNKHCRGSTPRC |
| 1-20 | Signal peptide |
| 21-612 | chimeric murine-human heavy chain-PLA2 fusion |
| SEQ ID NO: 79. LYST-LC-I9-6001, Lysostaphin-light | |
| chain chimeric murine-human fusion, nucleotide | |
| sequence, ID: 500742n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACGCCACCCACGAGCACTCCGCCCAGTGGCTGAACAACT |
| 101 | ACAAGAAGGGCTACGGCTACGGCCCCTACCCCCTGGGCATCAACGGCGGC |
| 151 | ATGCACTACGGCGTGGACTTCTTCATGAACATCGGCACCCCCGTGAAGGC |
| 201 | CATCTCCTCCGGCAAGATCGTGGAGGCCGGCTGGTCCAACTACGGCGGCG |
| 251 | GCAACCAGATCGGCCTGATCGAGAACGACGGCGTGCACCGCCAGTGGTAC |
| 301 | ATGCACCTGTCCAAGTACAACGTGAAGGTGGGCGACTACGTGAAGGCCGG |
| 351 | CCAGATCATCGGCTGGTCCGGCTCCACCGGCTACTCCACCGCCCCCCACC |
| 401 | TGCACTTCCAGCGCATGGTGAACTCCTTCTCCAACTCCACCGCCCAGGAC |
| 451 | CCCATGCCCTTCCTGAAGTCCGCCGGCTACGGCAAGGCCGGCGGCACCGT |
| 501 | GACCCCCACCCCCAACACCGGCTGGAAGACCAACAAGTACGGCACCCTGT |
| 551 | ACAAGTCCGAGTCCGCCTCCTTCACCCCCAACACCGACATCATCACCCGC |
| 601 | ACCACCGGCCCCTTCCGCTCCATGCCCCAGTCCGGCGTGCTGAAGGCCGG |
| 651 | CCAGACCATCCACTACGACGAGGTGATGAAGCAGGACGGCCACGTGTGGG |
| 701 | TGGGCTACACCGGCAACTCCGGCCAGCGCATCTACCTGCCCGTGCGCACC |
| 751 | TGGAACAAGTCCACCAACACCCTGGGCGTGCTGTGGGGCACCATCAAGGG |
| 801 | TGGTGGCGGTTCAGGCGGAGGTGGCTCTGGCGGTGGCGGATCcacgcgtG |
| 851 | ACATCCAGATGACTCAGTCTCCAGCCTCCCTATCTGCATCTGTGGGAGAA |
| 901 | ACTGTCACCATCACATGTCGAGCAAGTGGGAATATTCACAATTATTTAGC |
| 951 | ATGGTATCAGCAGAAACAGGGAAAATCTCCTCAGCTCCTGGTCTATAATG |
| 1001 | CAAAAACCTTAGCAGATGGTGTGCCATCAAGGTTCAGTGGCAGTGGATCA |
| 1051 | GGAACACAATATTCTCTCAAGATCAACAGCCTGCAGCCTGAAGATTTTGG |
| 1101 | GAGTTATTACTGTCAACATTTTTGGAGTACTCCGTGGACGTTCGGTGGAG |
| 1151 | GCACCAAGCTGGAAATCAAACGGACTGTGGCTGCACCATCTGTCTTCATC |
| 1201 | TTCCCGCCATCTGATGAGCAGTTGAAATCTGGAACTGCCTCTGTTGTGTG |
| 1251 | CCTGCTGAATAACTTCTATCCCAGAGAGGCCAAAGTACAGTGGAAGGTGG |
| 1301 | ATAACGCCCTCCAATCGGGTAACTCCCAGGAGAGTGTCACAGAGCAGGAC |
| 1351 | AGCAAGGACAGCACCTACAGCCTCAGCAGCACCCTGACGCTGAGCAAAGC |
| 1401 | AGACTACGAGAAACACAAAGTCTACGCCTGCGAAGTCACCCATCAGGGCC |
| 1451 | TGAGCTCGCCCGTCACAAAGAGCTTCAACAGGGGAGAGTGTTAG |
| 1-60 | Signal peptide |
| 61-1494 | Lysostaphin-Light chain fusion |
| SEQ ID NO: 80. LYST-LC-I9-6001, Lysostaphin-light | |
| chain fusion, murine-human chimeric, amino acid | |
| sequence, ID: 500742p | |
| | .........o.........o.........o.........o.........o |
| 1 | METDTLLLWVLLLWVPGSTGDATHEHSAQWLNNYKKGYGYGPYPLGINGG |
| 51 | MHYGVDFFMNIGTPVKAISSGKIVEAGWSNYGGGNQIGLIENDGVHRQWY |
| 101 | MHLSKYNVKVGDYVKAGQIIGWSGSTGYSTAPHLHFQRMVNSFSNSTAQD |
| 151 | PMPFLKSAGYGKAGGTVTPTPNTGWKTNKYGTLYKSESASFTPNTDIITR |
| 201 | TTGPFRSMPQSGVLKAGQTIHYDEVMKQDGHVWVGYTGNSGQRIYLPVRT |
| 251 | WNKSTNTLGVLWGTIKGGGGSGGGGSGGGGSTRDIQMTQSPASLSASVGE |
| 301 | TVTITCRASGNIHNYLAWYQQKQGKSPQLLVYNAKTLADGVPSRFSGSGS |
| 351 | GTQYSLKINSLQPEDFGSYYCQHFWSTPWTFGGGTKLEIKRTVAAPSVFI |
| 401 | FPPSDEQLKSGTASVVCLLNNFYPREAKVQWKVDNALQSGNSQESVTEQD |
| 451 | SKDSTYSLSSTLTLSKADYEKHKVYACEVTHQGLSSPVTKSFNRGEC |
| 1-20 | Signal peptide |
| 21-497 | Lysostaphin-Light chain fusion |
| SEQ ID NO: 81. I9-6001-HC-HBD2, heavy chain-HBD2 | |
| chimeric murine-human fusion, nucleotide sequence, | |
| ID: 500737n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACACGCGTCAGGTCCAGCTGCAGCAGTCTGGAGCTGAGC |
| 101 | TGGTAAGGCCTGGGACTTCAGTGAAGGTGTCCTGCAAGGCTTCTGGATAC |
| 151 | GCCTTCACTAATTACTTGATAGAGTGGGTAAAGCAGAGGCCTGGACAGGG |
| 201 | CCTTGAGTGGATTGGAGTGATTAATCCTGGAAGTGGTGGTACTAACTACA |
| 251 | ATGAGAAGTTCAAGGGCAAGGCAACACTGACTGCAGACAAATCCTCCAGC |
| 301 | ACTGCCTACATGCAGCTCAGCAGCCTGACATCTGATGACTCTGCGGTCTA |
| 351 | TTTCTGTGCAAGATGGGACTACGGTAGTAGCTACGAACGTGCTATGGACT |
| 401 | ACTGGGGTCAAGGAACCTCAGTCACCGTCTCCGCGTCGACCAAGGGCCCA |
| 451 | TCGGTCTTCCCCCTGGCACCCTCTAGCAAGAGCACCTCTGGGGGCACAGC |
| 501 | GGCCCTGGGCTGCCTGGTCAAGGACTACTTCCCCGAACCGGTGACGGTGT |
| 551 | CGTGGAACTCAGGCGCCCTGACCAGCGGCGTGCACACCTTCCCGGCTGTC |
| 601 | CTACAGTCCTCAGGACTCTACTCCCTCAGCAGCGTGGTGACCGTGCCCTC |
| 651 | CAGCAGCTTGGGCACCCAGACCTACATCTGCAACGTGAATCACAAGCCCA |
| 701 | GCAACACCAAGGTGGACAAGAGAGTTGAGCCCAAATCTTGTGACAAAACT |
| 751 | CACACATGCCCACCGTGCCCAGCACCTGAACTCCTGGGGGGACCGTCAGT |
| 801 | CTTCCTCTTCCCCCCAAAACCCAAGGACACCCTCATGATCTCCCGGACCC |
| 851 | CTGAGGTCACATGCGTGGTGGTGGACGTGAGCCACGAAGACCCTGAGGTC |
| 901 | AAGTTCAACTGGTACGTGGACGGCGTGGAGGTGCATAATGCCAAGACAAA |
| 951 | GCCGCGGGAGGAGCAGTACAACAGCACGTACCGTGTGGTCAGCGTCCTCA |
| 1001 | CCGTCCTGCACCAGGACTGGCTGAATGGCAAGGAGTACAAGTGCAAGGTC |
| 1051 | TCCAACAAAGCCCTCCCAGCCCCCATCGAGAAAACCATCTCCAAAGCCAA |
| 1101 | AGGGCAGCCCCGAGAACCACAGGTGTACACCCTGCCCCCATCCCGGGAGG |
| 1151 | AGATGACCAAGAACCAGGTCAGCCTGACCTGCCTGGTCAAAGGCTTCTAT |
| 1201 | CCCAGCGACATCGCCGTGGAGTGGGAGAGCAATGGGCAGCCGGAGAACAA |
| 1251 | CTACAAGACCACGCCTCCCGTGCTGGACTCCGACGGCTCCTTCTTCCTCT |
| 1301 | ATAGCAAGCTCACCGTGGACAAGAGCAGGTGGCAGCAGGGGAACGTCTTC |
| 1351 | TCATGCTCCGTGATGCATGAGGCTCTGCACAACCACTACACGCAGAAGAG |
| 1401 | CCTCTCCCTGTCTCCGGGTAAAGCGGCCGCAGGTGGTGGCGGTTCAGGCG |
| 1451 | GAGGTGGCTCTGGCGGTGGCGGATCCGGTATAGGCGATCCTGTTACCTGC |
| 1501 | CTTAAGAGTGGAGCCATATGTCATCCAGTCTTTTGCCCTAGAAGGTATAA |
| 1551 | ACAAATTGGCACCTGTGGTCTCCCTGGAACAAAATGCTGCAAAAAGCCAT |
| 1601 | GA |
| 1-60 | Signal peptide |
| 61-1602 | Chimeric murine-human Heavy chain-HBD2 fusion |
| SEQ ID NO: 82. I9-6001-HC-HBD2, heavy chain-HBD2 | |
| chimeric murine-human fusion, amino acid sequence, | |
| ID:500737p | |
| | .........o.........o.........o.........o.........o |
| 1 | METDTLLLWVLLLWVPGSTGDTRQVQLQQSGAELVRPGTSVKVSCKASGY |
| 51 | AFTNYLIEWVKQRPGQGLEWIGVINPGSGGTNYNEKFKGKATLTADKSSS |
| 101 | TAYMQLSSLTSDDSAVYFCARWDYGSSYERAMDYWGQGTSVTVSASTKGP |
| 151 | SVFPLAPSSKSTSGGTAALGCLVKDYFPEPVTVSWNSGALTSGVHTFPAV |
| 201 | LQSSGLYSLSSVVTVPSSSLGTQTYICNVNHKPSNTKVDKRVEPKSCDKT |
| 251 | HTCPPCPAPELLGGPSVFLFPPKPKDTLMISRTPEVTCVVVDVSHEDPEV |
| 301 | KFNWYVDGVEVHNAKTKPREEQYNSTYRVVSVLTVLHQDWLNGKEYKCKV |
| 351 | SNKALPAPIEKTISKAKGQPREPQVYTLPPSREEMTKNQVSLTCLVKGFY |
| 401 | PSDIAVEWESNGQPENNYKTTPPVLDSDGSFFLYSKLTVDKSRWQQGNVF |
| 451 | SCSVMHEALHNHYTQKSLSLSPGKAAAGGGGSGGGGSGGGGSGIGDPVTC |
| 501 | LKSGAICHPVFCPRRYKQIGTCGLPGTKCCKKP |
| 1-20 | Signal peptide |
| 21-533 | chimeric murine-human heavy chain HBD2 fusion |
| SEQ ID NO: 83. I9-6001-HC-HBD3, heavy chain-HBD3 | |
| chimeric murine-human fusion, nucleotide sequence, | |
| ID: 500738n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACACGCGTCAGGTCCAGCTGCAGCAGTCTGGAGCTGAGC |
| 101 | TGGTAAGGCCTGGGACTTCAGTGAAGGTGTCCTGCAAGGCTTCTGGATAC |
| 151 | GCCTTCACTAATTACTTGATAGAGTGGGTAAAGCAGAGGCCTGGACAGGG |
| 201 | CCTTGAGTGGATTGGAGTGATTAATCCTGGAAGTGGTGGTACTAACTACA |
| 251 | ATGAGAAGTTCAAGGGCAAGGCAACACTGACTGCAGACAAATCCTCCAGC |
| 301 | ACTGCCTACATGCAGCTCAGCAGCCTGACATCTGATGACTCTGCGGTCTA |
| 351 | TTTCTGTGCAAGATGGGACTACGGTAGTAGCTACGAACGTGCTATGGACT |
| 401 | ACTGGGGTCAAGGAACCTCAGTCACCGTCTCCGCGTCGACCAAGGGCCCA |
| 451 | TCGGTCTTCCCCCTGGCACCCTCTAGCAAGAGCACCTCTGGGGGCACAGC |
| 501 | GGCCCTGGGCTGCCTGGTCAAGGACTACTTCCCCGAACCGGTGACGGTGT |
| 551 | CGTGGAACTCAGGCGCCCTGACCAGCGGCGTGCACACCTTCCCGGCTGTC |
| 601 | CTACAGTCCTCAGGACTCTACTCCCTCAGCAGCGTGGTGACCGTGCCCTC |
| 651 | CAGCAGCTTGGGCACCCAGACCTACATCTGCAACGTGAATCACAAGCCCA |
| 701 | GCAACACCAAGGTGGACAAGAGAGTTGAGCCCAAATCTTGTGACAAAACT |
| 751 | CACACATGCCCACCGTGCCCAGCACCTGAACTCCTGGGGGGACCGTCAGT |
| 801 | CTTCCTCTTCCCCCCAAAACCCAAGGACACCCTCATGATCTCCCGGACCC |
| 851 | CTGAGGTCACATGCGTGGTGGTGGACGTGAGCCACGAAGACCCTGAGGTC |
| 901 | AAGTTCAACTGGTACGTGGACGGCGTGGAGGTGCATAATGCCAAGACAAA |
| 951 | GCCGCGGGAGGAGCAGTACAACAGCACGTACCGTGTGGTCAGCGTCCTCA |
| 1001 | CCGTCCTGCACCAGGACTGGCTGAATGGCAAGGAGTACAAGTGCAAGGTC |
| 1051 | TCCAACAAAGCCCTCCCAGCCCCCATCGAGAAAACCATCTCCAAAGCCAA |
| 1101 | AGGGCAGCCCCGAGAACCACAGGTGTACACCCTGCCCCCATCCCGGGAGG |
| 1151 | AGATGACCAAGAACCAGGTCAGCCTGACCTGCCTGGTCAAAGGCTTCTAT |
| 1201 | CCCAGCGACATCGCCGTGGAGTGGGAGAGCAATGGGCAGCCGGAGAACAA |
| 1251 | CTACAAGACCACGCCTCCCGTGCTGGACTCCGACGGCTCCTTCTTCCTCT |
| 1301 | ATAGCAAGCTCACCGTGGACAAGAGCAGGTGGCAGCAGGGGAACGTCTTC |
| 1351 | TCATGCTCCGTGATGCATGAGGCTCTGCACAACCACTACACGCAGAAGAG |
| 1401 | CCTCTCCCTGTCTCCGGGTAAAGCGGCCGCAGGTGGTGGCGGTTCAGGCG |
| 1451 | GAGGTGGCTCTGGCGGTGGCGGATCCGGAATCATAAACACATTACAGAAA |
| 1501 | TATTATTGCAGAGTCAGAGGCGGCCGGTGTGCTGTGCTCAGCTGCCTTCC |
| 1551 | AAAGGAGGAACAGATCGGCAAGTGCTCGACGCGTGGCCGAAAATGCTGCC |
| 1601 | GAAGAAAGAAATAA |
| 1-60 | Signal peptide |
| 61-1614 | Chimeric murine-human Heavy chain-HBD3 fusion |
| SEQ ID NO: 84. I9-6001-HC-HBD3, heavy chain-HBD3 | |
| chimeric murine-human fusion, amino acid sequence, | |
| ID: 500738p | |
| | .........o.........o.........o.........o.........o |
| 1 | METDTLLLWVLLLWVPGSTGDTRQVQLQQSGAELVRPGTSVKVSCKASGY |
| 51 | AFTNYLIEWVKQRPGQGLEWIGVINPGSGGTNYNEKFKGKATLTADKSSS |
| 101 | TAYMQLSSLTSDDSAVYFCARWDYGSSYERAMDYWGQGTSVTVSASTKGP |
| 151 | SVFPLAPSSKSTSGGTAALGCLVKDYFPEPVTVSWNSGALTSGVHTFPAV |
| 201 | LQSSGLYSLSSVVTVPSSSLGTQTYICNVNHKPSNTKVDKRVEPKSCDKT |
| 251 | HTCPPCPAPELLGGPSVFLFPPKPKDTLMISRTPEVTCVVVDVSHEDPEV |
| 301 | KFNWYVDGVEVHNAKTKPREEQYNSTYRVVSVLTVLHQDWLNGKEYKCKV |
| 351 | SNKALPAPIEKTISKAKGQPREPQVYTLPPSREEMTKNQVSLTCLVKGFY |
| 401 | PSDIAVEWESNGQPENNYKTTPPVLDSDGSFFLYSKLTVDKSRWQQGNVF |
| 451 | SCSVMHEALHNHYTQKSLSLSPGKAAAGGGGSGGGGSGGGGSGIINTLQK |
| 501 | YYCRVRGGRCAVLSCLPKEEQIGKCSTRGRKCCRRKK |
| 1-20 | Signal peptide |
| 21-537 | chimeric murine-human heavy chain HBD3 fusion |
| SEQ ID NO: 85. I9-6001-HC-LL37, heavy chain-LL37 | |
| chimeric murine-human fusion, nucleotide sequence, | |
| ID: 500736n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACACGCGTCAGGTCCAGCTGCAGCAGTCTGGAGCTGAGC |
| 101 | TGGTAAGGCCTGGGACTTCAGTGAAGGTGTCCTGCAAGGCTTCTGGATAC |
| 151 | GCCTTCACTAATTACTTGATAGAGTGGGTAAAGCAGAGGCCTGGACAGGG |
| 201 | CCTTGAGTGGATTGGAGTGATTAATCCTGGAAGTGGTGGTACTAACTACA |
| 251 | ATGAGAAGTTCAAGGGCAAGGCAACACTGACTGCAGACAAATCCTCCAGC |
| 301 | ACTGCCTACATGCAGCTCAGCAGCCTGACATCTGATGACTCTGCGGTCTA |
| 351 | TTTCTGTGCAAGATGGGACTACGGTAGTAGCTACGAACGTGCTATGGACT |
| 401 | ACTGGGGTCAAGGAACCTCAGTCACCGTCTCCGCGTCGACCAAGGGCCCA |
| 451 | TCGGTCTTCCCCCTGGCACCCTCTAGCAAGAGCACCTCTGGGGGCACAGC |
| 501 | GGCCCTGGGCTGCCTGGTCAAGGACTACTTCCCCGAACCGGTGACGGTGT |
| 551 | CGTGGAACTCAGGCGCCCTGACCAGCGGCGTGCACACCTTCCCGGCTGTC |
| 601 | CTACAGTCCTCAGGACTCTACTCCCTCAGCAGCGTGGTGACCGTGCCCTC |
| 651 | CAGCAGCTTGGGCACCCAGACCTACATCTGCAACGTGAATCACAAGCCCA |
| 701 | GCAACACCAAGGTGGACAAGAGAGTTGAGCCCAAATCTTGTGACAAAACT |
| 751 | CACACATGCCCACCGTGCCCAGCACCTGAACTCCTGGGGGGACCGTCAGT |
| 801 | CTTCCTCTTCCCCCCAAAACCCAAGGACACCCTCATGATCTCCCGGACCC |
| 851 | CTGAGGTCACATGCGTGGTGGTGGACGTGAGCCACGAAGACCCTGAGGTC |
| 901 | AAGTTCAACTGGTACGTGGACGGCGTGGAGGTGCATAATGCCAAGACAAA |
| 951 | GCCGCGGGAGGAGCAGTACAACAGCACGTACCGTGTGGTCAGCGTCCTCA |
| 1001 | CCGTCCTGCACCAGGACTGGCTGAATGGCAAGGAGTACAAGTGCAAGGTC |
| 1051 | TCCAACAAAGCCCTCCCAGCCCCCATCGAGAAAACCATCTCCAAAGCCAA |
| 1101 | AGGGCAGCCCCGAGAACCACAGGTGTACACCCTGCCCCCATCCCGGGAGG |
| 1151 | AGATGACCAAGAACCAGGTCAGCCTGACCTGCCTGGTCAAAGGCTTCTAT |
| 1201 | CCCAGCGACATCGCCGTGGAGTGGGAGAGCAATGGGCAGCCGGAGAACAA |
| 1251 | CTACAAGACCACGCCTCCCGTGCTGGACTCCGACGGCTCCTTCTTCCTCT |
| 1301 | ATAGCAAGCTCACCGTGGACAAGAGCAGGTGGCAGCAGGGGAACGTCTTC |
| 1351 | TCATGCTCCGTGATGCATGAGGCTCTGCACAACCACTACACGCAGAAGAG |
| 1401 | CCTCTCCCTGTCTCCGGGTAAAGCGGCCGCAGGTGGTGGCGGTTCAGGCG |
| 1451 | GAGGTGGCTCTGGCGGTGGCGGATCCCTGCTGGGGGATTTCTTCCGGAAG |
| 1501 | TCTAAAGAGAAGATTGGGAAAGAGTTTAAAAGAATTGTCCAGAGAATCAA |
| 1551 | GGATTTTTTGCGGAATCTTGTGCCCAGGACAGAATCCTAG |
| 1-60 | Signal peptide |
| 61-1590 | Chimeric murine-human Heavy chain-LL37 fusion |
| SEQ ID NO: 86. I9-6001-HC-LL37, heavy chain-LL37 | |
| chimeric murine-human fusion, amino acid sequence, | |
| ID: 500736p | |
| | .........o.........o.........o.........o.........o |
| 1 | METDTLLLWVLLLWVPGSTGDTRQVQLQQSGAELVRPGTSVKVSCKASGY |
| 51 | AFTNYLIEWVKQRPGQGLEWIGVINPGSGGTNYNEKFKGKATLTADKSSS |
| 101 | TAYMQLSSLTSDDSAVYFCARWDYGSSYERAMDYWGQGTSVTVSASTKGP |
| 151 | SVFPLAPSSKSTSGGTAALGCLVKDYFPEPVTVSWNSGALTSGVHTFPAV |
| 201 | LQSSGLYSLSSVVTVPSSSLGTQTYICNVNHKPSNTKVDKRVEPKSCDKT |
| 251 | HTCPPCPAPELLGGPSVFLFPPKPKDTLMISRTPEVTCVVVDVSHEDPEV |
| 301 | KFNWYVDGVEVHNAKTKPREEQYNSTYRVVSVLTVLHQDWLNGKEYKCKV |
| 351 | SNKALPAPIEKTISKAKGQPREPQVYTLPPSREEMTKNQVSLTCLVKGFY |
| 401 | PSDIAVEWESNGQPENNYKTTPPVLDSDGSFFLYSKLTVDKSRWQQGNVF |
| 451 | SCSVMHEALHNHYTQKSLSLSPGKAAAGGGGSGGGGSGGGGSLLGDFFRK |
| 501 | SKEKIGKEFKRIVQRIKDFLRNLVPRTES |
| 1-20 | Signal peptide |
| 21-529 | chimeric murine-human heavy chain LL37 fusion |
| SEQ ID NO: 87. I9-6001-HC-PLA2, heavy chain-PLA2 | |
| chimeric murine-human fusion, nucleotide sequence, | |
| ID: 500739n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACACGCGTCAGGTCCAGCTGCAGCAGTCTGGAGCTGAGC |
| 101 | TGGTAAGGCCTGGGACTTCAGTGAAGGTGTCCTGCAAGGCTTCTGGATAC |
| 151 | GCCTTCACTAATTACTTGATAGAGTGGGTAAAGCAGAGGCCTGGACAGGG |
| 201 | CCTTGAGTGGATTGGAGTGATTAATCCTGGAAGTGGTGGTACTAACTACA |
| 251 | ATGAGAAGTTCAAGGGCAAGGCAACACTGACTGCAGACAAATCCTCCAGC |
| 301 | ACTGCCTACATGCAGCTCAGCAGCCTGACATCTGATGACTCTGCGGTCTA |
| 351 | TTTCTGTGCAAGATGGGACTACGGTAGTAGCTACGAACGTGCTATGGACT |
| 401 | ACTGGGGTCAAGGAACCTCAGTCACCGTCTCCGCGTCGACCAAGGGCCCA |
| 451 | TCGGTCTTCCCCCTGGCACCCTCTAGCAAGAGCACCTCTGGGGGCACAGC |
| 501 | GGCCCTGGGCTGCCTGGTCAAGGACTACTTCCCCGAACCGGTGACGGTGT |
| 551 | CGTGGAACTCAGGCGCCCTGACCAGCGGCGTGCACACCTTCCCGGCTGTC |
| 601 | CTACAGTCCTCAGGACTCTACTCCCTCAGCAGCGTGGTGACCGTGCCCTC |
| 651 | CAGCAGCTTGGGCACCCAGACCTACATCTGCAACGTGAATCACAAGCCCA |
| 701 | GCAACACCAAGGTGGACAAGAGAGTTGAGCCCAAATCTTGTGACAAAACT |
| 751 | CACACATGCCCACCGTGCCCAGCACCTGAACTCCTGGGGGGACCGTCAGT |
| 801 | CTTCCTCTTCCCCCCAAAACCCAAGGACACCCTCATGATCTCCCGGACCC |
| 851 | CTGAGGTCACATGCGTGGTGGTGGACGTGAGCCACGAAGACCCTGAGGTC |
| 901 | AAGTTCAACTGGTACGTGGACGGCGTGGAGGTGCATAATGCCAAGACAAA |
| 951 | GCCGCGGGAGGAGCAGTACAACAGCACGTACCGTGTGGTCAGCGTCCTCA |
| 1001 | CCGTCCTGCACCAGGACTGGCTGAATGGCAAGGAGTACAAGTGCAAGGTC |
| 1051 | TCCAACAAAGCCCTCCCAGCCCCCATCGAGAAAACCATCTCCAAAGCCAA |
| 1101 | AGGGCAGCCCCGAGAACCACAGGTGTACACCCTGCCCCCATCCCGGGAGG |
| 1151 | AGATGACCAAGAACCAGGTCAGCCTGACCTGCCTGGTCAAAGGCTTCTAT |
| 1201 | CCCAGCGACATCGCCGTGGAGTGGGAGAGCAATGGGCAGCCGGAGAACAA |
| 1251 | CTACAAGACCACGCCTCCCGTGCTGGACTCCGACGGCTCCTTCTTCCTCT |
| 1301 | ATAGCAAGCTCACCGTGGACAAGAGCAGGTGGCAGCAGGGGAACGTCTTC |
| 1351 | TCATGCTCCGTGATGCATGAGGCTCTGCACAACCACTACACGCAGAAGAG |
| 1401 | CCTCTCCCTGTCTCCGGGTAAAGCGGCCGCAGGTGGTGGCGGTTCAGGCG |
| 1451 | GAGGTGGCTCTGGCGGTGGCGGATCGAATTTGGTGAATTTCCACAGAATG |
| 1501 | ATCAAGTTGACGACAGGAAAGGAAGCCGCACTCAGTTATGGCTTCTACGG |
| 1551 | CTGCCACTGTGGCGTGGGTGGCAGAGGATCCCCCAAGGATGCAACGGATC |
| 1601 | GCTGCTGTGTCACTCATGACTGTTGCTACAAACGTCTGGAGAAACGTGGA |
| 1651 | TGTGGCACCAAATTTCTGAGCTACAAGTTTAGCAACTCGGGGAGCAGAAT |
| 1701 | CACCTGTGCAAAACAGGACTCCTGCAGAAGTCAACTGTGTGAGTGTGATA |
| 1751 | AGGCTGCTGCCACCTGTTTTGCTAGAAACAAGACGACCTACAATAAAAAG |
| 1801 | TACCAGTACTATTCCAATAAACACTGCAGAGGGAGCACCCCTCGTTGCTG |
| 1851 | A |
| 1-60 | Signal peptide |
| 61-1851 | Chimeric murine-human Heavy chain-PLA2 fusion |
| SEQ ID NO: 88. I9-6001-HC-PLA2, heavy chain-PLA2 | |
| chimeric murine-human fusion, amino acid sequence, | |
| ID: 500739p | |
| | .........o.........o.........o.........o.........o |
| 1 | METDTLLLWVLLLWVPGSTGDTRQVQLQQSGAELVRPGTSVKVSCKASGY |
| 51 | AFTNYLIEWVKQRPGQGLEWIGVINPGSGGTNYNEKFKGKATLTADKSSS |
| 101 | TAYMQLSSLTSDDSAVYFCARWDYGSSYERAMDYWGQGTSVTVSASTKGP |
| 151 | SVFPLAPSSKSTSGGTAALGCLVKDYFPEPVTVSWNSGALTSGVHTFPAV |
| 201 | LQSSGLYSLSSVVTVPSSSLGTQTYICNVNHKPSNTKVDKRVEPKSCDKT |
| 251 | HTCPPCPAPELLGGPSVFLFPPKPKDTLMISRTPEVTCVVVDVSHEDPEV |
| 301 | KFNWYVDGVEVHNAKTKPREEQYNSTYRVVSVLTVLHQDWLNGKEYKCKV |
| 351 | SNKALPAPIEKTISKAKGQPREPQVYTLPPSREEMTKNQVSLTCLVKGFY |
| 401 | PSDIAVEWESNGQPENNYKTTPPVLDSDGSFFLYSKLTVDKSRWQQGNVF |
| 451 | SCSVMHEALHNHYTQKSLSLSPGKAAAGGGGSGGGGSGGGGSNLVNFHRM |
| 501 | IKLTTGKEAALSYGFYGCHCGVGGRGSPKDATDRCCVTHDCCYKRLEKRG |
| 551 | CGTKFLSYKFSNSGSRITCAKQDSCRSQLCECDKAAATCFARNKTTYNKK |
| 601 | YQYYSNKHCRGSTPRC |
| 1-20 | Signal peptide |
| 21-616 | chimeric murine-human heavy chain-PLA2 fusion |

### Examples for Fab Directed Biocides

| SEQ ID NO: 89. I9-6001-HC-F(ab)2-HBD3, heavy | |
|---|---|
| chain-HBD3 chimeric murine-human fusion, | |
| nucleotide sequence, ID: 500768n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACCATCACCATCACCATCACACGCGTCAGGTCCAGCTGC |
| 101 | AGCAGTCTGGAGCTGAGCTGGTAAGGCCTGGGACTTCAGTGAAGGTGTCC |
| 151 | TGCAAGGCTTCTGGATACGCCTTCACTAATTACTTGATAGAGTGGGTAAA |
| 201 | GCAGAGGCCTGGACAGGGCCTTGAGTGGATTGGAGTGATTAATCCTGGAA |
| 251 | GTGGTGGTACTAACTACAATGAGAAGTTCAAGGGCAAGGCAACACTGACT |
| 301 | GCAGACAAATCCTCCAGCACTGCCTACATGCAGCTCAGCAGCCTGACATC |
| 351 | TGATGACTCTGCGGTCTATTTCTGTGCAAGATGGGACTACGGTAGTAGCT |
| 401 | ACGAACGTGCTATGGACTACTGGGGTCAAGGAACCTCAGTCACCGTCTCC |
| 451 | GCGTCGACCAAGGGCCCATCGGTCTTCCCCCTGGCACCCTCCTCCAAGAG |
| 501 | CACCTCTGGGGGCACAGCGGCCCTGGGCTGCCTGGTCAAGGACTACTTCC |
| 551 | CCGAACCGGTGACGGTGTCGTGGAACTCAGGCGCCCTGACCAGCGGCGTG |
| 601 | CACACCTTCCCGGCTGTCCTACAGTCCTCAGGACTCTACTCCCTCAGCAG |
| 651 | CGTGGTGACCGTGCCCTCCAGCAGCTTGGGCACCCAGACCTACATCTGCA |
| 701 | ACGTGAATCACAAGCCCAGCAACACCAAGGTGGACAAGAAAGTTGAGCCC |
| 751 | AAATCTTGTGACAAAACTCACACATGCCCACCGTGCCCAGCGGCCGCAGG |
| 801 | TGGTGGCGGTTCAGGCGGAGGTGGCTCTGGCGGTGGCGGATCCGGAATCA |
| 851 | TAAACACATTACAGAAATATTATTGCAGAGTCAGAGGCGGCCGGTGTGCT |
| 901 | GTGCTCAGCTGCCTTCCAAAGGAGGAACAGATCGGCAAGTGCTCGACCCG |
| 951 | TGGCCGAAAATGCTGCCGAAGAAAGAAACATCACCATCACCATCACTAA |
| 1-60 | Signal peptide |
| 61-999 | Chimeric murine-human Heavy chain-HBD3 fusion |
| SEQ ID NO: 90. I9-6001-HC-F(ab)2-HBD3, heavy | |
| chain-HBD3 chimeric murine-human fusion, amino | |
| acid sequence, ID: 500768p | |
| | .........o.........o.........o.........o.........o |
| 1 | METDTLLLWVLLLWVPGSTGDHHHHHHTRQVQLQQSGAELVRPGTSVKVS |
| 51 | CKASGYAFTNYLIEWVKQRPGQGLEWIGVINPGSGGTNYNEKFKGKATLT |
| 101 | ADKSSSTAYMQLSSLTSDDSAVYFCARWDYGSSYERAMDYWGQGTSVTVS |
| 151 | ASTKGPSVFPLAPSSKSTSGGTAALGCLVKDYFPEPVTVSWNSGALTSGV |
| 201 | HTFPAVLQSSGLYSLSSVVTVPSSSLGTQTYICNVNHKPSNTKVDKKVEP |
| 251 | KSCDKTHTCPPCPAAAGGGGSGGGGSGGGGSGIINTLQKYYCRVRGGRCA |
| 301 | VLSCLPKEEQIGKCSTRGRKCCRRKKHHHHHH |
| 1-20 | Signal peptide |
| 21-332 | chimeric murine-human heavy chain F(ab)2 HBD3 |
| | fusion |
| SEQ ID NO: 91. I9-6001-HC-Fab-HBD3, heavy chain- | |
| HBD3 chimeric murine-human fusion, nucleotide | |
| sequence, ID: 500768n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACCATCACCATCACCATCACACGCGTCAGGTCCAGCTGC |
| 101 | AGCAGTCTGGAGCTGAGCTGGTAAGGCCTGGGACTTCAGTGAAGGTGTCC |
| 151 | TGCAAGGCTTCTGGATACGCCTTCACTAATTACTTGATAGAGTGGGTAAA |
| 201 | GCAGAGGCCTGGACAGGGCCTTGAGTGGATTGGAGTGATTAATCCTGGAA |
| 251 | GTGGTGGTACTAACTACAATGAGAAGTTCAAGGGCAAGGCAACACTGACT |
| 301 | GCAGACAAATCCTCCAGCACTGCCTACATGCAGCTCAGCAGCCTGACATC |
| 351 | TGATGACTCTGCGGTCTATTTCTGTGCAAGATGGGACTACGGTAGTAGCT |
| 401 | ACGAACGTGCTATGGACTACTGGGGTCAAGGAACCTCAGTCACCGTCTCC |
| 451 | GCGTCGACCAAGGGCCCATCGGTCTTCCCCCTGGCACCCTCCTCCAAGAG |
| 501 | CACCTCTGGGGGCACAGCGGCCCTGGGCTGCCTGGTCAAGGACTACTTCC |
| 551 | CCGAACCGGTGACGGTGTCGTGGAACTCAGGCGCCCTGACCAGCGGCGTG |
| 601 | CACACCTTCCCGGCTGTCCTACAGTCCTCAGGACTCTACTCCCTCAGCAG |
| 651 | CGTGGTGACCGTGCCCTCCAGCAGCTTGGGCACCCAGACCTACATCTGCA |
| 701 | ACGTGAATCACAAGCCCAGCAACACCAAGGTGGACAAGAAAGTTGAGCCC |
| 751 | AAATCTTGTGACAAAACTCACACATCCCCACCGTCCCCAGCGGCCGCAGG |
| 801 | TGGTGGCGGTTCAGGCGGAGGTGGCTCTGGCGGTGGCGGATCCGGAATCA |
| 851 | TAAACACATTACAGAAATATTATTGCAGAGTCAGAGGCGGCCGGTGTGCT |
| 901 | GTGCTCAGCTGCCTTCCAAAGGAGGAACAGATCGGCAAGTGCTCGACGCG |
| 951 | TGGCCGAAAATGCTGCCGAAGAAAGAAATAA |
| 1-60 | Signal peptide |
| 61-981 | Chimeric murine-human Heavy chain-HBD3 fusion |
| SEQ ID NO: 92. I9-6001-HC-Fab-HBD3, heavy chain- | |
| HBD3 chimeric murine-human fusion, amino acid | |
| sequence, ID: 500768p | |
| | .........o.........o.........o.........o.........o |
| 1 | METDTLLLWVLLLWVPGSTGDHHHHHHTRQVQLQQSGAELVRPGTSVKVS |
| 51 | CKASGYAFTNYLIEWVKQRPGQGLEWIGVINPGSGGTNYNEKFKGKATLT |
| 101 | ADKSSSTAYMQLSSLTSDDSAVYFCARWDYGSSYERAMDYWGQGTSVTVS |
| 151 | ASTKGPSVFPLAPSSKSTSGGTAALGCLVKDYFPEPVTVSWNSGALTSGV |
| 201 | HTFPAVLQSSGLYSLSSVVTVPSSSLGTQTYICNVNHKPSNTKVDKKVEP |
| 251 | KSCDKTHTSPPSPAAAGGGGSGGGGSGGGGSGIINTLQKYYCRVRGGRCA |
| 301 | VLSCLPKEEQIGKCSTRGRKCCRRKK |
| 1-20 | Signal peptide |
| 21-332 | chimeric murine-human heavy chain F(ab)2 HBD3 |
| | fusion |
| SEQ ID NO: 220. Linker-magainin 1, nucleotide | |
| sequence, ID: 500801n | |
| | .........o.........o.........o.........o.........o |
| 1 | GGTGGTGGCGGTTCAGGCGGAGGTGGCTCTGGCGGTGGCGGATCCGGCAT |
| 51 | CGGCAAGTTCCTGCACTCCGCCGGCAAGTTCGGCAAGGCCTTCGTGGGCG |
| 101 | AGATCATGAAGTCCTAG |
| 1-45 | Glycine-serine linker (G4S)3 |
| 46-117 | Magainin 1 |
| SEQ ID NO: 221. Linker-magainin 1, amino acid | |
| sequence, ID: 500801p | |
| | .........o.........o.........o.........o.........o |
| 1 | GGGGSGGGGSGGGGSGIGKFLHSAGKFGKAFVGEIMKS |
| 1-15 | Glycine-serine linker (G4S)3 |
| 16-38 | magainin 1 |
| SEQ ID NO: 222. Linker-magainin 2, nucleotide | |
| sequence, ID: 500802n | |
| | .........o.........o.........o.........o.........o |
| 1 | GGTGGTGGCGGTTCAGGCGGAGGTGGCTCTGGCGGTGGCGGATCCGGCAT |
| 51 | CGGCAAGTTCCTGCACTCCGCCAAGAAGTTCGGCAAGGCCTTCGTGGGCG |
| 101 | AGATCATGAACTCCTAG |
| 1-45 | Glycine-serine linker (G4S)3 |
| 46-117 | Magainin 2 |
| SEQ ID NO: 223. Linker-magainin 2, amino acid | |
| sequence, ID: 500802p | |
| | .........o.........o.........o.........o.........o |
| 1 | GGGGSGGGGSGGGGSGIGKFLHSAKKFGKAFVGEIMNS |
| 1-15 | Glycine-serine linker (G4S)3 |
| 16-38 | magainin 2 |

### Example 9

### Construction, Expression and Efficacy of Tethered Microbiocides

In addition to antibodies with variable region specificity for epitopes on Staphylococcus, recombinant fusion microbiocides were also constructed comprising immunoglobulins which do not have specific binding to S. aureus. A number of configurations of tethered microbiocides are shown in FIG. 6.

Recombinant immunoglobulin 1A9 was derived from a hybridoma which targets a surface epitope of Cryptosporidium parvum as described by Schaefer et al (Schaefer et al Infect Immun 2000 May; 68(5):2608-16). Antibody fusions were developed as described in U.S. application Ser. Nos. 12/686,879, 12/536,291, 11/545,601, and 11/254,500, each of which are incorporated herein by reference in their entirety. LYST-1A9-mVhc-LC-1A9-G1-HBD2-mVhC-HC (a human mouse chimera with human defensin B2 as a fusion microbiocide on the heavy chain and lysostaphin on the light chain) was shown to reduce the titer of Staphylococcus aureus as shown in Table 4 and FIGS. 3-5. The sequences for LYST-1A9-mVhc-LC-1A9-G1-HBD2-mVhC-HC are provided below.

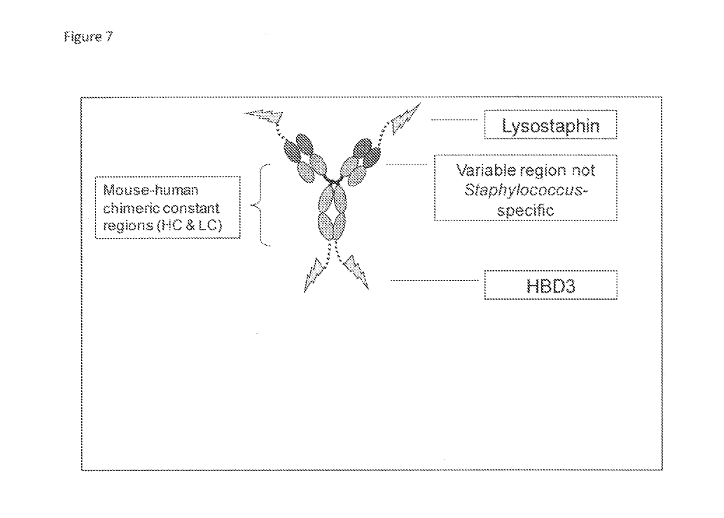

Recombinant immunoglobulin 277 binds to matrix protein 2 (M2) of Influenza A virus A/Puerto Rico/8-V24/1934(H1N1). The antibody was generated by injecting mice with recombinant hFc-M2 and was found to be specific for influenza PR8 M2 protein by multiple assays. The antibody was engineered into a chimeric mouse-human G1 antibody with lysostaphin attached to the N-terminus of the light chain and HBD3 (human biodefensin 3) attached to the C-terminus of the heavy chain (FIG. 7). The sequences for LYST-c277-chG1-HBD3 are provided below.

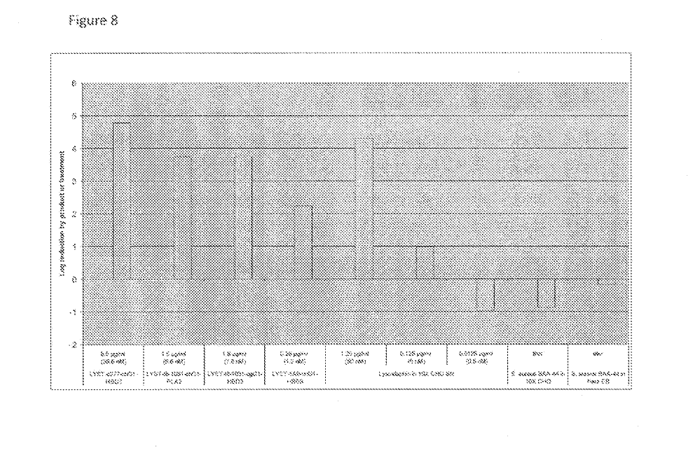

As can be seen in FIG. 8, LYST-c277-chG1-HBD3 is highly effective at killing MRSA strain BAA-44 at 40 nM, outperforming standalone lysostaphin.

The killing assay was done as follows: S. aureus BAA-44 was grown to log phase, harvested and washed then resuspended in a volume of 1×PBS to give a suspension of 2.02×107 CFU/ml (target cells=200,000 per well after blocking). 10 μl of 11 mg/ml P548 (hG1-CH2-CH3) were added to 0.99 ml of titered bacteria to block Protein A; mixture was incubated on ice for 30 min with occasional light vortexing.

Products were grown in culture flasks, harvested supernatants were concentrated 10× by volume using Amicon 30 kDa concentrators. This concentrate was quantified by ELISA and the numbers in table reflect the actual concentration applied per well. 10× concentrated CHO supernatant was used as a killing buffer for titer and the positive control lysostaphin treatments.

### Non-Staphylococcus-Specific Full Size Chimeric Antibody-Biocide Fusions

| SEQ ID NO: 230. c277-HC-HBD3, heavy chain-HBD3 | |
|---|---|
| chimeric murine-human fusion, nucleotide sequence, | |
| ID: 500545n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACACGCGTCAGGTCCAACTGCAGCAGTCTGGGCCTGAGG |
| 101 | TGGTGAGGCCTGGGGTCTCAGTGAAGATTTCCTGCAAGGGTTCCGGCTAC |
| 151 | ACATTCACTGATTATGCTATGCACTGGGTGAAGCAGAGTCATGCAAAGAG |
| 201 | GCAAGAGTGGATTGGAGTTATTGGTACTTACAATGGTAATACAAACTACA |
| 251 | ACCAGAAGTTTAAGGGCAAGGCCACAATGACTGTAGACAGATCCTCCAGC |
| 301 | ACAGCCTATATGGAACTTGCCGGTTTGACATCTGAGGATTCTGCCATCTA |
| 351 | TTACTGTGCAAGAAGGGGTGATTACGACGCCTGGTTTGCTTACTGGGGCC |
| 401 | AAGGGACTCTGGTCACTGTCTCTGCAGCGTCGACCAAGGGCCCATCGGTC |
| 451 | TTCCCCCTGGCACCCTCTAGCAAGAGCACCTCTGGGGGCACAGCGGCCCT |
| 501 | GGGCTGCCTGGTCAAGGACTACTTCCCCGAACCGGTGACGGTGTCGTGGA |
| 551 | ACTCAGGCGCCCTGACCAGCGGCGTGCACACCTTCCCGGCTGTCCTACAG |
| 601 | TCCTCAGGACTCTACTCCCTCAGCAGCGTGGTGACCGTGCCCTCCAGCAG |
| 651 | CTTGGGCACCCAGACCTACATCTGCAACGTGAATCACAAGCCCAGCAACA |
| 701 | CCAAGGTGGACAAGAGAGTTGAGCCCAAATCTTGTGACAAAACTCACACA |
| 751 | TGCCCACCGTGCCCAGCACCTGAACTCCTGGGGGGACCGTCAGTCTTCCT |
| 801 | CTTCCCCCCAAAACCCAAGGACACCCTCATGATCTCCCGGACCCCTGAGG |
| 851 | TCACATGCGTGGTGGTGGACGTGAGCCACGAAGACCCTGAGGTCAAGTTC |
| 901 | AACTGGTACGTGGACGGCGTGGAGGTGCATAATGCCAAGACAAAGCCGCG |
| 951 | GGAGGAGCAGTACAACAGCACGTACCGTGTGGTCAGCGTCCTCACCGTCC |
| 1001 | TGCACCAGGACTGGCTGAATGGCAAGGAGTACAAGTGCAAGGTCTCCAAC |
| 1051 | AAAGCCCTCCCAGCCCCCATCGAGAAAACCATCTCCAAAGCCAAAGGGCA |
| 1101 | GCCCCGAGAACCACAGGTGTACACCCTGCCCCCATCCCGGGAGGAGATGA |
| 1151 | CCAAGAACCAGGTCAGCCTGACCTGCCTGGTCAAAGGCTTCTATCCCAGC |
| 1201 | GACATCGCCGTGGAGTGGGAGAGCAATGGGCAGCCGGAGAACAACTACAA |
| 1251 | GACCACGCCTCCCGTGCTGGACTCCGACGGCTCCTTCTTCCTCTATAGCA |
| 1301 | AGCTCACCGTGGACAAGAGCAGGTGGCAGCAGGGGAACGTCTTCTCATGC |
| 1351 | TCCGTGATGCATGAGGCTCTGCACAACCACTACACGCAGAAGAGCCTCTC |
| 1401 | CCTGTCTCCGGGTAAAGCGGCCGCAGGTGGTGGCGGTTCAGGCGGAGGTG |
| 1451 | GCTCTGGCGGTGGCGGATCCGGAATCATAAACACATTACAGAAATATTAT |
| 1501 | TGCAGAGTCAGAGGCGGCCGGTGTGCTGTGCTCAGCTGCCTTCCAAAGGA |
| 1551 | GGAACAGATCGGCAAGTGCTCGACGCGTGGCCGAAAATGCTGCCGAAGAA |
| 1601 | AGAAATAA |
| 1-60 | Signal peptide |
| 61-1608 | Chimeric murine-human Heavy chain-HBD3 fusion |
| SEQ ID NO: 231. c277-HC-HBD3, heavy chain-HBD3 | |
| chimeric murine-human fusion, amino acid sequence, | |
| ID: 500545p | |
| | .........o.........o.........o.........o.........o |
| 1 | METDTLLLWVLLLWVPGSTGDTRQVQLQQSGPEVVRPGVSVKISCKGSGY |
| 51 | TFTDYAMHWVKQSHAKRQEWIGVIGTYNGNTNYNQKFKGKATMTVDRSSS |
| 101 | TAYMELAGLTSEDSAIYYCARRGDYDAWFAYWGQGTLVTVSAASTKGPSV |
| 151 | FPLAPSSKSTSGGTAALGCLVKDYFPEPVTVSWNSGALTSGVHTFPAVLQ |
| 201 | SSGLYSLSSVVTVPSSSLGTQTYICNVNHKPSNTKVDKRVEPKSCDKTHT |
| 251 | CPPCPAPELLGGPSVFLFPPKPKDTLMISRTPEVTCVVVDVSHEDPEVKF |
| 301 | NWYVDGVEVHNAKTKPREEQYNSTYRVVSVLTVLHQDWLNGKEYKCKVSN |
| 351 | KALPAPIEKTISKAKGQPREPQVYTLPPSREEMTKNQVSLTCLVKGFYPS |
| 401 | DIAVEWESNGQPENNYKTTPPVLDSDGSFFLYSKLTVDKSRWQQGNVFSC |
| 451 | SVMHEALHNHYTQKSLSLSPGKAAAGGGGSGGGGSGGGGSGIINTLQKYY |
| 501 | CRVRGGRCAVLSCLPKEEQIGKCSTRGRKCCRRKK |
| 1-20 | Signal peptide |
| 21-535 | chimeric murine-human heavy chain-HBD3 fusion |
| SEQ ID NO: 293. LYST-LC-c277, Lysostaphin-light | |
| chain chimeric murine-human fusion, nucleotide | |
| sequence, ID: 500786n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACGCCACCCACGAGCACTCCGCCCAGTGGCTGAACAACT |
| 101 | ACAAGAAGGGCTACGGCTACGGCCCCTACCCCCTGGGCATCAACGGCGGC |
| 151 | ATGCACTACGGCGTGGACTTCTTCATGAACATCGGCACCCCCGTGAAGGC |
| 201 | CATCTCCTCCGGCAAGATCGTGGAGGCCGGCTGGTCCAACTACGGCGGCG |
| 251 | GCAACCAGATCGGCCTGATCGAGAACGACGGCGTGCACCGCCAGTGGTAC |
| 301 | ATGCACCTGTCCAAGTACAACGTGAAGGTGGGCGACTACGTGAAGGCCGG |
| 351 | CCAGATCATCGGCTGGTCCGGCTCCACCGGCTACTCCACCGCCCCCCACC |
| 401 | TGCACTTCCAGCGCATGGTGAACTCCTTCTCCAACTCCACCGCCCAGGAC |
| 451 | CCCATGCCCTTCCTGAAGTCCGCCGGCTACGGCAAGGCCGGCGGCACCGT |
| 501 | GACCCCCACCCCCAACACCGGCTGGAAGACCAACAAGTACGGCACCCTGT |
| 551 | ACAAGTCCGAGTCCGCCTCCTTCACCCCCAACACCGACATCATCACCCGC |
| 601 | ACCACCGGCCCCTTCCGCTCCATGCCCCAGTCCGGCGTGCTGAAGGCCGG |
| 651 | CCAGACCATCCACTACGACGAGGTGATGAAGCAGGACGGCCACGTGTGGG |
| 701 | TGGGCTACACCGGCAACTCCGGCCAGCGCATCTACCTGCCCGTGCGCACC |
| 751 | TGGAACAAGTCCACCAACACCCTGGGCGTGCTGTGGGGCACCATCAAGGG |
| 801 | TGGTGGCGGTTCAGGCGGAGGTGGCTCTGGCGGTGGCGGATCcacgcgtG |
| 851 | ACATTGTGATGACCCaGTCTCAAAAATTCATGTCCACAcCAGTAGGAGAC |
| 901 | AGGGTCAGCGTCACCTGCAAGGCCAGTCAGAATGTGGGTACTTATGTAGC |
| 951 | CTGGTATCAACAGAAACCAGGGCAGTCTCCTAAAATACTGATTTATTCGG |
| 1001 | CATCCTACCGGTACAGTGGAGTCCCTGATCGCTTCACAGGCAGTGGCTCT |
| 1051 | GGGACAGATTTCACTCTCACCATCAGCAATGTGCAGTCTGAAGACTTGGC |
| 1101 | AGAGTATTTCTGTCAGCGATATAACAGCTATCCTCTCACGTTCGGTGCTG |
| 1151 | GGACCAAGCTGGAGCTGAAACGGACTGTGGCTGCACCATCTGTCTTCATC |
| 1201 | TTCCCGCCATCTGATGAGCAGTTGAAATCTGGAACTGCCTCTGTTGTGTG |
| 1251 | CCTGCTGAATAACTTCTATCCCAGAGAGGCCAAAGTACAGTGGAAGGTGG |
| 1301 | ATAACGCCCTCCAATCGGGTAACTCCCAGGAGAGTGTCACAGAGCAGGAC |
| 1351 | AGCAAGGACAGCACCTACAGCCTCAGCAGCACCCTGACGCTGAGCAAAGC |
| 1401 | AGACTACGAGAAACACAAAGTCTACGCCTGCGAAGTCACCCATCAGGGCC |
| 1451 | TGAGCTCGCCCGTCACAAAGAGCTTCAACAGGGGAGAGTGTTAG |
| 1-60 | Signal peptide |
| 61-735 | Lysostaphin |
| 736-1494 | Linker-LC |
| SEQ ID NO: 232. LYST-LC-c277, Lysostaphin-light | |
| chain fusion, murine-human chimeric, amino acid | |
| sequence, ID: 50086p | |
| | .........o.........o.........o.........o.........o |
| 1 | METDTLLLWVLLLWVPGSTGDATHEHSAQWLNNYKKGYGYGPYPLGINGG |
| 51 | MHYGVDFFMNIGTPVKAISSGKIVEAGWSNYGGGNQIGLIENDGVHRQWY |
| 101 | MHLSKYNVKVGDYVKAGQIIGWSGSTGYSTAPHLHFQRMVNSFSNSTAQD |
| 151 | PMPFLKSAGYGKAGGTVTPTPNTGWKTNKYGTLYKSESASFTPNTDIITR |
| 201 | TTGPFRSMPQSGVLKAGQTIHYDEVMKQDGHVWVGYTGNSGQRIYLPVRT |
| 251 | WNKSTNTLGVLWGTIKGGGGSGGGGSGGGGSTRDIVMTQSQKFMSTPVGD |
| 301 | RVSVTCKASQNVGTYVAWYQQKPGQSPKILIYSASYRYSGVPDRFTGSGS |
| 351 | GTDFTLTISNVQSEDLAEYFCQRYNSYPLTFGAGTKLELKRTVAAPSVFI |
| 401 | FPPSDEQLKSGTASVVCLLNNFYPREAKVQWKVDNALQSGNSQESVTEQD |
| 451 | SKDSTYSLSSTLTLSKADYEKHKVYACEVTHQGLSSPVTKSFNRGEC |
| 1-20 | Signal peptide |
| 21-266 | Lysostaphin |
| 267-497 | Linker-LC |
| SEQ ID NO: 233. 1A9-HC-HBD2, heavy chain-HBD2 | |
| chimeric murine-human fusion, nucleotide sequence, | |
| ID: 500305n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACACGCGTCAGATCCAGTTGGTGCAGTCTGGACCTGAGC |
| 101 | TGAAGAAGCCTGGAGAGACAGTCAAGATCTCCTGCAAGGCTTCTGGGTAT |
| 151 | ACCTTCACAAACTATGGAATGAACTGGGTGAAGCAGGCTCCAGGAAAGGG |
| 201 | TTTAAAGTGGATGGGCTGGATAAACACCAACACTGGAGAGCCAACATATG |
| 251 | CTGAAGAGTTCAAGGGGCGGTTTGCCTTCTCTTTGGAAACCTCTGCCAGC |
| 301 | ACTGCCTATTTGCAGATCAACAACCTCAAAAATGAGGACACGGCTACATA |
| 351 | TTTCTGTGCAAGACACGGTGGTAGGAGCTGGTACTTCGATGTCTGGGGCG |
| 401 | CAGGGACCACGGTCACCGTCTCCTCAGCGTCGACCAAGGGCCCATCGGTC |
| 451 | TTCCCCCTGGCACCCTCTAGCAAGAGCACCTCTGGGGGCACAGCGGCCCT |
| 501 | GGGCTGCCTGGTCAAGGACTACTTCCCCGAACCGGTGACGGTGTCGTGGA |
| 551 | ACTCAGGCGCCCTGACCAGCGGCGTGCACACCTTCCCGGCTGTCCTACAG |
| 601 | TCCTCAGGACTCTACTCCCTCAGCAGCGTGGTGACCGTGCCCTCCAGCAG |
| 651 | CTTGGGCACCCAGACCTACATCTGCAACGTGAATCACAAGCCCAGCAACA |
| 701 | CCAAGGTGGACAAGAGAGTTGAGCCCAAATCTTGTGACAAAACTCACACA |
| 751 | TGCCCACCGTGCCCAGCACCTGAACTCCTGGGGGGACCGTCAGTCTTCCT |
| 801 | CTTCCCCCCAAAACCCAAGGACACCCTCATGATCTCCCGGACCCCTGAGG |
| 851 | TCACATGCGTGGTGGTGGACGTGAGCCACGAAGACCCTGAGGTCAAGTTC |
| 901 | AACTGGTACGTGGACGGCGTGGAGGTGCATAATGCCAAGACAAAGCCGCG |
| 951 | GGAGGAGCAGTACAACAGCACGTACCGTGTGGTCAGCGTCCTCACCGTCC |
| 1001 | TGCACCAGGACTGGCTGAATGGCAAGGAGTACAAGTGCAAGGTCTCCAAC |
| 1051 | AAAGCCCTCCCAGCCCCCATCGAGAAAACCATCTCCAAAGCCAAAGGGCA |
| 1101 | GCCCCGAGAACCACAGGTGTACACCCTGCCCCCATCCCGGGAGGAGATGA |
| 1151 | CCAAGAACCAGGTCAGCCTGACCTGCCTGGTCAAAGGCTTCTATCCCAGC |
| 1201 | GACATCGCCGTGGAGTGGGAGAGCAATGGGCAGCCGGAGAACAACTACAA |
| 1251 | GACCACGCCTCCCGTGCTGGACTCCGACGGCTCCTTCTTCCTCTATAGCA |
| 1301 | AGCTCACCGTGGACAAGAGCAGGTGGCAGCAGGGGAACGTCTTCTCATGC |
| 1351 | TCCGTGATGCATGAGGCTCTGCACAACCACTACACGCAGAAGAGCCTCTC |
| 1401 | CCTGTCTCCGGGTAAAGCGGCCGCAGGTGGTGGCGGTTCAGGCGGAGGTG |
| 1451 | GCTCTGGCGGTGGCGGATCCGGTATAGGCGATCCTGTTACCTGCCTTAAG |
| 1501 | AGTGGAGCCATATGTCATCCAGTCTTTTGCCCTAGAAGGTATAAACAAAT |
| 1551 | TGGCACCTGTGGTCTCCCTGGAACAAAATGCTGCAAAAAGCCATGA |
| 1-60 | Signal peptide |
| 61-1596 | Chimeric murine-human Heavy chain-HBD2 fusion |
| SEQ ID NO: 234. 1A9-HC-HBD2, heavy chain-HBD2 | |
| chimeric murine-human fusion, amino acid sequence, | |
| ID: 500305p | |
| | .........o.........o.........o.........o.........o |
| 1 | METDTLLLWVLLLWVPGSTGDTRQIQLVQSGPELKKPGETVKISCKASGY |
| 51 | TFTNYGMNWVKQAPGKGLKWMGWINTNTGEPTYAEEFKGRFAFSLETSAS |
| 101 | TAYLQINNLKNEDTATYFCARHGGRSWYFDVWGAGTTVTVSSASTKGPSV |
| 151 | FPLAPSSKSTSGGTAALGCLVKDYFPEPVTVSWNSGALTSGVHTFPAVLQ |
| 201 | SSGLYSLSSVVTVPSSSLGTQTYICNVNHKPSNTKVDKRVEPKSCDKTHT |
| 251 | CPPCPAPELLGGPSVFLFPPKPKDTLMISRTPEVTCVVVDVSHEDPEVKF |
| 301 | NWYVDGVEVHNAKTKPREEQYNSTYRVVSVLTVLHQDWLNGKEYKCKVSN |
| 351 | KALPAPIEKTISKAKGQPREPQVYTLPPSREEMTKNQVSLTCLVKGFYPS |
| 401 | DIAVEWESNGQPENNYKTTPPVLDSDGSFFLYSKLTVDKSRWQQGNVFSC |
| 451 | SVMHEALHNHYTQKSLSLSPGKAAAGGGGSGGGGSGGGGSGIGDPVTCLK |
| 501 | SGAICHPVFCPRRYKQIGTCGLPGTKCCKKP |
| 1-20 | Signal peptide |
| 21-531 | chimeric murine-human heavy chain-HBD2 fusion |
| SEQ ID NO: 235. LYST-LC-1A9, Lysostaphin-light | |
| chain chimeric murine-human fusion, nucleotide | |
| sequence, ID: 500754n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACGCCACCCACGAGCACTCCGCCCAGTGGCTGAACAACT |
| 101 | ACAAGAAGGGCTACGGCTACGGCCCCTACCCCCTGGGCATCAACGGCGGC |
| 151 | ATGCACTACGGCGTGGACTTCTTCATGAACATCGGCACCCCCGTGAAGGC |
| 201 | CATCTCCTCCGGCAAGATCGTGGAGGCCGGCTGGTCCAACTACGGCGGCG |
| 251 | GCAACCAGATCGGCCTGATCGAGAACGACGGCGTGCACCGCCAGTGGTAC |
| 301 | ATGCACCTGTCCAAGTACAACGTGAAGGTGGGCGACTACGTGAAGGCCGG |
| 351 | CCAGATCATCGGCTGGTCCGGCTCCACCGGCTACTCCACCGCCCCCCACC |
| 401 | TGCACTTCCAGCGCATGGTGAACTCCTTCTCCAACTCCACCGCCCAGGAC |
| 451 | CCCATGCCCTTCCTGAAGTCCGCCGGCTACGGCAAGGCCGGCGGCACCGT |
| 501 | GACCCCCACCCCCAACACCGGCTGGAAGACCAACAAGTACGGCACCCTGT |
| 551 | ACAAGTCCGAGTCCGCCTCCTTCACCCCCAACACCGACATCATCACCCGC |
| 601 | ACCACCGGCCCCTTCCGCTCCATGCCCCAGTCCGGCGTGCTGAAGGCCGG |
| 651 | CCAGACCATCCACTACGACGAGGTGATGAAGCAGGACGGCCACGTGTGGG |
| 701 | TGGGCTACACCGGCAACTCCGGCCAGCGCATCTACCTGCCCGTGCGCACC |
| 751 | TGGAACAAGTCCACCAACACCCTGGGCGTGCTGTGGGGCACCATCAAGGG |
| 801 | TGGTGGCGGTTCAGGCGGAGGTGGCTCTGGCGGTGGCGGATCcaCGCGTG |
| 851 | ATGTTGTGATGACCCAAATTCCACTCTCCCTGCCTGTCAGTCTTGGAGAT |
| 901 | CAAGCCTCCATCTCTTGCAGATCTAGTCAGAGCCTTGTACACAGTAATGG |
| 951 | AAACACCTATTTACATTGGTACCTGCAGAAGCCAGGCCAGTCTCCAAAGC |
| 1001 | TCCTGATCTACAAAGTTTCCAACCGATTTTCTGGGGTCCCAGACAGGTTC |
| 1051 | AGTGGCAGTGGATCAGGGACAGATTTCACACTCAAGATCAGCAGAGTGGA |
| 1101 | GGCTGAGGATCTGGGAGTTTATTTCTGCTCTCAAAGTACACATGTTCCTC |
| 1151 | CGTGGACGTTTGGTGGAGGCACCAAGCTGGAAATCAAACGGACTGTGGCT |
| 1201 | GCACCATCTGTCTTCATCTTCCCGCCATCTGATGAGCAGTTGAAATCTGG |
| 1251 | AACTGCCTCTGTTGTGTGCCTGCTGAATAACTTCTATCCCAGAGAGGCCA |
| 1301 | AAGTACAGTGGAAGGTGGATAACGCCCTCCAATCGGGTAACTCCCAGGAG |
| 1351 | AGTGTCACAGAGCAGGACAGCAAGGACAGCACCTACAGCCTCAGCAGCAC |
| 1401 | CCTGACGCTGAGCAAAGCAGACTACGAGAAACACAAAGTCTACGCCTGCG |
| 1451 | AAGTCACCCATCAGGGCCTGAGCTCGCCCGTCACAAAGAGCTTCAACAGG |
| 1501 | GGAGAGTGTTAG |
| 1-60 | Signal peptide |
| 61-735 | Lysostaphin |
| 736-1512 | Linker-LC |
| SEQ ID NO: 236. LYST-LC-1A9, Lysostaphin-light | |
| chain fusion, murine-human chimeric, amino acid | |
| sequence, ID: 500754p | |
| | .........o.........o.........o.........o.........o |
| 1 | METDTLLLWVLLLWVPGSTGDATHEHSAQWLNNYKKGYGYGPYPLGINGG |
| 51 | MHYGVDFFMNIGTPVKAISSGKIVEAGWSNYGGGNQIGLIENDGVHRQWY |
| 101 | MHLSKYNVKVGDYVKAGQIIGWSGSTGYSTAPHLHFQRMVNSFSNSTAQD |
| 151 | PMPFLKSAGYGKAGGTVTPTPNTGWKTNKYGTLYKSESASFTPNTDIITR |
| 201 | TTGPFRSMPQSGVLKAGQTIHYDEVMKQDGHVWVGYTGNSGQRIYLPVRT |
| 251 | WNKSTNTLGVLWGTIKGGGGSGGGGSGGGGSTRDVVMTQIPLSLPVSLGD |
| 301 | QASISCRSSQSLVHSNGNTYLHWYLQKPGQSPKLLIYKVSNRFSGVPDRF |
| 351 | SGSGSGTDFTLKISRVEAEDLGVYFCSQSTHVPPWTFGGGTKLEIKRTVA |
| 401 | APSVFIFPPSDEQLKSGTASVVCLLNNFYPREAKVQWKVDNALQSGNSQE |
| 451 | SVTEQDSKDSTYSLSSTLTLSKADYEKHKVYACEVTHQGLSSPVTKSFNR |
| 501 | GEC |
| 1-20 | Signal peptide |
| 21-266 | Lysostaphin |
| 267-503 | Linker-LC |
| Seq. 237. I10-9004 light chain variable region, | |
| nucleotide sequence, ID: 500778n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACacgcgtGACATCTTGCTGACTCAGTCTCCAGCCATCC |
| 101 | TGTCTGTGAGTCCAGGAGAAAGAGTCAGTTTCTCCTGCAGGGCCAGTCAG |
| 151 | AGCATTGGCACAAGCATACACTGGTATCAGCAAAGAACAAATGGTTCTCC |
| 201 | AAGGCTTCTCATAAAGTATGCTTCTGAGTCTATCTCTGGGATCCCTTCCA |
| 251 | GGTTTAGTGGCAGTGGATCAGGGACAGATTTTACTCTTAGCATCAACAGT |
| 301 | GTGGAGTCTGAAGATATTGCAGATTATTACTGTCAACAAAGTAATAGCTG |
| 351 | GCCAACCACGTTCGGCTCGGGGACAAAGTTGGAAATAAAACGG |
| 1-60 | Signal peptide |
| 61-393 | Light chain variable region |
| Seq. 238. I10-9004 light chain variable region, | |
| amino acid sequence, ID: 500778p | |
| | .........o.........o.........o.........o.........o |
| 1 | METDTLLLWVLLLWVPGSTGDTRDILLTQSPAILSVSPGERVSFSCRASQ |
| 51 | SIGTSIHWYQQRTNGSPRLLIKYASESISGIPSRFSGSGSGTDFTLSINS |
| 101 | VESEDIADYYCQQSNSWPTTFGSGTKLEIKR |
| 1-20 | Signal peptide |
| 21-131 | Light chain variable region |
| Seq. 239. I10-9004 heavy chain variable region, | |
| nucleotide sequence, ID: 500779n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACacgcgtGAGGTCCAGCTGCAGCAGTCTGGACCTGAGC |
| 101 | TGGTAAAGCCTGGGGCTTCAGTGAAGATGTCCTGCAAGGCTTCTGGATAC |
| 151 | ACATTCACTAGCTATGTTATGCACTGGGTGAAGCAGAAGCCTGGGCAGGG |
| 201 | CCTTGAGTGGATTGGATATATTAATCCTTACAATGATGGTACTAAGTACA |
| 251 | ATGAGAAGTTCAAAGGCAAGGCCACACTGACTTCAGACAAATCCTCCAGC |
| 301 | ACAGCCTACATGGAGCTCAGCAGCCTGACCTCTGAGGACTCTGCGGTCTA |
| 351 | TTACTGTGCAAGAAGTGAGGGGGGGATCTACTATGATTACGATGTTGCTT |
| 401 | ACTGGGGCCAAGGGACTCTGGTCACTGTCTCT |
| 1-60 | Signal peptide |
| 61-432 | Heavy chain variable region |
| Seq. 240. I10-9004 heavy chain variable region, | |
| amino acid sequence, ID: 500779p | |
| | .........o.........o.........o.........o.........o |
| 1 | METDTLLLWVLLLWVPGSTGDTREVQLQQSGPELVKPGASVKMSCKASGY |
| 51 | TFTSYVMHWVKQKPGQGLEWIGYINPYNDGTKYNEKFKGKATLTSDKSSS |
| 101 | TAYMELSSLTSEDSAVYYCARSEGGIYYDYDVAYWGQGTLVTVS |
| 1-20 | Signal peptide |
| 21-144 | Heavy chain variable region |
| Seq. 241. I8-1351 light chain variable region, | |
| nucleotide sequence, ID: 500835n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACacgcgtCAAATTGTTCTCACCCAGTCTCCAGCAATCA |
| 101 | TGTCTGCATCTCCAGGGGAGAAGGTCACCATGACCTGCAGTGCCAGCTCA |
| 151 | AGTGTAAGTTACATGCACTGGTACCAGCAGAAGTCAGGCACCTCCCCCAA |
| 201 | AAGATGGATTTATGACACATCCAAACTGGCTTCTGGAGTCCCTGCTCGCT |
| 251 | TCAGTGGCAGTGGGTCTGGGACCTCTTACTCTCTCACAATCAGCAGCATG |
| 301 | GAGGCTGAAGATGCTGCCACTTATTACTGCCAGCAGTGGAGTAGTAACCC |
| 351 | ACCGACGTTCGGTGGAGGCACCAAGCTGGAAATCAAACGG |
| 1-60 | Signal peptide |
| 61-390 | Light chain variable region |
| Seq. 242. I8-1351 light chain variable region, | |
| amino acid sequence, ID: 500835p | |
| | .........o.........o.........o.........o.........o |
| 1 | METDTLLLWVLLLWVPGSTGDTRQIVLTQSPAIMSASPGEKVTMTCSASS |
| 51 | SVSYMHWYQQKSGTSPKRWIYDTSKLASGVPARFSGSGSGTSYSLTISSM |
| 101 | EAEDAATYYCQQWSSNPPTFGGGTKLEIKR |
| 1-20 | Signal peptide |
| 21-130 | Light chain variable region |
| Seq. 243. I8-1351 heavy chain variable region, | |
| nucleotide sequence, ID: 500836n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACacgcgtCAGGTCCAACTGCAGCAACCTGGGTCTGAGC |
| 101 | TGGTGAGGCCTGGAGCTTCAGTGAAGCTGTCCTGCAAGGCTTCTGGCTAC |
| 151 | ACATTCACCAGCTACTGGATGCACTGGGTGAAGCAGAGGCATGGACAAGG |
| 201 | CCTTGAGTGGATTGGAAATATTTATCCTGGTAGTGGTAGTACTAACTACG |
| 251 | ATGAGAAGTTCAAGAGCAAGGGCACACTGACTGTAGACACATCCTCCAGC |
| 301 | ACAGCCTACATGCACCTCAGCAGCCTGACATCTGAGGACTCTGCGGTCTA |
| 351 | TTACTGTACAAGAGGGGGATGGTTACTACTCGGCTACTGGTACTTCGATG |
| 401 | TCTGGGGCGCAGGGACCACGGTCACCGTCTCCTCA |
| 1-60 | Signal peptide |
| 61-435 | Heavy chain variable region |
| Seq. 244. I8-1351 heavy chain variable region, | |
| amino acid sequence, ID: 500836p | |
| | .........o.........o.........o.........o.........o |
| 1 | METDTLLLWVLLLWVPGSTGDTRQVQLQQPGSELVRPGASVKLSCKASGY |
| 51 | TFTSYWMHWVKQRHGQGLEWIGNIYPGSGSTNYDEKFKSKGTLTVDTSSS |
| 101 | TAYMHLSSLTSEDSAVYYCTRGGWLLLGYWYFDVWGAGTTVTVSS |
| 1-20 | Signal peptide |
| 21-145 | Heavy chain variable region |
| Seq. 245. I5-3002 light chain variable region, | |
| nucleotide sequence, ID: 500792n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACacgcgtCAAATTGTTCTCACCCAGTCTCCAGCAATCA |
| 101 | TGTCTGCATCTCCAGGGGAGAAGGTCACCATGACCTGCAGTGCCAGCTCA |
| 151 | AGTGTAAGTTACATGCACTGGTACCAGCAGAAGTCAGGCACCTCCCCCAA |
| 201 | AAGATGGATTTATGACACATCCAAACTGGCTTCTGGAGTCCCTGCTCGCT |
| 251 | TCAGTGGCAGTGGGTCTGGGACCTCTTACTCTCTCACAATCAGCAGCATG |
| 301 | GAGGCTGAAGATGCTGCCACTTATTACTGCCAGCAGTGGAGTAGTAACCC |
| 351 | ACCCACGTTCGGTGCTGGGACCAAGCTGGAGCTGAAACGG |
| 1-60 | Signal peptide |
| 61-390 | Light chain variable region |
| Seq. 246. I5-3002 light chain variable region, | |
| amino acid sequence, ID: 500792p | |
| | .........o.........o.........o.........o.........o |
| 1 | METDTLLLWVLLLWVPGSTGDTRQIVLTQSPAIMSASPGEKVTMTCSASS |
| 51 | SVSYMHWYQQKSGTSPKRWIYDTSKLASGVPARFSGSGSGTSYSLTISSM |
| 101 | EAEDAATYYCQQWSSNPPTFGAGTKLELKR |
| 1-20 | Signal peptide |
| 21-130 | Light chain variable region |
| Seq. 247. I5-3002 heavy chain variable region, | |
| nucleotide sequence, ID: 500793n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACacgcgtGAGGTCCAGCTGCAGCAGTCTGGACCTGAGC |
| 101 | TGGTAAAGCCTGGGGCTTCAGTGAAGATGTCCTGCAAGGCTTCTGGATAC |
| 151 | ACATTCACTAGCTATGTTATGCACTGGGTGAAGCAGAAGCCTGGGCAGGG |
| 201 | CCTTGAGTGGATTGGATATATTAATCCTTACAATGATGGTACTAAGTACA |
| 251 | ATGAGAAGTTCAAAGGCAAGGCCACACTGACTTCAGACAAATCCTCCAGC |
| 301 | ACAGCCTACATGGAGCTCAGCAGCCTGACCTCTGAGGACTCTGCGGTCTA |
| 351 | TTACaGTGCAAGACTAGCAAGGTTTGCTTACTGGGGCCAAGGGACTCTGG |
| 401 | TCACTGTCTCT |
| 1-60 | Signal peptide |
| 61-411 | Heavy chain variable region |
| Seq. 248. I5-3002 heavy chain variable region, | |
| amino acid sequence, ID: 500793p | |
| | .........o.........o.........o.........o.........o |
| 1 | METDTLLLWVLLLWVPGSTGDTREVQLQQSGPELVKPGASVKMSCKASGY |
| 51 | TFTSYVMHWVKQKPGQGLEWIGYINPYNDGTKYNEKFKGKATLTSDKSSS |
| 101 | TAYMELSSLTSEDSAVYYSARLARFAYWGQGTLVTVS |
| 1-20 | Signal peptide |
| 21-137 | Heavy chain variable region |
| Seq. 249. I9-6014 light chain variable region, | |
| nucleotide sequence, ID: 500760n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACacgcgtGATGTTGTGATGACCCAAAtTCCACTCTCCC |
| 101 | TGCCTGTCAGTCTTGGAGATCAAGCCTCCATCTCTTGCAGATCTAGTCAG |
| 151 | AgCCTTGTACACAGTAATGGAAACACCTATTTACATTGGTACCTGCAGAA |
| 201 | GCCAGGCCAGTCTCCAAAGCTCCTGATCTACAAAGTTTCCAACCGATTTT |
| 251 | CTGGGGTCCCAGACAGGTTCAGTGGCAGTGGATCAGGGACAGATTTCACA |
| 301 | CTCAAGATCAGCAGAGTGGAGGCTGAGGATCTGGGAGTTTATTTCTGCTC |
| 351 | TCAAAGTACACATGTTCCtccGtggACGTTtGGTGGAGGCACCAAGCTGG |
| 401 | AAATCAAACGG |
| 1-60 | Signal peptide |
| 61-411 | Light chain variable region |
| Seq. 250. I9-6014 light chain variable region, | |
| amino acid sequence, ID: 500760p | |
| | .........o.........o.........o.........o.........o |
| 1 | METDTLLLWVLLLWVPGSTGDTRDVVMTQIPLSLPVSLGDQASISCRSSQ |
| 51 | SLVHSNGNTYLHWYLQKPGQSPKLLIYKVSNRFSGVPDRFSGSGSGTDFT |
| 101 | LKISRVEAEDLGVYFCSQSTHVPPWTFGGGTKLEIKR |
| 1-20 | Signal peptide |
| 21-137 | Light chain variable region |
| Seq. 251. I9-6014 heavy chain variable region, | |
| nucleotide sequence, ID: 500761n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACacgcgtGAGGTTCAGCTGCAGCAGTCTGGGGCTGAGC |
| 101 | TTGTGAGGCCAGGGGCCTTAGTCAGGTTGTCCTGCAAAGCTTCTGGCTTC |
| 151 | AACATTAAAGACTACTATATGAACTGGGTGAAGCAGAGGCCTGAACAGGG |
| 201 | CCTGGAGTGGATTGGATGGATTGATCCTGAGAATGGTAATACTATATATG |
| 251 | ACCCGAAGTTCCAGGGCAAGGCCAGTATAATAGCAGACATATCCTCCAAC |
| 301 | ACAGCCTACCTACAGCTCAGCAGCCTGACATCTGAGGACACAGCCGTCTA |
| 351 | TTATTGTGCTAGATGGTACCACTATGTTATGGACTACTGGGGTCAAGGAA |
| 401 | CCTCAGTCACCGTCTCCTCA |
| 1-60 | Signal peptide |
| 61-420 | Heavy chain variable region |
| Seq. 252. I9-6014 heavy chain variable region, | |
| amino acid sequence, ID: 500761p | |
| | .........o.........o.........o.........o.........o |
| 1 | METDTLLLWVLLLWVPGSTGDTREVQLQQSGAELVRPGALVRLSCKASGF |
| 51 | NIKDYYMNWVKQRPEQGLEWIGWIDPENGNTIYDPKFQGKASIIADISSN |
| 101 | TAYLQLSSLTSEDTAVYYCARWYHYVMDYWGQGTSVTVSS |
| 1-20 | Signal peptide |
| 21-140 | Heavy chain variable region |
| Seq. 253. I5-3023 light chain variable region, | |
| nucleotide sequence, ID: 500812n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACacgcgtCAAATTGTTCTCTCCCAGTCTCCAGCAATCC |
| 101 | TGTCTGCATCTCCAGGGGAGAAGGTCACAATGACTTGCAGGGCCACCTCA |
| 151 | AGTGTAAGTTACATGCACTGGTACCAGCAGAAGCCAGGATCCTCCCCCAA |
| 201 | ACCCTGGATTTATGCCACATCCAACCTGGCTTCTGGAGTCCCTGCTCGCT |
| 251 | TCAGTGGCAGTGGGTCTGGGACCTCTTACTCTCTCACATTCAGCAGAGTG |
| 301 | GAGGCTGAAGATGCTGCCACTTATTACTGCCAGCAGTGGAGTAGTAACCC |
| 351 | ACCCACGTTCGGAGGGGGGACCAGGCTGGAAATAACACGG |
| 1-60 | Signal peptide |
| 61-390 | Light chain variable region |
| Seq. 254. I5-3023 light chain variable region, | |
| amino acid sequence, ID: 500812p | |
| | .........o.........o.........o.........o.........o |
| 1 | METDTLLLWVLLLWVPGSTGDTRQIVLSQSPAILSASPGEKVTMTCRATS |
| 51 | SVSYMHWYQQKPGSSPKPWIYATSNLASGVPARFSGSGSGTSYSLTFSRV |
| 101 | EAEDAATYYCQQWSSNPPTFGGGTRLEITR |
| 1-20 | Signal peptide |
| 21-130 | Light chain variable region |
| Seq. 255. I5-3023 heavy chain variable region, | |
| nucleotide sequence, ID: 500813n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACacgcgtGAGGTGCAACTTGTTGAGTCTGGTGGAGGAT |
| 101 | TGGTGCAGCCTAAAGGGTCATTGAAACTCTCATGTGCAGCCTCTGGATTC |
| 151 | ACCTTCAATATCTACGCCATGAACTGGGTCCGCCAGGCTCCAGGAAAGGG |
| 201 | TTTGGAATGGGTTGCTCGCATAAGATCTAAAAGTAATAATTTTGCAACAT |
| 251 | ATTATGGCGATTCAGTGAGAGACAGGTTCACCATCTCCAGAGATGATTCA |
| 301 | CAGAGCATGCTCTATCTGCAAATGAACAACTTGAAAGCTGAAGACACAGC |
| 351 | CATGTATTACTGTGTGAGACGGGGGGGTGGTAGCCATTACTATGCTATGG |
| 401 | ACTACTGGGGTCAAGGAACCTCAGTCACCGTCTCCTCA |
| 1-60 | Signal peptide |
| 61-438 | Heavy chain variable region |
| Seq. 256. I5-3023 heavy chain variable region, | |
| amino acid sequence, ID: 500813p | |
| | .........o.........o.........o.........o.........o |
| 1 | METDTLLLWVLLLWVPGSTGDTREVQLVESGGGLVQPKGSLKLSCAASGF |
| 51 | TFNIYAMNWVRQAPGKGLEWVARIRSKSNNFATYYGDSVRDRFTISRDDS |
| 101 | QSMLYLQMNNLKAEDTAMYYCVRRGGGSHYYAMDYWGQGTSVTVSS |
| 1-20 | Signal peptide |
| 21-146 | Heavy chain variable region |
| Seq. 257. I8-1033 light chain variable region, | |
| nucleotide sequence, ID: 500828n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACacgcgtCAAATTGTTCTCACCCAGTCTCCAGCACTCA |
| 101 | TGTCTGCATCTCCAGGGGAGAAGGTCACCATGACCTGCAGTGCCAGCTCA |
| 151 | AGTGTAAGTTACATGTACTGGTACCAGCAGAAGCCAAGATCCTCCCCCAA |
| 201 | ACCCTGGATTTATCTCACATCCAACCTGGCTTCTGGAGTCCCTGCTCGCT |
| 251 | TCAGTGGCAGTGGGTCTGGGACCTCTTACTCTCTCACAATCAGCAGCATG |
| 301 | GAGGCTGAAGATGCTGCCACTTATTACTGCCAGCAGTGGAGTAGTAACCC |
| 351 | ACCCACGTTCGGTGCTGGGACCAAGCTGGAGCTGAAACGG |
| 1-60 | Signal peptide |
| 61-390 | Light chain variable region |
| Seq. 258. I8-1033 light chain variable region, | |
| amino acid sequence, ID: 500828p | |
| | .........o.........o.........o.........o.........o |
| 1 | METDTLLLWVLLLWVPGSTGDTRQIVLTQSPALMSASPGEKVTMTCSASS |
| 51 | SVSYMYWYQQKPRSSPKPWIYLTSNLASGVPARFSGSGSGTSYSLTISSM |
| 101 | EAEDAATYYCQQWSSNPPTFGAGTKLELKR |
| 1-20 | Signal peptide |
| 21-130 | Light chain variable region |
| Seq. 259. I8-1033 heavy chain variable region, | |
| nucleotide sequence, ID: 500829n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACacgcgtCAGGTCCAGCTGCAGCAGTCTGGACCTGAGC |
| 101 | TGGTGAAGCCTGGGGCTTCAGTGAGGATATCCTGCAAGGCTTCTGGCTAC |
| 151 | ACCTTCACAAGCTACTATATACACTGGGTGAAGCAGAGGCCTGGACAGGG |
| 201 | ACTTGAGTGGATTGGATGGATTTATCCTGGAAATGTTAATACTAAGTACA |
| 251 | ATGAGAAGTTCAAGGGCAAGGCCACACTGACTGCAGACAAATCCTCCAGC |
| 301 | ACAGCCTACATGCAGCTCAGCAGCCTGACCTCTGAGGACTCTGCGGTCTA |
| 351 | TTTCTGTGCAAGACGGGCGGGGGGCTACTGGTACTTCGATGTCTGGGGCG |
| 401 | CAGGGACCACGGTCACCGTCTCCTCA |
| 1-60 | Signal peptide |
| 61-426 | Heavy chain variable region |
| Seq. 260. I8-1033 heavy chain variable region, | |
| amino acid sequence, ID: 500829p | |
| | .........o.........o.........o.........o.........o |
| 1 | METDTLLLWVLLLWVPGSTGDTRQVQLQQSGPELVKPGASVRISCKASGY |
| 51 | TFTSYYIHWVKQRPGQGLEWIGWIYPGNVNTKYNEKFKGKATLTADKSSS |
| 101 | TAYMQLSSLTSEDSAVYFCARRAGGYWYFDVWGAGTTVTVSS |
| 1-20 | Signal peptide |
| 21-142 | Heavy chain variable region |
| Seq. 261. I8-1030 light chain variable region, | |
| nucleotide sequence, ID: 500821n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACacgcgtCAAATTGTTCTCACCCAGTCTCCAGCAATCA |
| 101 | TGTCTGCATCTCTAGGGGAACGGGTCACCATGACCTGCACTGCCAGCTCA |
| 151 | AGTGTAAGTTCCAGTTACTTGCACTGGTACCAGCAGAAGCCAGGATCCTC |
| 201 | CCCCAAACTCTGGATTTATAGCACATCCAACCTGGCTTCTGGAGTCCCAG |
| 251 | CTCGCTTCAGTGGCAGTGGGTCTGGGACCTCTTACTCTCTCACAATCAGC |
| 301 | AGCATGGAGGCTGAAGATGCTGCCACTTATTACTGCCACCAGTATCATCG |
| 351 | TTCCCCATTCACGTTCGGCTCGGGGACAAAGTTGGAAATAAAACGG |
| 1-60 | Signal peptide |
| 61-396 | Light chain variable region |
| Seq. 262. I8-1030 light chain variable region, | |
| amino acid sequence, ID: 500821p | |
| | .........o.........o.........o.........o.........o |
| 1 | METDTLLLWVLLLWVPGSTGDTRQIVLTQSPAIMSASLGERVTMTCTASS |
| 51 | SVSSSYLHWYQQKPGSSPKLWIYSTSNLASGVPARFSGSGSGTSYSLTIS |
| 101 | SMEAEDAATYYCHQYHRSPFTFGSGTKLEIKR |
| 1-20 | Signal peptide |
| 21-132 | Light chain variable region |
| Seq. 263. I8-1030 heavy chain variable region, | |
| nucleotide sequence, ID: 500822n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACacgcgtGAGGTTCAGCTGCAGCAGTCTGGGGCAGAGC |
| 101 | TTGTGAAGCCAGGGGCCTCAGTCAAGTTGTCCTGCACAGCTTCTGGCTTC |
| 151 | AACATTAAAGACACCTATATGCACTGGGTGAAGCAGAGGCCTGAACAGGG |
| 201 | CCTGGAGTGGATTGGAAGGATTGATCCTGCGAATGGTAATACTAAATATG |
| 251 | ACCCGAAGTTCCAGGGCAAGGCCACTATAACAGCAGACACATCCTCCAAC |
| 301 | ACAGCCTACCTGCAGCTCAGCAGCCTGACATCTGAGGACACTGCCGTCTA |
| 351 | TTACTGTGCTAGAAGGTTCGATGGTTACTTTCGCTGGTTTGCTTACTGGG |
| 401 | GCCAAGGGACTCTGGTCACTGTCTCT |
| 1-60 | Signal peptide |
| 61-426 | Heavy chain variable region |
| Seq. 264. I8-1030 heavy chain variable region, | |
| amino acid sequence, ID: 500822p | |
| | .........o.........o.........o.........o.........o |
| 1 | METDTLLLWVLLLWVPGSTGDTREVQLQQSGAELVKPGASVKLSCTASGF |
| 51 | NIKDTYMHWVKQRPEQGLEWIGRIDPANGNTKYDPKFQGKATITADTSSN |
| 101 | TAYLQLSSLTSEDTAVYYCARRFDGYFRWFAYWGQGTLVTVS |
| 1-20 | Signal peptide |
| 21-142 | Heavy chain variable region |
| Seq. 265. I10-9005 light chain variable region, | |
| nucleotide sequence, ID: 500866n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACacgcgtGACATTGTGATGTCACAGTCTCCATCCTCCC |
| 101 | TGGCTGTGTCAGCAGGAGAGAAGGTCACTATGAGCTGCAAATCCAGTCAG |
| 151 | AGTCTGCTCAACAGTAGAACCCGAAAGAACTACTTGGCTTGGTACCAGCA |
| 201 | GAAACCAGGGCAGTCTCCTAAACTGCTGATCTACTGGGCATCCACTAGGG |
| 251 | AATCTGGGGTCCCTGATCGCTTCACAGGCAGTGGATCTGGGACAGATTTC |
| 301 | ACTCTCACCATCAGCAGTGTGCAGGCTGAAGACCTGGCAGTTTATTACTG |
| 351 | CAAGCAATCTTATAATCTGTACACGTTCGGAGGGGGGACCAAGCTGGAAA |
| 401 | TAAAACGG |
| 1-60 | Signal peptide |
| 61-408 | Light chain variable region |
| Seq. 266. I10-9005 light chain variable region, | |
| amino acid sequence, ID: 500866p | |
| | .........o.........o.........o.........o.........o |
| 1 | METDTLLLWVLLLWVPGSTGDTRDIVMSQSPSSLAVSAGEKVTMSCKSSQ |
| 51 | SLLNSRTRKNYLAWYQQKPGQSPKLLIYWASTRESGVPDRFTGSGSGTDF |
| 101 | TLTISSVQAEDLAVYYCKQSYNLYTFGGGTKLEIKR |
| 1-20 | Signal peptide |
| 21-136 | Light chain variable region |
| Seq. 267. I10-9005 heavy chain variable region, | |
| nucleotide sequence, ID: 500867n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACacgcgtGAGGTTCAGCTGCAGCAGTCTGGGGCTGAGC |
| 101 | TTGTGAGGCCAGGGGCCTTAGTCAGGTTGTCCTGCAAAGCTTCTGGCTTC |
| 151 | AACATTAAAGACTACTATATGAACTGGGTGAAGCAGAGGCCTGAACAGGG |
| 201 | CCTGGAGTGGATTGGATGGATTGATCCTGAGAATGGTAATACTATATATG |
| 251 | ACCCGAAGTTCCAGGGCAAGGCCAGTATAATAGCAGACATATCCTCCAAC |
| 301 | ACAGCCTACCTACAGCTCAGCAGCCTGACATCTGAGGACACAGCCGTCTA |
| 351 | TTATTGTGCTAGATGGTACCACTATGTTATGGACTACTGGGGTCAAGGAA |
| 401 | CCTCAGTCACCGTCTCCTCA |
| 1-60 | Signal peptide |
| 61-420 | Heavy chain variable region |
| Seq. 268. I10-9005 heavy chain variable region, | |
| amino acid sequence, ID: 500867p | |
| | .........o.........o.........o.........o.........o |
| 1 | METDTLLLWVLLLWVPGSTGDTREVQLQQSGAELVRPGALVRLSCKASGF |
| 51 | NIKDYYMNWVKQRPEQGLEWIGWIDPENGNTIYDPKFQGKASIIADISSN |
| 101 | TAYLQLSSLTSEDTAVYYCARWYHYVMDYWGQGTSVTVSS |
| 1-20 | Signal peptide |
| 21-140 | Heavy chain variable region |
| Seq. 269. I10-9015 light chain variable region, | |
| nucleotide sequence, ID: 500846n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACacgcgtGATGTTGTGATGACCCAGACTCCACTCACTT |
| 101 | TGTCGGTTACCATTGGACAACCAGCCTCCATCTCTTGCAAGTCAAGTCAG |
| 151 | AGCCTCTTAGATAGTGATGGAAAGACATATTTTAGTTGGTTGTTACAGAG |
| 201 | GCCAGGCCAGTCTCCAAAGCGCCTAATCTATCTGGTGTCTAAACTGGACT |
| 251 | CTGGAGTCCCTGACAGGTTCACTGGCAGTGGATCAGGGACAGATTTCACA |
| 301 | CTTAAAATCAGCAGAGTGGAGGCTGAGGATTTGGGAGTTTATTTTTGCTG |
| 351 | GCAAGGTACACATTTTCCTCACACATTCGGAGGGGGGACCAAGCTGGAGA |
| 401 | TAAAACGG |
| 1-60 | Signal peptide |
| 61-408 | Light chain variable region |
| Seq. 270. I10-9015 light chain variable region, | |
| amino acid sequence, ID: 500846p | |
| | .........o.........o.........o.........o.........o |
| 1 | METDTLLLWVLLLWVPGSTGDTRDVVMTQTPLTLSVTIGQPASISCKSSQ |
| 51 | SLLDSDGKTYFSWLLQRPGQSPKRLIYLVSKLDSGVPDRFTGSGSGTDFT |
| 101 | LKISRVEAEDLGVYFCWQGTHFPHTFGGGTKLEIKR |
| 1-20 | Signal peptide |
| 21-136 | Light chain variable region |
| Seq. 271. I10-9015 heavy chain variable region, | |
| nucleotide sequence, ID: 500847n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACacgcgtGAAGTGCAGCTGGTGGAGTCTGGGGGAGGCT |
| 101 | TAGTGAAGCCTGGAGAGTCCCTGACACTCTCCTGTACAACCTCTGGATTC |
| 151 | ACTTTCAGTGACTATTACATGTATTGGGTTCGCCAGACTCCGGAAAAGAG |
| 201 | GCTGGAGTGGGTCGCAACCATTAATCGTGATGGTAGTTATACCTACTTTC |
| 251 | CAGACAATTTTAAGGGGCGATTCACCATCTCCAGAGACAATGCCAAGAAC |
| 301 | AACCTGTACCTGCAAATGAGCAGTCTGAAGTCTGAGGACACAGCCATGTA |
| 351 | TTACTGTTCAACCATGCTGTTTGCTTACTGGGGCCAAGGGACTCTGGTCA |
| 401 | CTGTCTCT |
| 1-60 | Signal peptide |
| 61-408 | Heavy chain variable region |
| Seq. 272. I10-9015 heavy chain variable region, | |
| amino acid sequence, ID: 500847p | |
| | .........o.........o.........o.........o.........o |
| 1 | METDTLLLWVLLLWVPGSTGDTREVQLVESGGGLVKPGESLTLSCTTSGF |
| 51 | TFSDYYMYWVRQTPEKRLEWVATINRDGSYTYFPDNFKGRFTISRDNAKN |
| 101 | NLYLQMSSLKSEDTAMYYCSTMLFAYWGQGTLVTVS |
| 1-20 | Signal peptide |
| 21-146 | Heavy chain variable region |
| Seq. 273. I5-3004 light chain variable region, | |
| nucleotide sequence, ID: 500803n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACacgcgtGATATTGTGCTAACTCAGTCTCCAGCCACCC |
| 101 | TGTCTGTGACTCCAGGAGATAGCGTCAGTCTTTCCTGCAGGGCCAGCCAA |
| 151 | AGTATTAGCAACAACCTACACTGGTATCAACAAAAATCACATGAGTCTCC |
| 201 | AAGGCTTCTCATCAAGTATGCTTCCCAGTCCATCTCTGGGATCCCCTCCA |
| 251 | GGTTCAGTGGCAGcGGATCAGGGACAGATTTCACTCTCAGTATCAACAGT |
| 301 | GTGGAGACTGAAGATTTTGGAATGTATTTCTGTCAACAGAGTAACAGCTG |
| 351 | GCCGCTCACGTTCGGTGCTGGGACCAAGCTGGAGCTGAAACGG |
| 1-60 | Signal peptide |
| 61-393 | Light chain variable region |
| Seq. 274. I5-3004 light chain variable region, | |
| amino acid sequence, ID: 500803p | |
| | .........o.........o.........o.........o.........o |
| 1 | METDTLLLWVLLLWVPGSTGDTRDIVLTQSPATLSVTPGDSVSLSCRASQ |
| 51 | SISNNLHWYQQKSHESPRLLIKYASQSISGIPSRFSGSGSGTDFTLSINS |
| 101 | VETEDFGMYFCQQSNSWPLTFGAGTKLELKR |
| 1-20 | Signal peptide |
| 21-131 | Light chain variable region |
| Seq. 275. I5-3004 heavy chain variable region, | |
| nucleotide sequence, ID: 500804n | |
| | .........o.........o.........o.........o.........o |
| 1 | ATGGAGACAGACACACTCCTGCTATGGGTACTGCTGCTCTGGGTTCCAGG |
| 51 | TTCCACTGGTGACacgcgtCAGGTGCAGCTGAAGGAGTCAGGACCTGGCC |
| 101 | TGGTGGCGCCCTCACAGAGCCTGTCCATCACATGCACTGTCTCAGGGTTC |
| 151 | TCATTAACCAGCTATGGTGTAAGCTGGGTTCGCCAGCCTCCAGGAAAGGG |
| 201 | TCTGGAGTGGCTGGGAGTAATATGGGGTGACGGGAGCACAAATTATCATT |
| 251 | CAGCTCTCATATCCAGACTGAGCATCAGCAAGGATAACTCCAAGAGCCAA |
| 301 | GTTTTCTTAAAACTGAACAGTCTGCAAACTGATGACACAGCCACGTACTA |
| 351 | CTGTGCCATATTACGACGGACGGTATCCTTTGCTTACTGGGGCCAAGGGA |
| 401 | CTCTGGTCACTGTCTCT |
| 1-60 | Signal peptide |
| 61-417 | Heavy chain variable region |
| Seq. 276. I5-3004 heavy chain variable region, | |
| amino acid sequence, ID: 500804p | |
| | .........o.........o.........o.........o.........o |
| 1 | METDTLLLWVLLLWVPGSTGDTRQVQLKESGPGLVAPSQSLSITCTVSGF |
| 51 | SLTSYGVSWVRQPPGKGLEWLGVIWGDGSTNYHSALISRLSISKDNSKSQ |
| 101 | VFLKLNSLQTDDTATYYCAILRRTVSFAYWGQGTLVTVS |
| 1-20 | Signal peptide |
| 21-139 | Heavy chain variable region |

### Example 10

### Ex Vivo Efficiency Determination

Groups of 4 mice (6-8 weeks old) are injected once intraperitoneally at a dose of 15 mg/kg. At various time points post injection, mice are terminally bled by eye enucleation and blood collected into a heparinized container. Shortly after blood collection the blood samples are spun at 5000×g for 10 min and the supernatant is collected (plasma). The concentration of product present in the plasma fraction is measured by ELISA using goat anti-human Fc antibody as capture and goat anti-human light chain as secondary antibody (horseradish peroxidase-conjugated). Based on the concentration of product detected, the plasma sample is diluted to be used in the MIC assay.

MIC assay. The procedure for testing in vitro efficacy is based on the standard MIC (minimal inhibitory concentration) assay as described in detail in the CSLI (Clinical Laboratory Standards Institute) protocols, and by Steinberg and Lehrer (Steinberg, D., and R. I. Lehrer, 1997, Designer assays for antimicrobial peptides. Methods Mol. Biol. 78:169-186) and by Turner (Turner, J., Y. Cho, N-N. Dinh, A. J. Waring, and R. I. Lehrer. 1998. Activities of LL-37, a cathelin-associated antimicrobial peptide of human neutrophils. Antimicrob. Agents Chemother. 42:2206-2214).

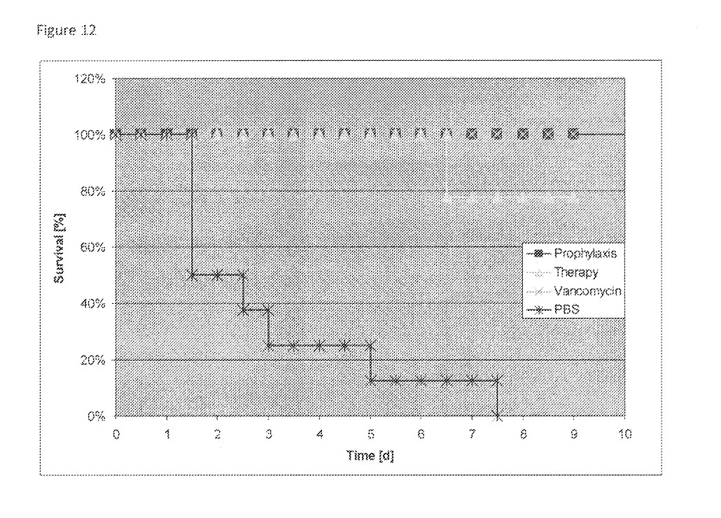

Briefly, log phase S. aureus cells are grown to an OD600 of 0.15-0.3, harvested and adjusted to the proper concentration for use in the MIC assay based on previously determined growth curves. The protein A binding sites on the Staph cells are blocked by adsorption to recombinant huFc portion produced in our laboratory. Staph target cells are then seeded into the wells of 96-well microtiter plates (chilled on ice) containing a dilution series of test substances. In-assay concentration of test substances typically range from 200 nM to 0.4 nM. The assay plate is then incubated at 37° C. for 1 h. At this point, 100 ul of 2× culture medium (TSB, tryptic soy broth) is added to each well and the plate is incubated overnight at 37° C. on a shaker. The next day, each well of the plate is inspected for the presence of bacterial growth. The lowest concentration of product in nM that shows no visible bacterial growth is considered the MIC of that particular sample. As controls plasma from PBS injected mice, PBS only and recombinant lysostaphin (Sigma, L2898) are used. Typical results are presented in FIG. 11.

### Example 11

### In Vivo Efficacy Testing of Recombinant Lysostaphin Products in Murine Bacteremia Model

Mice are kept in a BSL2 biocontainment animal room in accordance with the PHS Guide for the Care and Use of Laboratory Animals. Groups of 8 6-8 week old BALB/c mice are used for this experiment. Mice are randomly assigned to 2 microisolator cages of 4 mice for each treatment group. For testing therapeutic or prophylactic efficacy, mice are injected with 5×10⁷ cfu/ml Staphylococcus aureus Strain USA300-NRS384 (obtained from the Network on Antimicrobial Resistance in Staphylococcus aureus (NARSA) via tail vein injection. This optimal challenge dose has been determined earlier in a separate challenge dose titration trial. One hour later, mice are treated with test substances via i.p. injection at 30 mg/kg/d or less, for one or more days. If determining the prophylactic potency of a test substance, the treatment is given 4 h prior to challenge. During the course of the experiment animals are closely monitored for signs of sickness and scored for their physical appearance and behavior as follows:

| Score | Physical Appearance |
|---|---|
| 1 | Normal |
| 2 | Lack of grooming |
| 3 | Rough hair coat, nasal/ocular discharge |
| 4 | Very rough coat, abnormal posture (head tucked into abdomen) |

| Score | Behavior |
|---|---|
| 1 | Normal |
| 2 | Minor changes: limping, favoring inoculated leg |
| 3 | Abnormal: reduced mobility, inactive |
| 4 | Unsolicited vocalization, self mutilation, restless or immobile |

Moribund (requires euthanasia)

1. Lack of responsiveness to manual stimulation

2. Immobility; and/or an inability to eat or drink.

Animals considered moribund are euthanized using CO2 asphyxiation. Survival is the primary readout for this experiment and provides evidence as to how well a test substance can prevent death. Results of the survival study are typically presented using the Kaplan-Meier survival plot. See FIG. 12.

### Example 12

### Lysostaphin Products with Glycosylation Site Mutation

The glycosylation site at position 125 of the lysostaphin gene was removed by site directed mutagenesis PCR followed by cloning of the mutated gene into the existing fusion construct, N-terminal of the gene for the antibody light chain using standard molecular techniques. The finished construct was sequenced for quality control and used in our retrovector mammalian expression system to generate clonal CHO cell lines producing the glycosylation site negative lysostaphin-antibody fusion protein. Product made from these cell lines was compared to product containing wild type lysostaphin using MIC assay both directly and ex vivo as described above.

Table 8 shows MIC results comparing wild-type and glycosylation mutant products both culture-derived product and product obtained from plasma after circulation in a mouse for different time points. No significant difference in in vitro efficacy between glycosylation site 125 negative and wild type variant was observed for 4 different products tested.

TABLE 8
MIC using USA300 S. aureus target cells

| | | MIC |
|---|---|---|
| Product | Variant | [nM] |
| LYST(1-246′)-I8-1017-HBD3 | glycosylation site 125 removed | 12.5 |
| LYST(1-246)-I8-1017-HBD3 | wild type | 3.55 |
| LYST(1-246′)-I8-1017-PLA2 | glycosylation site 125 removed | 4.7 |
| LYST(1-246)-I8-1017-PLA2 | wild type | 3.9 |
| LYST(1-246′)-I9-7002-HBD3 | glycosylation site 125 removed | 6.3 |
| LYST(1-246)-I9-7002-HBD3 | wild type | 4.7 |
| LYST(1-246′)-I9-7002-PLA2 | glycosylation site 125 removed | 1.6 |
| LYST(1-246′-I9-7002-PLA2 | wild type | 3.125 |

### Example 13

### Lysostaphin Constructs

The following example provides fusion protein constructs comprising wild-type and mutant lysostaphin fused to an immunoglobulin.

| SEQ ID NO: 277. LYST-LC-I5-3023, Lysostaphin-light chain | |
|---|---|
| chimeric murine-human fusion, nucleotide sequence, | |
| ID: 500820n | |
| ATGGAGACAGACACACTCCTGCTATGGGTACTGCTGCTCTGGGTTCCAGGTTCCACT | |
| GGTGACGCCACCCACGAGCACTCCGCCCAGTGGCTGAACAACTACAAGAAGGGCTAC | |
| GGCTACGGCCCCTACCCCCTGGGCATCAACGGCGGCATGCACTACGGCGTGGACTTC | |
| TTCATGAACATCGGCACCCCCGTGAAGGCCATCTCCTCCGGCAAGATCGTGGAGGCC | |
| GGCTGGTCCAACTACGGCGGCGGCAACCAGATCGGCCTGATCGAGAACGACGGCGTG | |
| CACCGCCAGTGGTACATGCACCTGTCCAAGTACAACGTGAAGGTGGGCGACTACGTG | |
| AAGGCCGGCCAGATCATCGGCTGGTCCGGCTCCACCGGCTACTCCACCGCCCCCCAC | |
| CTGCACTTCCAGCGCATGGTGAACTCCTTCTCCAACTCCACCGCCCAGGACCCCATG | |
| CCCTTCCTGAAGTCCGCCGGCTACGGCAAGGCCGGCGGCACCGTGACCCCCACCCCC | |
| AACACCGGCTGGAAGACCAACAAGTACGGCACCCTGTACAAGTCCGAGTCCGCCTCC | |
| TTCACCCCCAACACCGACATCATCACCCGCACCACCGGCCCCTTCCGCTCCATGCCC | |
| CAGTCCGGCGTGCTGAAGGCCGGCCAGACCATCCACTACGACGAGGTGATGAAGCAG | |
| GACGGCCACGTGTGGGTGGGCTACACCGGCAACTCCGGCCAGCGCATCTACCTGCCC | |
| GTGCGCACCTGGAACAAGTCCACCAACACCCTGGGCGTGCTGTGGGGCACCATCAAG | |
| GGTGGTGGCGGTTCAGGCGGAGGTGGCTCTGGCGGTGGCGGATCcacgcgtCAAATT | |
| GTTCTCTCCCAGTCTCCAGCAATCCTGTCTGCATCTCCAGGGGAGAAGGTCACAATG | |
| ACTTGCAGGGCCACCTCAAGTGTAAGTTACATGCACTGGTACCAGCAGAAGCCAGGA | |
| TCCTCCCCCAAACCCTGGATTTATGCCACATCCAACCTGGCTTCTGGAGTCCCTGCT | |
| CGCTTCAGTGGCAGTGGGTCTGGGACCTCTTACTCTCTCACATTCAGCAGAGTGGAG | |
| GCTGAAGATGCTGCCACTTATTACTGCCAGCAGTGGAGTAGTAACCCACCCACGTTC | |
| GGAGGGGGGACCAGGCTGGAAATAACACGGACTGTGGCTGCACCATCTGTCTTCATC | |
| TTCCCGCCATCTGATGAGCAGTTGAAATCTGGAACTGCCTCTGTTGTGTGCCTGCTG | |
| AATAACTTCTATCCCAGAGAGGCCAAAGTACAGTGGAAGGTGGATAACGCCCTCCAA | |
| TCGGGTAACTCCCAGGAGAGTGTCACAGAGCAGGACAGCAAGGACAGCACCTACAGC | |
| CTCAGCAGCACCCTGACGCTGAGCAAAGCAGACTACGAGAAACACAAAGTCTACGCC | |
| TGCGAAGTCACCCATCAGGGCCTGAGCTCGCCCGTCACAAAGAGCTTCAACAGGGGA | |
| GAGTGTTAG | |
| 1-60 | Signal peptide |
| 61-1491 | Chimeric murine-human Light chain |
| SEQ ID NO: 278. LYST-LC-I5-3023, Lysostaphin-light chain | |
| chimeric murine-human fusion, amino acid sequence, | |
| ID: 500820p | |
| METDTLLLWVLLLWVPGSTGDATHEHSAQWLNNYKKGYGYGPYPLGINGGMHYGVDF | |
| FMNIGTPVKAISSGKIVEAGWSNYGGGNQIGLIENDGVHRQWYMHLSKYNVKVGDYV | |
| KAGQIIGWSGSTGYSTAPHLHFQRMVNSFSNSTAQDPMPFLKSAGYGKAGGTVTPTP | |
| NTGWKTNKYGTLYKSESASFTPNTDIITRTTGPFRSMPQSGVLKAGQTIHYDEVMKQ | |
| DGHVWVGYTGNSGQRIYLPVRTWNKSTNTLGVLWGTIKGGGGSGGGGSGGGGSTRQI | |
| VLSQSPAILSASPGEKVTMTCRATSSVSYMHWYQQKPGSSPKPWIYATSNLASGVPA | |
| RFSGSGSGTSYSLTFSRVEAEDAATYYCQQWSSNPPTFGGGTRLEITRTVAAPSVFI | |
| FPPSDEQLKSGTASVVCLLNNFYPREAKVQWKVDNALQSGNSQESVTEQDSKDSTYS | |
| LSSTLTLSKADYEKHKVYACEVTHQGLSSPVTKSFNRGEC | |
| 1-20 | Signal peptide |
| 21-266 | Lysostaphin |
| 267-496 | Linker-LC |
| SEQ ID NO: 279. LYST(N125Q)-LC-I5-3023, Lysostaphin-light | |
| chain chimeric murine-human fusion, nucleotide sequence, | |
| ID: 500893n | |
| ATGGAGACAGACACACTCCTGCTATGGGTACTGCTGCTCTGGGTTCCAGGTTCCACT | |
| GGTGACGCCACCCACGAGCACTCCGCCCAGTGGCTGAACAACTACAAGAAGGGCTAC | |
| GGCTACGGCCCCTACCCCCTGGGCATCAACGGCGGCATGCACTACGGCGTGGACTTC | |
| TTCATGAACATCGGCACCCCCGTGAAGGCCATCTCCTCCGGCAAGATCGTGGAGGCC | |
| GGCTGGTCCAACTACGGCGGCGGCAACCAGATCGGCCTGATCGAGAACGACGGCGTG | |
| CACCGCCAGTGGTACATGCACCTGTCCAAGTACAACGTGAAGGTGGGCGACTACGTG | |
| AAGGCCGGCCAGATCATCGGCTGGTCCGGCTCCACCGGCTACTCCACCGCCCCCCAC | |
| CTGCACTTCCAGCGCATGGTGAACTCCTTCTCCcagTCCACCGCCCAGGACCCCATG | |
| CCCTTCCTGAAGTCCGCCGGCTACGGCAAGGCCGGCGGCACCGTGACCCCCACCCCC | |
| AACACCGGCTGGAAGACCAACAAGTACGGCACCCTGTACAAGTCCGAGTCCGCCTCC | |
| TTCACCCCCAACACCGACATCATCACCCGCACCACCGGCCCCTTCCGCTCCATGCCC | |
| CAGTCCGGCGTGCTGAAGGCCGGCCAGACCATCCACTACGACGAGGTGATGAAGCAG | |
| GACGGCCACGTGTGGGTGGGCTACACCGGCAACTCCGGCCAGCGCATCTACCTGCCC | |
| GTGCGCACCTGGAACAAGTCCACCAACACCCTGGGCGTGCTGTGGGGCACCATCAAG | |
| GGTGGTGGCGGTTCAGGCGGAGGTGGCTCTGGCGGTGGCGGATCcacgcgtCAAATT | |
| GTTCTCTCCCAGTCTCCAGCAATCCTGTCTGCATCTCCAGGGGAGAAGGTCACAATG | |
| ACTTGCAGGGCCACCTCAAGTGTAAGTTACATGCACTGGTACCAGCAGAAGCCAGGA | |
| TCCTCCCCCAAACCCTGGATTTATGCCACATCCAACCTGGCTTCTGGAGTCCCTGCT | |
| CGCTTCAGTGGCAGTGGGTCTGGGACCTCTTACTCTCTCACATTCAGCAGAGTGGAG | |
| GCTGAAGATGCTGCCACTTATTACTGCCAGCAGTGGAGTAGTAACCCACCCACGTTC | |
| GGAGGGGGGACCAGGCTGGAAATAACACGGACTGTGGCTGCACCATCTGTCTTCATC | |
| TTCCCGCCATCTGATGAGCAGTTGAAATCTGGAACTGCCTCTGTTGTGTGCCTGCTG | |
| AATAACTTCTATCCCAGAGAGGCCAAAGTACAGTGGAAGGTGGATAACGCCCTCCAA | |
| TCGGGTAACTCCCAGGAGAGTGTCACAGAGCAGGACAGCAAGGACAGCACCTACAGC | |
| CTCAGCAGCACCCTGACGCTGAGCAAAGCAGACTACGAGAAACACAAAGTCTACGCC | |
| TGCGAAGTCACCCATCAGGGCCTGAGCTCGCCCGTCACAAAGAGCTTCAACAGGGGA | |
| GAGTGTTAG | |
| 1-60 | Signal peptide |
| 61-1491 | Chimeric murine-human Light chain |
| SEQ ID NO: 280. LYST(N125Q)-LC-I5-3023, Lysostaphin-light | |
| chain chimeric murine-human fusion, amino acid sequence, | |
| ID: 500893p | |
| METDTLLLWVLLLWVPGSTGDATHEHSAQWLNNYKKGYGYGPYPLGINGGMHYGVDF | |
| FMNIGTPVKAISSGKIVEAGWSNYGGGNQIGLIENDGVHRQWYMHLSKYNVKVGDYV | |
| KAGQIIGWSGSTGYSTAPHLHFQRMVNSFSQSTAQDPMPFLKSAGYGKAGGTVTPTP | |
| NTGWKTNKYGTLYKSESASFTPNTDIITRTTGPFRSMPQSGVLKAGQTIHYDEVMKQ | |
| DGHVWVGYTGNSGQRIYLPVRTWNKSTNTLGVLWGTIKGGGGSGGGGSGGGGSTRQI | |
| VLSQSPAILSASPGEKVTMTCRATSSVSYMHWYQQKPGSSPKPWIYATSNLASGVPA | |
| RFSGSGSGTSYSLTFSRVEAEDAATYYCQQWSSNPPTFGGGTRLEITRTVAAPSVFI | |
| FPPSDEQLKSGTASVVCLLNNFYPREAKVQWKVDNALQSGNSQESVTEQDSKDSTYS | |
| LSSTLTLSKADYEKHKVYACEVTHQGLSSPVTKSFNRGEC | |
| 1-20 | Signal peptide |
| 21-266 | Lysostaphin |
| 267-496 | Linker-LC |
| SEQ ID NO: 281. LYST(N125Q,N232Q)-LC-I5-3023, Lysostaphin- | |
| light chain chimeric murine-human fusion, nucleotide | |
| sequence, ID: 500926n | |
| ATGGAGACAGACACACTCCTGCTATGGGTACTGCTGCTCTGGGTTCCAGGTTCCACT | |
| GGTGACGCCACCCACGAGCACTCCGCCCAGTGGCTGAACAACTACAAGAAGGGCTAC | |
| GGCTACGGCCCCTACCCCCTGGGCATCAACGGCGGCATGCACTACGGCGTGGACTTC | |
| TTCATGAACATCGGCACCCCCGTGAAGGCCATCTCCTCCGGCAAGATCGTGGAGGCC | |
| GGCTGGTCCAACTACGGCGGCGGCAACCAGATCGGCCTGATCGAGAACGACGGCGTG | |
| CACCGCCAGTGGTACATGCACCTGTCCAAGTACAACGTGAAGGTGGGCGACTACGTG | |
| AAGGCCGGCCAGATCATCGGCTGGTCCGGCTCCACCGGCTACTCCACCGCCCCCCAC | |
| CTGCACTTCCAGCGCATGGTGAACTCCTTCTCCcagTCCACCGCCCAGGACCCCATG | |
| CCCTTCCTGAAGTCCGCCGGCTACGGCAAGGCCGGCGGCACCGTGACCCCCACCCCC | |
| AACACCGGCTGGAAGACCAACAAGTACGGCACCCTGTACAAGTCCGAGTCCGCCTCC | |
| TTCACCCCCAACACCGACATCATCACCCGCACCACCGGCCCCTTCCGCTCCATGCCC | |
| CAGTCCGGCGTGCTGAAGGCCGGCCAGACCATCCACTACGACGAGGTGATGAAGCAG | |
| GACGGCCACGTGTGGGTGGGCTACACCGGCAACTCCGGCCAGCGCATCTACCTGCCC | |
| GTGCGCACCTGGcagAAGTCCACCAACACCCTGGGCGTGCTGTGGGGCACCATCAAG | |
| GGTGGTGGCGGTTCAGGCGGAGGTGGCTCTGGCGGTGGCGGATCcacgcgtCAAATT | |
| GTTCTCTCCCAGTCTCCAGCAATCCTGTCTGCATCTCCAGGGGAGAAGGTCACAATG | |
| ACTTGCAGGGCCACCTCAAGTGTAAGTTACATGCACTGGTACCAGCAGAAGCCAGGA | |
| TCCTCCCCCAAACCCTGGATTTATGCCACATCCAACCTGGCTTCTGGAGTCCCTGCT | |
| CGCTTCAGTGGCAGTGGGTCTGGGACCTCTTACTCTCTCACATTCAGCAGAGTGGAG | |
| GCTGAAGATGCTGCCACTTATTACTGCCAGCAGTGGAGTAGTAACCCACCCACGTTC | |
| GGAGGGGGGACCAGGCTGGAAATAACACGGACTGTGGCTGCACCATCTGTCTTCATC | |
| TTCCCGCCATCTGATGAGCAGTTGAAATCTGGAACTGCCTCTGTTGTGTGCCTGCTG | |
| AATAACTTCTATCCCAGAGAGGCCAAAGTACAGTGGAAGGTGGATAACGCCCTCCAA | |
| TCGGGTAACTCCCAGGAGAGTGTCACAGAGCAGGACAGCAAGGACAGCACCTACAGC | |
| CTCAGCAGCACCCTGACGCTGAGCAAAGCAGACTACGAGAAACACAAAGTCTACGCC | |
| TGCGAAGTCACCCATCAGGGCCTGAGCTCGCCCGTCACAAAGAGCTTCAACAGGGGA | |
| GAGTGTTAG | |
| 1-60 | Signal peptide |
| 61-1491 | Chimeric murine-human Light chain |
| SEQ ID NO: 282. LYST(N125Q,N232Q)-LC-I5-3023, Lysostaphin- | |
| light chain chimeric murine-human fusion, amino acid | |
| sequence, ID: 500926p | |
| METDTLLLWVLLLWVPGSTGDATHEHSAQWLNNYKKGYGYGPYPLGINGGMHYGVDF | |
| FMNIGTPVKAISSGKIVEAGWSNYGGGNQIGLIENDGVHRQWYMHLSKYNVKVGDYV | |
| KAGQIIGWSGSTGYSTAPHLHFQRMVNSFSQSTAQDPMPFLKSAGYGKAGGTVTPTP | |
| NTGWKTNKYGTLYKSESASFTPNTDIITRTTGPFRSMPQSGVLKAGQTIHYDEVMKQ | |
| DGHVWVGYTGNSGQRIYLPVRTWQKSTNTLGVLWGTIKGGGGSGGGGSGGGGSTRQI | |
| VLSQSPAILSASPGEKVTMTCRATSSVSYMHWYQQKPGSSPKPWIYATSNLASGVPA | |
| RFSGSGSGTSYSLTFSRVEAEDAATYYCQQWSSNPPTFGGGTRLEITRTVAAPSVFI | |
| FPPSDEQLKSGTASVVCLLNNFYPREAKVQWKVDNALQSGNSQESVTEQDSKDSTYS | |
| LSSTLTLSKADYEKHKVYACEVTHQGLSSPVTKSFNRGEC | |
| 1-20 | Signal peptide |
| 21-266 | Lysostaphin |
| 267-496 | Linker-LC |
| SEQ ID NO: 283. LYST-LC-I9-7002, Lysostaphin-light chain | |
| chimeric murine-human fusion, nucleotide sequence, | |
| ID: 500752n | |
| ATGGAGACAGACACACTCCTGCTATGGGTACTGCTGCTCTGGGTTCCAGGTTCCACT | |
| GGTGACGCCACCCACGAGCACTCCGCCCAGTGGCTGAACAACTACAAGAAGGGCTAC | |
| GGCTACGGCCCCTACCCCCTGGGCATCAACGGCGGCATGCACTACGGCGTGGACTTC | |
| TTCATGAACATCGGCACCCCCGTGAAGGCCATCTCCTCCGGCAAGATCGTGGAGGCC | |
| GGCTGGTCCAACTACGGCGGCGGCAACCAGATCGGCCTGATCGAGAACGACGGCGTG | |
| CACCGCCAGTGGTACATGCACCTGTCCAAGTACAACGTGAAGGTGGGCGACTACGTG | |
| AAGGCCGGCCAGATCATCGGCTGGTCCGGCTCCACCGGCTACTCCACCGCCCCCCAC | |
| CTGCACTTCCAGCGCATGGTGAACTCCTTCTCCAACTCCACCGCCCAGGACCCCATG | |
| CCCTTCCTGAAGTCCGCCGGCTACGGCAAGGCCGGCGGCACCGTGACCCCCACCCCC | |
| AACACCGGCTGGAAGACCAACAAGTACGGCACCCTGTACAAGTCCGAGTCCGCCTCC | |
| TTCACCCCCAACACCGACATCATCACCCGCACCACCGGCCCCTTCCGCTCCATGCCC | |
| CAGTCCGGCGTGCTGAAGGCCGGCCAGACCATCCACTACGACGAGGTGATGAAGCAG | |
| GACGGCCACGTGTGGGTGGGCTACACCGGCAACTCCGGCCAGCGCATCTACCTGCCC | |
| GTGCGCACCTGGAACAAGTCCACCAACACCCTGGGCGTGCTGTGGGGCACCATCAAG | |
| GGTGGTGGCGGTTCAGGCGGAGGTGGCTCTGGCGGTGGCGGATCcacgcgtAGTATT | |
| GTGATGACCCAGACTCCCAAATTCCTGCTTGTATCAGCAGGAGACAGGGTTACCATA | |
| ACCTGCAAGGCCAGTCAGAGTGTGAGTAATGATGTAGCTTGGTACCAACAGAAGCCA | |
| GGGCAGTCTCCTAAACTGCTGATATACTATGCATCCAATCGCTACACTGGAGTCCCT | |
| GATCGCTTCACTGGCAGTGGATATGGGACGGATTTCACTTTCACCATCAGCACTGTG | |
| CAGGCTGAAGACCTGGCAGTTTATTTCTGTCAGCAGGATTATAGCTCTCCTCTCACG | |
| TTCGGCTCGGGGACAAAGTTGGAAATAAAACGGACTGTGGCTGCACCATCTGTCTTC | |
| ATCTTCCCGCCATCTGATGAGCAGTTGAAATCTGGAACTGCCTCTGTTGTGTGCCTG | |
| CTGAATAACTTCTATCCCAGAGAGGCCAAAGTACAGTGGAAGGTGGATAACGCCCTC | |
| CAATCGGGTAACTCCCAGGAGAGTGTCACAGAGCAGGACAGCAAGGACAGCACCTAC | |
| AGCCTCAGCAGCACCCTGACGCTGAGCAAAGCAGACTACGAGAAACACAAAGTCTAC | |
| GCCTGCGAAGTCACCCATCAGGGCCTGAGCTCGCCCGTCACAAAGAGCTTCAACAGG | |
| GGAGAGTGTtag | |
| 1-60 | Signal peptide |
| 61-1494 | Chimeric murine-human Light chain |
| SEQ ID NO: 284. LYST-LC-I9-7002, Lysostaphin-light chain | |
| chimeric murine-human fusion, amino acid sequence, | |
| ID: 500752p | |
| METDTLLLWVLLLWVPGSTGDATHEHSAQWLNNYKKGYGYGPYPLGINGGMHYGVDF | |
| FMNIGTPVKAISSGKIVEAGWSNYGGGNQIGLIENDGVHRQWYMHLSKYNVKVGDYV | |
| KAGQIIGWSGSTGYSTAPHLHFQRMVNSFSNSTAQDPMPFLKSAGYGKAGGTVTPTP | |
| NTGWKTNKYGTLYKSESASFTPNTDIITRTTGPFRSMPQSGVLKAGQTIHYDEVMKQ | |
| DGHVWVGYTGNSGQRIYLPVRTWNKSTNTLGVLWGTIKGGGGSGGGGSGGGGSTRSI | |
| VMTQTPKFLLVSAGDRVTITCKASQSVSNDVAWYQQKPGQSPKLLIYYASNRYTGVP | |
| DRFTGSGYGTDFTFTISTVQAEDLAVYFCQQDYSSPLTFGSGTKLEIKRTVAAPSVF | |
| IFPPSDEQLKSGTASVVCLLNNFYPREAKVQWKVDNALQSGNSQESVTEQDSKDSTY | |
| SLSSTLTLSKADYEKHKVYACEVTHQGLSSPVTKSFNRGEC | |
| 1-20 | Signal peptide |
| 21-266 | Lysostaphin |
| 267-497 | Linker-LC |
| SEQ ID NO: 285. LYST(N125Q)-LC-I9-7002, Lysostaphin-light | |
| chain chimeric murine-human fusion, nucleotide sequence, | |
| ID: 500895n | |
| ATGGAGACAGACACACTCCTGCTATGGGTACTGCTGCTCTGGGTTCCAGGTTCCACT | |
| GGTGACGCCACCCACGAGCACTCCGCCCAGTGGCTGAACAACTACAAGAAGGGCTAC | |
| GGCTACGGCCCCTACCCCCTGGGCATCAACGGCGGCATGCACTACGGCGTGGACTTC | |
| TTCATGAACATCGGCACCCCCGTGAAGGCCATCTCCTCCGGCAAGATCGTGGAGGCC | |
| GGCTGGTCCAACTACGGCGGCGGCAACCAGATCGGCCTGATCGAGAACGACGGCGTG | |
| CACCGCCAGTGGTACATGCACCTGTCCAAGTACAACGTGAAGGTGGGCGACTACGTG | |
| AAGGCCGGCCAGATCATCGGCTGGTCCGGCTCCACCGGCTACTCCACCGCCCCCCAC | |
| CTGCACTTCCAGCGCATGGTGAACTCCTTCTCCcagTCCACCGCCCAGGACCCCATG | |
| CCCTTCCTGAAGTCCGCCGGCTACGGCAAGGCCGGCGGCACCGTGACCCCCACCCCC | |
| AACACCGGCTGGAAGACCAACAAGTACGGCACCCTGTACAAGTCCGAGTCCGCCTCC | |
| TTCACCCCCAACACCGACATCATCACCCGCACCACCGGCCCCTTCCGCTCCATGCCC | |
| CAGTCCGGCGTGCTGAAGGCCGGCCAGACCATCCACTACGACGAGGTGATGAAGCAG | |
| GACGGCCACGTGTGGGTGGGCTACACCGGCAACTCCGGCCAGCGCATCTACCTGCCC | |
| GTGCGCACCTGGAACAAGTCCACCAACACCCTGGGCGTGCTGTGGGGCACCATCAAG | |
| GGTGGTGGCGGTTCAGGCGGAGGTGGCTCTGGCGGTGGCGGATCcacgcgtAGTATT | |
| GTGATGACCCAGACTCCCAAATTCCTGCTTGTATCAGCAGGAGACAGGGTTACCATA | |
| ACCTGCAAGGCCAGTCAGAGTGTGAGTAATGATGTAGCTTGGTACCAACAGAAGCCA | |
| GGGCAGTCTCCTAAACTGCTGATATACTATGCATCCAATCGCTACACTGGAGTCCCT | |
| GATCGCTTCACTGGCAGTGGATATGGGACGGATTTCACTTTCACCATCAGCACTGTG | |
| CAGGCTGAAGACCTGGCAGTTTATTTCTGTCAGCAGGATTATAGCTCTCCTCTCACG | |
| TTCGGCTCGGGGACAAAGTTGGAAATAAAACGGACTGTGGCTGCACCATCTGTCTTC | |
| ATCTTCCCGCCATCTGATGAGCAGTTGAAATCTGGAACTGCCTCTGTTGTGTGCCTG | |
| CTGAATAACTTCTATCCCAGAGAGGCCAAAGTACAGTGGAAGGTGGATAACGCCCTC | |
| CAATCGGGTAACTCCCAGGAGAGTGTCACAGAGCAGGACAGCAAGGACAGCACCTAC | |
| AGCCTCAGCAGCACCCTGACGCTGAGCAAAGCAGACTACGAGAAACACAAAGTCTAC | |
| GCCTGCGAAGTCACCCATCAGGGCCTGAGCTCGCCCGTCACAAAGAGCTTCAACAGG | |
| GGAGAGTGTtag | |
| 1-60 | Signal peptide |
| 61-1494 | Chimeric murine-human Light chain |
| SEQ ID NO: 286. LYST(N125Q)-LC-I9-7002, Lysostaphin-light | |
| chain chimeric murine-human fusion, amino acid sequence, | |
| ID: 500895p | |
| METDTLLLWVLLLWVPGSTGDATHEHSAQWLNNYKKGYGYGPYPLGINGGMHYGVDF | |
| FMNIGTPVKAISSGKIVEAGWSNYGGGNQIGLIENDGVHRQWYMHLSKYNVKVGDYV | |
| KAGQIIGWSGSTGYSTAPHLHFQRMVNSFSQSTAQDPMPFLKSAGYGKAGGTVTPTP | |
| NTGWKTNKYGTLYKSESASFTPNTDIITRTTGPFRSMPQSGVLKAGQTIHYDEVMKQ | |
| DGHVWVGYTGNSGQRIYLPVRTWNKSTNTLGVLWGTIKGGGGSGGGGSGGGGSTRSI | |
| VMTQTPKFLLVSAGDRVTITCKASQSVSNDVAWYQQKPGQSPKLLIYYASNRYTGVP | |
| DRFTGSGYGTDFTFTISTVQAEDLAVYFCQQDYSSPLTFGSGTKLEIKRTVAAPSVF | |
| IFPPSDEQLKSGTASVVCLLNNFYPREAKVQWKVDNALQSGNSQESVTEQDSKDSTY | |
| SLSSTLTLSKADYEKHKVYACEVTHQGLSSPVTKSFNRGEC | |
| 1-20 | Signal peptide |
| 21-266 | Lysostaphin |
| 267-497 | Linker-LC |
| SEQ ID NO: 287. LYST(N125Q, N232Q)-LC-I9-7002, Lysostaphin- | |
| light chain chimeric murine-human fusion, nucleotide | |
| sequence, ID: 500927n | |
| ATGGAGACAGACACACTCCTGCTATGGGTACTGCTGCTCTGGGTTCCAGGTTCCACT | |
| GGTGACGCCACCCACGAGCACTCCGCCCAGTGGCTGAACAACTACAAGAAGGGCTAC | |
| GGCTACGGCCCCTACCCCCTGGGCATCAACGGCGGCATGCACTACGGCGTGGACTTC | |
| TTCATGAACATCGGCACCCCCGTGAAGGCCATCTCCTCCGGCAAGATCGTGGAGGCC | |
| GGCTGGTCCAACTACGGCGGCGGCAACCAGATCGGCCTGATCGAGAACGACGGCGTG | |
| CACCGCCAGTGGTACATGCACCTGTCCAAGTACAACGTGAAGGTGGGCGACTACGTG | |
| AAGGCCGGCCAGATCATCGGCTGGTCCGGCTCCACCGGCTACTCCACCGCCCCCCAC | |
| CTGCACTTCCAGCGCATGGTGAACTCCTTCTCCcagTCCACCGCCCAGGACCCCATG | |
| CCCTTCCTGAAGTCCGCCGGCTACGGCAAGGCCGGCGGCACCGTGACCCCCACCCCC | |
| AACACCGGCTGGAAGACCAACAAGTACGGCACCCTGTACAAGTCCGAGTCCGCCTCC | |
| TTCACCCCCAACACCGACATCATCACCCGCACCACCGGCCCCTTCCGCTCCATGCCC | |
| CAGTCCGGCGTGCTGAAGGCCGGCCAGACCATCCACTACGACGAGGTGATGAAGCAG | |
| GACGGCCACGTGTGGGTGGGCTACACCGGCAACTCCGGCCAGCGCATCTACCTGCCC | |
| GTGCGCACCTGGcagAAGTCCACCAACACCCTGGGCGTGCTGTGGGGCACCATCAAG | |
| GGTGGTGGCGGTTCAGGCGGAGGTGGCTCTGGCGGTGGCGGATCcacgcgtAGTATT | |
| GTGATGACCCAGACTCCCAAATTCCTGCTTGTATCAGCAGGAGACAGGGTTACCATA | |
| ACCTGCAAGGCCAGTCAGAGTGTGAGTAATGATGTAGCTTGGTACCAACAGAAGCCA | |
| GGGCAGTCTCCTAAACTGCTGATATACTATGCATCCAATCGCTACACTGGAGTCCCT | |
| GATCGCTTCACTGGCAGTGGATATGGGACGGATTTCACTTTCACCATCAGCACTGTG | |
| CAGGCTGAAGACCTGGCAGTTTATTTCTGTCAGCAGGATTATAGCTCTCCTCTCACG | |
| TTCGGCTCGGGGACAAAGTTGGAAATAAAACGGACTGTGGCTGCACCATCTGTCTTC | |
| ATCTTCCCGCCATCTGATGAGCAGTTGAAATCTGGAACTGCCTCTGTTGTGTGCCTG | |
| CTGAATAACTTCTATCCCAGAGAGGCCAAAGTACAGTGGAAGGTGGATAACGCCCTC | |
| CAATCGGGTAACTCCCAGGAGAGTGTCACAGAGCAGGACAGCAAGGACAGCACCTAC | |
| AGCCTCAGCAGCACCCTGACGCTGAGCAAAGCAGACTACGAGAAACACAAAGTCTAC | |
| GCCTGCGAAGTCACCCATCAGGGCCTGAGCTCGCCCGTCACAAAGAGCTTCAACAGG | |
| GGAGAGTGTtag | |
| 1-60 | Signal peptide |
| 61-1494 | Chimeric murine-human Light chain |
| SEQ ID NO: 288. LYST(N125Q, N232Q)-LC-I9-7002, Lysostaphin- | |
| light chain chimeric murine-human fusion, amino acid | |
| sequence, ID: 500927p | |
| METDTLLLWVLLLWVPGSTGDATHEHSAQWLNNYKKGYGYGPYPLGINGGMHYGVDF | |
| FMNIGTPVKAISSGKIVEAGWSNYGGGNQIGLIENDGVHRQWYMHLSKYNVKVGDYV | |
| KAGQIIGWSGSTGYSTAPHLHFQRMVNSFSQSTAQDPMPFLKSAGYGKAGGTVTPTP | |
| NTGWKTNKYGTLYKSESASFTPNTDIITRTTGPFRSMPQSGVLKAGQTIHYDEVMKQ | |
| DGHVWVGYTGNSGQRIYLPVRTWQKSTNTLGVLWGTIKGGGGSGGGGSGGGGSTRSI | |
| VMTQTPKFLLVSAGDRVTITCKASQSVSNDVAWYQQKPGQSPKLLIYYASNRYTGVP | |
| DRFTGSGYGTDFTFTISTVQAEDLAVYFCQQDYSSPLTFGSGTKLEIKRTVAAPSVF | |
| IFPPSDEQLKSGTASVVCLLNNFYPREAKVQWKVDNALQSGNSQESVTEQDSKDSTY | |
| SLSSTLTLSKADYEKHKVYACEVTHQGLSSPVTKSFNRGEC | |
| 1-20 | Signal peptide |
| 21-266 | Lysostaphin |
| 267-497 | Linker-LC |
| SEQ ID NO: 289. LYST-LC-I8-1017, Lysostaphin-light chain | |
| chimeric murine-human fusion, nucleotide sequence, | |
| ID: 500665n | |
| ATGGAGACAGACACACTCCTGCTATGGGTACTGCTGCTCTGGGTTCCAGGTTCCACT | |
| GGTGACGCCACCCACGAGCACTCCGCCCAGTGGCTGAACAACTACAAGAAGGGCTAC | |
| GGCTACGGCCCCTACCCCCTGGGCATCAACGGCGGCATGCACTACGGCGTGGACTTC | |
| TTCATGAACATCGGCACCCCCGTGAAGGCCATCTCCTCCGGCAAGATCGTGGAGGCC | |
| GGCTGGTCCAACTACGGCGGCGGCAACCAGATCGGCCTGATCGAGAACGACGGCGTG | |
| CACCGCCAGTGGTACATGCACCTGTCCAAGTACAACGTGAAGGTGGGCGACTACGTG | |
| AAGGCCGGCCAGATCATCGGCTGGTCCGGCTCCACCGGCTACTCCACCGCCCCCCAC | |
| CTGCACTTCCAGCGCATGGTGAACTCCTTCTCCAACTCCACCGCCCAGGACCCCATG | |
| CCCTTCCTGAAGTCCGCCGGCTACGGCAAGGCCGGCGGCACCGTGACCCCCACCCCC | |
| AACACCGGCTGGAAGACCAACAAGTACGGCACCCTGTACAAGTCCGAGTCCGCCTCC | |
| TTCACCCCCAACACCGACATCATCACCCGCACCACCGGCCCCTTCCGCTCCATGCCC | |
| CAGTCCGGCGTGCTGAAGGCCGGCCAGACCATCCACTACGACGAGGTGATGAAGCAG | |
| GACGGCCACGTGTGGGTGGGCTACACCGGCAACTCCGGCCAGCGCATCTACCTGCCC | |
| GTGCGCACCTGGAACAAGTCCACCAACACCCTGGGCGTGCTGTGGGGCACCATCAAG | |
| GGTGGTGGCGGTTCAGGCGGAGGTGGCTCTGGCGGTGGCGGATCcacgcgtGACATT | |
| GTGATGTCACAGTCTCCATCCTCCCTGGCTGTGTCAGCAGGAGAGAAGGTCACTATG | |
| AGCTGCAAATCCAGTCAGAGTCTGCTCAACAGTAGAACCCGAAAGAACTACTTGGCT | |
| TGGTACCAGCAGAAACCAGGGCAGTCTCCTAAACTGCTGATCTACTGGGCATCCACT | |
| AGGGAATCTGGGGTCCCTGATCGCTTCACAGGCAGTGGATCTGGGACAGATTTCACT | |
| CTCACCATCAGCAGTGTGCAGGCTGAAGACCTGGCAGTTTATTACTGCAAGCAATCT | |
| TATAATCTGTGGACGTTCGGTGGAGGCACCAAGCTGGAAATCAAACGGACTGTGGCT | |
| GCACCATCTGTCTTCATCTTCCCGCCATCTGATGAGCAGTTGAAATCTGGAACTGCC | |
| TCTGTTGTGTGCCTGCTGAATAACTTCTATCCCAGAGAGGCCAAAGTACAGTGGAAG | |
| GTGGATAACGCCCTCCAATCGGGTAACTCCCAGGAGAGTGTCACAGAGCAGGACAGC | |
| AAGGACAGCACCTACAGCCTCAGCAGCACCCTGACGCTGAGCAAAGCAGACTACGAG | |
| AAACACAAAGTCTACGCCTGCGAAGTCACCCATCAGGGCCTGAGCTCGCCCGTCACA | |
| AAGAGCTTCAACAGGGGAGAGTGTTAG | |
| 1-60 | Signal peptide |
| 61-1509 | Chimeric murine-human Light chain |
| SEQ ID NO: 290. LYST-LC-I8-1017, Lysostaphin-light chain | |
| chimeric murine-human fusion, amino acid sequence, | |
| ID: 500665p | |
| METDTLLLWVLLLWVPGSTGDATHEHSAQWLNNYKKGYGYGPYPLGINGGMHYGVDF | |
| FMNIGTPVKAISSGKIVEAGWSNYGGGNQIGLIENDGVHRQWYMHLSKYNVKVGDYV | |
| KAGQIIGWSGSTGYSTAPHLHFQRMVNSFSNSTAQDPMPFLKSAGYGKAGGTVTPTP | |
| NTGWKTNKYGTLYKSESASFTPNTDIITRTTGPFRSMPQSGVLKAGQTIHYDEVMKQ | |
| DGHVWVGYTGNSGQRIYLPVRTWNKSTNTLGVLWGTIKGGGGSGGGGSGGGGSTRDI | |
| VMSQSPSSLAVSAGEKVTMSCKSSQSLLNSRTRKNYLAWYQQKPGQSPKLLIYWAST | |
| RESGVPDRFTGSGSGTDFTLTISSVQAEDLAVYYCKQSYNLWTFGGGTKLEIKRTVA | |
| APSVFIFPPSDEQLKSGTASVVCLLNNFYPREAKVQWKVDNALQSGNSQESVTEQDS | |
| KDSTYSLSSTLTLSKADYEKHKVYACEVTHQGLSSPVTKSFNRGEC | |
| 1-20 | Signal peptide |
| 21-266 | Lysostaphin |
| 267-502 | Linker-LC |
| SEQ ID NO: 291. LYST(N125Q)-LC-I8-1017, Lysostaphin-light | |
| chain chimeric murine-human fusion, nucleotide sequence, | |
| ID: 500894n | |
| ATGGAGACAGACACACTCCTGCTATGGGTACTGCTGCTCTGGGTTCCAGGTTCCACT | |
| GGTGACGCCACCCACGAGCACTCCGCCCAGTGGCTGAACAACTACAAGAAGGGCTAC | |
| GGCTACGGCCCCTACCCCCTGGGCATCAACGGCGGCATGCACTACGGCGTGGACTTC | |
| TTCATGAACATCGGCACCCCCGTGAAGGCCATCTCCTCCGGCAAGATCGTGGAGGCC | |
| GGCTGGTCCAACTACGGCGGCGGCAACCAGATCGGCCTGATCGAGAACGACGGCGTG | |
| CACCGCCAGTGGTACATGCACCTGTCCAAGTACAACGTGAAGGTGGGCGACTACGTG | |
| AAGGCCGGCCAGATCATCGGCTGGTCCGGCTCCACCGGCTACTCCACCGCCCCCCAC | |
| CTGCACTTCCAGCGCATGGTGAACTCCTTCTCCcagTCCACCGCCCAGGACCCCATG | |
| CCCTTCCTGAAGTCCGCCGGCTACGGCAAGGCCGGCGGCACCGTGACCCCCACCCCC | |
| AACACCGGCTGGAAGACCAACAAGTACGGCACCCTGTACAAGTCCGAGTCCGCCTCC | |
| TTCACCCCCAACACCGACATCATCACCCGCACCACCGGCCCCTTCCGCTCCATGCCC | |
| CAGTCCGGCGTGCTGAAGGCCGGCCAGACCATCCACTACGACGAGGTGATGAAGCAG | |
| GACGGCCACGTGTGGGTGGGCTACACCGGCAACTCCGGCCAGCGCATCTACCTGCCC | |
| GTGCGCACCTGGAACAAGTCCACCAACACCCTGGGCGTGCTGTGGGGCACCATCAAG | |
| GGTGGTGGCGGTTCAGGCGGAGGTGGCTCTGGCGGTGGCGGATCcacgcgtGACATT | |
| GTGATGTCACAGTCTCCATCCTCCCTGGCTGTGTCAGCAGGAGAGAAGGTCACTATG | |
| AGCTGCAAATCCAGTCAGAGTCTGCTCAACAGTAGAACCCGAAAGAACTACTTGGCT | |
| TGGTACCAGCAGAAACCAGGGCAGTCTCCTAAACTGCTGATCTACTGGGCATCCACT | |
| AGGGAATCTGGGGTCCCTGATCGCTTCACAGGCAGTGGATCTGGGACAGATTTCACT | |
| CTCACCATCAGCAGTGTGCAGGCTGAAGACCTGGCAGTTTATTACTGCAAGCAATCT | |
| TATAATCTGTGGACGTTCGGTGGAGGCACCAAGCTGGAAATCAAACGGACTGTGGCT | |
| GCACCATCTGTCTTCATCTTCCCGCCATCTGATGAGCAGTTGAAATCTGGAACTGCC | |
| TCTGTTGTGTGCCTGCTGAATAACTTCTATCCCAGAGAGGCCAAAGTACAGTGGAAG | |
| GTGGATAACGCCCTCCAATCGGGTAACTCCCAGGAGAGTGTCACAGAGCAGGACAGC | |
| AAGGACAGCACCTACAGCCTCAGCAGCACCCTGACGCTGAGCAAAGCAGACTACGAG | |
| AAACACAAAGTCTACGCCTGCGAAGTCACCCATCAGGGCCTGAGCTCGCCCGTCACA | |
| AAGAGCTTCAACAGGGGAGAGTGTTAG | |
| 1-60 | Signal peptide |
| 61-1509 | Chimeric murine-human Light chain |
| SEQ ID NO: 292. LYST(N125Q)-LC-I8-1017, Lysostaphin-light | |
| chain chimeric murine-human fusion, amino acid sequence, | |
| ID: 500894p | |
| METDTLLLWVLLLWVPGSTGDATHEHSAQWLNNYKKGYGYGPYPLGINGGMHYGVDF | |
| FMNIGTPVKAISSGKIVEAGWSNYGGGNQIGLIENDGVHRQWYMHLSKYNVKVGDYV | |
| KAGQIIGWSGSTGYSTAPHLHFQRMVNSFSQSTAQDPMPFLKSAGYGKAGGTVTPTP | |
| NTGWKTNKYGTLYKSESASFTPNTDIITRTTGPFRSMPQSGVLKAGQTIHYDEVMKQ | |
| DGHVWVGYTGNSGQRIYLPVRTWNKSTNTLGVLWGTIKGGGGSGGGGSGGGGSTRDI | |
| VMSQSPSSLAVSAGEKVTMSCKSSQSLLNSRTRKNYLAWYQQKPGQSPKLLIYWAST | |
| RESGVPDRFTGSGSGTDFTLTISSVQAEDLAVYYCKQSYNLWTFGGGTKLEIKRTVA | |
| APSVFIFPPSDEQLKSGTASVVCLLNNFYPREAKVQWKVDNALQSGNSQESVTEQDS | |
| KDSTYSLSSTLTLSKADYEKHKVYACEVTHQGLSSPVTKSFNRGEC | |
| 1-20 | Signal peptide |
| 21-266 | Lysostaphin |
| 267-502 | Linker-LC |

### REFERENCE LIST

1. S. Stefani et al., Meticillin-resistant Staphylococcus aureus (MRSA): global epidemiology and harmonisation of typing methods. International journal of antimicrobial agents 39, 273 (April, 2012).
2. M. C. Ramirez, M. Marchessault, C. Govednik-Horny, D. Jupiter, H. T. Papaconstantinou, The Impact of MRSA Colonization on Surgical Site Infection Following Major Gastrointestinal Surgery. Journal of gastrointestinal surgery: official journal of the Society for Surgery of the Alimentary Tract, (Sep. 5, 2012).
3. L. A. Hamilton et al., Treatment of methicillin-resistant Staphylococcus aureus ventilator-associated pneumonia with high-dose vancomycin or linezolid. The journal of trauma and acute care surgery 72, 1478 (June, 2012).
4. D. J. Rodriguez, A. Afzal, R. Evonich, D. E. Haines, The prevalence of methicillin resistant organisms among pacemaker and defibrillator implant recipients. American journal of cardiovascular disease 2, 116 (2012).
5. H. M. Ghanem, A. M. Abou-Alia, S. A. Alsirafy, Prevalence of Methicillin-Resistant Staphylococcus aureus Colonization and Infection in Hospitalized Palliative Care Patients With Cancer. The American journal of hospice & palliative care, (Jul. 9, 2012).
6. P. Tandon, A. Delisle, J. E. Topal, G. Garcia-Tsao, High Prevalence of Antibiotic-Resistant Bacterial Infections Among Patients With Cirrhosis at a US Liver Center. Clinical gastroenterology and hepatology: the official clinical practice journal of the American Gastroenterological Association, (Aug. 17, 2012).
7. I. M. Gould, E. K. Girvan, R. A. Browning, F. M. MacKenzie, G. F. Edwards, Report of a hospital neonatal unit outbreak of community-associated methicillin-resistant Staphylococcus aureus. Epidemiology and infection 137, 1242 (September, 2009).
8. B. Edwards, I. Gould, Meticillin-resistant Staphylococcus aureus pneumonia. British journal of hospital medicine 73, 386 (July, 2012).
9. C. H. Hsiao et al., Methicillin-resistant Staphylococcus aureus ocular infection: a 10-year hospital-based study. Ophthalmology 119, 522 (March 2012).
10. A. W. Zhou, M. C. Lee, C. J. Rudnisky, Ocular microbiology trends in Edmonton, Alberta: a 10-year review. Canadian journal of ophthalmology. Journal canadien d'ophtalmologie 47, 301 (June, 2012).
11. A. van der Zee, W. D. Hendriks, L. Roorda, J. M. Ossewaarde, J. Buitenwerf, Review of a major epidemic of methicillin resistant Staphylococcus aureus: The costs of screening and consequences of outbreak management. American journal of infection control, (Aug. 21, 2012).
12. T. Kim, P. I. Oh, A. E. Simor, The economic impact of methicillin-resistant Staphylococcus aureus in Canadian hospitals. Infection control and hospital epidemiology: the official journal of the Society of Hospital Epidemiologists of America 22, 99 (February, 2001).
13. B. Y. Lee et al., The economic burden of community-associated methicillin-resistant Staphylococcus aureus (CA-MRSA). Clinical microbiology and infection: the official publication of the European Society of Clinical Microbiology and Infectious Diseases, (May 21, 2012).
14. T. Ganz, Antimicrobial polypeptides. J Leukoc. Biol. 75, 34 (January 2004, 2004).
15. H. Komatsuzawa et al., Innate defences against methicillin-resistant Staphylococcus aureus (MRSA) infection. J Pathol. 208, 249 (January 2006, 2006).
16. A. Tossi, L. Sandri, A. Giangaspero, Amphipathic, alpha-helical antimicrobial peptides. Biopolymers 55, 4 (2000, 2000).
17. D. M. Bowdish et al., Impact of LL-37 on anti-infective immunity. J. Leukoc. Biol. 77, 451 (April 2005, 2005).
18. R. E. Hancock, H. G. Sahl, Antimicrobial and host-defense peptides as new anti-infective therapeutic strategies. Nat. Biotechnol. 24, 1551 (December 2006, 2006).
19. R. Gennaro, M. Zanetti, Structural features and biological activities of the cathelicidin-derived antimicrobial peptides. Biopolymers 55, 31 (2000, 2000).
20. J. W. Larrick, M. Hirata, J. Zhong, S. C. Wright, Anti-microbial activity of human CAP18 peptides. Immunotechnology. 1, 65 (May 1995, 1995).
21. S. M. Travis et al., Bactericidal activity of mammalian cathelicidin-derived peptides. Infect. Immun. 68, 2748 (May 2000, 2000).
22. J. Turner, Y. Cho, N. N. Dinh, A. J. Waring, R. I. Lehrer, Activities of LL-37, a cathelin-associated antimicrobial peptide of human neutrophils. Antimicrob. Agents Chemother. 42, 2206 (September 1998, 1998).
23. M. Imboden, M. W. Riggs, D. A. Schaefer, E. J. Homan, R. D. Bremel, Antibodies fused to innate immune molecules reduce initiation of Cryptosporidium parvum infection in mice. Antimicrobial agents and chemotherapy 54, 1385 (April, 2010).
24. J. Wehkamp, J. Schauber, E. F. Stange, Defensins and cathelicidins in gastrointestinal infections. Curr. Opin. Gastroenterol. 23, 32 (January 2007, 2007).
25. R. I. Lehrer et al., Interaction of human defensins with Escherichia coli. Mechanism of bactericidal activity. J. Clin. Invest 84, 553 (August 1989, 1989).
26. R. I. Lehrer, A. K. Lichtenstein, T. Ganz, Defensins: antimicrobial and cytotoxic peptides of mammalian cells. Annu. Rev. Immunol. 11, 105 (1993, 1993).
27. J. Harder, J. Bartels, E. Christophers, J. M. Schroder, Isolation and characterization of human beta-defensin-3, a novel human inducible peptide antibiotic. J Biol Chem 276, 5707 (Feb. 23, 2001).
28. J. Harder, J. Bartels, E. Christophers, J. M. Schroder, A peptide antibiotic from human skin. Nature 387, 861 (Jun. 26, 1997).
29. X. D. Qu, K. C. Lloyd, J. H. Walsh, R. I. Lehrer, Secretion of type II phospholipase A2 and cryptdin by rat small intestinal Paneth cells. Infect. Immun. 64, 5161 (December 1996, 1996).
30. X. D. Qu, R. I. Lehrer, Secretory phospholipase A2 is the principal bactericide for staphylococci and other gram-positive bacteria in human tears. Infect Immun 66, 2791 (June 1998, 1998).
31. K. M. Saari, V. Aho, V. Paavilainen, T. J. Nevalainen, Group II PLA(2) content of tears in normal subjects. Invest Ophthalmol. Vis. Sci. 42, 318 (February 2001, 2001).
32. S. S. Harwig et al., Bactericidal properties of murine intestinal phospholipase A2. J. Clin. Invest 95, 603 (February 1995, 1995).
33. R. S. Koduri et al., Action of human group IIa secreted phospholipase A2 on cell membranes. Vesicle but not heparinoid binding determines rate of fatty acid release by exogenously added enzyme. J. Biol. Chem. 273, 32142 (Nov. 27, 1998, 1998).
34. A. Dubouix et al., Bactericidal properties of group IIa secreted phospholipase A(2) against Pseudomonas aeruginosa clinical isolates. J Med Microbiol 52, 1039 (December 2003, 2003).
35. A. P. Gimenez et al., High bactericidal efficiency of type iia phospholipase A2 against Bacillus anthracis and inhibition of its secretion by the lethal toxin. J Immunol. 173, 521 (Jul. 1, 2004, 2004).
36. Y. Weinrauch, C. Abad, N. S. Liang, S. F. Lowry, J. Weiss, Mobilization of potent plasma bactericidal activity during systemic bacterial challenge. Role of group IIA phospholipase A2. J Clin. Invest 102, 633 (Aug. 1, 1998, 1998).
37. J. O. Gronroos, V. J. Laine, T. J. Nevalainen, Bactericidal group IIA phospholipase A2 in serum of patients with bacterial infections. J Infect Dis 185, 1767 (Jun. 15, 2002, 2002).
38. J. O. Gronroos, V. J. Laine, M. J. Janssen, M. R. Egmond, T. J. Nevalainen, Bactericidal properties of group IIA and group V phospholipases A2. J Immunol. 166, 4029 (Mar. 15, 2001, 2001).
39. D. P. Clark, S. Durell, W. L. Maloy, M. Zasloff, Ranalexin. A novel antimicrobial peptide from bullfrog (Rana catesbeiana) skin, structurally related to the bacterial antibiotic, polymyxin. The Journal of biological chemistry 269, 10849 (Apr. 8, 1994).
40. M. Zasloff, Magainins, a class of antimicrobial peptides from Xenopus skin: isolation, characterization of two active forms, and partial cDNA sequence of a precursor. Proceedings of the National Academy of Sciences of the United States of America 84, 5449 (August, 1987).
41. A. Shah, J. Mond, S. Walsh, Lysostaphin-coated catheters eradicate Staphylococcus aureus challenge and block surface colonization. Antimicrobial agents and chemotherapy 48, 2704 (July, 2004).
42. S. Walsh, A. Shah, J. Mond, Improved pharmacokinetics and reduced antibody reactivity of lysostaphin conjugated to polyethylene glycol. Antimicrobial agents and chemotherapy 47, 554 (February, 2003).
43. C. Kelly-Quintos, L. A. Cavacini, M. R. Posner, D. Goldmann, G. B. Pier, Characterization of the opsonic and protective activity against Staphylococcus aureus of fully human monoclonal antibodies specific for the bacterial surface polysaccharide poly-N-acetylglucosamine Infection and immunity 74, 2742 (May, 2006).
44. Y. Feng et al., Evolution and pathogenesis of Staphylococcus aureus: lessons learned from genotyping and comparative genomics. FEMS microbiology reviews 32, 23 (January, 2008).
45. L. Sanguinetti, S. Toti, V. Reguzzi, F. Bagnoli, C. Donati, A Novel Computational Method Identifies Intra- and Inter-Species Recombination Events in Staphylococcus aureus and Streptococcus pneumoniae. PLoS computational biology 8, e1002668 (September, 2012).
46. S. K. Mazmanian, H. Ton-That, O. Schneewind, Sortase-catalysed anchoring of surface proteins to the cell wall of Staphylococcus aureus. Molecular microbiology 40, 1049 (June, 2001).
47. W. Van den Broeck, A. Derore, P. Simoens, Anatomy and nomenclature of murine lymph nodes: Descriptive study and nomenclatory standardization in BALB/cAnNCrl mice. J. Immunol. Methods 312, 12 (May 30, 2006, 2006).
48. Heinrich P, Rosenstein R, Bohmer M, Sonner P, Gotz F. 1987. The molecular organization of the lysostaphin gene and its sequences repeated in tandem. Mol. Gen. Genet. 209:563-569.
49. Recsei P A, Gruss A D, Novick R P. 1987. Cloning, sequence, and expression of the lysostaphin gene from Staphylococcus simulans. Proc Natl Acad Sci USA 84:1127-1131.
50. Thumm G, Gotz F. 1997. Studies on prolysostaphin processing and characterization of the lysostaphin immunity factor (Lif) of Staphylococcus simulans biovar staphylolyticus. Molecular microbiology 23:1251-1265.
51. Wu J A, Kusuma C, Mond J J, Kokai-Kun J F. 2003. Lysostaphin disrupts Staphylococcus aureus and Staphylococcus epidermidis biofilms on artificial surfaces. Antimicrob. Agents Chemother. 47:3407-3414.
52. Kokai-Kun J F, Chanturiya T, Mond J J. 2007. Lysostaphin as a treatment for systemic Staphylococcus aureus infection in a mouse model. The Journal of antimicrobial chemotherapy 60:1051-1059.
53. Kokai-Kun J F, Walsh S M, Chanturiya T, Mond J J. 2003. Lysostaphin cream eradicates Staphylococcus aureus nasal colonization in a cotton rat model. Antimicrob. Agents Chemother. 47:1589-1597.
54. Kusuma C, Jadanova A, Chanturiya T, Kokai-Kun J F. 2007. Lysostaphin-resistant variants of Staphylococcus aureus demonstrate reduced fitness in vitro and in vivo. Antimicrobial agents and chemotherapy 51:475-482.
55. Mierau I, Leij P, van S, I, Blommestein B, Floris E, Mond J, Smid E J. 2005. Industrial-scale production and purification of a heterologous protein in Lactococcus lactis using the nisin-controlled gene expression system NICE: the case of lysostaphin. Microb. Cell Fact. 4:15.
56. Mierau I, Olieman K, Mond J, Smid E J. 2005. Optimization of the Lactococcus lactis nisin-controlled gene expression system NICE for industrial applications. Microb. Cell Fact. 4:16.
57. Williamson C M, Bramley A J, Lax A J. 1994. Expression of the lysostaphin gene of Staphylococcus simulans in a eukaryotic system. Appl Environ Microbiol 60:771-776.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be within the scope of the following claims.

1-76. (canceled)

77. A recombinant fusion protein comprising an active recombinant lysostaphin protein fused to a fusion partner protein.

78. The recombinant fusion protein of claim 77, wherein said recombinant fusion polypeptide is secreted by a mammalian cell.

79. The recombinant fusion protein of claim 77, wherein said fusion protein partner is an immunoglobulin molecule or fragment thereof.

80. The recombinant fusions protein of claim 79, wherein said lysostaphin protein is fused to the N terminal of said immunoglobulin molecule or fragment thereof.

81. The recombinant fusion protein of claim 79, wherein said lysostaphin protein is fused to the C terminal of said immunoglobulin molecule or fragment thereof.

82. The recombinant fusion protein of claim 79, wherein said lysostaphin protein is connected to said immunoglobulin molecule or fragment thereof via a peptide linker.

83. The recombinant fusion protein of claim 79, wherein said active recombinant lysostaphin is encoded by an amino acid sequence found in hosts which naturally express lysostaphin.

84. The recombinant fusion protein of claim 83, wherein said active recombinant lysostaphin is encoded by an amino acid sequence selected from the group consisting of amino acids 21-266 of SEQ ID NO:278, amino acids 21-266 of SEQ ID NO:284, and amino acids 21-266 of SEQ ID NO: 290.

85. The recombinant fusion protein of claim 77, wherein said active recombinant lysostaphin is encoded by an amino acid sequence altered from that found in a host naturally secreting lysostaphin.

86. The recombinant fusion protein of claim 85, wherein said amino acid sequence of said active recombinant lysostaphin has been altered to eliminate one or more glycosylation sites.

87. The recombinant fusion protein of claim 86, wherein said active recombinant lysostaphin is encoded by an amino acid sequence selected from the group consisting of amino acids 21-266 of Seq 280, amino acids 21-266 of Seq 282, amino acids 21-266 of Seq 286, amino acids 21-266 of Seq 288, and amino acids 21-266 of Seq 292.

88. The recombinant fusion protein of claim 78, wherein said protein is expressed by mammalian cells and harvested from supernatant at more than about 1 ug/ml.

89. The recombinant fusion protein of claim 77, wherein said lysostaphin protein comprises a preprolysostaphin.

90. The recombinant fusion protein of claim 77, wherein said lysostaphin protein is a mature lysostaphin.

91. The recombinant fusion protein of claim 77, wherein said active lysostaphin is bactericidal.

92. The recombinant fusion protein of claim 77, wherein said active lysostaphin is bacteriacidal at a MIC of 1-100 nanomolar.

93. The recombinant fusion protein of claim 77, wherein said active lysostaphin is bactericidal to methicillin resistant S. aureus at a MIC of 1-100 nanomolar.

94. The recombinant fusion protein of claim 77, wherein the enzymatically active half-life of said active recombinant lysostaphin in vivo is greater than 1 hour.

95. The recombinant fusion protein of claim 77 wherein said recombinant fusion protein is stable at 2-8 C for over 3 months.

96. The recombinant fusion protein of claim 77 wherein said active lysostaphin is bactericidal to methicillin resistant S. aureus at a MIC of 1-100 nanomolar.

97-99. (canceled)


SEQUENCE LISTING (293 sequences)

SEQ ID NO: 1 - DNA, 408 bp, Artificial Sequence
  Synthetic
atggagacagacacactcctgctatgggtactgctgctctgggttccaggttccactggtgacacgcgtgatgttttgatgacccaaactccactctccctgcctgtcagtcttggagatcaagcctccatctcttgcagatctagtcagagcattgtacatactaatggaaacacctatttagaatggtacctgcagaaaccgggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatattccgtggacgttcggtggaggcaccaagctggaaatcaaacgg

SEQ ID NO: 2 - protein, 136 aa, Artificial Sequence
  Synthetic
METDTLLLWVLLLWVPGSTGDTRDVLMTQTPLSLPVSLGDQASISCRSSQSIVHTNGNTYLEWYLQKPGQSPKLLIYKVSNRFSGVPDRFSGSGSGTDFTLKISRVEAEDLGVYYCFQGSHIPWTFGGGTKLEIKR

SEQ ID NO: 3 - DNA, 417 bp, Artificial Sequence
  Synthetic
atggagacagacacactcctgctatgggtactgctgctctgggttccaggttccactggtgacacgcgtcaggtccaactgcagcagcctggggctgagctggtgaggcctggggcttcagtgaacctgtcctgcagggcttctggctacaccttcaccacctactggatgatctgggtgaagcagaggcctggacaaggccttgaatggattggtatgattgatccttcagacagtgagactcactacaatcaaatgttcaaggacaaggccacattgactgtagacaaatcctccaccacagcctacatgcagttcagcagcctaacatctgaggactctgcggtctattactgtgcaagatggaacttcggtaagggctactggggccaaggcaccactctcacggtctcctca

SEQ ID NO: 4 - protein, 139 aa, Artificial Sequence
  Synthetic
METDTLLLWVLLLWVPGSTGDTRQVQLQQPGAELVRPGASVNLSCRASGYTFTTYWMIWVKQRPGQGLEWIGMIDPSDSETHYNQMFKDKATLTVDKSSTTAYMQFSSLTSEDSAVYYCARWNFGKGYWGQGTTLTVSS

SEQ ID NO: 5 - DNA, 408 bp, Artificial Sequence
  Synthetic
atggagacagacacactcctgctatgggtactgctgctctgggttccaggttccactggtgacacgcgtgacattgtgatgtcacagtctccatcctccctggctgtgtcagcaggagagaaggtcactatgagctgcaaatccagtcagagtctgctcaacagtagaacccgaaagaactacttggcttggtaccagcagaaaccagggcagtctcctaaactgctgatctactgggcatccactagggaatctggggtccctgatcgcttcacaggcagtggatctgggacagatttcactctcaccatcagcagtgtgcaggctgaagacctggcagtttattactgcaagcaatcttataatctgtggacgttcggtggaggcaccaagctggaaatcaaacgg

SEQ ID NO: 6 - protein, 136 aa, Artificial Sequence
  Synthetic
METDTLLLWVLLLWVPGSTGDTRDIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYNLWTFGGGTKLEIKR

SEQ ID NO: 7 - DNA, 414 bp, Artificial Sequence
  Synthetic
atggagacagacacactcctgctatgggtactgctgctctgggttccaggttccactggtgacacgcgtgaggttcagctgcagcagtctggggcagagcttgtgaagccaggggcctcagtcaagttgtcctgcacagcttctggcttcaacattaaagacacctatatgcactgggtgaagcagaggcctgaacagggcctggagtggattggaaggattgatcctgcgaatggtaatactaaatatgacccgaagttccagggcaaggccactataacagcagacacatcctccaacacagcctacctgcagctcagcagcctgacatctgaggacactgccgtctattactgtgctggtaaccactactttgactactggggccaaggcaccactctcacagtctcctca

SEQ ID NO: 8 - protein, 138 aa, Artificial Sequence
  Synthetic
METDTLLLWVLLLWVPGSTGDTREVQLQQSGAELVKPGASVKLSCTASGFNIKDTYMHWVKQRPEQGLEWIGRIDPANGNTKYDPKFQGKATITADTSSNTAYLQLSSLTSEDTAVYYCAGNHYFDYWGQGTTLTVSS

SEQ ID NO: 9 - DNA, 393 bp, Artificial Sequence
  Synthetic
atggagacagacacactcctgctatgggtactgctgctctgggttccaggttccactggtgacacgcgtgacattgtgatgacccagtctcaaaaattcatgtccacatcagtaggagacagggtcagcgtcacctgcaaggccagtcagaatgtgggtactaatgtagcctggtatcaacagaaaccagggcaatctcctaaagcactgatttactcggcatcctaccggtacagtggagtccctgatcgcttcacaggcagtggatctgggacagatttcactctcaccatcagcaatgtgcagtctgaagacttggcagagtatttctgtcagcaatataacagctatcctctcacgttcggtgctgggaccaagctggagctgaaacgg

SEQ ID NO: 10 - protein, 131 aa, Artificial Sequence
  Synthetic
METDTLLLWVLLLWVPGSTGDTRDIVMTQSQKFMSTSVGDRVSVTCKASQNVGTNVAWYQQKPGQSPKALIYSASYRYSGVPDRFTGSGSGTDFTLTISNVQSEDLAEYFCQQYNSYPLTFGAGTKLELKR

SEQ ID NO: 11 - DNA, 420 bp, Artificial Sequence
  Synthetic
atggagacagacacactcctgctatgggtactgctgctctgggttccaggttccactggtgacacgcgtgaagtgaagctggtggagtctgggggaggtttagtgcagcctggagggtccctgaaactctcctgtgcagcctctggattcactttcagtagctataccatgtcttgggttcgccagactccagagaagaggctggagtgggtcgcatacattagtaatggtggtggtagcacctactatccagacactgtaaagggccgattcaccatctccagagacaatgccaagaacaccctgtacctgcaaatgagcagtctgaagtctgaggacacggccatgtattactgtgcaagacaggtacgacgggggatggactactggggtcaaggaacctcagtcaccgtctcctca

SEQ ID NO: 12 - protein, 140 aa, Artificial Sequence
  Synthetic
METDTLLLWVLLLWVPGSTGDTREVKLVESGGGLVQPGGSLKLSCAASGFTFSSYTMSWVRQTPEKRLEWVAYISNGGGSTYYPDTVKGRFTISRDNAKNTLYLQMSSLKSEDTAMYYCARQVRRGMDYWGQGTSVTVSS

SEQ ID NO: 13 - DNA, 411 bp, Artificial Sequence
  Synthetic
atggagacagacacactcctgctatgggtactgctgctctgggttccaggttccactggtgacacgcgtgacattgtgatgacacagtctccatcctccctgactgtgacagcaggagagaaggtcactatgagctgcaagtccagtcagagtctgttaaacagtggaaatcaaaagaactacttgacctggtaccagcagaaaccagggcagcctcctaaactgttgatctactgggcatccactagggaatctggggtccctgatcgcttcacaggcagtggatctggaacagatttcactctcaccatcagcagtgtgcaggctgaagacctggcagtttattactgtcagaatgattatagttatcctttcacgttcggctcggggacaaagttggaaataaaacgg

SEQ ID NO: 14 - protein, 137 aa, Artificial Sequence
  Synthetic
METDTLLLWVLLLWVPGSTGDTRDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKR

SEQ ID NO: 15 - DNA, 420 bp, Artificial Sequence
  Synthetic
atggagacagacacactcctgctatgggtactgctgctctgggttccaggttccactggtgacacgcgtgaggtccagctgcagcagtctggacctgagctagtgaagactggggcttcagtgaagatatcctgcaaggcttctggttactcattcactggttactacatgcactgggtcaagcagagccatggaaagagccttgagtggattggatatattagttgttacaatggtgctactagctacaaccagaagttcaagggcaaggccacatttactgtagacacatcctccagcacagcctacatgcagttcaacagcctgacatctgaagactctgcggtctattactgtgcaagatcgaggactggagcctggtttgcttactggggccaagggactctggtcactgtctct

SEQ ID NO: 16 - protein, 140 aa, Artificial Sequence
  Synthetic
METDTLLLWVLLLWVPGSTGDTREVQLQQSGPELVKTGASVKISCKASGYSFTGYYMHWVKQSHGKSLEWIGYISCYNGATSYNQKFKGKATFTVDTSSSTAYMQFNSLTSEDSAVYYCARSRTGAWFAYWGQGTLVTVS

SEQ ID NO: 17 - DNA, 393 bp, Artificial Sequence
  Synthetic
atggagacagacacactcctgctatgggtactgctgctctgggttccaggttccactggtgacacgcgtgatatccagatgacacagactacatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggagcaagaagatattgccacttacttttgccaacagggtaatacgcttccgtggacgttcggtggaggtaccaagctggaaatcaaacgg

SEQ ID NO: 18 - protein, 131 aa, Artificial Sequence
  Synthetic
METDTLLLWVLLLWVPGSTGDTRDIQMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIKR

SEQ ID NO: 19 - DNA, 432 bp, Artificial Sequence
  Synthetic
atggagacagacacactcctgctatgggtactgctgctctgggttccaggttccactggtgacacgcgtgaagtgaagctggtggagtctgggggaggcttagtgaagcctggagggtccctgaaactctcctgtgcagcctctggattcgctttcagtagctatgacatgtcttgggttcgccagactccggagaagaggctggagtgggtcgcaaccattagtagtggtggtagttacacctactatccagacagtgtgaagggccgattcaccatctccagagacaatgccaggaacaccctgtacctgcaaatgagcagtctgaggtctgaggacacggccttgtattactgtgcaagaccacggttacagctcgggtcgcctgcctggtttgcttactggggccaagggactctggtcactgtctct

SEQ ID NO: 20 - protein, 144 aa, Artificial Sequence
  Synthetic
METDTLLLWVLLLWVPGSTGDTREVKLVESGGGLVKPGGSLKLSCAASGFAFSSYDMSWVRQTPEKRLEWVATISSGGSYTYYPDSVKGRFTISRDNARNTLYLQMSSLRSEDTALYYCARPRLQLGSPAWFAYWGQGTLVTVS

SEQ ID NO: 21 - DNA, 393 bp, Artificial Sequence
  Synthetic
atggagacagacacactcctgctatgggtactgctgctctgggttccaggttccactggtgacacgcgtgacatcctgatgacccaatctccatcctccatgtctgtatctctgggagacacagtcagcatcacttgccatgcaagtcagggcattagcagtaatatagggtggttgcagcagaaaccagggaaatcatttaagggcctgatctatcatggaaccaacttggaagatggagttccatcaaggttcagtggcagtggatctggagcagattattctctcaccatcagcagcctggaatctgaagattttgcagactattactgtgtacagtatgctcagtttcctcggacgttcggtggaggcaccaagctggaaatcaaacgg

SEQ ID NO: 22 - protein, 131 aa, Artificial Sequence
  Synthetic
METDTLLLWVLLLWVPGSTGDTRDILMTQSPSSMSVSLGDTVSITCHASQGISSNIGWLQQKPGKSFKGLIYHGTNLEDGVPSRFSGSGSGADYSLTISSLESEDFADYYCVQYAQFPRTFGGGTKLEIKR

SEQ ID NO: 23 - DNA, 408 bp, Artificial Sequence
  Synthetic
atggagacagacacactcctgctatgggtactgctgctctgggttccaggttccactggtgacacgcgtcaggtgcagctgaagcagtcaggacctggcctagtgcagccctcacagagcctgtccataacctgcacagtctctggtttctcattaactagctatggtgtacactgggttcgccagtctccaggaaagggtctggagtggctgggagtgatatggagaggtggaagcacagactacaatgcagctttcatgtccagactgagcatcaccaaggacaactccaagagccaagttttctttaaaatgaacagtctgcaagctgatgacactgccatatactactgtgccaaagaggacttgcttgcttactggggccaagggactctggtcactgtctct

SEQ ID NO: 24 - protein, 136 aa, Artificial Sequence
  Synthetic
METDTLLLWVLLLWVPGSTGDTRQVQLKQSGPGLVQPSQSLSITCTVSGFSLTSYGVHWVRQSPGKGLEWLGVIWRGGSTDYNAAFMSRLSITKDNSKSQVFFKMNSLQADDTAIYYCAKEDLLAYWGQGTLVTVS

SEQ ID NO: 25 - DNA, 393 bp, Artificial Sequence
  Synthetic
atggagacagacacactcctgctatgggtactgctgctctgggttccaggttccactggtgacacgcgtgacatccagatgactcagtctccagcctccctatctgcatctgtgggagaaactgtcaccatcacatgtcgagcaagtgggaatattcacaattatttagcatggtatcagcagaaacagggaaaatctcctcagctcctggtctataatgcaaaaaccttagcagatggtgtgccatcaaggttcagtggcagtggatcaggaacacaatattctctcaagatcaacagcctgcagcctgaagattttgggagttattactgtcaacatttttggagtactccgtggacgttcggtggaggcaccaagctggaaatcaaacgg

SEQ ID NO: 26 - protein, 131 aa, Artificial Sequence
  Synthetic
METDTLLLWVLLLWVPGSTGDTRDIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIKR

SEQ ID NO: 27 - DNA, 432 bp, Artificial Sequence
  Synthetic
atggagacagacacactcctgctatgggtactgctgctctgggttccaggttccactggtgacacgcgtcaggtccagctgcagcagtctggagctgagctggtaaggcctgggacttcagtgaaggtgtcctgcaaggcttctggatacgccttcactaattacttgatagagtgggtaaagcagaggcctggacagggccttgagtggattggagtgattaatcctggaagtggtggtactaactacaatgagaagttcaagggcaaggcaacactgactgcagacaaatcctccagcactgcctacatgcagctcagcagcctgacatctgatgactctgcggtctatttctgtgcaagatgggactacggtagtagctacgaacgtgctatggactactggggtcaaggaacctcagtcaccgtctcc

SEQ ID NO: 28 - protein, 144 aa, Artificial Sequence
  Synthetic
METDTLLLWVLLLWVPGSTGDTRQVQLQQSGAELVRPGTSVKVSCKASGYAFTNYLIEWVKQRPGQGLEWIGVINPGSGGTNYNEKFKGKATLTADKSSSTAYMQLSSLTSDDSAVYFCARWDYGSSYERAMDYWGQGTSVTVS

SEQ ID NO: 29 - DNA, 393 bp, Artificial Sequence
  Synthetic
atggagacagacacactcctgctatgggtactgctgctctgggttccaggttccactggtgacacgcgtagtattgtgatgacccagactcccaaattcctgcttgtatcagcaggagacagggttaccataacctgcaaggccagtcagagtgtgagtaatgatgtagcttggtaccaacagaagccagggcagtctcctaaactgctgatatactatgcatccaatcgctacactggagtccctgatcgcttcactggcagtggatatgggacggatttcactttcaccatcagcactgtgcaggctgaagacctggcagtttatttctgtcagcaggattatagctctcctctcacgttcggctcggggacaaagttggaaataaaacgg

SEQ ID NO: 30 - protein, 131 aa, Artificial Sequence
  Synthetic
METDTLLLWVLLLWVPGSTGDTRSIVMTQTPKFLLVSAGDRVTITCKASQSVSNDVAWYQQKPGQSPKLLIYYASNRYTGVPDRFTGSGYGTDFTFTISTVQAEDLAVYFCQQDYSSPLTFGSGTKLEIKR

SEQ ID NO: 31 - DNA, 432 bp, Artificial Sequence
  Synthetic
atggagacagacacactcctgctatgggtactgctgctctgggttccaggttccactggtgacacgcgtcagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacacctacactggagagccaacatatgctgatgacttcaagggacggtttgccttctctttggaaacctctgccagcactgcctatttgcagatcaacaacctcaaaaatgaggacatggctacatatttctgtgcaagaacggcggatctactatggttacgacgtcggtttgcttactggggccaagggactctggtcactgtctct

SEQ ID NO: 32 - protein, 144 aa, Artificial Sequence
  Synthetic
METDTLLLWVLLLWVPGSTGDTRQIQLVQSGPELKKPGETVKISCKASGYTFTNYGMNWVKQAPGKGLKWMGWINTYTGEPTYADDFKGRFAFSLETSASTAYLQINNLKNEDMATYFCARTADLLWLRRRFAYWGQGTLVTVS

SEQ ID NO: 33 - DNA, 842 bp, Artificial Sequence
  Synthetic
atggagacagacacactcctgctatgggtactgctgctctgggttccaggttccactggtgacgccacccacgagcactccgcccagtggctgaacaactacaagaagggctacggctacggcccctaccccctgggcatcaacggcggcatgcactacggcgtggacttcttcatgaacatcggcacccccgtgaaggccatctcctccggcaagatcgtggaggccggctggtccaactacggcggcggcaaccagatcggcctgatcgagaacgacggcgtgcaccgccagtggtacatgcacctgtccaagtacaacgtgaaggtgggcgactacgtgaaggccggccagatcatcggctggtccggctccaccggctactccaccgccccccacctgcacttccagcgcatggtgaactccttctccaactccaccgcccaggaccccatgcccttcctgaagtccgccggctacggcaaggccggcggcaccgtgacccccacccccaacaccggctggaagaccaacaagtacggcaccctgtacaagtccgagtccgcctccttcacccccaacaccgacatcatcacccgcaccaccggccccttccgctccatgccccagtccggcgtgctgaaggccggccagaccatccactacgacgaggtgatgaagcaggacggccacgtgtgggtgggctacaccggcaactccggccagcgcatctacctgcccgtgcgcacctggaacaagtccaccaacaccctgggcgtgctgtggggcaccatcaagggtggtggcggttcaggcggaggtggctctggcggtggcggatc

SEQ ID NO: 34 - protein, 281 aa, Artificial Sequence
  Synthetic
METDTLLLWVLLLWVPGSTGDATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKGGGGSGGGGSGGGGS

SEQ ID NO: 35 - DNA, 321 bp, Artificial Sequence
  Synthetic
actgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 36 - protein, 106 aa, Artificial Sequence
  Synthetic
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - DNA, 993 bp, Artificial Sequence
  Synthetic
gcgtcgaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 38 - protein, 330 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - DNA, 168 bp, Artificial Sequence
  Synthetic
gcggccgcaggtggtggcggttcaggcggaggtggctctggcggtggcggatccctgctgggggatttcttccggaagtctaaagagaagattgggaaagagtttaaaagaattgtccagagaatcaaggattttttgcggaatcttgtgcccaggacagaatcctag

SEQ ID NO: 40 - protein, 55 aa, Artificial Sequence
  Synthetic
AAAGGGGSGGGGSGGGGSLLGDFFRKSKEKIGKEFKRIVQRIKDFLRNLVPRTES

SEQ ID NO: 41 - DNA, 180 bp, Artificial Sequence
  Synthetic
gcggccgcaggtggtggcggttcaggcggaggtggctctggcggtggcggatccggtataggcgatcctgttacctgccttaagagtggagccatatgtcatccagtcttttgccctagaaggtataaacaaattggcacctgtggtctccctggaacaaaatgctgcaaaaagccatga

SEQ ID NO: 42 - protein, 55 aa, Artificial Sequence; identical to SEQ ID NO: 40
  Synthetic

SEQ ID NO: 43 - DNA, 192 bp, Artificial Sequence
  Synthetic
gcggccgcaggtggtggcggttcaggcggaggtggctctggcggtggcggatccggaatcataaacacattacagaaatattattgcagagtcagaggcggccggtgtgctgtgctcagctgccttccaaaggaggaacagatcggcaagtgctcgacgcgtggccgaaaatgctgccgaagaaagaaataa

SEQ ID NO: 44 - protein, 63 aa, Artificial Sequence
  Synthetic
AAAGGGGSGGGGSGGGGSGIINTLQKYYCRVRGGRCAVLSCLPKEEQIGKCSTRGRKCCRRKK

SEQ ID NO: 45 - DNA, 429 bp, Artificial Sequence
  Synthetic
gcggccgcaggtggtggcggttcaggcggaggtggctctggcggtggcggatcgaatttggtgaatttccacagaatgatcaagttgacgacaggaaaggaagccgcactcagttatggcttctacggctgccactgtggcgtgggtggcagaggatcccccaaggatgcaacggatcgctgctgtgtcactcatgactgttgctacaaacgtctggagaaacgtggatgtggcaccaaatttctgagctacaagtttagcaactcggggagcagaatcacctgtgcaaaacaggactcctgcagaagtcaactgtgtgagtgtgataaggctgctgccacctgttttgctagaaacaagacgacctacaataaaaagtaccagtactattccaataaacactgcagagggagcacccctcgttgctga

SEQ ID NO: 46 - protein, 142 aa, Artificial Sequence
  Synthetic
AAAGGGGSGGGGSGGGGSNLVNFHRMIKLTTGKEAALSYGFYGCHCGVGGRGSPKDATDRCCVTHDCCYKRLEKRGCGTKFLSYKFSNSGSRITCAKQDSCRSQLCECDKAAATCFARNKTTYNKKYQYYSNKHCRGSTPRC

SEQ ID NO: 47 - DNA, 411 bp, Artificial Sequence
  Synthetic
atggagacagacacactcctgctatgggtactgctgctctgggttccaggttccactggtgacacgcgtgatgttgtgatgacccaaattccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttcctccgtggacgtttggtggaggcaccaagctggaaatcaaacgg

SEQ ID NO: 48 - protein, 137 aa, Artificial Sequence
  Synthetic
METDTLLLWVLLLWVPGSTGDTRDVVMTQIPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPPWTFGGGTKLEIKR

SEQ ID NO: 49 - DNA, 429 bp, Artificial Sequence
  Synthetic
atggagacagacacactcctgctatgggtactgctgctctgggttccaggttccactggtgacacgcgtcagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacaccaacactggagagccaacatatgctgaagagttcaaggggcggtttgccttctctttggaaacctctgccagcactgcctatttgcagatcaacaacctcaaaaatgaggacacggctacatatttctgtgcaagacacggtggtaggagctggtacttcgatgtctggggcgcagggaccacggtcaccgtctcctcagcg

SEQ ID NO: 50 - protein, 143 aa, Artificial Sequence
  Synthetic
METDTLLLWVLLLWVPGSTGDTRQIQLVQSGPELKKPGETVKISCKASGYTFTNYGMNWVKQAPGKGLKWMGWINTNTGEPTYAEEFKGRFAFSLETSASTAYLQINNLKNEDTATYFCARHGGRSWYFDVWGAGTTVTVSSA

SEQ ID NO: 51 - DNA, 1509 bp, Artificial Sequence
  Synthetic
atggagacagacacactcctgctatgggtactgctgctctgggttccaggttccactggtgacgccacccacgagcactccgcccagtggctgaacaactacaagaagggctacggctacggcccctaccccctgggcatcaacggcggcatgcactacggcgtggacttcttcatgaacatcggcacccccgtgaaggccatctcctccggcaagatcgtggaggccggctggtccaactacggcggcggcaaccagatcggcctgatcgagaacgacggcgtgcaccgccagtggtacatgcacctgtccaagtacaacgtgaaggtgggcgactacgtgaaggccggccagatcatcggctggtccggctccaccggctactccaccgccccccacctgcacttccagcgcatggtgaactccttctccaactccaccgcccaggaccccatgcccttcctgaagtccgccggctacggcaaggccggcggcaccgtgacccccacccccaacaccggctggaagaccaacaagtacggcaccctgtacaagtccgagtccgcctccttcacccccaacaccgacatcatcacccgcaccaccggccccttccgctccatgccccagtccggcgtgctgaaggccggccagaccatccactacgacgaggtgatgaagcaggacggccacgtgtgggtgggctacaccggcaactccggccagcgcatctacctgcccgtgcgcacctggaacaagtccaccaacaccctgggcgtgctgtggggcaccatcaagggtggtggcggttcaggcggaggtggctctggcggtggcggatccacgcgtgatgttttgatgacccaaactccactctccctgcctgtcagtcttggagatcaagcctccatctcttgcagatctagtcagagcattgtacatactaatggaaacacctatttagaatggtacctgcagaaaccgggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatattccgtggacgttcggtggaggcaccaagctggaaatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 52 - protein, 502 aa, Artificial Sequence
  Synthetic
METDTLLLWVLLLWVPGSTGDATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKGGGGSGGGGSGGGGSTRDVLMTQTPLSLPVSLGDQASISCRSSQSIVHTNGNTYLEWYLQKPGQSPKLLIYKVSNRFSGVPDRFSGSGSGTDFTLKISRVEAEDLGVYYCFQGSHIPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - DNA, 2199 bp, Artificial Sequence
  Synthetic
atggagacagacacactcctgctatgggtactgctgctctgggttccaggttccactggtgacacgcgtcaggtccaactgcagcagcctggggctgagctggtgaggcctggggcttcagtgaacctgtcctgcagggcttctggctacaccttcaccacctactggatgatctgggtgaagcagaggcctggacaaggccttgaatggattggtatgattgatccttcagacagtgagactcactacaatcaaatgttcaaggacaaggccacattgactgtagacaaatcctccaccacagcctacatgcagttcagcagcctaacatctgaggactctgcggtctattactgtgcaagatggaacttcggtaagggctactggggccaaggcaccactctcacggtctcctcagcgtcgaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaagcggccgcaggtggtggcggttcaggcggaggtggctctggcggtggcggatccgccacccacgagcactccgcccagtggctgaacaactacaagaagggctacggctacggcccctaccccctgggcatcaacggcggcatgcactacggcgtggacttcttcatgaacatcggcacccccgtgaaggccatctcctccggcaagatcgtggaggccggctggtccaactacggcggcggcaaccagatcggcctgatcgagaacgacggcgtgcaccgccagtggtacatgcacctgtccaagtacaacgtgaaggtgggcgactacgtgaaggccggccagatcatcggctggtccggctccaccggctactccaccgccccccacctgcacttccagcgcatggtgaactccttctccaactccaccgcccaggaccccatgcccttcctgaagtccgccggctacggcaaggccggcggcaccgtgacccccacccccaacaccggctggaagaccaacaagtacggcaccctgtacaagtccgagtccgcctccttcacccccaacaccgacatcatcacccgcaccaccggccccttccgctccatgccccagtccggcgtgctgaaggccggccagaccatccactacgacgaggtgatgaagcaggacggccacgtgtgggtgggctacaccggcaactccggccagcgcatctacctgcccgtgcgcacctggaacaagtccaccaacaccctgggcgtgctgtggggcaccatcaagtga

SEQ ID NO: 54 - protein, 732 aa, Artificial Sequence
  Synthetic
METDTLLLWVLLLWVPGSTGDTRQVQLQQPGAELVRPGASVNLSCRASGYTFTTYWMIWVKQRPGQGLEWIGMIDPSDSETHYNQMFKDKATLTVDKSSTTAYMQFSSLTSEDSAVYYCARWNFGKGYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAAAGGGGSGGGGSGGGGSATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 55 - DNA, 1836 bp, Artificial Sequence
  Synthetic
atggagacagacacactcctgctatgggtactgctgctctgggttccaggttccactggtgacacgcgtcaggtccaactgcagcagcctggggctgagctggtgaggcctggggcttcagtgaacctgtcctgcagggcttctggctacaccttcaccacctactggatgatctgggtgaagcagaggcctggacaaggccttgaatggattggtatgattgatccttcagacagtgagactcactacaatcaaatgttcaaggacaaggccacattgactgtagacaaatcctccaccacagcctacatgcagttcagcagcctaacatctgaggactctgcggtctattactgtgcaagatggaacttcggtaagggctactggggccaaggcaccactctcacggtctcctcagcgtcgaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaagcggccgcaggtggtggcggttcaggcggaggtggctctggcggtggcggatcgaatttggtgaatttccacagaatgatcaagttgacgacaggaaaggaagccgcactcagttatggcttctacggctgccactgtggcgtgggtggcagaggatcccccaaggatgcaacggatcgctgctgtgtcactcatgactgttgctacaaacgtctggagaaacgtggatgtggcaccaaatttctgagctacaagtttagcaactcggggagcagaatcacctgtgcaaaacaggactcctgcagaagtcaactgtgtgagtgtgataaggctgctgccacctgttttgctagaaacaagacgacctacaataaaaagtaccagtactattccaataaacactgcagagggagcacccctcgttgctga

SEQ ID NO: 56 - protein, 611 aa, Artificial Sequence
  Synthetic
METDTLLLWVLLLWVPGSTGDTRQVQLQQPGAELVRPGASVNLSCRASGYTFTTYWMIWVKQRPGQGLEWIGMIDPSDSETHYNQMFKDKATLTVDKSSTTAYMQFSSLTSEDSAVYYCARWNFGKGYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAAAGGGGSGGGGSGGGGSNLVNFHRMIKLTTGKEAALSYGFYGCHCGVGGRGSPKDATDRCCVTHDCCYKRLEKRGCGTKFLSYKFSNSGSRITCAKQDSCRSQLCECDKAAATCFARNKTTYNKKYQYYSNKHCRGSTPRC

SEQ ID NO: 57 - DNA, 1836 bp, Artificial Sequence; identical to SEQ ID NO: 55
  Synthetic

SEQ ID NO: 58 - protein, 611 aa, Artificial Sequence; identical to SEQ ID NO: 56
  Synthetic

SEQ ID NO: 59 - DNA, 1494 bp, Artificial Sequence
  Synthetic
atggagacagacacactcctgctatgggtactgctgctctgggttccaggttccactggtgacgccacccacgagcactccgcccagtggctgaacaactacaagaagggctacggctacggcccctaccccctgggcatcaacggcggcatgcactacggcgtggacttcttcatgaacatcggcacccccgtgaaggccatctcctccggcaagatcgtggaggccggctggtccaactacggcggcggcaaccagatcggcctgatcgagaacgacggcgtgcaccgccagtggtacatgcacctgtccaagtacaacgtgaaggtgggcgactacgtgaaggccggccagatcatcggctggtccggctccaccggctactccaccgccccccacctgcacttccagcgcatggtgaactccttctccaactccaccgcccaggaccccatgcccttcctgaagtccgccggctacggcaaggccggcggcaccgtgacccccacccccaacaccggctggaagaccaacaagtacggcaccctgtacaagtccgagtccgcctccttcacccccaacaccgacatcatcacccgcaccaccggccccttccgctccatgccccagtccggcgtgctgaaggccggccagaccatccactacgacgaggtgatgaagcaggacggccacgtgtgggtgggctacaccggcaactccggccagcgcatctacctgcccgtgcgcacctggaacaagtccaccaacaccctgggcgtgctgtggggcaccatcaagggtggtggcggttcaggcggaggtggctctggcggtggcggatccacgcgtgacattgtgatgacccagtctcaaaaattcatgtccacatcagtaggagacagggtcagcgtcacctgcaaggccagtcagaatgtgggtactaatgtagcctggtatcaacagaaaccagggcaatctcctaaagcactgatttactcggcatcctaccggtacagtggagtccctgatcgcttcacaggcagtggatctgggacagatttcactctcaccatcagcaatgtgcagtctgaagacttggcagagtatttctgtcagcaatataacagctatcctctcacgttcggtgctgggaccaagctggagctgaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 60 - protein, 497 aa, Artificial Sequence
  Synthetic
METDTLLLWVLLLWVPGSTGDATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKGGGGSGGGGSGGGGSTRDIVMTQSQKFMSTSVGDRVSVTCKASQNVGTNVAWYQQKPGQSPKALIYSASYRYSGVPDRFTGSGSGTDFTLTISNVQSEDLAEYFCQQYNSYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - DNA, 1590 bp, Artificial Sequence
  Synthetic
atggagacagacacactcctgctatgggtactgctgctctgggttccaggttccactggtgacacgcgtgaagtgaagctggtggagtctgggggaggtttagtgcagcctggagggtccctgaaactctcctgtgcagcctctggattcactttcagtagctataccatgtcttgggttcgccagactccagagaagaggctggagtgggtcgcatacattagtaatggtggtggtagcacctactatccagacactgtaaagggccgattcaccatctccagagacaatgccaagaacaccctgtacctgcaaatgagcagtctgaagtctgaggacacggccatgtattactgtgcaagacaggtacgacgggggatggactactggggtcaaggaacctcagtcaccgtctcctcagcgtcgaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaagcggccgcaggtggtggcggttcaggcggaggtggctctggcggtggcggatccggtataggcgatcctgttacctgccttaagagtggagccatatgtcatccagtcttttgccctagaaggtataaacaaattggcacctgtggtctccctggaacaaaatgctgcaaaaagccatga

SEQ ID NO: 62 - protein, 529 aa, Artificial Sequence
  Synthetic
METDTLLLWVLLLWVPGSTGDTREVKLVESGGGLVQPGGSLKLSCAASGFTFSSYTMSWVRQTPEKRLEWVAYISNGGGSTYYPDTVKGRFTISRDNAKNTLYLQMSSLKSEDTAMYYCARQVRRGMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAAAGGGGSGGGGSGGGGSGIGDPVTCLKSGAICHPVFCPRRYKQIGTCGLPGTKCCKKP

SEQ ID NO: 63 - DNA, 1602 bp, Artificial Sequence
  Synthetic
atggagacagacacactcctgctatgggtactgctgctctgggttccaggttccactggtgacacgcgtgaagtgaagctggtggagtctgggggaggtttagtgcagcctggagggtccctgaaactctcctgtgcagcctctggattcactttcagtagctataccatgtcttgggttcgccagactccagagaagaggctggagtgggtcgcatacattagtaatggtggtggtagcacctactatccagacactgtaaagggccgattcaccatctccagagacaatgccaagaacaccctgtacctgcaaatgagcagtctgaagtctgaggacacggccatgtattactgtgcaagacaggtacgacgggggatggactactggggtcaaggaacctcagtcaccgtctcctcagcgtcgaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaagcggccgcaggtggtggcggttcaggcggaggtggctctggcggtggcggatccggaatcataaacacattacagaaatattattgcagagtcagaggcggccggtgtgctgtgctcagctgccttccaaaggaggaacagatcggcaagtgctcgacgcgtggccgaaaatgctgccgaagaaagaaataa

SEQ ID NO: 64 - protein, 533 aa, Artificial Sequence
  Synthetic
METDTLLLWVLLLWVPGSTGDTREVKLVESGGGLVQPGGSLKLSCAASGFTFSSYTMSWVRQTPEKRLEWVAYISNGGGSTYYPDTVKGRFTISRDNAKNTLYLQMSSLKSEDTAMYYCARQVRRGMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAAAGGGGSGGGGSGGGGSGIINTLQKYYCRVRGGRCAVLSCLPKEEQIGKCSTRGRKCCRRKK

SEQ ID NO: 65 - DNA, 1578 bp, Artificial Sequence
  Synthetic
atggagacagacacactcctgctatgggtactgctgctctgggttccaggttccactggtgacacgcgtgaagtgaagctggtggagtctgggggaggtttagtgcagcctggagggtccctgaaactctcctgtgcagcctctggattcactttcagtagctataccatgtcttgggttcgccagactccagagaagaggctggagtgggtcgcatacattagtaatggtggtggtagcacctactatccagacactgtaaagggccgattcaccatctccagagacaatgccaagaacaccctgtacctgcaaatgagcagtctgaagtctgaggacacggccatgtattactgtgcaagacaggtacgacgggggatggactactggggtcaaggaacctcagtcaccgtctcctcagcgtcgaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaagcggccgcaggtggtggcggttcaggcggaggtggctctggcggtggcggatccctgctgggggatttcttccggaagtctaaagagaagattgggaaagagtttaaaagaattgtccagagaatcaaggattttttgcggaatcttgtgcccaggacagaatcctag

SEQ ID NO: 66 - protein, 525 aa, Artificial Sequence
  Synthetic
METDTLLLWVLLLWVPGSTGDTREVKLVESGGGLVQPGGSLKLSCAASGFTFSSYTMSWVRQTPEKRLEWVAYISNGGGSTYYPDTVKGRFTISRDNAKNTLYLQMSSLKSEDTAMYYCARQVRRGMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAAAGGGGSGGGGSGGGGSLLGDFFRKSKEKIGKEFKRIVQRIKDFLRNLVPRTES

SEQ ID NO: 67 - DNA, 1839 bp, Artificial Sequence
  Synthetic
atggagacagacacactcctgctatgggtactgctgctctgggttccaggttccactggtgacacgcgtgaagtgaagctggtggagtctgggggaggtttagtgcagcctggagggtccctgaaactctcctgtgcagcctctggattcactttcagtagctataccatgtcttgggttcgccagactccagagaagaggctggagtgggtcgcatacattagtaatggtggtggtagcacctactatccagacactgtaaagggccgattcaccatctccagagacaatgccaagaacaccctgtacctgcaaatgagcagtctgaagtctgaggacacggccatgtattactgtgcaagacaggtacgacgggggatggactactggggtcaaggaacctcagtcaccgtctcctcagcgtcgaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaagcggccgcaggtggtggcggttcaggcggaggtggctctggcggtggcggatcgaatttggtgaatttccacagaatgatcaagttgacgacaggaaaggaagccgcactcagttatggcttctacggctgccactgtggcgtgggtggcagaggatcccccaaggatgcaacggatcgctgctgtgtcactcatgactgttgctacaaacgtctggagaaacgtggatgtggcaccaaatttctgagctacaagtttagcaactcggggagcagaatcacctgtgcaaaacaggactcctgcagaagtcaactgtgtgagtgtgataaggctgctgccacctgttttgctagaaacaagacgacctacaataaaaagtaccagtactattccaataaacactgcagagggagcacccctcgttgctga

SEQ ID NO: 68 - protein, 612 aa, Artificial Sequence
  Synthetic
METDTLLLWVLLLWVPGSTGDTREVKLVESGGGLVQPGGSLKLSCAASGFTFSSYTMSWVRQTPEKRLEWVAYISNGGGSTYYPDTVKGRFTISRDNAKNTLYLQMSSLKSEDTAMYYCARQVRRGMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAAAGGGGSGGGGSGGGGSNLVNFHRMIKLTTGKEAALSYGFYGCHCGVGGRGSPKDATDRCCVTHDCCYKRLEKRGCGTKFLSYKFSNSGSRITCAKQDSCRSQLCECDKAAATCFARNKTTYNKKYQYYSNKHCRGSTPRC

SEQ ID NO: 69 - DNA, 1512 bp, Artificial Sequence
  Synthetic
atggagacagacacactcctgctatgggtactgctgctctgggttccaggttccactggtgacgccacccacgagcactccgcccagtggctgaacaactacaagaagggctacggctacggcccctaccccctgggcatcaacggcggcatgcactacggcgtggacttcttcatgaacatcggcacccccgtgaaggccatctcctccggcaagatcgtggaggccggctggtccaactacggcggcggcaaccagatcggcctgatcgagaacgacggcgtgcaccgccagtggtacatgcacctgtccaagtacaacgtgaaggtgggcgactacgtgaaggccggccagatcatcggctggtccggctccaccggctactccaccgccccccacctgcacttccagcgcatggtgaactccttctccaactccaccgcccaggaccccatgcccttcctgaagtccgccggctacggcaaggccggcggcaccgtgacccccacccccaacaccggctggaagaccaacaagtacggcaccctgtacaagtccgagtccgcctccttcacccccaacaccgacatcatcacccgcaccaccggccccttccgctccatgccccagtccggcgtgctgaaggccggccagaccatccactacgacgaggtgatgaagcaggacggccacgtgtgggtgggctacaccggcaactccggccagcgcatctacctgcccgtgcgcacctggaacaagtccaccaacaccctgggcgtgctgtggggcaccatcaagggtggtggcggttcaggcggaggtggctctggcggtggcggatccacgcgtgacattgtgatgacacagtctccatcctccctgactgtgacagcaggagagaaggtcactatgagctgcaagtccagtcagagtctgttaaacagtggaaatcaaaagaactacttgacctggtaccagcagaaaccagggcagcctcctaaactgttgatctactgggcatccactagggaatctggggtccctgatcgcttcacaggcagtggatctggaacagatttcactctcaccatcagcagtgtgcaggctgaagacctggcagtttattactgtcagaatgattatagttatcctttcacgttcggctcggggacaaagttggaaataaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 70 - protein, 503 aa, Artificial Sequence
  Synthetic
METDTLLLWVLLLWVPGSTGDATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKGGGGSGGGGSGGGGSTRDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - DNA, 1590 bp, Artificial Sequence
  Synthetic
atggagacagacacactcctgctatgggtactgctgctctgggttccaggttccactggtgacacgcgtgaggtccagctgcagcagtctggacctgagctagtgaagactggggcttcagtgaagatatcctgcaaggcttctggttactcattcactggttactacatgcactgggtcaagcagagccatggaaagagccttgagtggattggatatattagttgttacaatggtgctactagctacaaccagaagttcaagggcaaggccacatttactgtagacacatcctccagcacagcctacatgcagttcaacagcctgacatctgaagactctgcggtctattactgtgcaagatcgaggactggagcctggtttgcttactggggccaagggactctggtcactgtctctgcgtcgaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaagcggccgcaggtggtggcggttcaggcggaggtggctctggcggtggcggatccggtataggcgatcctgttacctgccttaagagtggagccatatgtcatccagtcttttgccctagaaggtataaacaaattggcacctgtggtctccctggaacaaaatgctgcaaaaagccatga

SEQ ID NO: 72 - protein, 529 aa, Artificial Sequence
  Synthetic
METDTLLLWVLLLWVPGSTGDTREVQLQQSGPELVKTGASVKISCKASGYSFTGYYMHWVKQSHGKSLEWIGYISCYNGATSYNQKFKGKATFTVDTSSSTAYMQFNSLTSEDSAVYYCARSRTGAWFAYWGQGTLVTVSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAAAGGGGSGGGGSGGGGSGIGDPVTCLKSGAICHPVFCPRRYKQIGTCGLPGTKCCKKP

SEQ ID NO: 73 - DNA, 1602 bp, Artificial Sequence
  Synthetic
atggagacagacacactcctgctatgggtactgctgctctgggttccaggttccactggtgacacgcgtgaggtccagctgcagcagtctggacctgagctagtgaagactggggcttcagtgaagatatcctgcaaggcttctggttactcattcactggttactacatgcactgggtcaagcagagccatggaaagagccttgagtggattggatatattagttgttacaatggtgctactagctacaaccagaagttcaagggcaaggccacatttactgtagacacatcctccagcacagcctacatgcagttcaacagcctgacatctgaagactctgcggtctattactgtgcaagatcgaggactggagcctggtttgcttactggggccaagggactctggtcactgtctctgcgtcgaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaagcggccgcaggtggtggcggttcaggcggaggtggctctggcggtggcggatccggaatcataaacacattacagaaatattattgcagagtcagaggcggccggtgtgctgtgctcagctgccttccaaaggaggaacagatcggcaagtgctcgacgcgtggccgaaaatgctgccgaagaaagaaataa

SEQ ID NO: 74 - protein, 533 aa, Artificial Sequence
  Synthetic
METDTLLLWVLLLWVPGSTGDTREVQLQQSGPELVKTGASVKISCKASGYSFTGYYMHWVKQSHGKSLEWIGYISCYNGATSYNQKFKGKATFTVDTSSSTAYMQFNSLTSEDSAVYYCARSRTGAWFAYWGQGTLVTVSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAAAGGGGSGGGGSGGGGSGIINTLQKYYCRVRGGRCAVLSCLPKEEQIGKCSTRGRKCCRRKK

SEQ ID NO: 75 - DNA, 1578 bp, Artificial Sequence
  Synthetic
atggagacagacacactcctgctatgggtactgctgctctgggttccaggttccactggtgacacgcgtgaggtccagctgcagcagtctggacctgagctagtgaagactggggcttcagtgaagatatcctgcaaggcttctggttactcattcactggttactacatgcactgggtcaagcagagccatggaaagagccttgagtggattggatatattagttgttacaatggtgctactagctacaaccagaagttcaagggcaaggccacatttactgtagacacatcctccagcacagcctacatgcagttcaacagcctgacatctgaagactctgcggtctattactgtgcaagatcgaggactggagcctggtttgcttactggggccaagggactctggtcactgtctctgcgtcgaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaagcggccgcaggtggtggcggttcaggcggaggtggctctggcggtggcggatccctgctgggggatttcttccggaagtctaaagagaagattgggaaagagtttaaaagaattgtccagagaatcaaggattttttgcggaatcttgtgcccaggacagaatcctag

SEQ ID NO: 76 - protein, 525 aa, Artificial Sequence
  Synthetic
METDTLLLWVLLLWVPGSTGDTREVQLQQSGPELVKTGASVKISCKASGYSFTGYYMHWVKQSHGKSLEWIGYISCYNGATSYNQKFKGKATFTVDTSSSTAYMQFNSLTSEDSAVYYCARSRTGAWFAYWGQGTLVTVSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAAAGGGGSGGGGSGGGGSLLGDFFRKSKEKIGKEFKRIVQRIKDFLRNLVPRTES

SEQ ID NO: 77 - DNA, 1839 bp, Artificial Sequence
  Synthetic
atggagacagacacactcctgctatgggtactgctgctctgggttccaggttccactggtgacacgcgtgaggtccagctgcagcagtctggacctgagctagtgaagactggggcttcagtgaagatatcctgcaaggcttctggttactcattcactggttactacatgcactgggtcaagcagagccatggaaagagccttgagtggattggatatattagttgttacaatggtgctactagctacaaccagaagttcaagggcaaggccacatttactgtagacacatcctccagcacagcctacatgcagttcaacagcctgacatctgaagactctgcggtctattactgtgcaagatcgaggactggagcctggtttgcttactggggccaagggactctggtcactgtctctgcgtcgaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaagcggccgcaggtggtggcggttcaggcggaggtggctctggcggtggcggatcgaatttggtgaatttccacagaatgatcaagttgacgacaggaaaggaagccgcactcagttatggcttctacggctgccactgtggcgtgggtggcagaggatcccccaaggatgcaacggatcgctgctgtgtcactcatgactgttgctacaaacgtctggagaaacgtggatgtggcaccaaatttctgagctacaagtttagcaactcggggagcagaatcacctgtgcaaaacaggactcctgcagaagtcaactgtgtgagtgtgataaggctgctgccacctgttttgctagaaacaagacgacctacaataaaaagtaccagtactattccaataaacactgcagagggagcacccctcgttgctga

SEQ ID NO: 78 - protein, 612 aa, Artificial Sequence
  Synthetic
METDTLLLWVLLLWVPGSTGDTREVQLQQSGPELVKTGASVKISCKASGYSFTGYYMHWVKQSHGKSLEWIGYISCYNGATSYNQKFKGKATFTVDTSSSTAYMQFNSLTSEDSAVYYCARSRTGAWFAYWGQGTLVTVSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAAAGGGGSGGGGSGGGGSNLVNFHRMIKLTTGKEAALSYGFYGCHCGVGGRGSPKDATDRCCVTHDCCYKRLEKRGCGTKFLSYKFSNSGSRITCAKQDSCRSQLCECDKAAATCFARNKTTYNKKYQYYSNKHCRGSTPRC

SEQ ID NO: 79 - DNA, 1494 bp, Artificial Sequence
  Synthetic
atggagacagacacactcctgctatgggtactgctgctctgggttccaggttccactggtgacgccacccacgagcactccgcccagtggctgaacaactacaagaagggctacggctacggcccctaccccctgggcatcaacggcggcatgcactacggcgtggacttcttcatgaacatcggcacccccgtgaaggccatctcctccggcaagatcgtggaggccggctggtccaactacggcggcggcaaccagatcggcctgatcgagaacgacggcgtgcaccgccagtggtacatgcacctgtccaagtacaacgtgaaggtgggcgactacgtgaaggccggccagatcatcggctggtccggctccaccggctactccaccgccccccacctgcacttccagcgcatggtgaactccttctccaactccaccgcccaggaccccatgcccttcctgaagtccgccggctacggcaaggccggcggcaccgtgacccccacccccaacaccggctggaagaccaacaagtacggcaccctgtacaagtccgagtccgcctccttcacccccaacaccgacatcatcacccgcaccaccggccccttccgctccatgccccagtccggcgtgctgaaggccggccagaccatccactacgacgaggtgatgaagcaggacggccacgtgtgggtgggctacaccggcaactccggccagcgcatctacctgcccgtgcgcacctggaacaagtccaccaacaccctgggcgtgctgtggggcaccatcaagggtggtggcggttcaggcggaggtggctctggcggtggcggatccacgcgtgacatccagatgactcagtctccagcctccctatctgcatctgtgggagaaactgtcaccatcacatgtcgagcaagtgggaatattcacaattatttagcatggtatcagcagaaacagggaaaatctcctcagctcctggtctataatgcaaaaaccttagcagatggtgtgccatcaaggttcagtggcagtggatcaggaacacaatattctctcaagatcaacagcctgcagcctgaagattttgggagttattactgtcaacatttttggagtactccgtggacgttcggtggaggcaccaagctggaaatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 80 - protein, 497 aa, Artificial Sequence
  Synthetic
METDTLLLWVLLLWVPGSTGDATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKGGGGSGGGGSGGGGSTRDIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - DNA, 1602 bp, Artificial Sequence
  Synthetic
atggagacagacacactcctgctatgggtactgctgctctgggttccaggttccactggtgacacgcgtcaggtccagctgcagcagtctggagctgagctggtaaggcctgggacttcagtgaaggtgtcctgcaaggcttctggatacgccttcactaattacttgatagagtgggtaaagcagaggcctggacagggccttgagtggattggagtgattaatcctggaagtggtggtactaactacaatgagaagttcaagggcaaggcaacactgactgcagacaaatcctccagcactgcctacatgcagctcagcagcctgacatctgatgactctgcggtctatttctgtgcaagatgggactacggtagtagctacgaacgtgctatggactactggggtcaaggaacctcagtcaccgtctccgcgtcgaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaagcggccgcaggtggtggcggttcaggcggaggtggctctggcggtggcggatccggtataggcgatcctgttacctgccttaagagtggagccatatgtcatccagtcttttgccctagaaggtataaacaaattggcacctgtggtctccctggaacaaaatgctgcaaaaagccatga

SEQ ID NO: 82 - protein, 533 aa, Artificial Sequence
  Synthetic
METDTLLLWVLLLWVPGSTGDTRQVQLQQSGAELVRPGTSVKVSCKASGYAFTNYLIEWVKQRPGQGLEWIGVINPGSGGTNYNEKFKGKATLTADKSSSTAYMQLSSLTSDDSAVYFCARWDYGSSYERAMDYWGQGTSVTVSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAAAGGGGSGGGGSGGGGSGIGDPVTCLKSGAICHPVFCPRRYKQIGTCGLPGTKCCKKP

SEQ ID NO: 83 - DNA, 1614 bp, Artificial Sequence
  Synthetic
atggagacagacacactcctgctatgggtactgctgctctgggttccaggttccactggtgacacgcgtcaggtccagctgcagcagtctggagctgagctggtaaggcctgggacttcagtgaaggtgtcctgcaaggcttctggatacgccttcactaattacttgatagagtgggtaaagcagaggcctggacagggccttgagtggattggagtgattaatcctggaagtggtggtactaactacaatgagaagttcaagggcaaggcaacactgactgcagacaaatcctccagcactgcctacatgcagctcagcagcctgacatctgatgactctgcggtctatttctgtgcaagatgggactacggtagtagctacgaacgtgctatggactactggggtcaaggaacctcagtcaccgtctccgcgtcgaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaagcggccgcaggtggtggcggttcaggcggaggtggctctggcggtggcggatccggaatcataaacacattacagaaatattattgcagagtcagaggcggccggtgtgctgtgctcagctgccttccaaaggaggaacagatcggcaagtgctcgacgcgtggccgaaaatgctgccgaagaaagaaataa

SEQ ID NO: 84 - protein, 537 aa, Artificial Sequence
  Synthetic
METDTLLLWVLLLWVPGSTGDTRQVQLQQSGAELVRPGTSVKVSCKASGYAFTNYLIEWVKQRPGQGLEWIGVINPGSGGTNYNEKFKGKATLTADKSSSTAYMQLSSLTSDDSAVYFCARWDYGSSYERAMDYWGQGTSVTVSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAAAGGGGSGGGGSGGGGSGIINTLQKYYCRVRGGRCAVLSCLPKEEQIGKCSTRGRKCCRRKK

SEQ ID NO: 85 - DNA, 1590 bp, Artificial Sequence
  Synthetic
atggagacagacacactcctgctatgggtactgctgctctgggttccaggttccactggtgacacgcgtcaggtccagctgcagcagtctggagctgagctggtaaggcctgggacttcagtgaaggtgtcctgcaaggcttctggatacgccttcactaattacttgatagagtgggtaaagcagaggcctggacagggccttgagtggattggagtgattaatcctggaagtggtggtactaactacaatgagaagttcaagggcaaggcaacactgactgcagacaaatcctccagcactgcctacatgcagctcagcagcctgacatctgatgactctgcggtctatttctgtgcaagatgggactacggtagtagctacgaacgtgctatggactactggggtcaaggaacctcagtcaccgtctccgcgtcgaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaagcggccgcaggtggtggcggttcaggcggaggtggctctggcggtggcggatccctgctgggggatttcttccggaagtctaaagagaagattgggaaagagtttaaaagaattgtccagagaatcaaggattttttgcggaatcttgtgcccaggacagaatcctag

SEQ ID NO: 86 - protein, 529 aa, Artificial Sequence
  Synthetic
METDTLLLWVLLLWVPGSTGDTRQVQLQQSGAELVRPGTSVKVSCKASGYAFTNYLIEWVKQRPGQGLEWIGVINPGSGGTNYNEKFKGKATLTADKSSSTAYMQLSSLTSDDSAVYFCARWDYGSSYERAMDYWGQGTSVTVSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAAAGGGGSGGGGSGGGGSLLGDFFRKSKEKIGKEFKRIVQRIKDFLRNLVPRTES

SEQ ID NO: 87 - DNA, 1851 bp, Artificial Sequence
  Synthetic
atggagacagacacactcctgctatgggtactgctgctctgggttccaggttccactggtgacacgcgtcaggtccagctgcagcagtctggagctgagctggtaaggcctgggacttcagtgaaggtgtcctgcaaggcttctggatacgccttcactaattacttgatagagtgggtaaagcagaggcctggacagggccttgagtggattggagtgattaatcctggaagtggtggtactaactacaatgagaagttcaagggcaaggcaacactgactgcagacaaatcctccagcactgcctacatgcagctcagcagcctgacatctgatgactctgcggtctatttctgtgcaagatgggactacggtagtagctacgaacgtgctatggactactggggtcaaggaacctcagtcaccgtctccgcgtcgaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaagcggccgcaggtggtggcggttcaggcggaggtggctctggcggtggcggatcgaatttggtgaatttccacagaatgatcaagttgacgacaggaaaggaagccgcactcagttatggcttctacggctgccactgtggcgtgggtggcagaggatcccccaaggatgcaacggatcgctgctgtgtcactcatgactgttgctacaaacgtctggagaaacgtggatgtggcaccaaatttctgagctacaagtttagcaactcggggagcagaatcacctgtgcaaaacaggactcctgcagaagtcaactgtgtgagtgtgataaggctgctgccacctgttttgctagaaacaagacgacctacaataaaaagtaccagtactattccaataaacactgcagagggagcacccctcgttgctga

SEQ ID NO: 88 - protein, 616 aa, Artificial Sequence
  Synthetic
METDTLLLWVLLLWVPGSTGDTRQVQLQQSGAELVRPGTSVKVSCKASGYAFTNYLIEWVKQRPGQGLEWIGVINPGSGGTNYNEKFKGKATLTADKSSSTAYMQLSSLTSDDSAVYFCARWDYGSSYERAMDYWGQGTSVTVSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAAAGGGGSGGGGSGGGGSNLVNFHRMIKLTTGKEAALSYGFYGCHCGVGGRGSPKDATDRCCVTHDCCYKRLEKRGCGTKFLSYKFSNSGSRITCAKQDSCRSQLCECDKAAATCFARNKTTYNKKYQYYSNKHCRGSTPRC

SEQ ID NO: 89 - DNA, 999 bp, Artificial Sequence
  Synthetic
atggagacagacacactcctgctatgggtactgctgctctgggttccaggttccactggtgaccatcaccatcaccatcacacgcgtcaggtccagctgcagcagtctggagctgagctggtaaggcctgggacttcagtgaaggtgtcctgcaaggcttctggatacgccttcactaattacttgatagagtgggtaaagcagaggcctggacagggccttgagtggattggagtgattaatcctggaagtggtggtactaactacaatgagaagttcaagggcaaggcaacactgactgcagacaaatcctccagcactgcctacatgcagctcagcagcctgacatctgatgactctgcggtctatttctgtgcaagatgggactacggtagtagctacgaacgtgctatggactactggggtcaaggaacctcagtcaccgtctccgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcggccgcaggtggtggcggttcaggcggaggtggctctggcggtggcggatccggaatcataaacacattacagaaatattattgcagagtcagaggcggccggtgtgctgtgctcagctgccttccaaaggaggaacagatcggcaagtgctcgacccgtggccgaaaatgctgccgaagaaagaaacatcaccatcaccatcactaa

SEQ ID NO: 90 - protein, 332 aa, Artificial Sequence
  Synthetic
METDTLLLWVLLLWVPGSTGDHHHHHHTRQVQLQQSGAELVRPGTSVKVSCKASGYAFTNYLIEWVKQRPGQGLEWIGVINPGSGGTNYNEKFKGKATLTADKSSSTAYMQLSSLTSDDSAVYFCARWDYGSSYERAMDYWGQGTSVTVSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAAAGGGGSGGGGSGGGGSGIINTLQKYYCRVRGGRCAVLSCLPKEEQIGKCSTRGRKCCRRKKHHHHHH

SEQ ID NO: 91 - DNA, 981 bp, Artificial Sequence
  Synthetic
atggagacagacacactcctgctatgggtactgctgctctgggttccaggttccactggtgaccatcaccatcaccatcacacgcgtcaggtccagctgcagcagtctggagctgagctggtaaggcctgggacttcagtgaaggtgtcctgcaaggcttctggatacgccttcactaattacttgatagagtgggtaaagcagaggcctggacagggccttgagtggattggagtgattaatcctggaagtggtggtactaactacaatgagaagttcaagggcaaggcaacactgactgcagacaaatcctccagcactgcctacatgcagctcagcagcctgacatctgatgactctgcggtctatttctgtgcaagatgggactacggtagtagctacgaacgtgctatggactactggggtcaaggaacctcagtcaccgtctccgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatccccaccgtccccagcggccgcaggtggtggcggttcaggcggaggtggctctggcggtggcggatccggaatcataaacacattacagaaatattattgcagagtcagaggcggccggtgtgctgtgctcagctgccttccaaaggaggaacagatcggcaagtgctcgacgcgtggccgaaaatgctgccgaagaaagaaataa

SEQ ID NO: 92 - protein, 326 aa, Artificial Sequence
  Synthetic
METDTLLLWVLLLWVPGSTGDHHHHHHTRQVQLQQSGAELVRPGTSVKVSCKASGYAFTNYLIEWVKQRPGQGLEWIGVINPGSGGTNYNEKFKGKATLTADKSSSTAYMQLSSLTSDDSAVYFCARWDYGSSYERAMDYWGQGTSVTVSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAAAGGGGSGGGGSGGGGSGIINTLQKYYCRVRGGRCAVLSCLPKEEQIGKCSTRGRKCCRRKK

SEQ ID NO: 93 - protein, 64 aa, Staphylococcus aureus
KDVVNRNQATDPHPTGSSLKPFLAYGPAIENMKWATNHAIQDESSYQVDGSTFRNYDTKSHGTV

SEQ ID NO: 94 - protein, 83 aa, Staphylococcus aureus
VKMTNAGQSVTYYFTDVKAPTVTVGNQTIEVGKTMNPIVLTTTDNGTGTVTNTVTGLPSGLSYDSATNSIIGTPTKIGQSTVT

SEQ ID NO: 95 - protein, 32 aa, Staphylococcus aureus
KMDTKKITERDKKDFWIQLHPKKAKAMMTKEQ

SEQ ID NO: 96 - protein, 48 aa, Staphylococcus aureus
TTETDENGKYRFDNLDSGKYKVIFEKPAGLTQTGTNTTEDDKDADGGE

SEQ ID NO: 97 - protein, 68 aa, Staphylococcus aureus
GLGTENGHGNYDVIEEIEENSHVDIKSELGYEGGQNSGNQSFEEDTEEDKPKYEQGGNIVDIDFDSVP

SEQ ID NO: 98 - protein, 34 aa, Staphylococcus aureus
AALPEEVRQERGITFGLFRLSVGLEDPDELIADI

SEQ ID NO: 99 - protein, 55 aa, Staphylococcus aureus
DGETTPITKTATYKVVRTVPKHVFETARGVLYPGVSDMYDAKQYVKPVNNSWSTN

SEQ ID NO: 100 - protein, 51 aa, Staphylococcus aureus
VVLSPILLITALLIKMESPGPAIFKQKRPTINNELFNIYKFRSMKIDTPNV

SEQ ID NO: 101 - protein, 65 aa, Staphylococcus aureus
KNGNNGGKSNSKKNRNVKRTIIKIIGFMIIAFFVVLLLGILLFAYYAWKAPAFTEAKLQDPIPAK

SEQ ID NO: 102 - protein, 41 aa, Staphylococcus aureus
LATEDNRFYEHGALDYKRLFGAIGKNLTGGFGSEGASTLTQ

SEQ ID NO: 103 - protein, 27 aa, Staphylococcus aureus
QEYSKDDIFQVYLNKIYYSDGVTGIKA

SEQ ID NO: 104 - protein, 60 aa, Staphylococcus aureus
NRNQATDPHPTGSSLKPFLAYGPAIENMKWATNHAIQDESSYQVDGSTFRNYDTKSHGTV

SEQ ID NO: 105 - protein, 30 aa, Staphylococcus aureus
ELSNTKLYPGQVWTIADLLQITVSNSSNAA

SEQ ID NO: 106 - protein, 19 aa, Staphylococcus aureus
VIELGLKTASTWKKFEVYE

SEQ ID NO: 107 - protein, 82 aa, Staphylococcus aureus
SEENSESVMDGFVEHPFYTATLNGQKYVVMKTKDDSYWKDLIVEGKRVTTVSKDPKNNSRTLIFPYIPDKAVYNAIVKVVVA

SEQ ID NO: 108 - protein, 43 aa, Staphylococcus aureus
EIVQNTINKRINFIFGVIVFIFAVLVLRLGYLQIAQGSHYKQI

SEQ ID NO: 109 - protein, 44 aa, Staphylococcus aureus
QNGEPRVNSTYIGYAPIDDPKLAFSIVYTNQPVPPPWLTGGDLG

SEQ ID NO: 110 - protein, 48 aa, Staphylococcus pseudintermedius
DTQDIADELKNRRATLVNLQRIDKVSAKRIIDFLSGTVYAIGGDIQRV

SEQ ID NO: 111 - protein, 76 aa, Staphylococcus aureus
IQDRKIKKVSKNKKRVDAQYKIKTNYGNIDRNVQFNFVKEDGMWKLDWDHSVIIPGMQKDQSIHIENLKSERGKIL

SEQ ID NO: 112 - protein, 101 aa, Staphylococcus aureus
AHTLIEKKKKDGKDIQLTIDAKVQKSIYNNMKNDYGSGTAIHPQTGELLALVSTPSYDVYPFMYGMSNEEYNKLTEDKKEPLLNKFQITTSPGSTQKILTA

SEQ ID NO: 113 - protein, 255 aa, Staphylococcus aureus
IHPQTGELLALVSTPSYDVYPFMYGMSNEEYNKLTEDKKEPLLNKFQITTSPGSTQKILTAMIGLNNKTLDDKTSYKIDGKGWQKDKSWGGYNVTRYEVVNGNIDLKQAIESSDNIFFARVALELGSKKFEKGMKKLGVGEDIPSDYPFYNAQISNKNLDNEILLADSGYGQGEILINPVQILSIYSALENNGNINAPHLLKDTKNKVWKKNIISKENINLLTDGMQQVVNKTHKEDIYRSYANLIGKSGTAELK

SEQ ID NO: 114 - protein, 71 aa, Staphylococcus aureus
NNKTLDDKTSYKIDGKGWQKDKSWGGYNVTRYEVVNGNIDLKQAIESSDNIFFARVALELGSKKFEKGMKK

SEQ ID NO: 115 - protein, 61 aa, Staphylococcus aureus
KLGVGEDIPSDYPFYNAQISNKNLDNEILLADSGYGQGEILINPVQILSIYSALENNGNIN

SEQ ID NO: 116 - protein, 56 aa, Staphylococcus aureus
IGKSGTAELKMKQGETGRQIGWFISYDKDNPNMMMAINVKDVQDKGMASYNAKISG

SEQ ID NO: 117 - protein, 25 aa, Staphylococcus aureus
LILIVVVVGFGIYFYASKDKEINNT

SEQ ID NO: 118 - protein, 31 aa, Staphylococcus aureus
VSTPSYDVYPFMYGMSNEEYNKLTEDKKEPL

SEQ ID NO: 119 - protein, 61 aa, Staphylococcus aureus
ETTNDDYWKDFMVEGQRVRTISKDAKNNTRTIIFPYVEGKTLYDAIVKVHVKTIDYDGQYH

SEQ ID NO: 120 - protein, 36 aa, Staphylococcus aureus
KQKIENKAAKQKKHPKVLILMGVPGSYLVATDKSYI

SEQ ID NO: 121 - protein, 43 aa, Staphylococcus aureus; identical to SEQ ID NO: 108

SEQ ID NO: 122 - protein, 44 aa, Staphylococcus aureus; identical to SEQ ID NO: 109

SEQ ID NO: 123 - protein, 22 aa, Staphylococcus aureus
FFNDGNMLKPWFVNSVENPVSK

SEQ ID NO: 124 - protein, 64 aa, Bos taurus
MRLHHLLLALLFLVLSAGSGFTQGVRNSQSCRRNKGICVPIRCPGSMRQIGTCLGAQVKCCRRK

SEQ ID NO: 125 - protein, 24 aa, Xenopus laevis
GVLSNVIGYLKKLGTGALNAVLKQ

SEQ ID NO: 126 - protein, 81 aa, Xenopus laevis
MYKGIFLCVLLAVICANSLATPSSDADEDNDEVERYVRGWASKIGQTLGKIAKVGLKELIQPKREAMLRSAEAQGKRPWIL

SEQ ID NO: 127 - protein, 23 aa, Xenopus laevis
GIGKFLHSAKKFGKAFVGEIMNS

SEQ ID NO: 128 - protein, 17 aa, Tachypleus gigas
KWCFRVCYRGICYRRCR

SEQ ID NO: 129 - protein, 17 aa, Tachypleus gigas
RWCFRVCYRGICYRKCR

SEQ ID NO: 130 - protein, 129 aa, Bufo bufo gagarizans
MSGRGKQGGKVRAKAKTRSSRAGLQFPVGRVHRLLRKGNYAQRVGAGAPVYLAAVLEYLTAEILELAGNAARDNKKTRIIPRHLQLAVRNDEELNKLLGGVTIAQGGVLPNIQAVLLPKTESSKPAKSK

SEQ ID NO: 131 - protein, 21 aa, Bufo bufo gagarizans
TRSSRAGLQFPVGRVHRLLRK

SEQ ID NO: 132 - protein, 63 aa, Bombyx mori
MNFVRILSFVFALVLALGAVSAAPEPRWKLFKKIEKVGRNVRDGLIKAGPAIAVIGQAKSLGK

SEQ ID NO: 133 - protein, 63 aa, Bombyx mori
MNFAKILSFVFALVLALSMTSAAPEPRWKIFKKIEKMGRNIRDGIVKAGPAIEVLGSAKAIGK

SEQ ID NO: 134 - protein, 63 aa, Drosophila melanogaster
MNFYKIFVFVALILAISIGQSEAGWLKKLGKRIERIGQHTRDATIQGLGIAQQAANVAATARG

SEQ ID NO: 135 - protein, 31 aa, Sus scrofa
SWLSKTAKKLENSAKKRISEGIAIAIQGGPR

SEQ ID NO: 136 - protein, 13 aa, Bos taurus
ILPWKWPWWPWRR

SEQ ID NO: 137 - protein, 34 aa, Lactococcus lactis
ITSISLCTPGCKTGALMGCNMKTATCHCSIHVSK

SEQ ID NO: 138 - protein, 20 aa, Rana catesbeiana
FLGGLIKIVPAMICAVTKKC

SEQ ID NO: 139 - protein, 25 aa, Bos taurus
FKCRRWQWRMKKLGAPSITCVRRAF

SEQ ID NO: 140 - protein, 19 aa, Sus scrofa
  misc_feature (19)..(19): Xaa can be any naturally occurring amino acid
RGGRLCYCRRRFCVCVGRX

SEQ ID NO: 141 - protein, 16 aa, Sus scrofa
GGRLCYCRRRFCICVG

SEQ ID NO: 142 - protein, 51 aa, Homo sapiens
MKFFVFALILALMLSMTGADSHAKRHHGYKRKFHEKHHSHRGYRSNYLYDN

SEQ ID NO: 143 - protein, 38 aa, Macaca fascicularis
DSHEERHHGRHGHHKYGRKFHEKHHSHRGYRSNYLYDN

SEQ ID NO: 144 - protein, 33 aa, Phyllomedusa sauvagei
ALWKTMLKKLGTMALHAGKAALGAAADTISQTQ

SEQ ID NO: 145 - protein, 34 aa, Phyllomedusa sauvagei
ALWFTMLKKLGTMALHAGKAALGAAANTISQGTQ

SEQ ID NO: 146 - protein, 30 aa, Phyllomedusa sauvagei
ALWKNMLKGIGKLAGKAALGAVKKLVGAES

SEQ ID NO: 147 - protein, 21 aa, Misgurnus anguillicaudatus
RQRVEELSKFSKKGAAARRRK

SEQ ID NO: 148 - protein, 27 aa, Apis mellifera
GIGAVLKVLTTGLPALISWISRKKRQQ

SEQ ID NO: 149 - protein, 33 aa, Pardachirus pavoninus
GFFALIPKIISSPLFKTLLSAVGSALSSSGEQE

SEQ ID NO: 150 - protein, 33 aa, Pardachirus pavoninus
GFFALIPKIISSPIFKTLLSAVGSALSSSGGQE

SEQ ID NO: 151 - protein, 176 aa, Bos taurus
METQRASLSLGRCSLWLLLLGLVLPSASAQALSYREAVLRAVDQFNERSSEANLYRLLELDPTPNDDLDPGTRKPVSFRVKETDCPRTSQQPLEQCDFKENGLVKQCVGTVTLDPSNDQFDINCNELQSVRFRPPIRRPPIRPPFYPPFRPPIRPPIFPPIRPPFRPPLGPFPGRR

SEQ ID NO: 152 - protein, 155 aa, Bos taurus
METPRASLSLGRWSLWLLLLGLALPSASAQALSYREAVLRAVDQLNEQSSEPNIYRLLELDQPPQDDEDPDSPKRVSFRVKETVCSRTTQQPPEQCDFKENGLLKRCEGTVTLDQVRGNFDITCNNHQSIRITKQPWAPPQAARLCRIVVIRVCR

SEQ ID NO: 153 - protein, 29 aa, Ceratitis capitata
SIGSALKKALPVAKKIGKIALPIAKAALP

SEQ ID NO: 154 - protein, 29 aa, Ceratitis capitata
SIGSAFKKALPVAKKIGKAALPIAKAALP

SEQ ID NO: 155 - protein, 170 aa, Homo sapiens
MKTQRNGHSLGRWSLVLLLLGLVMPLAIIAQVLSYKEAVLRAIDGINQRSSDANLYRLLDLDPRPTMDGDPDTPKPVSFTVKETVCPRTTQQSPEDCDFKKDGLVKRCMGTVTLNQARGSFDISCDKDNKRFALLGDFFRKSKEKIGKEFKRIVQRIKDFLRNLVPRTES

SEQ ID NO: 156 - protein, 170 aa, Equus caballus
METQRNTRCLGRWSPLLLLLGLVIPPATTQALSYKEAVLRAVDGLNQRSSDENLYRLLELDPLPKGDKDSDTPKPVSFMVKETVCPRIMKQTPEQCDFKENGLVKQCVGTVILDPVKDYFDASCDEPQRVKRFHSVGSLIQRHQQMIRDKSEATRHGIRIITRPKLLLAS

SEQ ID NO: 157 - protein, 159 aa, Bos taurus
METQRASLSLGRWSLWLLLLGLALPSASAQALSYREAVLRAVDQLNEKSSEANLYRLLELDPPPKEDDENPNIPKPVSFRVKETVCPRTSQQSPEQCDFKENGLLKECVGTVTLDQVGSNFDITCAVPQSVGGLRSLGRKILRAWKKYGPIIVPIIRIG

SEQ ID NO: 158 - protein, 156 aa, Equus caballus
METQRNTRCLGRWSPLLLLLGLVIPPATTQALSYKEAVLRAVDGLNQRSSDENLYRLLELDPLPKGDKDSDTPKPVSFMVKETVCPRIMKQTPEQCDFKENGLVKQCVGTVILGPVKDHFDVSCGEPQRVKRFGRLAKSFLRMRILLPRRKILLAS

SEQ ID NO: 159 - protein, 160 aa, Ovis aries
METQRASLSLGRCSLWLLLLGLALPSASAQVLSYREAVLRAADQLNEKSSEANLYRLLELDPPPKQDDENSNIPKPVSFRVKETVCPRTSQQPAEQCDFKENGLLKECVGTVTLDQVRNNFDITCAEPQSVRGLRRLGRKIAHGVKKYGPTVLRIIRIAG

SEQ ID NO: 160 - protein, 12 aa, Bos taurus
RLCRIVVIRVCR

SEQ ID NO: 161 - protein, 30 aa, Homo sapiens
ACYCRIPACIAGERRYGTCIYQGRLWAFCC

SEQ ID NO: 162 - protein, 29 aa, Homo sapiens
CYCRIPACIAGERRYGTCIYQGRLWAFCC

SEQ ID NO: 163 - protein, 30 aa, Homo sapiens
DCYCRIPACIAGERRYGTCIYQGRLWAFCC

SEQ ID NO: 164 - protein, 33 aa, Homo sapiens
VCSCRLVFCRRTELRVGNCLIGGVSFTYCCTRV

SEQ ID NO: 165 - protein, 33 aa, Oryctolagus cuniculus
VVCACRRALCLPRERRAGFCRIRGRIHPLCCRR

SEQ ID NO: 166 - protein, 33 aa, Oryctolagus cuniculus
VVCACRRALCLPLERRAGFCRIRGRIHPLCCRR

SEQ ID NO: 167 - protein, 34 aa, Oryctolagus cuniculus
GICACRRRFCPNSERFSGYCRVNGARYVRCCSRR

SEQ ID NO: 168 - protein, 34 aa, Oryctolagus cuniculus
GRCVCRKQLLCSYRERRIGDCKIRGVRFPFCCPR

SEQ ID NO: 169 - protein, 34 aa, Oryctolagus cuniculus
VSCTCRRFSCGFGERASGSCTVNGGVRHTLCCRR

SEQ ID NO: 170 - protein, 33 aa, Oryctolagus cuniculus
VFCTCRGFLCGSGERASGSCTINGVRHTLCCRR

SEQ ID NO: 171 - protein, 32 aa, Rattus norvegicus
VTCYCRRTRCGFRERLSGACGYRGRIYRLCCR

SEQ ID NO: 172 - protein, 30 aa, Rattus norvegicus
CSCRYSSCRFGERLLSGACRLNGRIYRLCC

SEQ ID NO: 173 - protein, 31 aa, Rattus norvegicus
ACTCRIGACVSGERLTGACGLNGRIYRLCCR

SEQ ID NO: 174 - protein, 31 aa, Cavia porcellus
RRCICTTRTCRFPYRRLGTCIFQNRVYTFCC

SEQ ID NO: 175 - protein, 45 aa, Homo sapiens
GIINTLQKYYCRVRGGRCAVLSCLPKEEQIGKCSTRGRKCCRRKK

SEQ ID NO: 176 - protein, 18 aa, Macaca mulatta
RCICTRGFCRCLCRRGVC

SEQ ID NO: 177 - protein, 78 aa, Helianthus annuus
MKSSMKMFAALLLVVMCLLANEMGGPLVVEARTCESQSHKFKGTCLSDTNCANVCHSERFSGGKCRGFRRRCFCTTHC

SEQ ID NO: 178 - protein, 78 aa, Helianthus annuus; identical to SEQ ID NO: 177

SEQ ID NO: 179 - protein, 30 aa, Macaca mulatta
ACYCRIPACLAGERRYGTCFYMGRVWAFCC

SEQ ID NO: 180 - protein, 37 aa, Androctonus australis hector
GFGCPFNQGACHRHCRSIRRRGGYCAGLFKQTCTCYR

SEQ ID NO: 181 - protein, 38 aa, Mytilus galloprovincialis
  misc_feature (28)..(28): Xaa can be any naturally occurring amino acid
GFGCPNNYQCHRHCKSIPGRCGGYCGGXHRLRCTCYRC

SEQ ID NO: 182 - protein, 54 aa, Heuchera sanguinea
DGVKLCDVPSGTWSGHCGSSSKCSQQCKDREHFAYGGACHYQFPSVKCFCKRQC

SEQ ID NO: 183 - protein, 49 aa, Clitoria ternatea
NLCERASLTWTGNCGNTGHCDTQCRNWESAKHGACHKRGNWKCFCYFNC

SEQ ID NO: 184 - protein, 91 aa, Mus musculus
MKKLVLLFALVLLAFQVQADSIQNTDEETKTEEQPGEKDQAVSVSFGDPQGSALQDAALGWGRRCPQCPRCPSCPSCPRCPRCPRCKCNPK

SEQ ID NO: 185 - protein, 40 aa, Bos taurus
QGVRNFVTCRINRGFCVPIRCPGHRRQIGTCLGPQIKCCR

SEQ ID NO: 186 - protein, 40 aa, Bos taurus
QGVRNFVTCRINRGFCVPIRCPGHRRQIGTCLGPRIKCCR

SEQ ID NO: 187 - protein, 42 aa, Bos taurus
QGVRNHVTCRIYGGFCVPIRCPGRTRQIGTCFGRPVKCCRRW

SEQ ID NO: 188 - protein, 40 aa, Bos taurus
QVVRNPQSCRWNMGVCIPISCPGNMRQIGTCFGPRVPCCR

SEQ ID NO: 189 - protein, 41 aa, Bos taurus
QRVRNPQSCRWNMGVCIPFLCRVGMRQIGTCFGPRVPCCRR

SEQ ID NO: 190 - protein, 42 aa, Bos taurus
QGVRNHVTCRINRGFCVPIRCPGRTRQIGTCFGPRIKCCRSW

SEQ ID NO: 191 - protein, 40 aa, Bos taurus
QGVRSYLSCWGNRGICLLNRCPGRMRQIGTCLAPRVKCCR

SEQ ID NO: 192 - protein, 42 aa, Bos taurus
SGISGPLSCGRNGGVCIPIRCPVPMRQIGTCFGRPVKCCRSW

SEQ ID NO: 193 - protein, 38 aa, Bos taurus
DFASCHTNGGICLPNRCPGHMIQIGICFRPRVKCCRSW

SEQ ID NO: 194 - protein, 74 aa, Zophobas atratus
SLQGGAPNFPQPSQQNGGWQVSPDLGRDDKGNTRGQIEIQNKGKDHDFNAGWGKVIRGPNKAKPTWHVGGTYRR

SEQ ID NO: 195 - protein, 45 aa, Homo sapiens; identical to SEQ ID NO: 175

SEQ ID NO: 196 - protein, 40 aa, Aedes aegypti
ATCDLLSGFGVGDSACAAHCIARGNRGGYCNSKKVCVCRN

SEQ ID NO: 197 - protein, 35 aa, Mytilus edulis
  misc_feature (28)..(28): Xaa can be any naturally occurring amino acid
GFGCPNDYPCHRHCKSIPGRYGGYCGGXHRLRCTC

SEQ ID NO: 198 - protein, 40 aa, Sarcophaga peregrina
ATCDLLSGIGVQHSACALHCVFRGNRGGYCTGKGICVCRN

SEQ ID NO: 199 - protein, 95 aa, Oryctolagus cuniculus
MRTLALLAAILLVALQAQAEHVSVSIDEVVDQQPPQAEDQDVAIYVKEHESSALEALGVKAGVVCACRRALCLPRERRAGFCRIRGRIHPLCCRR

SEQ ID NO: 200 - protein, 92 aa, Mus musculus
MKPLVLLSALVLLSFQVQADPIQNTDEETKTEEQSGEEDQAVSVSFGDREGASLQEESLRDLVCYCRTRGCKRRERMNGTCRKGHLMYTLCC

SEQ ID NO: 201 - protein, 93 aa, Mus musculus
MKTFVLLSALVLLAFQVQADPIHKTDEETNTEEQPGEEDQAVSISFGGQEGSALHEELSKKLICYCRIRGCKRRERVFGTCRNLFLTFVFCCS

SEQ ID NO: 202 - protein, 35 aa, Mus musculus
LRDLVCYCRARGCKGRERMNGTCRKGHLLYMLCCR

SEQ ID NO: 203 - protein, 43 aa, Pyrrhocoris apterus
ATCDILSFQSQWVTPNHAGCALHCVIKGYKGGQCKITVCHCRR

SEQ ID NO: 204 - protein, 32 aa, Rattus norvegicus
VTCYCRSTRCGFRERLSGACGYRGRIYRLCCR

SEQ ID NO: 205 - protein, 31 aa, Rattus norvegicus
VTCSCRTSSCRFGERLSGACRLNGRIYRLCC

SEQ ID NO: 206 - protein, 34 aa, Oryctolagus cuniculus
GICACRRRFCLNFEQFSGYCRVNGARYVRCCSRR

SEQ ID NO: 207 - protein, 64 aa, Pan troglodytes
MRVLYLLFSFLFIFLMPLPGVFGGISDPVTCLKSGAICHPVFCPRRYKQIGTCGLPGTKCCKKP

SEQ ID NO: 208 - protein, 41 aa, Homo sapiens
GIGDPVTCLKSGAICHPVFCPRRYKQIGTCGLPGTKCCKKP

SEQ ID NO: 209 - protein, 68 aa, Homo sapiens
MRTSYLLLFTLCLLLSEMASGGNFLTGLGHRSDHYNCVSSGGQCLYSACPIFTKIQGTCYRGKAKCCK

SEQ ID NO: 210 - protein, 64 aa, Capra hircus
MRLHHLLLVLFFLVLSAGSGFTQGIRSRRSCHRNKGVCALTRCPRNMRQIGTCFGPPVKCCRKK

SEQ ID NO: 211 - protein, 64 aa, Capra hircus
MRLHHLLLALFFLVLSAGSGFTQGIINHRSCYRNKGVCAPARCPRNMRQIGTCHGPPVKCCRKK

SEQ ID NO: 212 - protein, 23 aa, Xenopus laevis
GIGKFLHSAGKFGKAFVGEIMKS

SEQ ID NO: 213 - protein, 96 aa, Macaca mulatta
MRTLVILAAILLVALQAQAEPLQARTDEATAAQEQIPTDNPEVVVSLAWDESLAPKDSVPGLRKNMACYCRIPACLAGERRYGTCFYRRRVWAFCC

SEQ ID NO: 214 - protein, 96 aa, Macaca mulatta
MRTLVILAAILLVALQAQAEPLQARTDEATAAQEQIPTDNPEVVVSLAWDESLAPKDSVPGLRKNMACYCRIPACLAGERRYGTCFYLGRVWAFCC

SEQ ID NO: 215 - protein, 33 aa, Mesocricetus auratus
VTCFCRRRGCASRERHIGYCRFGNTIYRLCCRR

SEQ ID NO: 216 - protein, 31 aa, Mesocricetus auratus
CFCKRPVCDSGETQIGYCRLGNTFYRLCCRQ

SEQ ID NO: 217 - protein, 39 aa, Gallus gallus
GRKSDCFRKNGFCAFLKCPYLTLISGKCSRFHLCCKRIW

SEQ ID NO: 218 - protein, 43 aa, Allomyrina dichotoma
VTCDLLSFEAKGFAANHSLCAAHCLAIGRRGGSCERGVCICRR

SEQ ID NO: 219 - protein, 31 aa, Cavia porcellus; identical to SEQ ID NO: 174

SEQ ID NO: 220 - DNA, 117 bp, Artificial Sequence
  Synthetic
ggtggtggcggttcaggcggaggtggctctggcggtggcggatccggcatcggcaagttcctgcactccgccggcaagttcggcaaggccttcgtgggcgagatcatgaagtcctag

SEQ ID NO: 221 - protein, 38 aa, Artificial Sequence
  Synthetic
GGGGSGGGGSGGGGSGIGKFLHSAGKFGKAFVGEIMKS

SEQ ID NO: 222 - DNA, 117 bp, Artificial Sequence
  Synthetic
ggtggtggcggttcaggcggaggtggctctggcggtggcggatccggcatcggcaagttcctgcactccgccaagaagttcggcaaggccttcgtgggcgagatcatgaactcctag

SEQ ID NO: 223 - protein, 38 aa, Artificial Sequence
  Synthetic
GGGGSGGGGSGGGGSGIGKFLHSAKKFGKAFVGEIMNS

SEQ ID NO: 224 - protein, 68 aa, Staphylococcus aureus
MVDEPLHFQGGLTKRSYFNKNGHVTINDKQALMHSSNVYMFKTALKLAGDPYYSGMALPSDISSPAQK

SEQ ID NO: 225 - protein, 7 aa, Artificial Sequence
  Synthetic
KLAKKLA

SEQ ID NO: 226 - protein, 7 aa, Artificial Sequence
  Synthetic
KLAKLAK

SEQ ID NO: 227 - protein, 21 aa, Artificial Sequence
  Synthetic
KALKALKKALKALKKALKALK

SEQ ID NO: 228 - protein, 7 aa, Artificial Sequence
  Synthetic
KLGKKLG

SEQ ID NO: 229 - protein, 7 aa, Artificial Sequence
  Synthetic
KAAKKAA

SEQ ID NO: 230 - DNA, 1608 bp, Artificial Sequence
  Synthetic
atggagacagacacactcctgctatgggtactgctgctctgggttccaggttccactggtgacacgcgtcaggtccaactgcagcagtctgggcctgaggtggtgaggcctggggtctcagtgaagatttcctgcaagggttccggctacacattcactgattatgctatgcactgggtgaagcagagtcatgcaaagaggcaagagtggattggagttattggtacttacaatggtaatacaaactacaaccagaagtttaagggcaaggccacaatgactgtagacagatcctccagcacagcctatatggaacttgccggtttgacatctgaggattctgccatctattactgtgcaagaaggggtgattacgacgcctggtttgcttactggggccaagggactctggtcactgtctctgcagcgtcgaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaagcggccgcaggtggtggcggttcaggcggaggtggctctggcggtggcggatccggaatcataaacacattacagaaatattattgcagagtcagaggcggccggtgtgctgtgctcagctgccttccaaaggaggaacagatcggcaagtgctcgacgcgtggccgaaaatgctgccgaagaaagaaataa

SEQ ID NO: 231 - protein, 535 aa, Artificial Sequence
  Synthetic
METDTLLLWVLLLWVPGSTGDTRQVQLQQSGPEVVRPGVSVKISCKGSGYTFTDYAMHWVKQSHAKRQEWIGVIGTYNGNTNYNQKFKGKATMTVDRSSSTAYMELAGLTSEDSAIYYCARRGDYDAWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAAAGGGGSGGGGSGGGGSGIINTLQKYYCRVRGGRCAVLSCLPKEEQIGKCSTRGRKCCRRKK

SEQ ID NO: 232 - protein, 497 aa, Artificial Sequence
  Synthetic
METDTLLLWVLLLWVPGSTGDATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKGGGGSGGGGSGGGGSTRDIVMTQSQKFMSTPVGDRVSVTCKASQNVGTYVAWYQQKPGQSPKILIYSASYRYSGVPDRFTGSGSGTDFTLTISNVQSEDLAEYFCQRYNSYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 233 - DNA, 1596 bp, Artificial Sequence
  Synthetic
atggagacagacacactcctgctatgggtactgctgctctgggttccaggttccactggtgacacgcgtcagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacaccaacactggagagccaacatatgctgaagagttcaaggggcggtttgccttctctttggaaacctctgccagcactgcctatttgcagatcaacaacctcaaaaatgaggacacggctacatatttctgtgcaagacacggtggtaggagctggtacttcgatgtctggggcgcagggaccacggtcaccgtctcctcagcgtcgaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaagcggccgcaggtggtggcggttcaggcggaggtggctctggcggtggcggatccggtataggcgatcctgttacctgccttaagagtggagccatatgtcatccagtcttttgccctagaaggtataaacaaattggcacctgtggtctccctggaacaaaatgctgcaaaaagccatga

SEQ ID NO: 234 - protein, 531 aa, Artificial Sequence
  Synthetic
METDTLLLWVLLLWVPGSTGDTRQIQLVQSGPELKKPGETVKISCKASGYTFTNYGMNWVKQAPGKGLKWMGWINTNTGEPTYAEEFKGRFAFSLETSASTAYLQINNLKNEDTATYFCARHGGRSWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAAAGGGGSGGGGSGGGGSGIGDPVTCLKSGAICHPVFCPRRYKQIGTCGLPGTKCCKKP

SEQ ID NO: 235 - DNA, 1512 bp, Artificial Sequence
  Synthetic
atggagacagacacactcctgctatgggtactgctgctctgggttccaggttccactggtgacgccacccacgagcactccgcccagtggctgaacaactacaagaagggctacggctacggcccctaccccctgggcatcaacggcggcatgcactacggcgtggacttcttcatgaacatcggcacccccgtgaaggccatctcctccggcaagatcgtggaggccggctggtccaactacggcggcggcaaccagatcggcctgatcgagaacgacggcgtgcaccgccagtggtacatgcacctgtccaagtacaacgtgaaggtgggcgactacgtgaaggccggccagatcatcggctggtccggctccaccggctactccaccgccccccacctgcacttccagcgcatggtgaactccttctccaactccaccgcccaggaccccatgcccttcctgaagtccgccggctacggcaaggccggcggcaccgtgacccccacccccaacaccggctggaagaccaacaagtacggcaccctgtacaagtccgagtccgcctccttcacccccaacaccgacatcatcacccgcaccaccggccccttccgctccatgccccagtccggcgtgctgaaggccggccagaccatccactacgacgaggtgatgaagcaggacggccacgtgtgggtgggctacaccggcaactccggccagcgcatctacctgcccgtgcgcacctggaacaagtccaccaacaccctgggcgtgctgtggggcaccatcaagggtggtggcggttcaggcggaggtggctctggcggtggcggatccacgcgtgatgttgtgatgacccaaattccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttcctccgtggacgtttggtggaggcaccaagctggaaatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 236 - protein, 503 aa, Artificial Sequence
  Synthetic
METDTLLLWVLLLWVPGSTGDATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKGGGGSGGGGSGGGGSTRDVVMTQIPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 237 - DNA, 393 bp, Artificial Sequence
  Synthetic
atggagacagacacactcctgctatgggtactgctgctctgggttccaggttccactggtgacacgcgtgacatcttgctgactcagtctccagccatcctgtctgtgagtccaggagaaagagtcagtttctcctgcagggccagtcagagcattggcacaagcatacactggtatcagcaaagaacaaatggttctccaaggcttctcataaagtatgcttctgagtctatctctgggatcccttccaggtttagtggcagtggatcagggacagattttactcttagcatcaacagtgtggagtctgaagatattgcagattattactgtcaacaaagtaatagctggccaaccacgttcggctcggggacaaagttggaaataaaacgg

SEQ ID NO: 238 - protein, 131 aa, Artificial Sequence
  Synthetic
METDTLLLWVLLLWVPGSTGDTRDILLTQSPAILSVSPGERVSFSCRASQSIGTSIHWYQQRTNGSPRLLIKYASESISGIPSRFSGSGSGTDFTLSINSVESEDIADYYCQQSNSWPTTFGSGTKLEIKR

SEQ ID NO: 239 - DNA, 432 bp, Artificial Sequence
  Synthetic
atggagacagacacactcctgctatgggtactgctgctctgggttccaggttccactggtgacacgcgtgaggtccagctgcagcagtctggacctgagctggtaaagcctggggcttcagtgaagatgtcctgcaaggcttctggatacacattcactagctatgttatgcactgggtgaagcagaagcctgggcagggccttgagtggattggatatattaatccttacaatgatggtactaagtacaatgagaagttcaaaggcaaggccacactgacttcagacaaatcctccagcacagcctacatggagctcagcagcctgacctctgaggactctgcggtctattactgtgcaagaagtgagggggggatctactatgattacgatgttgcttactggggccaagggactctggtcactgtctct

SEQ ID NO: 240 - protein, 144 aa, Artificial Sequence
  Synthetic
METDTLLLWVLLLWVPGSTGDTREVQLQQSGPELVKPGASVKMSCKASGYTFTSYVMHWVKQKPGQGLEWIGYINPYNDGTKYNEKFKGKATLTSDKSSSTAYMELSSLTSEDSAVYYCARSEGGIYYDYDVAYWGQGTLVTVS

SEQ ID NO: 241 - DNA, 390 bp, Artificial Sequence
  Synthetic
atggagacagacacactcctgctatgggtactgctgctctgggttccaggttccactggtgacacgcgtcaaattgttctcacccagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactggcttctggagtccctgctcgcttcagtggcagtgggtctgggacctcttactctctcacaatcagcagcatggaggctgaagatgctgccacttattactgccagcagtggagtagtaacccaccgacgttcggtggaggcaccaagctggaaatcaaacgg

SEQ ID NO: 242 - protein, 130 aa, Artificial Sequence
  Synthetic
METDTLLLWVLLLWVPGSTGDTRQIVLTQSPAIMSASPGEKVTMTCSASSSVSYMHWYQQKSGTSPKRWIYDTSKLASGVPARFSGSGSGTSYSLTISSMEAEDAATYYCQQWSSNPPTFGGGTKLEIKR

SEQ ID NO: 243 - DNA, 435 bp, Artificial Sequence
  Synthetic
atggagacagacacactcctgctatgggtactgctgctctgggttccaggttccactggtgacacgcgtcaggtccaactgcagcaacctgggtctgagctggtgaggcctggagcttcagtgaagctgtcctgcaaggcttctggctacacattcaccagctactggatgcactgggtgaagcagaggcatggacaaggccttgagtggattggaaatatttatcctggtagtggtagtactaactacgatgagaagttcaagagcaagggcacactgactgtagacacatcctccagcacagcctacatgcacctcagcagcctgacatctgaggactctgcggtctattactgtacaagagggggatggttactactcggctactggtacttcgatgtctggggcgcagggaccacggtcaccgtctcctca

SEQ ID NO: 244 - protein, 145 aa, Artificial Sequence
  Synthetic
METDTLLLWVLLLWVPGSTGDTRQVQLQQPGSELVRPGASVKLSCKASGYTFTSYWMHWVKQRHGQGLEWIGNIYPGSGSTNYDEKFKSKGTLTVDTSSSTAYMHLSSLTSEDSAVYYCTRGGWLLLGYWYFDVWGAGTTVTVSS

SEQ ID NO: 245 - DNA, 390 bp, Artificial Sequence
  Synthetic
atggagacagacacactcctgctatgggtactgctgctctgggttccaggttccactggtgacacgcgtcaaattgttctcacccagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactggcttctggagtccctgctcgcttcagtggcagtgggtctgggacctcttactctctcacaatcagcagcatggaggctgaagatgctgccacttattactgccagcagtggagtagtaacccacccacgttcggtgctgggaccaagctggagctgaaacgg

SEQ ID NO: 246 - protein, 130 aa, Artificial Sequence
  Synthetic
METDTLLLWVLLLWVPGSTGDTRQIVLTQSPAIMSASPGEKVTMTCSASSSVSYMHWYQQKSGTSPKRWIYDTSKLASGVPARFSGSGSGTSYSLTISSMEAEDAATYYCQQWSSNPPTFGAGTKLELKR

SEQ ID NO: 247 - DNA, 411 bp, Artificial Sequence
  Synthetic
atggagacagacacactcctgctatgggtactgctgctctgggttccaggttccactggtgacacgcgtgaggtccagctgcagcagtctggacctgagctggtaaagcctggggcttcagtgaagatgtcctgcaaggcttctggatacacattcactagctatgttatgcactgggtgaagcagaagcctgggcagggccttgagtggattggatatattaatccttacaatgatggtactaagtacaatgagaagttcaaaggcaaggccacactgacttcagacaaatcctccagcacagcctacatggagctcagcagcctgacctctgaggactctgcggtctattacagtgcaagactagcaaggtttgcttactggggccaagggactctggtcactgtctct

SEQ ID NO: 248 - protein, 137 aa, Artificial Sequence
  Synthetic
METDTLLLWVLLLWVPGSTGDTREVQLQQSGPELVKPGASVKMSCKASGYTFTSYVMHWVKQKPGQGLEWIGYINPYNDGTKYNEKFKGKATLTSDKSSSTAYMELSSLTSEDSAVYYSARLARFAYWGQGTLVTVS

SEQ ID NO: 249 - DNA, 411 bp, Artificial Sequence; identical to SEQ ID NO: 47
  Synthetic

SEQ ID NO: 250 - protein, 137 aa, Artificial Sequence; identical to SEQ ID NO: 48
  Synthetic

SEQ ID NO: 251 - DNA, 420 bp, Artificial Sequence
  Synthetic
atggagacagacacactcctgctatgggtactgctgctctgggttccaggttccactggtgacacgcgtgaggttcagctgcagcagtctggggctgagcttgtgaggccaggggccttagtcaggttgtcctgcaaagcttctggcttcaacattaaagactactatatgaactgggtgaagcagaggcctgaacagggcctggagtggattggatggattgatcctgagaatggtaatactatatatgacccgaagttccagggcaaggccagtataatagcagacatatcctccaacacagcctacctacagctcagcagcctgacatctgaggacacagccgtctattattgtgctagatggtaccactatgttatggactactggggtcaaggaacctcagtcaccgtctcctca

SEQ ID NO: 252 - protein, 140 aa, Artificial Sequence
  Synthetic
METDTLLLWVLLLWVPGSTGDTREVQLQQSGAELVRPGALVRLSCKASGFNIKDYYMNWVKQRPEQGLEWIGWIDPENGNTIYDPKFQGKASIIADISSNTAYLQLSSLTSEDTAVYYCARWYHYVMDYWGQGTSVTVSS

SEQ ID NO: 253 - DNA, 390 bp, Artificial Sequence
  Synthetic
atggagacagacacactcctgctatgggtactgctgctctgggttccaggttccactggtgacacgcgtcaaattgttctctcccagtctccagcaatcctgtctgcatctccaggggagaaggtcacaatgacttgcagggccacctcaagtgtaagttacatgcactggtaccagcagaagccaggatcctcccccaaaccctggatttatgccacatccaacctggcttctggagtccctgctcgcttcagtggcagtgggtctgggacctcttactctctcacattcagcagagtggaggctgaagatgctgccacttattactgccagcagtggagtagtaacccacccacgttcggaggggggaccaggctggaaataacacgg

SEQ ID NO: 254 - protein, 130 aa, Artificial Sequence
  Synthetic
METDTLLLWVLLLWVPGSTGDTRQIVLSQSPAILSASPGEKVTMTCRATSSVSYMHWYQQKPGSSPKPWIYATSNLASGVPARFSGSGSGTSYSLTFSRVEAEDAATYYCQQWSSNPPTFGGGTRLEITR

SEQ ID NO: 255 - DNA, 438 bp, Artificial Sequence
  Synthetic
atggagacagacacactcctgctatgggtactgctgctctgggttccaggttccactggtgacacgcgtgaggtgcaacttgttgagtctggtggaggattggtgcagcctaaagggtcattgaaactctcatgtgcagcctctggattcaccttcaatatctacgccatgaactgggtccgccaggctccaggaaagggtttggaatgggttgctcgcataagatctaaaagtaataattttgcaacatattatggcgattcagtgagagacaggttcaccatctccagagatgattcacagagcatgctctatctgcaaatgaacaacttgaaagctgaagacacagccatgtattactgtgtgagacgggggggtggtagccattactatgctatggactactggggtcaaggaacctcagtcaccgtctcctca

SEQ ID NO: 256 - protein, 146 aa, Artificial Sequence
  Synthetic
METDTLLLWVLLLWVPGSTGDTREVQLVESGGGLVQPKGSLKLSCAASGFTFNIYAMNWVRQAPGKGLEWVARIRSKSNNFATYYGDSVRDRFTISRDDSQSMLYLQMNNLKAEDTAMYYCVRRGGGSHYYAMDYWGQGTSVTVSS

SEQ ID NO: 257 - DNA, 390 bp, Artificial Sequence
  Synthetic
atggagacagacacactcctgctatgggtactgctgctctgggttccaggttccactggtgacacgcgtcaaattgttctcacccagtctccagcactcatgtctgcatctccaggggagaaggtcaccatgacctgcagtgccagctcaagtgtaagttacatgtactggtaccagcagaagccaagatcctcccccaaaccctggatttatctcacatccaacctggcttctggagtccctgctcgcttcagtggcagtgggtctgggacctcttactctctcacaatcagcagcatggaggctgaagatgctgccacttattactgccagcagtggagtagtaacccacccacgttcggtgctgggaccaagctggagctgaaacgg

SEQ ID NO: 258 - protein, 130 aa, Artificial Sequence
  Synthetic
METDTLLLWVLLLWVPGSTGDTRQIVLTQSPALMSASPGEKVTMTCSASSSVSYMYWYQQKPRSSPKPWIYLTSNLASGVPARFSGSGSGTSYSLTISSMEAEDAATYYCQQWSSNPPTFGAGTKLELKR

SEQ ID NO: 259 - DNA, 426 bp, Artificial Sequence
  Synthetic
atggagacagacacactcctgctatgggtactgctgctctgggttccaggttccactggtgacacgcgtcaggtccagctgcagcagtctggacctgagctggtgaagcctggggcttcagtgaggatatcctgcaaggcttctggctacaccttcacaagctactatatacactgggtgaagcagaggcctggacagggacttgagtggattggatggatttatcctggaaatgttaatactaagtacaatgagaagttcaagggcaaggccacactgactgcagacaaatcctccagcacagcctacatgcagctcagcagcctgacctctgaggactctgcggtctatttctgtgcaagacgggcggggggctactggtacttcgatgtctggggcgcagggaccacggtcaccgtctcctca

SEQ ID NO: 260 - protein, 142 aa, Artificial Sequence
  Synthetic
METDTLLLWVLLLWVPGSTGDTRQVQLQQSGPELVKPGASVRISCKASGYTFTSYYIHWVKQRPGQGLEWIGWIYPGNVNTKYNEKFKGKATLTADKSSSTAYMQLSSLTSEDSAVYFCARRAGGYWYFDVWGAGTTVTVSS

SEQ ID NO: 261 - DNA, 396 bp, Artificial Sequence
  Synthetic
atggagacagacacactcctgctatgggtactgctgctctgggttccaggttccactggtgacacgcgtcaaattgttctcacccagtctccagcaatcatgtctgcatctctaggggaacgggtcaccatgacctgcactgccagctcaagtgtaagttccagttacttgcactggtaccagcagaagccaggatcctcccccaaactctggatttatagcacatccaacctggcttctggagtcccagctcgcttcagtggcagtgggtctgggacctcttactctctcacaatcagcagcatggaggctgaagatgctgccacttattactgccaccagtatcatcgttccccattcacgttcggctcggggacaaagttggaaataaaacgg

SEQ ID NO: 262 - protein, 132 aa, Artificial Sequence
  Synthetic
METDTLLLWVLLLWVPGSTGDTRQIVLTQSPAIMSASLGERVTMTCTASSSVSSSYLHWYQQKPGSSPKLWIYSTSNLASGVPARFSGSGSGTSYSLTISSMEAEDAATYYCHQYHRSPFTFGSGTKLEIKR

SEQ ID NO: 263 - DNA, 426 bp, Artificial Sequence
  Synthetic
atggagacagacacactcctgctatgggtactgctgctctgggttccaggttccactggtgacacgcgtgaggttcagctgcagcagtctggggcagagcttgtgaagccaggggcctcagtcaagttgtcctgcacagcttctggcttcaacattaaagacacctatatgcactgggtgaagcagaggcctgaacagggcctggagtggattggaaggattgatcctgcgaatggtaatactaaatatgacccgaagttccagggcaaggccactataacagcagacacatcctccaacacagcctacctgcagctcagcagcctgacatctgaggacactgccgtctattactgtgctagaaggttcgatggttactttcgctggtttgcttactggggccaagggactctggtcactgtctct

SEQ ID NO: 264 - protein, 142 aa, Artificial Sequence
  Synthetic
METDTLLLWVLLLWVPGSTGDTREVQLQQSGAELVKPGASVKLSCTASGFNIKDTYMHWVKQRPEQGLEWIGRIDPANGNTKYDPKFQGKATITADTSSNTAYLQLSSLTSEDTAVYYCARRFDGYFRWFAYWGQGTLVTVS

SEQ ID NO: 265 - DNA, 408 bp, Artificial Sequence
  Synthetic
atggagacagacacactcctgctatgggtactgctgctctgggttccaggttccactggtgacacgcgtgacattgtgatgtcacagtctccatcctccctggctgtgtcagcaggagagaaggtcactatgagctgcaaatccagtcagagtctgctcaacagtagaacccgaaagaactacttggcttggtaccagcagaaaccagggcagtctcctaaactgctgatctactgggcatccactagggaatctggggtccctgatcgcttcacaggcagtggatctgggacagatttcactctcaccatcagcagtgtgcaggctgaagacctggcagtttattactgcaagcaatcttataatctgtacacgttcggaggggggaccaagctggaaataaaacgg

SEQ ID NO: 266 - protein, 136 aa, Artificial Sequence
  Synthetic
METDTLLLWVLLLWVPGSTGDTRDIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYNLYTFGGGTKLEIKR

SEQ ID NO: 267 - DNA, 420 bp, Artificial Sequence; identical to SEQ ID NO: 251
  Synthetic

SEQ ID NO: 268 - protein, 140 aa, Artificial Sequence; identical to SEQ ID NO: 252
  Synthetic

SEQ ID NO: 269 - DNA, 408 bp, Artificial Sequence
  Synthetic
atggagacagacacactcctgctatgggtactgctgctctgggttccaggttccactggtgacacgcgtgatgttgtgatgacccagactccactcactttgtcggttaccattggacaaccagcctccatctcttgcaagtcaagtcagagcctcttagatagtgatggaaagacatattttagttggttgttacagaggccaggccagtctccaaagcgcctaatctatctggtgtctaaactggactctggagtccctgacaggttcactggcagtggatcagggacagatttcacacttaaaatcagcagagtggaggctgaggatttgggagtttatttttgctggcaaggtacacattttcctcacacattcggaggggggaccaagctggagataaaacgg

SEQ ID NO: 270 - protein, 136 aa, Artificial Sequence
  Synthetic
METDTLLLWVLLLWVPGSTGDTRDVVMTQTPLTLSVTIGQPASISCKSSQSLLDSDGKTYFSWLLQRPGQSPKRLIYLVSKLDSGVPDRFTGSGSGTDFTLKISRVEAEDLGVYFCWQGTHFPHTFGGGTKLEIKR

SEQ ID NO: 271 - DNA, 408 bp, Artificial Sequence
  Synthetic
atggagacagacacactcctgctatgggtactgctgctctgggttccaggttccactggtgacacgcgtgaagtgcagctggtggagtctgggggaggcttagtgaagcctggagagtccctgacactctcctgtacaacctctggattcactttcagtgactattacatgtattgggttcgccagactccggaaaagaggctggagtgggtcgcaaccattaatcgtgatggtagttatacctactttccagacaattttaaggggcgattcaccatctccagagacaatgccaagaacaacctgtacctgcaaatgagcagtctgaagtctgaggacacagccatgtattactgttcaaccatgctgtttgcttactggggccaagggactctggtcactgtctct

SEQ ID NO: 272 - protein, 136 aa, Artificial Sequence
  Synthetic
METDTLLLWVLLLWVPGSTGDTREVQLVESGGGLVKPGESLTLSCTTSGFTFSDYYMYWVRQTPEKRLEWVATINRDGSYTYFPDNFKGRFTISRDNAKNNLYLQMSSLKSEDTAMYYCSTMLFAYWGQGTLVTVS

SEQ ID NO: 273 - DNA, 393 bp, Artificial Sequence
  Synthetic
atggagacagacacactcctgctatgggtactgctgctctgggttccaggttccactggtgacacgcgtgatattgtgctaactcagtctccagccaccctgtctgtgactccaggagatagcgtcagtctttcctgcagggccagccaaagtattagcaacaacctacactggtatcaacaaaaatcacatgagtctccaaggcttctcatcaagtatgcttcccagtccatctctgggatcccctccaggttcagtggcagcggatcagggacagatttcactctcagtatcaacagtgtggagactgaagattttggaatgtatttctgtcaacagagtaacagctggccgctcacgttcggtgctgggaccaagctggagctgaaacgg

SEQ ID NO: 274 - protein, 131 aa, Artificial Sequence
  Synthetic
METDTLLLWVLLLWVPGSTGDTRDIVLTQSPATLSVTPGDSVSLSCRASQSISNNLHWYQQKSHESPRLLIKYASQSISGIPSRFSGSGSGTDFTLSINSVETEDFGMYFCQQSNSWPLTFGAGTKLELKR

SEQ ID NO: 275 - DNA, 417 bp, Artificial Sequence
  Synthetic
atggagacagacacactcctgctatgggtactgctgctctgggttccaggttccactggtgacacgcgtcaggtgcagctgaaggagtcaggacctggcctggtggcgccctcacagagcctgtccatcacatgcactgtctcagggttctcattaaccagctatggtgtaagctgggttcgccagcctccaggaaagggtctggagtggctgggagtaatatggggtgacgggagcacaaattatcattcagctctcatatccagactgagcatcagcaaggataactccaagagccaagttttcttaaaactgaacagtctgcaaactgatgacacagccacgtactactgtgccatattacgacggacggtatcctttgcttactggggccaagggactctggtcactgtctct

SEQ ID NO: 276 - protein, 139 aa, Artificial Sequence
  Synthetic
METDTLLLWVLLLWVPGSTGDTRQVQLKESGPGLVAPSQSLSITCTVSGFSLTSYGVSWVRQPPGKGLEWLGVIWGDGSTNYHSALISRLSISKDNSKSQVFLKLNSLQTDDTATYYCAILRRTVSFAYWGQGTLVTVS

SEQ ID NO: 277 - DNA, 1491 bp, Artificial Sequence
  Synthetic
atggagacagacacactcctgctatgggtactgctgctctgggttccaggttccactggtgacgccacccacgagcactccgcccagtggctgaacaactacaagaagggctacggctacggcccctaccccctgggcatcaacggcggcatgcactacggcgtggacttcttcatgaacatcggcacccccgtgaaggccatctcctccggcaagatcgtggaggccggctggtccaactacggcggcggcaaccagatcggcctgatcgagaacgacggcgtgcaccgccagtggtacatgcacctgtccaagtacaacgtgaaggtgggcgactacgtgaaggccggccagatcatcggctggtccggctccaccggctactccaccgccccccacctgcacttccagcgcatggtgaactccttctccaactccaccgcccaggaccccatgcccttcctgaagtccgccggctacggcaaggccggcggcaccgtgacccccacccccaacaccggctggaagaccaacaagtacggcaccctgtacaagtccgagtccgcctccttcacccccaacaccgacatcatcacccgcaccaccggccccttccgctccatgccccagtccggcgtgctgaaggccggccagaccatccactacgacgaggtgatgaagcaggacggccacgtgtgggtgggctacaccggcaactccggccagcgcatctacctgcccgtgcgcacctggaacaagtccaccaacaccctgggcgtgctgtggggcaccatcaagggtggtggcggttcaggcggaggtggctctggcggtggcggatccacgcgtcaaattgttctctcccagtctccagcaatcctgtctgcatctccaggggagaaggtcacaatgacttgcagggccacctcaagtgtaagttacatgcactggtaccagcagaagccaggatcctcccccaaaccctggatttatgccacatccaacctggcttctggagtccctgctcgcttcagtggcagtgggtctgggacctcttactctctcacattcagcagagtggaggctgaagatgctgccacttattactgccagcagtggagtagtaacccacccacgttcggaggggggaccaggctggaaataacacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 278 - protein, 496 aa, Artificial Sequence
  Synthetic
METDTLLLWVLLLWVPGSTGDATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKGGGGSGGGGSGGGGSTRQIVLSQSPAILSASPGEKVTMTCRATSSVSYMHWYQQKPGSSPKPWIYATSNLASGVPARFSGSGSGTSYSLTFSRVEAEDAATYYCQQWSSNPPTFGGGTRLEITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 279 - DNA, 1491 bp, Artificial Sequence
  Synthetic
atggagacagacacactcctgctatgggtactgctgctctgggttccaggttccactggtgacgccacccacgagcactccgcccagtggctgaacaactacaagaagggctacggctacggcccctaccccctgggcatcaacggcggcatgcactacggcgtggacttcttcatgaacatcggcacccccgtgaaggccatctcctccggcaagatcgtggaggccggctggtccaactacggcggcggcaaccagatcggcctgatcgagaacgacggcgtgcaccgccagtggtacatgcacctgtccaagtacaacgtgaaggtgggcgactacgtgaaggccggccagatcatcggctggtccggctccaccggctactccaccgccccccacctgcacttccagcgcatggtgaactccttctcccagtccaccgcccaggaccccatgcccttcctgaagtccgccggctacggcaaggccggcggcaccgtgacccccacccccaacaccggctggaagaccaacaagtacggcaccctgtacaagtccgagtccgcctccttcacccccaacaccgacatcatcacccgcaccaccggccccttccgctccatgccccagtccggcgtgctgaaggccggccagaccatccactacgacgaggtgatgaagcaggacggccacgtgtgggtgggctacaccggcaactccggccagcgcatctacctgcccgtgcgcacctggaacaagtccaccaacaccctgggcgtgctgtggggcaccatcaagggtggtggcggttcaggcggaggtggctctggcggtggcggatccacgcgtcaaattgttctctcccagtctccagcaatcctgtctgcatctccaggggagaaggtcacaatgacttgcagggccacctcaagtgtaagttacatgcactggtaccagcagaagccaggatcctcccccaaaccctggatttatgccacatccaacctggcttctggagtccctgctcgcttcagtggcagtgggtctgggacctcttactctctcacattcagcagagtggaggctgaagatgctgccacttattactgccagcagtggagtagtaacccacccacgttcggaggggggaccaggctggaaataacacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 280 - protein, 496 aa, Artificial Sequence
  Synthetic
METDTLLLWVLLLWVPGSTGDATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSQSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKGGGGSGGGGSGGGGSTRQIVLSQSPAILSASPGEKVTMTCRATSSVSYMHWYQQKPGSSPKPWIYATSNLASGVPARFSGSGSGTSYSLTFSRVEAEDAATYYCQQWSSNPPTFGGGTRLEITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 281 - DNA, 1491 bp, Artificial Sequence
  Synthetic
atggagacagacacactcctgctatgggtactgctgctctgggttccaggttccactggtgacgccacccacgagcactccgcccagtggctgaacaactacaagaagggctacggctacggcccctaccccctgggcatcaacggcggcatgcactacggcgtggacttcttcatgaacatcggcacccccgtgaaggccatctcctccggcaagatcgtggaggccggctggtccaactacggcggcggcaaccagatcggcctgatcgagaacgacggcgtgcaccgccagtggtacatgcacctgtccaagtacaacgtgaaggtgggcgactacgtgaaggccggccagatcatcggctggtccggctccaccggctactccaccgccccccacctgcacttccagcgcatggtgaactccttctcccagtccaccgcccaggaccccatgcccttcctgaagtccgccggctacggcaaggccggcggcaccgtgacccccacccccaacaccggctggaagaccaacaagtacggcaccctgtacaagtccgagtccgcctccttcacccccaacaccgacatcatcacccgcaccaccggccccttccgctccatgccccagtccggcgtgctgaaggccggccagaccatccactacgacgaggtgatgaagcaggacggccacgtgtgggtgggctacaccggcaactccggccagcgcatctacctgcccgtgcgcacctggcagaagtccaccaacaccctgggcgtgctgtggggcaccatcaagggtggtggcggttcaggcggaggtggctctggcggtggcggatccacgcgtcaaattgttctctcccagtctccagcaatcctgtctgcatctccaggggagaaggtcacaatgacttgcagggccacctcaagtgtaagttacatgcactggtaccagcagaagccaggatcctcccccaaaccctggatttatgccacatccaacctggcttctggagtccctgctcgcttcagtggcagtgggtctgggacctcttactctctcacattcagcagagtggaggctgaagatgctgccacttattactgccagcagtggagtagtaacccacccacgttcggaggggggaccaggctggaaataacacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 282 - protein, 496 aa, Artificial Sequence
  Synthetic
METDTLLLWVLLLWVPGSTGDATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSQSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWQKSTNTLGVLWGTIKGGGGSGGGGSGGGGSTRQIVLSQSPAILSASPGEKVTMTCRATSSVSYMHWYQQKPGSSPKPWIYATSNLASGVPARFSGSGSGTSYSLTFSRVEAEDAATYYCQQWSSNPPTFGGGTRLEITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 283 - DNA, 1494 bp, Artificial Sequence
  Synthetic
atggagacagacacactcctgctatgggtactgctgctctgggttccaggttccactggtgacgccacccacgagcactccgcccagtggctgaacaactacaagaagggctacggctacggcccctaccccctgggcatcaacggcggcatgcactacggcgtggacttcttcatgaacatcggcacccccgtgaaggccatctcctccggcaagatcgtggaggccggctggtccaactacggcggcggcaaccagatcggcctgatcgagaacgacggcgtgcaccgccagtggtacatgcacctgtccaagtacaacgtgaaggtgggcgactacgtgaaggccggccagatcatcggctggtccggctccaccggctactccaccgccccccacctgcacttccagcgcatggtgaactccttctccaactccaccgcccaggaccccatgcccttcctgaagtccgccggctacggcaaggccggcggcaccgtgacccccacccccaacaccggctggaagaccaacaagtacggcaccctgtacaagtccgagtccgcctccttcacccccaacaccgacatcatcacccgcaccaccggccccttccgctccatgccccagtccggcgtgctgaaggccggccagaccatccactacgacgaggtgatgaagcaggacggccacgtgtgggtgggctacaccggcaactccggccagcgcatctacctgcccgtgcgcacctggaacaagtccaccaacaccctgggcgtgctgtggggcaccatcaagggtggtggcggttcaggcggaggtggctctggcggtggcggatccacgcgtagtattgtgatgacccagactcccaaattcctgcttgtatcagcaggagacagggttaccataacctgcaaggccagtcagagtgtgagtaatgatgtagcttggtaccaacagaagccagggcagtctcctaaactgctgatatactatgcatccaatcgctacactggagtccctgatcgcttcactggcagtggatatgggacggatttcactttcaccatcagcactgtgcaggctgaagacctggcagtttatttctgtcagcaggattatagctctcctctcacgttcggctcggggacaaagttggaaataaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 284 - protein, 497 aa, Artificial Sequence
  Synthetic
METDTLLLWVLLLWVPGSTGDATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKGGGGSGGGGSGGGGSTRSIVMTQTPKFLLVSAGDRVTITCKASQSVSNDVAWYQQKPGQSPKLLIYYASNRYTGVPDRFTGSGYGTDFTFTISTVQAEDLAVYFCQQDYSSPLTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 285 - DNA, 1494 bp, Artificial Sequence
  Synthetic
atggagacagacacactcctgctatgggtactgctgctctgggttccaggttccactggtgacgccacccacgagcactccgcccagtggctgaacaactacaagaagggctacggctacggcccctaccccctgggcatcaacggcggcatgcactacggcgtggacttcttcatgaacatcggcacccccgtgaaggccatctcctccggcaagatcgtggaggccggctggtccaactacggcggcggcaaccagatcggcctgatcgagaacgacggcgtgcaccgccagtggtacatgcacctgtccaagtacaacgtgaaggtgggcgactacgtgaaggccggccagatcatcggctggtccggctccaccggctactccaccgccccccacctgcacttccagcgcatggtgaactccttctcccagtccaccgcccaggaccccatgcccttcctgaagtccgccggctacggcaaggccggcggcaccgtgacccccacccccaacaccggctggaagaccaacaagtacggcaccctgtacaagtccgagtccgcctccttcacccccaacaccgacatcatcacccgcaccaccggccccttccgctccatgccccagtccggcgtgctgaaggccggccagaccatccactacgacgaggtgatgaagcaggacggccacgtgtgggtgggctacaccggcaactccggccagcgcatctacctgcccgtgcgcacctggaacaagtccaccaacaccctgggcgtgctgtggggcaccatcaagggtggtggcggttcaggcggaggtggctctggcggtggcggatccacgcgtagtattgtgatgacccagactcccaaattcctgcttgtatcagcaggagacagggttaccataacctgcaaggccagtcagagtgtgagtaatgatgtagcttggtaccaacagaagccagggcagtctcctaaactgctgatatactatgcatccaatcgctacactggagtccctgatcgcttcactggcagtggatatgggacggatttcactttcaccatcagcactgtgcaggctgaagacctggcagtttatttctgtcagcaggattatagctctcctctcacgttcggctcggggacaaagttggaaataaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 286 - protein, 497 aa, Artificial Sequence
  Synthetic
METDTLLLWVLLLWVPGSTGDATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSQSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKGGGGSGGGGSGGGGSTRSIVMTQTPKFLLVSAGDRVTITCKASQSVSNDVAWYQQKPGQSPKLLIYYASNRYTGVPDRFTGSGYGTDFTFTISTVQAEDLAVYFCQQDYSSPLTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 287 - DNA, 1494 bp, Artificial Sequence
  Synthetic
atggagacagacacactcctgctatgggtactgctgctctgggttccaggttccactggtgacgccacccacgagcactccgcccagtggctgaacaactacaagaagggctacggctacggcccctaccccctgggcatcaacggcggcatgcactacggcgtggacttcttcatgaacatcggcacccccgtgaaggccatctcctccggcaagatcgtggaggccggctggtccaactacggcggcggcaaccagatcggcctgatcgagaacgacggcgtgcaccgccagtggtacatgcacctgtccaagtacaacgtgaaggtgggcgactacgtgaaggccggccagatcatcggctggtccggctccaccggctactccaccgccccccacctgcacttccagcgcatggtgaactccttctcccagtccaccgcccaggaccccatgcccttcctgaagtccgccggctacggcaaggccggcggcaccgtgacccccacccccaacaccggctggaagaccaacaagtacggcaccctgtacaagtccgagtccgcctccttcacccccaacaccgacatcatcacccgcaccaccggccccttccgctccatgccccagtccggcgtgctgaaggccggccagaccatccactacgacgaggtgatgaagcaggacggccacgtgtgggtgggctacaccggcaactccggccagcgcatctacctgcccgtgcgcacctggcagaagtccaccaacaccctgggcgtgctgtggggcaccatcaagggtggtggcggttcaggcggaggtggctctggcggtggcggatccacgcgtagtattgtgatgacccagactcccaaattcctgcttgtatcagcaggagacagggttaccataacctgcaaggccagtcagagtgtgagtaatgatgtagcttggtaccaacagaagccagggcagtctcctaaactgctgatatactatgcatccaatcgctacactggagtccctgatcgcttcactggcagtggatatgggacggatttcactttcaccatcagcactgtgcaggctgaagacctggcagtttatttctgtcagcaggattatagctctcctctcacgttcggctcggggacaaagttggaaataaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 288 - protein, 497 aa, Artificial Sequence
  Synthetic
METDTLLLWVLLLWVPGSTGDATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSQSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWQKSTNTLGVLWGTIKGGGGSGGGGSGGGGSTRSIVMTQTPKFLLVSAGDRVTITCKASQSVSNDVAWYQQKPGQSPKLLIYYASNRYTGVPDRFTGSGYGTDFTFTISTVQAEDLAVYFCQQDYSSPLTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 289 - DNA, 1509 bp, Artificial Sequence
  Synthetic
atggagacagacacactcctgctatgggtactgctgctctgggttccaggttccactggtgacgccacccacgagcactccgcccagtggctgaacaactacaagaagggctacggctacggcccctaccccctgggcatcaacggcggcatgcactacggcgtggacttcttcatgaacatcggcacccccgtgaaggccatctcctccggcaagatcgtggaggccggctggtccaactacggcggcggcaaccagatcggcctgatcgagaacgacggcgtgcaccgccagtggtacatgcacctgtccaagtacaacgtgaaggtgggcgactacgtgaaggccggccagatcatcggctggtccggctccaccggctactccaccgccccccacctgcacttccagcgcatggtgaactccttctccaactccaccgcccaggaccccatgcccttcctgaagtccgccggctacggcaaggccggcggcaccgtgacccccacccccaacaccggctggaagaccaacaagtacggcaccctgtacaagtccgagtccgcctccttcacccccaacaccgacatcatcacccgcaccaccggccccttccgctccatgccccagtccggcgtgctgaaggccggccagaccatccactacgacgaggtgatgaagcaggacggccacgtgtgggtgggctacaccggcaactccggccagcgcatctacctgcccgtgcgcacctggaacaagtccaccaacaccctgggcgtgctgtggggcaccatcaagggtggtggcggttcaggcggaggtggctctggcggtggcggatccacgcgtgacattgtgatgtcacagtctccatcctccctggctgtgtcagcaggagagaaggtcactatgagctgcaaatccagtcagagtctgctcaacagtagaacccgaaagaactacttggcttggtaccagcagaaaccagggcagtctcctaaactgctgatctactgggcatccactagggaatctggggtccctgatcgcttcacaggcagtggatctgggacagatttcactctcaccatcagcagtgtgcaggctgaagacctggcagtttattactgcaagcaatcttataatctgtggacgttcggtggaggcaccaagctggaaatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 290 - protein, 502 aa, Artificial Sequence
  Synthetic
METDTLLLWVLLLWVPGSTGDATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKGGGGSGGGGSGGGGSTRDIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYNLWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 291 - DNA, 1509 bp, Artificial Sequence
  Synthetic
atggagacagacacactcctgctatgggtactgctgctctgggttccaggttccactggtgacgccacccacgagcactccgcccagtggctgaacaactacaagaagggctacggctacggcccctaccccctgggcatcaacggcggcatgcactacggcgtggacttcttcatgaacatcggcacccccgtgaaggccatctcctccggcaagatcgtggaggccggctggtccaactacggcggcggcaaccagatcggcctgatcgagaacgacggcgtgcaccgccagtggtacatgcacctgtccaagtacaacgtgaaggtgggcgactacgtgaaggccggccagatcatcggctggtccggctccaccggctactccaccgccccccacctgcacttccagcgcatggtgaactccttctcccagtccaccgcccaggaccccatgcccttcctgaagtccgccggctacggcaaggccggcggcaccgtgacccccacccccaacaccggctggaagaccaacaagtacggcaccctgtacaagtccgagtccgcctccttcacccccaacaccgacatcatcacccgcaccaccggccccttccgctccatgccccagtccggcgtgctgaaggccggccagaccatccactacgacgaggtgatgaagcaggacggccacgtgtgggtgggctacaccggcaactccggccagcgcatctacctgcccgtgcgcacctggaacaagtccaccaacaccctgggcgtgctgtggggcaccatcaagggtggtggcggttcaggcggaggtggctctggcggtggcggatccacgcgtgacattgtgatgtcacagtctccatcctccctggctgtgtcagcaggagagaaggtcactatgagctgcaaatccagtcagagtctgctcaacagtagaacccgaaagaactacttggcttggtaccagcagaaaccagggcagtctcctaaactgctgatctactgggcatccactagggaatctggggtccctgatcgcttcacaggcagtggatctgggacagatttcactctcaccatcagcagtgtgcaggctgaagacctggcagtttattactgcaagcaatcttataatctgtggacgttcggtggaggcaccaagctggaaatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 292 - protein, 502 aa, Artificial Sequence
  Synthetic
METDTLLLWVLLLWVPGSTGDATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSQSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKGGGGSGGGGSGGGGSTRDIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYNLWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 293 - DNA, 1494 bp, Artificial Sequence
  Synthetic
atggagacagacacactcctgctatgggtactgctgctctgggttccaggttccactggtgacgccacccacgagcactccgcccagtggctgaacaactacaagaagggctacggctacggcccctaccccctgggcatcaacggcggcatgcactacggcgtggacttcttcatgaacatcggcacccccgtgaaggccatctcctccggcaagatcgtggaggccggctggtccaactacggcggcggcaaccagatcggcctgatcgagaacgacggcgtgcaccgccagtggtacatgcacctgtccaagtacaacgtgaaggtgggcgactacgtgaaggccggccagatcatcggctggtccggctccaccggctactccaccgccccccacctgcacttccagcgcatggtgaactccttctccaactccaccgcccaggaccccatgcccttcctgaagtccgccggctacggcaaggccggcggcaccgtgacccccacccccaacaccggctggaagaccaacaagtacggcaccctgtacaagtccgagtccgcctccttcacccccaacaccgacatcatcacccgcaccaccggccccttccgctccatgccccagtccggcgtgctgaaggccggccagaccatccactacgacgaggtgatgaagcaggacggccacgtgtgggtgggctacaccggcaactccggccagcgcatctacctgcccgtgcgcacctggaacaagtccaccaacaccctgggcgtgctgtggggcaccatcaagggtggtggcggttcaggcggaggtggctctggcggtggcggatccacgcgtgacattgtgatgacccagtctcaaaaattcatgtccacaccagtaggagacagggtcagcgtcacctgcaaggccagtcagaatgtgggtacttatgtagcctggtatcaacagaaaccagggcagtctcctaaaatactgatttattcggcatcctaccggtacagtggagtccctgatcgcttcacaggcagtggctctgggacagatttcactctcaccatcagcaatgtgcagtctgaagacttggcagagtatttctgtcagcgatataacagctatcctctcacgttcggtgctgggaccaagctggagctgaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag